(19) United States
(12) Patent Application Publication
(10) Pub. No.: US 2007/0141566 A1
(43) Pub. Date: Jun. 21, 2007

(54) Methods and materials relating to novel polypeptides and polynucleotides

(76) Inventors: Fabio Rupp, Sunnyvale, CA (US); Jian Rui Wang, San Jose, CA (US); Ping Zho, Cupertino, CA (US); Tom Wehman, Stanford, CA (US); Zhiwei Wang, Minneapolis, MN (US); Yuanhua T. Tang, Beverly, MA (US)

Correspondence Address: NUVELO, INC, 201 INDUSTRIAL ROAD, SUITE 310, SAN CARLOS, CA, 94070, US

(21) Appl. No.: 10/520,299

(22) PCT Filed: Jul. 9, 2003
(86) PCT No.: PCT/US03/21703

§ 371 Date: Oct. 14, 2005

Related U.S. Application Data
(60) Provisional application No. 60/395,402, filed on Jul. 12, 2002.

(51) Int. Cl. C12Q 1/68; C07H 21/04; C12P 21/06; C07K 14/82; C07K 16/30

(52) U.S. Cl. 435/6; 536/23.5; 530/350; 530/388.8; 435/69.1; 435/320.1; 435/325

(57) ABSTRACT

The invention provides novel polynucleotides and polypeptides encoded by such polynucleotides and mutants or variants thereof that correspond to the novel polynucleotides and polypeptides. Other aspects of the invention include vectors containing processes for producing novel polypeptides, and antibodies specific for such polypeptides.

Related subject matter is disclosed in the following co-owned, co-pending applications:
1) U.S. Provisional Application Ser. No. 60/395,402 filed Jul. 12, 2002, entitled “Methods and Materials Relatintg to Carcinoembryonic Antigen-like Polypeptides and Polynucleotides,” Attorney Docket No. HYS-63, which in turn contains material related to PCT Application Serial No. PCT/US02/22858 filed Jul. 19, 2002, entitled “Novel Nucleic Acids and Secreted Polypeptides,” Attorney Docket No. 805A/PCT, which in turn is a continuation-in-part application of U.S. application Ser. No. 10/112,944 filed Mar. 28, 2002, entitled “Novel Nucleic Acids and Secreted Polypeptides,” Attorney Docket No. 805A, which in turn claims the priority benefit of U.S. Provisional Application Ser. No. 60/306,971 (now expired) filed Jul. 21, 2001, entitled “Novel Nucleic Acids and Secreted Polypeptides,” Attorney Docket No. 805, which in turn contains related material disclosed in U.S. application Ser. No. 10/276,781 filed Nov. 18, 2002, entitled “Novel Nucleic Acids and Polypeptides,” Attorney Docket No. 785CIP2A-C/US, which in turn is a National Stage Application of PCT Application Serial No. PCT/US01/02687 filed Jan. 25, 2001, entitled “Novel Nucleic Acids and Polypeptides,” Attorney Docket No. 785CIP2A-C/PCT, which in turn the priority benefit of U.S. application Ser. No. 09/491,404 (now abandoned) filed Jan. 25, 2000, entitled “Novel Contigs Obtained from Various Libraries,” Attorney Docket No. 785;
2) U.S. Provisional Application Ser. No. 60/416,261 filed Oct. 3, 2002, entitled “Methods and Materials Relating to Chemokine-like Polypeptides and Polynucleotides,” Attorney Docket No. HYS-64, which in turn contains related material disclosed in: U.S. application Ser. No. 10/273,573 filed Oct. 18, 2002, entitled “Novel Nucleic Acids and Polypeptides,” Attorney Docket No. 791CIP5, which in turn is a National Stage application of PCT Application Serial No. PCT/US01/08656 filed Apr. 18, 2001, entitled “Novel Nucleic Acids and Polypeptides,” Attorney Docket No. 791CIP3/PCT, which in turn claims the priority benefit of U.S. Ser. No. 09/552,929 (now abandoned) filed Apr. 18, 2000, entitled “Novel Nucleic Acids and Polypeptides,” Attorney Docket No. 791; U.S. application Ser. No. 10/276,774 filed Nov. 18, 2002, entitled “Novel Nucleic Acids and Polypeptides,” Attorney Docket No. 787CIP3/US, which is a National Stage application of PCT Application Serial No. PCT/US01/03800 filed Feb. 5, 2001, entitled “Novel Nucleic Acids and Polypeptides,” Attorney Docket No. 787CIP3/PCT, which in turn claims the priority benefit of U.S. application Ser. No. 09/496,914 (now abandoned) filed Feb. 3, 2000, entitled “Novel Contigs Obtained from Various Libraries,” Attorney Docket No. 787; U.S. application Ser. No. 10/276,781 filed Nov. 18, 2002 entitled “Novel Nucleic Acids and Polypeptides,” Attorney Docket No. 785CIP2A-C/US which is a National Stage application of PCT Application Serial No. PCT/US01/02687 filed Jan. 25; 2001, entitled “Novel Nucleic Acids and Polypeptides,” Attorney Docket No. 785CIP2A-C/PCT, which in turn claims the priority benefit of U.S. application Ser. No. 09/491,404 (now abandoned) filed Jan. 25, 2000, entitled “Novel Contigs Obtained from Various Libraries,” Attorney Docket No. 785; U.S. application Ser. No. 10/296,115 filed Nov. 18, 2002, entitled “Novel Nucleic Acids and Polypeptides,” Attorney Docket No. 784CIP3A/US, which is a National Stage application of PCT Application Serial No. PCT/US00/35017 filed Dec. 22, 2000, entitled “Novel Nucleic Acids and Polypeptides,” Attorney Docket No. 784CIP3A/PCT, which in turn is a continuation-in-part application of U.S. Ser. No. 09/488,725 filed Jan. 21, 2000, entitled “Novel Contigs Obtained from Various Libraries,” Attorney Docket No. 784;
3) U.S. Provisional Application Ser. No. 60/418,132 filed Oct. 11, 2002, entitled “Methods and Materials Relating to Novel Secreted Adiponectin-like Polypeptides and Polynucleotides,” Attorney Docket No. HYS-65, which contains material related to PCT Application Serial No. PCT/US02/39555 filed Dec. 10, 2002, entitled “Novel Nucleic Acids and Polypeptides,” Attorney Docket No. 820/PCT, which in turn contains material disclosed in U.S. Provisional Application Ser. No. 60/365,091 (now expired) filed Mar. 14, 2002, entitled “Novel Nucleic Acids and Polypeptides,” Attorney Docket No. 815;
4) U.S. Provisional Application Ser. No. 60/425,158 filed Nov. 8, 2002, entitled “Methods and Materials Relating to Ly-6-like Polypeptides and Polynucleotides,” Attorney Docket No. HYS-66, which contains related material disclosed in PCT Application Serial No. PCT/US02/29636 filed Sep. 18, 2002 entitled “Novel Nucleic Acids and Secreted Polypeptides”, Attorney Docket No. 808ACIP/PCT, which claims priority to U.S. Provisional Application Ser. No. 60/323,349 filed Sep. 18, 2001 entitled “Novel Nucleic Acids and Secreted Polypeptides”, Attorney Docket No. 808, which is a continuation-in-part application of U.S. application Ser. No. 10/296,115 filed Nov. 18, 2002 entitled “Novel Nucleic Acids and Polypeptides,” Attorney Docket No. 784CIP3A/US, which is a National Stage application of PCT Application Serial No. PCT/US00/35017 filed Dec. 22, 2000 entitled “Novel Nucleic Acids and Polypeptides”, Attorney Docket No. 784CIP3A/PCT, which in turn is a continuation-in-part application of U.S. application Ser. No. 09/552,317 (now abandoned) filed Apr. 25, 2000 entitled “Novel Nucleic Acids and Polypeptides”, Attorney Docket No. 784CIP, which in turn is a continuation-in-part application of U.S. application Ser. No. 09/488,725 filed Jan. 21, 2000 entitled “Novel Contigs Obtained from Various Liabraries”, Attorney Docket No. 784; U.S. application Ser. No. 10/275,027 filed Oct. 30, 2002, entitled “Novel Nucleic Acids and Polypeptides,” Attorney Docket No. 785CIP3/US, which is a National Stage application of PCT Application Serial No. PCT/US01/02623 filed Jan. 25, 2001 entitled “Novel Nucleic Acids and Polypeptides”, Attorney Docket No. 785CIP3/PCT, which in turn is a continuation-in-part application of U.S. application Ser. No. 09/491,404 filed Jan. 25, 2000 entitled “Novel Contigs Obtained from Various Libraries”, Attorney Docket No. 785; U.S. application Ser. No. 10/276,774 filed Nov. 18, 2002, entitled “Novel Nucleic Acids and Polypeptides,” Attorney Docket No. 787CIP3/US, which is a National Stage application of PCT Application Serial No. PCT/US01/03800 filed Feb. 5, 2001 entitled “Novel Nucleic Acids and Polypeptides”, Attorney Docket No. 787CIP3/PCT, which in turn is a continuation-in-part application of U.S. application Ser. No. 09/560,875 filed Apr. 27, 2000 entitled “Novel Nucleic Acids and Polypeptides”, Attorney Docket No. 787CIP, which in turn is a continuation-in-part application of U.S. application Ser. No. 09/496,914 (now abandoned) filed Feb. 03, 2000 entitled “Novel Contigs Obtained from Various Libraries”, Attorney Docket No. 787; U.S. application Ser. No. 10/220,366 filed Aug. 28, 2002 entitled “Novel Nucleic Acids and Polypeptides,” Attorney Docket No. 788CIP3/US, which is a National Stage application of PCT Application Serial No. PCT/US01/04927 filed Feb. 26, 2001 entitled “Novel Nucleic Acids and Polypeptides”, Attorney Docket No. 788CIP3/PCT, which in turn is a continuation-in-part application of U.S. application Ser. No. 09/577,409 (now abandoned) filed May 18, 2000 entitled “Novel Nucleic Acids and Polypeptides”, Attorney Docket No. 788CIP, which in turn is a continuation-in-part application of U.S. application Ser. No. 09/515,126 (now abandoned) filed Feb. 28, 2000 entitled “Novel Contigs Obtained from Various Libraries”, Attorney Docket No. 788; U.S. application Ser. No. 10/221,279 filed May 28, 2003, entitled “Novel Nucleic Acids and Polypeptides,” Attorney Docket No. 789CIP3/US which is a National Stage application of PCT Application Serial No. PCT/US01/04941 filed Mar. 5, 2001 entitled “Novel Nucleic Acids and Polypeptides”, Attorney Docket No. 789CIP3/PCT, which in turn is a continuation-in-part application of U.S. application Ser. No. 09/574,454 (now abandoned) filed May 19, 2000 entitled “Novel Nucleic Acids and Polypeptides”, Attorney Docket No. 789CIP, which in turn is a continuation-in-part application of U.S. application Ser. No. 09/519,705 (now abandoned) filed Mar. 7, 2000 entitled “Novel Contigs Obtained from Various Libraries”, Attorney Docket No. 789; U.S. application Ser. No. 10/450,763 filed Sep. 27, 2002, entitled “Novel Nucleic Acids and Polypeptides,” Attorney Docket No. 790CIP3/US, which is a National Stage application of PCT Application Serial No. PCT/US01/08631 filed Mar. 30, 2001 entitled “Novel Nucleic Acids and Polypeptides”, Attorney Docket No. 790CIP3/PCT, which in turn is a continuation-in-part application of U.S. application Ser. No. 09/649,167 (now abandoned) filed Aug. 23, 2000 entitled “Novel Nucleic Acids and Polypeptides”, Attorney Docket No. 790CIP, which in turn is a continuation-in-part application of U.S. application Ser. No. 09/540,217 (now abandoned) filed Mar. 31, 2000 entitled “Novel Nucleic Acids and Polypeptides”, Attorney Docket No. 790; U.S. application Ser. No. 10/273,573 filed Oct. 18, 2002, entitled “Novel Nucleic Acids and Polypeptides,” Attorney Docket No. 791CIP5 which is a National Stage application of PCT Application Serial No. PCT/US01/08656 filed Apr. 18, 2001 entitled “Novel Nucleic Acids and Polypeptides”, Attorney Docket No. 791CIP3/PCT, which in turn is a continuation-in-part application of U.S. application Ser. No. 09/770,160 filed Jan. 26,2001 entitled “Novel Nucleic Acids and Polypeptides”, Attorney Docket No. 791CIP, which is in turn a continuation-in-part application of U.S. application Ser. No. 09/552,929 (now abandoned) filed Apr. 18, 2000 entitled “Novel Nucleic Acids and Polypeptides”, Attorney Docket No. 791; and U.S. application Ser. No. 10/276,817 filed Nov. 18, 2002, entitled “Novel Nucleic Acids and Polypeptides,” Attorney Docket No. 792CIP3/US, which is a National Stage application of PCT Application Serial No. PCT/US01/14827 filed May 16, 2001 entitled “Novel Nucleic Acids and Polypeptides”, Attorney Docket No. 792CIP3/PCT, which in turn is a continuation-in-part application of U.S. application Ser. No. 09/577,408 filed May 18, 2000 entitled “Novel Nucleic Acids and Polypeptides”, Attorney Docket No. 792; all of which are herein incorporated by reference in their entirety.

### 1. BACKGROUND

1. Technical Field

The present invention provides novel polynucleotides and proteins encoded by such polynucleotides, along with uses for these polynucleotides and proteins, for example in therapeutic, diagnostic and research methods.

2. Background Art

Technology aimed at the discovery of protein factors (including e.g., cytokines, such as lymphokines, interferons, CSFs, chemokines, and interleukins) has matured rapidly over the past decade. The now routine hybridization cloning and expression cloning techniques clone novel polynucleotides “directly” in the sense that they rely on information directly related to the discovered protein (i.e., partial DNA/amino acid sequence of the protein in the case of hybridization cloning; activity of the protein in the case of expression cloning). More recent “indirect” cloning techniques such as signal sequence cloning, which isolates DNA sequences based on the presence of a now well-recognized secretory leader sequence motif, as well as various PCR-based or low stringency hybridization-based cloning techniques, have advanced the state of the art by making available large numbers of DNA/amino acid sequences for proteins that are known to have biological activity, for example, by virtue of their secreted nature in the case of leader sequence cloning, by virtue of their cell or tissue source in the case of PCR-based techniques, or by virtue of structural similarity to other genes of known biological activity.

Identified polynucleotide and polypeptide sequences have numerous applications in, for example, diagnostics, forensics, gene mapping, identification of mutations responsible for genetic disorders or other traits, to assess biodiversity, and to produce many other types of data and products dependent on DNA and amino acid sequences. Proteins are known to have biological activity, for example, by virtue of their secreted nature in the case of leader sequence cloning, by virtue of their cell or tissue source in the case of PCR-based techniques, or by virtue of structural similarity to other genes of known biological activity. It is to these polypeptides and the polynucleotides encoding them that the present invention is directed.

### 2. SUMMARY OF THE INVENTION

This invention is based on the discovery of novel polypeptides, novel isolated polynucleotides encoding such polypeptides, including recombinant DNA molecules, cloned genes or degenerate variants thereof, especially naturally occurring variants such as allelic variants, antisense polynucleotide molecules, and antibodies that specifically recognize one or more epitopes present on such polypeptides, as well as hybridomas producing such antibodies. The compositions of the present invention additionally include vectors such as expression vectors containing the polynucleotides of the invention, cells genetically engineered to contain such polynucleotides, and cells genetically engineered to express such polynucleotides.

The compositions of the invention provide isolated polynucleotides that include, but are not limited to, a polynucleotide comprising the nucleotide sequence set forth in SEQ ID NO: 1-3, 5, 8, 10, 21, 23, 25, 27, 29, 31, 33, 35, 37 40, 43, 45, 49, 51, 53, 56-57, 59, 76-77, 79, 82, 84, 88-89, 91, 95-96, or 98; or a fragment thereof that retains a desired biological activity, a polynucleotide comprising the full length protein coding sequence of SEQ ID NO: 1-3, 5, 8, 10, 21, 23, 25, 27, 29, 31, 33, 35, 37-40, 43, 45, 49, 51, 53, 56-57, 59, 76-77, 79, 82, 84, 88-89, 91, 95-96, or 98 (for example, the open reading frame of SEQ ID NO: 4, 7, 9, 12, 22, 24, 26, 28, 30, 32, 34, 44, 46, 50, 58, 61, 78, 81, 83, 86, 90, 93, 97, or 100); and a polynucleotide comprising the nucleotide sequence of the mature protein coding sequence of any of SEQ ID NO: 1-3, 5, 8, 10, 21, 23, 25, 27, 29, 31, 33, 35, 37-40, 43, 45, 49, 51, 53, 56-57, 59, 76-77, 79, 82, 84, 88-89, 91, 95-96, or 98. The polynucleotides of the present invention also include, but are not limited to, a polynucleotide that hybridizes under stringent hybridization conditions to (a) the complement of any of the nucleotide sequences set forth in SEQ ID NO: 1-3, 5, 8, 10, 21, 23, 25, 27, 29, 31, 33, 35, 37-40, 43, 45, 49, 51, 53, 56-57, 59, 76-77, 79, 82, 84, 88-89, 91, 95-96, or 98; (b) a nucleotide sequence encoding any of the amino acid sequences set forth in SEQ ID NO: 4, 6-7, 9, 11-12, 22, 24, 26, 28, 30, 32, 34, 36, 44, 46-48, 50, 52-53, 58, 60-62, 78, 80-81, 83, 85-87, 90, 92-94, 97, or 99-101; a polynucleotide which is an allelic variant of any polynucleotides recited above having at least 70% polynucleotide sequence identity to the polynucleotides; a polynucleotide which encodes a species homolog (e.g. orthologs) of any of the peptides recited above; or a polynucleotide that encodes a polypeptide comprising a specific domain or truncation of the polypeptide of SEQ ID NO: 4, 6-7, 9, 11-12, 22, 24, 26, 28, 30, 32, 34, 36, 44, 46-48, 50, 52-53, 58, 60-62, 78, 80-81, 83, 85-87, 90, 92-94, 97, or 99-101.

A collection as used in this application can be a collection of only one polynucleotide. The collection of sequence information or unique identifying information of each sequence can be provided on a nucleic acid array. In one embodiment, segments of sequence information are provided on a nucleic acid array to detect the polynucleotide that contains the segment. The array can be designed to detect full-match or mismatch to the polynucleotide that contains the segment The collection can also be provided in a computer-readable format.

This invention further provides cloning or expression vectors comprising at least a fragment of the polynucleotides set forth above and host cells or organisms transformed with these expression vectors. Useful vectors include plasmids, cosmids, lambda phage derivatives, phagemids, and the like, that are well known in the art. Accordingly, the invention also provides a vector including a polynucleotide of the invention and a host cell containing the polynucleotide. In general, the vector contains an origin of replication functional in at least one organism, convenient restriction endonuclease sites, and a selectable marker for the host cell. Vectors according to the invention include expression vectors, replication vectors, probe generation vectors, and sequencing vectors. A host cell according to the invention can be a prokaryotic or eukaryotic cell and can be a unicellular organism or part of a multicellular organism.

The compositions of the present invention include polypeptides comprising, but not limited to, an isolated polypeptide selected from the group comprising the amino acid sequence of SEQ ID NO: 4, 6-7, 9, 11-12, 22, 24, 26, 28, 30, 32, 34, 36, 44, 46-48, 50, 52-53, 58, 60-62, 78, 80-81, 83, 85-87, 90, 92-94, 97, or 99-101; or the corresponding full length or mature protein. Polypeptides of the invention also include polypeptides with biological activity that are encoded by (a) any of the polynucleotides having a nucleotide sequence set forth in SEQ ID NO: 1-3, 5, 8, 10, 21, 23, 25, 27, 29, 31, 33, 35, 37-40, 43, 45, 49, 51, 53, 56-57, 59, 76-77, 79, 82, 84, 88-89, 91, 95-96, or 98; or (b) polynucleotides that hybridize to the complement of the polynucleotides of (a) under stringent hybridization conditions. Biologically or immunologically active variants of any of the protein sequences listed as SEQ ID NO: 4, 6-7, 9, 11-12, 22, 24, 26, 28, 30, 32, 34, 36, 44, 46-48, 50, 52-53, 58, 60-62, 78, 80-81, 83, 85-87, 90, 92-94, 97, or 99-101 and substantial equivalents thereof that retain biological or immunological activity are also contemplated. The polypeptides of the invention may be wholly or partially chemically synthesized but are preferably produced by recombinant means using the genetically engineered cells (e.g. host cells) of the invention.

The invention also provides compositions comprising a polypeptide of the invention. Pharmaceutical compositions of the invention may comprise a polypeptide of the invention and an acceptable carrier, such as a hydrophilic, e.g., pharmaceutically acceptable, carrier.

The invention also relates to methods for producing a polypeptide of the invention comprising culturing host cells comprising an expression vector containing at least a fragment of a polynucleotide encoding the polypeptide of the invention in a suitable culture medium under conditions permitting expression of the desired polypeptide, and purifying the protein or peptide from the culture or from the host cells. Preferred embodiments include those in which the protein produced by such a process is a mature form of the protein.

Polynucleotides according to the invention have numerous applications in a variety of techniques known to those skilled in the art of molecular biology. These techniques include use as hybridization probes, use as oligomers, or primers, for PCR, use in an array, use in computer-readable media, use for chromosome and gene mapping, use in the recombinant production of protein, and use in generation of antisense DNA or RNA, their chemical analogs and the like. For example, when the expression of an mRNA is largely restricted to a particular cell or tissue type, polynucleotides of the invention can be used as hybridization probes to detect the presence of the particular cell or tissue mRNA in a sample using, e.g., in situ hybridization.

In other exemplary embodiments, the polynucleotides are used in diagnostics as expressed sequence tags for identifying expressed genes or, as well known in the art and exemplified by Vollrath et al., Science 258:52-59 (1992), as expressed sequence tags for physical mapping of the human genome.

The polypeptides according to the invention can be used in a variety of conventional procedures and methods that are currently applied to other proteins. For example, a polypeptide of the invention can be used to generate an antibody that specifically binds the polypeptide. Such antibodies, particularly monoclonal antibodies, are useful for detecting or quantitating the polypeptide in tissue. The polypeptides of the invention can also be used as molecular weight markers, and as a food supplement.

Methods are also provided for preventing, treating, or ameliorating a medical condition which comprises the step of administering to a mammalian subject a therapeutically effective amount of a composition comprising a peptide of the present invention and a pharmaceutically acceptable carrier.

The methods of the invention also provide methods for the treatment of disorders as recited herein which comprise the administration of a therapeutically effective amount of a composition comprising a polynucleotide or polypeptide of the invention and a pharmaceutically acceptable carrier to a mammalian subject exhibiting symptoms or tendencies related to disorders as recited herein. In addition, the invention encompasses methods for treating diseases or disorders as recited herein comprising the step of administering a composition comprising compounds and other substances that modulate the overall activity of the target gene products and a pharmaceutically acceptable carrier. Compounds and other substances can effect such modulation either on the level of target gene/protein expression or target protein activity. Specifically, methods are provided for preventing, treating or ameliorating a medical condition, including viral diseases, which comprises administering to a mammalian subject, including but not limited to humans, a therapeutically effective amount of a composition comprising a polypeptide of the invention or a therapeutically effective amount of a composition comprising a binding partner of (e.g., antibody specifically reactive for) the polypeptides of the invention. The mechanics of the particular condition or pathology will dictate whether the polypeptides of the invention or binding partners (or inhibitors) of these would be beneficial to the individual in need of treatment.

According to this method, polypeptides of the invention can be administered to produce an in vitro or in vivo inhibition of cellular function. A polypeptide of the invention can be administered in vivo alone or as an adjunct to other therapies. Conversely, protein or other active ingredients of the present invention may be included in formulations of a particular agent to minimize side effects of such an agent.

The invention further provides methods for manufacturing medicaments useful in the above-described methods.

The present invention further relates to methods for detecting the presence of the polynucleotides or polypeptides of the invention in a sample (e.g., tissue or sample). Such methods can, for example, be utilized as part of prognostic and diagnostic evaluation of disorders as recited herein and for the identification of subjects exhibiting a predisposition to such conditions.

The invention provides a method for detecting a polypeptide of the invention in a sample comprising contacting the sample with a compound that binds to and forms a complex with the polypeptide under conditions and for a period sufficient to form the complex and detecting formation of the complex, so that if a complex is formed, the polypeptide is detected.

The invention also provides kits comprising polynucleotide probes and/or monoclonal antibodies, and optionally quantitative standards, for carrying out methods of the invention. Furthermore, the invention provides methods for evaluating the efficacy of drugs, and monitoring the progress of patients, involved in clinical trials for the treatment of disorders as recited above.

The invention also provides methods for the identification of compounds that modulate (i.e., increase or decrease) the expression or activity of the polynucleotides and/or polypeptides of the invention. Such methods can be utilized, for example, for the identification of compounds that can ameliorate symptoms of disorders as recited herein. Such methods can include, but are not limited to, assays for identifying compounds and other substances that interact with (e.g., bind to) the polypeptides of the invention.

The invention provides a method for identifying a compound that binds to the polypeptide of the present invention comprising contacting the compound with the polypeptide under conditions and for a time sufficient to form a polypeptide/compound complex and detecting the complex, so that if the polypeptide/compound complex is detected, a compound that binds to the polypeptide of the invention is identified.

Also provided is a method for identifying a compound that binds to a polypeptide of the invention comprising contacting the compound with a polypeptide of the invention in a cell for a time sufficient to form a polypeptide/compound complex wherein the complex drives expression of a reporter gene sequence in the cell and detecting the complex by detecting reporter gene sequence expression so that if the polypeptide/compound complex is detected a compound that binds to the polypeptide of the invention is identified.

### 3. BRIEF DESCRIPTION OF THE DRAWINGS

FIG. 1 shows a BLASTP amino acid sequence alignment between a CEA-like polypeptide (SEQ ID NO: 4) and another member of the family, mouse CEA-related cell adhesion molecule 1 (SEQ ID NO: 13).

FIG. 2 shows a BLASTP amino acid sequence alignment between a second CEA-like polypeptide (SEQ ID NO: 8) and another member of the family, a mouse protein similar to CEA-related cell adhesion molecule 6 precursor (SEQ ID NO: 14).

FIG. 3 shows a ClustalW multiple sequence alignment between the two CEA-like polypeptides of the invention (SEQ ID NO: 4 and 9).

FIG. 4 shows a multiple sequence alignment between chemokine-like polypeptides of the invention (SEQ ID NO: 18, 22, 26, 30, and 34) and the chemolines MCP-3 (SEQ ID NO: 41) and MIP-1a (SEQ ID NO: 42).

FIG. 5 shows the BLASTP amino acid sequence alignment between adiponectin-like polypeptide (SEQ ID NO: 44) and adiponectin/Apm1 (SEQ ID NO: 55) (gi4757760).

FIG. 6 shows the BLASTP amino acid sequence alignment between adiponectin-like polypeptide (SEQ ID NO: 44) and adiponectin family member C1q-related factor (SEQ ID NO: 54) (gi3747097).

FIG. 7 shows the modular structures of both adiponectin (SEQ ID NO: 55) (gi4757760) and SEQ ID NO: 44. Both the sequences have a leading signal peptide, a unique domain followed by a collagen-like domain and the globular C1q domain.

FIG. 8 shows the BLASTP amino acid sequence alignment between adiponectin-like polypeptide (SEQ ID NO: 50) and adiponectin/Apm1 (SEQ ID NO: 55).

FIG. 9 shows the BLASTP amino acid sequence alignment between adiponectin-like polypeptide (SEQ ID NO: 50) and adiponectin family member C1q-related factor (SEQ ID NO: 54).

FIG. 10 shows a multiple sequence alignment between the two adiponectin-like polypeptides of the invention (SEQ ID NO: 44 and 50) and adiponectin/Apm1 (SEQ ID NO: 55).

FIG. 11 shows a multiple sequence alignment of Ly-6-like polypeptides SEQ ID NO: 58, 65, and 71 and human PATE (expressed in prostate and testis, SEQ ID NO: 103), a member of the Ly-6 superfamily.

FIG. 12 shows a multiple sequence alignment of Ly-6-like polypeptides SEQ ID NO: 78, 83, and 90 and human sperm antigen SP-10 (SEQ ID NO: 104), a member of the Ly-6 superfamily.

FIG. 13 shows a sequence alignment of Ly-6-like polypeptide SEQ ID NO: 97 and mouse “similar to Ly-6H” (SEQ ID NO: 105), a member of the Ly-6 superfamily.

FIG. 14 shows a multiple sequence alignment of the uPAR/Ly-6 domains of the Ly-6-like polypeptides (SEQ ID NO: 62, 69, 75, 87, 94, 101) with the uPAR/Ly-6 domains of PATE (SEQ ID NO: 103), SP-10 (SEQ ID NO: 104) and the three uPAR/Ly-6 domains of human urokinase-type plasminogen activator receptor (uPAR, SEQ ID NO: 102).

FIG. 15 shows the consensus sequence for the uPAR/Ly-6 cysteine-rich domain defining the Ly-6 superfamily. The brackets represent the disulfide bond connectivity. Only the conserved identities are shown, but when the spacing between equivalent cysteines is conserved, the distance in amino acid residues is represented by the number of hyphens (-). The vertical double bars (//) represent insertions or deletions. The plus signs (+) indicate disulfide bond pairs.

FIG. 16 depicts a schematic of the common structural features of the Ly-6-like polypeptides of the invention. The common structure of the family is as follows: signal peptide (hatched boxes), followed by a (GEXXS)n (SEQ ID NO: 106) repeat region, wherein G=Glycine, E=Glutamic Acid, X=any amino acid, S=serine, and n can be from 0 to 1 to dozens (solid boxes), followed by the uPAR/Ly-6 cysteine-rich domain (checkered boxes).

### 4. DETAILED DESCRIPTION OF THE INVENTION

Table 1 is a correlation table of the novel polynucleotide sequences (1-3, 5, 8, 10, 21, 23, 25, 27, 29, 31, 33, 35, 37-40, 43, 45, 49, 51, 53, 56-57, 59, 76-77, 79, 82, 84, 88-89, 91, 95-96, and 98) and the novel polypeptides (4, 6-7, 9, 11-12, 22, 24, 26, 28, 30, 32, 34, 36, 44, 46-48, 50, 52-53, 58, 60-62, 78, 80-81, 83, 85-87, 90, 92-94, 97, and 99-101) and the corresponding SEQ ID NO: in which the sequence was filed in the following priority U.S. Patent Applications bearing the Serial No. of: 60/395,402 filed on Jul. 2, 2002, 60/416,261 filed on Oct. 3, 2002, 60/418,132 filed on Oct. 11, 2002, and 60/425,158 filed Nov. 8, 2002,

TABLE 1

| | Identification of Priority Application |
|---|---|
| | that sequence was filed (Attorney |
| SEQ ID NO: | Docket No._SEQ ID NO.)* |
| 2 | HYS-63_1 |
| 3 | HYS-63_2 |
| 4 | HYS-63_3 |
| 5 | HYS-63_4 |
| 6 | HYS-63_5 |
| 7 | HYS-63_6 |
| 8 | HYS-63_7 |
| 9 | HYS-63_8 |
| 10 | HYS-63_9 |
| 11 | HYS-63_10 |
| 12 | HYS-63_11 |
| 13 | HYS-63_12 |
| 14 | HYS-63_13 |
| 17 | HYS-64_1 |
| 18 | HYS-64_2 |
| 19 | HYS-64_3 |
| 20 | HYS-64_4 |
| 21 | HYS-64_5 |
| 22 | HYS-64_6 |
| 23 | HYS-64_7 |
| 24 | HYS-64_8 |
| 25 | HYS-64_9 |
| 26 | HYS-64_10 |
| 27 | HYS-64_11 |
| 28 | HYS-64_12 |
| 29 | HYS-64_13 |
| 30 | HYS-64_14 |
| 31 | HYS-64_15 |
| 32 | HYS-64_16 |
| 33 | HYS-64_17 |
| 34 | HYS-64_18 |
| 35 | HYS-64_19 |
| 36 | HYS-64_20 |
| 37 | HYS-64_21 |
| 38 | HYS-64_22 |
| 39 | HYS-64_23 |
| 40 | HYS-64_24 |
| 43 | HYS-65_1 |
| 44 | HYS-65_2 |
| 45 | HYS-65_3 |
| 46 | HYS-65_4 |
| 47 | HYS-65_5 |
| 48 | HYS-65_6 |
| 49 | HYS-65_7 |
| 50 | HYS-65_8 |
| 51 | HYS-65_9 |
| 52 | HYS-65_10 |
| 53 | HYS-65_11 |
| 54 | HYS-65_12 |
| 55 | HYS-65_13 |
| 56 | HYS-66_1 |
| 57 | HYS-66_2 |
| 58 | HYS-66_3 |
| 59 | HYS-66_4 |
| 60 | HYS-66_5 |
| 61 | HYS-66_6 |
| 62 | HYS-66_7 |
| 63 | HYS-66_8 |
| 64 | HYS-66_9 |
| 65 | HYS-66_10 |
| 66 | HYS-66_11 |
| 67 | HYS-66_12 |
| 68 | HYS-66_13 |
| 69 | HYS-66_14 |
| 70 | HYS-66_15 |
| 71 | HYS-66_17 |
| 72 | HYS-66_18 |
| 73 | HYS-66_19 |
| 75 | HYS-66_20 |
| 76 | HYS-66_21 |
| 77 | HYS-66_22 |
| 78 | HYS-66_23 |
| 79 | HYS-66_24 |
| 80 | HYS-66_25 |
| 81 | HYS-66_26 |
| 82 | HYS-66_27 |
| 83 | HYS-66_28 |
| 84 | HYS-66_29 |
| 85 | HYS-66_30 |
| 86 | HYS-66_31 |
| 87 | HYS-66_32 |
| 88 | HYS-66_33 |
| 89 | HYS-66_34 |
| 90 | HYS-66_35 |
| 91 | HYS-66_36 |
| 92 | HYS-66_37 |
| 93 | HYS-66_38 |
| 94 | HYS-66_39 |
| 95 | HYS-66_40 |
| 96 | HYS-66_41 |
| 97 | HYS-66_42 |
| 98 | HYS-66_43 |
| 99 | HYS-66_44 |
| 100 | HYS-66_45 |
| 101 | HYS-66_46 |
| 102 | HYS-66_47 |
| 103 | HYS-66_48 |
| 104 | HYS-66_49 |
| 105 | HYS-66_50 |

*HYS-63_XXX = SEQ ID NO: XXX of Attorney Docket No. HYS-63, U.S. Serial No. 60/395,402 filed Jul. 12, 2002, the entire disclosure of which, including sequence listing, is incorporated herein by reference.
HYS-64_XXX = SEQ ID NO: XXX of Attorney Docket No. HYS-64, U.S. Serial No. 60/416,261 filed Oct. 03, 2002, the entire disclosure of which, including sequence listing, is incorporated herein by reference.
HYS-65_XXX = SEQ ID NO: XXX of Attorney Docket No. HYS-65, U.S. Serial No. 60/418,132 filed Oct. 11, 2002, the entire disclosure of which, including sequence listing, is incorporated herein by reference.
HYS-66_XXX = SEQ ID NO: XXX of Attorney Docket No. HYS-66, U.S. Serial No. 60/425,158 filed Nov. 08, 2002, the entire disclosure of which, including sequence listing, is incorporated herein by reference.

4.1 CEA-Like Polypeptides and Polynucleotides

Many tumors express genes whose products are required for either inducing or maintaining the malignant state (Abbas et al. (2000) Cellular and Molecular Immunology, Saunders (Publisher) pp 386, incorporated herein by reference). These proteins could be used as markers for tumor detection or as therapeutic targets. Carcinoembryonic antigens (CEAs, e.g. CD66a-CD66d) were first identified as antigens that are expressed on many carcinomas, including colon, pancreas, stomach, and breast. Subsequently, with the development of more sensitive detection techniques, these proteins are now also known to be expressed during fetal development, inflammation, and, in some cases, in minute quantities in normal tissues.

Carcinoembryonic antigens are integral membrane glycoproteins belonging to immunoglobulin (Ig) superfamily of receptors. CEA cell adhesion molecule (CEA-CAM), also known as billiary glycoprotein (BGP) or CD66a, is a protein of about 85 kDa and, is highly glycosylated and exhibits at least two tissue specific, alternatively spliced, variants (Hammarstrom, Semin. Cancer Biol. 9:67-81 (1999), incorporated herein by reference).

The immunoglobulin superfamily members that serve as receptors are classified into three groups according to their cytoplasmic domain characteristics. Transmembrane molecules with immunoreceptor tyrosine activation motifs (ITAMs) (YxxL (SEQ ID NO: 15), where x is any amino acid) are usually activating receptors. Those possessing immunoreceptor tyrosine inhibition motifs (ITIMs) (I/L/VxxYxxL/V (SEQ ID NO: 16), where x is any amino acid) are inhibitory in nature (Isakov, Immunol. Res. 16:85-100 (1997), incorporated herein by reference). There appears to be a third class of short transmembrane receptors like LIR-4, or alternately spliced soluble forms of FDF03, that have no known activating or inhibitory motifs. These molecules by virtue of their extracellular MHC binding domain are thought to function as a “molecular sink” and could inhibit the functions of cognate transmembrane receptors.

Several functions have been attributed to CEA. The first Ig domain of CD66a serves as an adhesive module to bind E-selectin and initiate the inflammatory cascade. The mouse CEAs are known to be the receptors for mouse hepatitis virus, whereas human CEA has been shown to be a receptor for bacterial proteins from Neisseria gonorrhoeae, Salmonella enterica, and Escherichia coli. This indicates that CEA may play a role in the internalization of viruses and bacteria. CEA has also been shown to act as a negative regulator, and could therefore function as a tumor suppressor for colonic, prostate and breast carcinomas (Huber et al., J. Biol. Chem. 274:335-344 (1999), incorporated herein by reference).

The cytoplasmic region of CEA-CAM has also been shown to link CEA-CAM to a role in signal transduction. Several physiological events promote the phosphorylation of tyrosines in the cytoplasmic domain of CEA. It is also reported that stimulation of BGP1 in neutrophils leads to activation of Rac1, PAK, and Jun N-terminal kinase. Similarly, it is reported that the ITIM sequences in the BGP1 cytoplasmic domains interact with protein-tyrosine phosphatases SHP-1 and SHP-2 in epithelial cells. Since CEA is involved in the negative regulation of tumor cell growth, CEA may function in generating and/or modulating signals leading to growth arrest (Luo et al., Oncogene 14:1697-1704 (1997), incorporated herein by reference; Huber et al., J. Biol. Chem. 274:335-344 (1999), incorporated herein by reference).

The over-expression of CEA in colon, pancreatic, gastric, and breast carcinomas creates a detectable rise in serum CEA levels. These changes in serum levels are used to monitor the occurrence or recurrence of metastatic carcinoma after primary treatment. In its role as an intercellular adhesion, CEA promotes the binding of tumor cells to one another, and therefore could be used to modulate the interaction of tumor cells with themselves and with the tissue in which the tumor cells are growing.

CEA appears to be involved in cell adhesion and subsequent signal transduction during normal fetal development, inflammation, and carcinogenesis. Polynucleotides encoding CEA and polypeptides thereof could serve as potential therapeutics in the treatment of breast, prostate, colon and other cancers. CEA and compounds which bind to CEA could also be useful in treating disorders relating to inflammation and autoimmunity. Soluble CEA could also be used as immunosuppressantin organ transplant patients. Soluble CEA molecule could serve as a decoy receptor in above-mentioned bacterial and viral infections.

The CEA-like polypeptide of SEQ ID NO: 4 is an approximately 270-amino acid protein with a predicted molecular mass of approximately 30-kDa unglycosylated. The initial methionine starts at position 335 of SEQ ID NO: 3 and the putative stop codon begins at position 1145 of SEQ ID NO: 3. A signal peptide of 33 residues is predicted from approximately residue 1 to residue 33 of SEQ ID NO: 4. The extracellular portion is useful on its own. The signal peptide region was predicted using the Neural Network SignaP V1.1 program (Nielsen et al., Int. J. Neural Syst. 8:581-599 (1997)). One of skill in the art will recognize that the actual cleavage site may be different than that predicted by the computer program.

Using the the TMpred program (Hofmann and Stoffel, Biol. Chem. Hoppe-Seyler 374:166 (1993), herein incorporated by reference in its entirety), CEA-like polypeptide is predicted to have a transmembrane domain from approximately residue 241 to residue 263 of SEQ ID NO: 4. Removal of the transmembrane domain renders soluble fragments that can be used to inhibit receptor activity. An exemplary extracellular domain spans approximately residue 1 to residue 240 of SEQ ID NO: 4. One of skill in the art will recognize that the actual transmembrane domain may be different than that predicted by the computer program.

Protein database searches with the BLASTP algorithm (Altschul S. F. et al., J. Mol. Evol. 36:290-300 (1993) and Altschul S. F. et al., J. Mol. Biol. 21:403-10 (1990), herein incorporated by reference) indicate that SEQ ID NO: 4 is homologous to carcinoembryonic antigen (CEA)-like proteins.

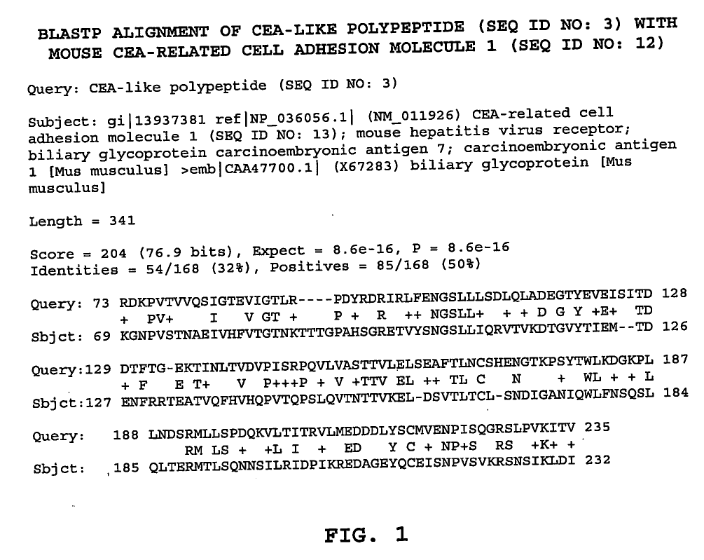

FIG. 1 shows the BLASTP amino acid sequence alignment between CEA-like polypeptide SEQ ID NO: 4 and mouse CEA-related cell adhesion molecule 1 (SEQ ID NO: 13), indicating that the two sequences share 50% similarity and 32% identity over 168 amino acid residues, wherein A=Alanine, C=Cysteine, D=Aspartic Acid, E=Glutamic Acid, F=Phenylalanine, G=Glycine, H=Histidine, I=Isoleucine, K=Lysine, L=Leucine, M=Methionine, N=Asparagine, P=Proline, Q Glutamine, R=Arginine, S=Serine, T=Threonine, V=Valine, W=Tryptophan, Y=Tyrosine. Gaps are presented as dashes.

Using the Pfam software program (Sonnhammer et al., Nucleic Acids Res., 26:320-322 (1998) herein incorporated by reference) SEQ ID NO: 4 was examined for domains with homology to known conserved peptide domains. Table 2 shows the name of the Pfam model found, the description, the e-value, Pfam score, number of repeats, and position of the domain(s) within SEQ ID NO: 4 for the identified model within the sequence as follows:

TABLE 2

| Model | Description | E-value | Score | Repeats | Position |
|---|---|---|---|---|---|
| Ig | Immunoglobulin | 6.7e−10 | 46.3 | 2 | 48-124 |
| | domain | | | | 161-219 |

Using the eMATRIX software package (Stanford University, Stanford, Calif.) (Wu et al., J. Comp. Biol., 6:219-235 (1999), herein incorporated by reference), the CEA-like polypeptide of SEQ ID NO: 4 was determined to have following the eMATRIX domain hits. The results in Table 3 describe: the eMATRIX domain name, the corresponding p-value, Signature ID number, and the corresponding position of the domain within SEQ ID NO: 4:

TABLE 3

| | Signature ID | | |
|---|---|---|---|
| Name | NO | p-value | Position |
| CARCINOEMBRYONIC ANTIGEN | DM00372B | 8.356e−08 | 82-126 |
| PRECURSOR AMINO-TERMINAL | | | |
| DOMAIN | | | |

The CEA-like polypeptide of SEQ ID NO: 9 is an approximately 416-amino acid protein with a predicted molecular mass of approximately 46-kDa unglycosylated. The initial methionine starts at position 335 of SEQ ID NO: 8 and the putative stop codon begins at position 1583 of SEQ ID NO: 8. A signal peptide of 33 residues is predicted from approximately residue 1 to residue 33 of SEQ ID NO: 9. The extracellular portion is useful on its own. The signal peptide region was predicted using the Neural Network SignalP V1.1 program (Nielsen et al, Int. J. Neural Syst. 8:581-599 (1997), herein incorporated by reference in its entirety). One of skill in the art will recognize that the actual cleavage site may be different than that predicted by the computer program.

Using the the TMpred program (Hofmann and Stoffel, Biol. Chem. Hoppe-Seyler 374:166 (1993), herein incorporated by reference in its entirety), CEA-like polypeptide is predicted to have a transmembrane domain from approximately residue 241 to residue 263 of SEQ ID NO: 9. Removal of the transmembrane domain renders soluble fragments that can be used to inhibit receptor activity. An exemplary extracellular domain spans approximately residue 1 to residue 240 of SEQ ID NO: 9. One of skill in the art will recognize that the actual transmembrane domain may be different than that predicted by the computer program.

Protein database searches with the BLASTP algorithm (Altschul S. F. et al., J. Mol. Evol. 36:290-300 (1993) and Altschul S. F. et al., J. Mol. Biol. 21:403-10 (1990), herein incorporated by reference) indicate that SEQ ID NO: 9 is homologous to carcinoembryonic antigen (CEA)-like proteins.

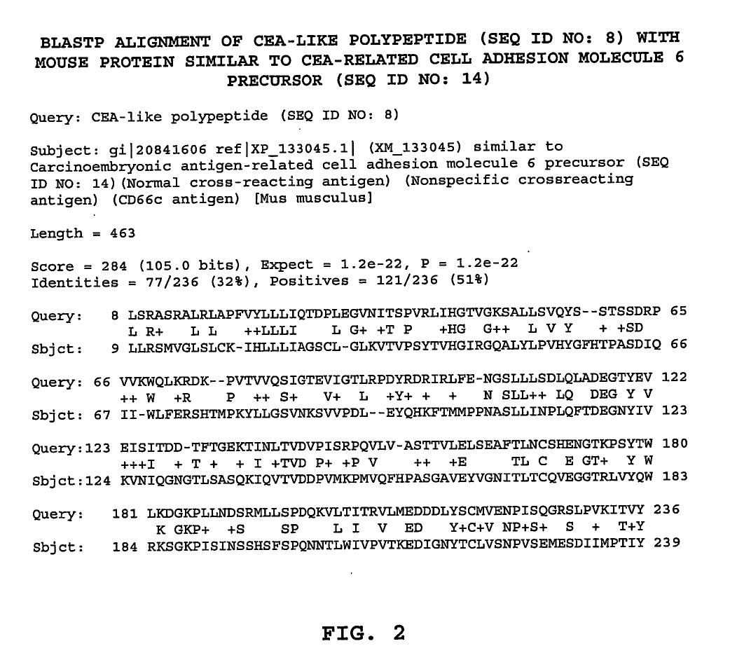

FIG. 2 shows the BLASTP amino acid sequence alignment between CEA-like polypeptide SEQ ID NO: 9 and a mouse protein similar to CEA-related cell adhesion molecule 6 precursor (SEQ ID NO: 14), indicating that the two sequences share 51% similarity and 32% identity over 236 amino acid residues, wherein A=Alanine, C=Cysteine, D=Aspartic Acid, E=Glutamic Acid, F=Phenylalanine, G=Glycine, H=Histidine, I=Isoleucine, K=Lysine, L=Leucine, M=Methionine, N=Asparagine, P=Proline, Q=Glutamine, R=Arginine, S=Serine, T=Threonine, V=Valine, W=Tryptophan, Y=Tyrosine. Gaps are presented as dashes.

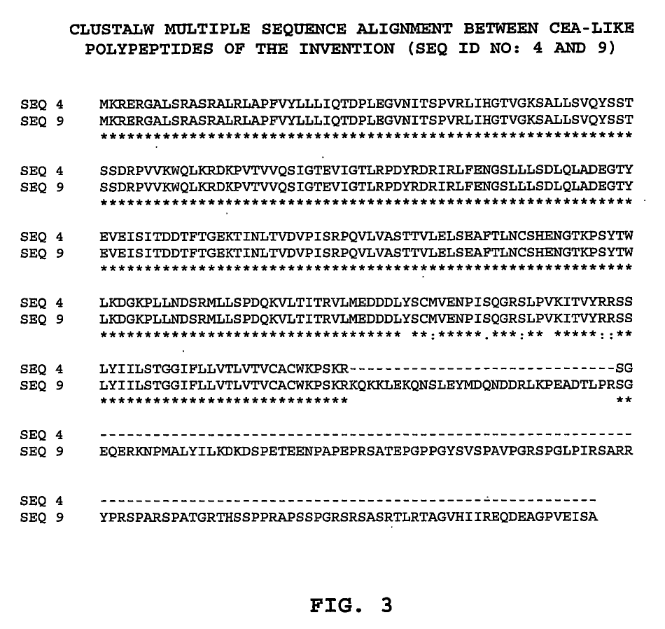

FIG. 3 shows a ClustalW multiple sequence alignment of the two CEA-like polypepetides of the invention (SEQ ID NO: 4 and 9), wherein asterisks (*) represent identical residues, colons (:) represent conservative substitutions, and periods (.) represent semi-conservative substitutions. Gaps are represented as dashes.

Using the Pfam software program (Sonnhammer et al., Nucleic Acids Res., 26:320-322 (1998) herein incorporated by reference) SEQ ID NO: 9 was examined for domains with homology to known conserved peptide domains. Table 4 shows the name of the Pfam model found, the description, the e-value, Pfam score, number of repeats, and position of the domain(s) within SEQ ID NO: 9 for the identified model within the sequence as follows:

TABLE 4

| Model | Description | E-value | Score | Repeats | Position |
|---|---|---|---|---|---|
| Ig | Immunoglobulin | 6.7e−10 | 46.3 | 2 | 48-124 |
| | domain | | | | 161-219 |

Using the eMATRIX software package (Stanford University, Stanford, Calif.) (Wu et al., J. Comp. Biol., 6:219-235 (1999), herein incorporated by reference), the CEA-like polypeptide of SEQ ID NO: 9 was determined to have following the eMATRIX domain hits. The results Table 5 describe: the eMATRIX domain name, the corresponding p-value, Signature ID number, and the corresponding position of the domain within SEQ ID NO: 9:

TABLE 5

| | Signature | | |
|---|---|---|---|
| Name | ID NO | p-value | Position |
| CORONAVIRUS | DM01206B | 1.942e−08 | 375-394 |
| NUCLEOCAPSID PROTEIN | | | |
| DOPAMINE D4 RECEPTOR | PR00569C | 6.516e−08 | 372-386 |
| SIGNATURE | | | |
| CARCINOEMBRYONIC | DM00372B | 8.356e−08 | 82-126 |
| ANTIGEN PRECURSOR | | | |
| AMINO-TERMINAL DOMAIN | | | |

CEA-like polypeptides of the present invention may be involved in cell adhesion and subsequent signal transduction during normal fetal development and also during inflammation and carcinogenesis. Polynucleotides encoding CEA and CEA-like proteins and polypeptides thereof could serve as potential therapeutics in the treatment of breast cancer, ovarian cancer, lung cancer, brain cancer, colon cancer, prostate cancer, pancreatic cancer, gastric cancer and other cancers. CEA-like proteins and compounds which bind to CEA-like proteins could also be useful in treating disorders relating to inflammation and autoimmunity. Soluble CEA-like proteins could also be used as immunosuppressants in organ transplant patients and could serve as decoy receptors in certain bacterial and viral infections.

4.2 Chemokine-Like Polypeptides, and Polynucleotides

Chemokines are a collection of small (approximately 8-14 kDa) structurally related proteins that regulate cell trafficking of various types of leukocytes through interactions with a subset of seven-transmembrane, G protein-coupled recetors (Zlotnik et al, Immunity; 12:121-127 (2000), incorporated herein by reference). Over 40 chemokines have been identified in humans, and they can be categorized into four major families (CC, CXC, C and CX₃C) according to the pattern of cysteine residues near the NH2-terminus. They mainly act on neutrophils, moncytes, lymphocytes, and eosinophils, and play a central role in host defense mechanisms (Zlotnik et al, Immunity; 12:121-127 (2000), incorporated herein by reference). Much effort has gone into characterizing the functions carried out by chemokines.

Chemokines play key roles in several biological functions, including leukocyte chemotaxis, integrin activation during leukocyte-endothelial interactions, leukocyte degranulation, and angiogenesis or angiostasis Mackay, Nature Immunology; 2:95-101 (2001), incorporated herein by reference). Chemokines can provide directional cues for leukocyte motility through the formation of gradients that migrating cells can sense. Consequently, migrating cells can undergo a profound transformation that results in a redistribution of chemokine receptors, integrins, cytoskeletal proteins and intracellular regulatory molecules. For example, chemokines (including CCL20 and CXCL13) and chemokine receptors (including CSCR4) direct the movement of developing B cells which presumably allow these cells to migrate from bone marrow to spleen and then to other lymphoid microenvironments. (Mackay, Nature Immunology; 2:95-101 (2001), incorporated herein by reference). Another function for chemolines is their involvement in signaling events for integrin activation during the multi-step process of leukocyte-endothelial cell interactions (Springer, Cell; 76:301-314 (1994), incorporated herein by reference). Chemokines also play roles in stimulating leukocyte degranulation. For instance, CCL2 (MCP-1) is a potent stimulator of histamine release by basophils and CXCL8 stimulates exocytosis of neutrophil granules (Mackay, Nature Immunology; 2:95-101 (2001), incorporated herein by reference). Some chemolines also stimulate angiogenesis or angiostasis. The “ELR” CXC chemolines and CCL2 possess angiogenic properties, whereas CXCR3 ligands, such as CXCL10 and CCL21 (SLC), possess angiostatic properties. The biological relevance of angiogenic or angiostatic properties of chemolines could related to tumor suppression or to inflammatory responses where angiogenesis is an important requirement (Mackay, Nature Immunology; 2:95-101 (2001), incorporated herein by reference).

Based on data from clinical observations or animal data, chemolines could be involved in a variety of disease states, including autoimmune diseases, graft rejection, infection, inflammation or allergy, neoplasia, and vascular diseases (Gerard et al., Nature Immunology; 2:108-115 (2001), incorporated herein by reference). Autoimmune diseases include rheumatoid arthritis, systemic lupus erythematosis, and multiple sclerosis. Graft rejection includes heart allograft rejection and kidney allograft rejection. Infection includes acute and chronic bacterial and viral infections (especially HIV and myobacteria) and sepsis. Neoplasia includes leukocyte recruitment in cancer and angiogenesis. Vascular disease includes atherosclerosis, hypertension, and ischemia-reperfusion.

This invention relates to five chemokine-like polypeptides. The first chemokine-like polypeptide of SEQ ID NO: 18 is an approximately 133 amino acid protein with a predicted molecular mass of approximately 14.6-kDa unmodified. The initial methionine codon starts at position 217 of SEQ ID NO: 17 and the putative stop codon begins at position 616 of SEQ ID NO: 17. A signal peptide of twenty-five residues is predicted from approximately residue 1 to residue 25 of SEQ ID NO: 18. The mature portion is useful on its own. The signal peptide region was predicted using the Neural Network SignalP V1.1 program (Nielsen et al., Int. J. Neural Syst. 8:581-599 (1997), herein incorporated by reference in its entirety). One of skill in the art will recognize that the actual cleavage site may be different than that predicted by the computer program.

The second chemokine-like polypeptide of SEQ ID NO: 22 is an approximately 131 amino acid protein with a predicted molecular mass of approximately 14.4-kDa unmodified. The initial methionine codon starts at position 201 of SEQ ID NO: 21 and the putative stop codon begins at position 594 of SEQ ID NO: 21. A signal peptide of thirty residues is predicted from approximately residue 1 to residue 30 of SEQ ID NO: 22. The mature portion is useful on its own. The signal peptide region was predicted using the Neural Network SignalP V1.1 program (Nielsen et al., Int. J. Neural Syst. 8, 581-599 (1997), herein incorporated by reference in its entirety). One of skill in the art will recognize that the actual cleavage site may be different than that predicted by the computer program.

The third chemokine-like polypeptide of SEQ ID NO: 26 is an approximately 133 amino acid protein with a predicted molecular mass of approximately 14.6-kDa unmodified. The initial methionine codon starts at position 70 of SEQ ID NO: 25 and the putative stop codon begins at position 469 of SEQ ID NO: 25. A signal peptide of thirty residues is predicted from approximately residue 1 to residue 30 of SEQ ID NO: 26. The mature portion is useful on its own. The signal peptide region was predicted using the Neural Network SignalP V1.1 program (Nielsen et al., Int. J. Neural Syst. 8:581-599 (1997), herein incorporated by reference in its entirety). One of skill in the art will recognize that the actual cleavage site may be different than that predicted by the computer program.

The fourth chemokine-like polypeptide of SEQ ID NO: 30 is an approximately 125 amino acid protein with a predicted molecular mass of approximately 13.8-kDa unmodified. The initial methionine codon starts at position 150 of SEQ ID NO: 29 and the putative stop codon begins at position 525 of SEQ ID NO: 29. A signal peptide of twenty-five residues is predicted from approximately residue 1 to residue 25 of SEQ ID NO: 30. The mature portion is useful on its own. The signal peptide region was predicted using the Neural Network SignalP V1.1 program (Nielsen et al., Int. J. Neural Syst. 8:581-599 (1997), herein incorporated by reference in its entirety). One of skill in the art will recognize that the actual cleavage site may be different than that predicted by the computer program.

The fifth chemokine-like polypeptide of SEQ ID NO: 34 is an approximately 140 amino acid protein with a predicted molecular mass of approximately 15.4-kDa unmodified. The initial methionine codon starts at position 466 of SEQ ID NO: 33 and the putative stop codon begins at position 886 of SEQ ID NO: 33. A signal peptide of thirty-four residues is predicted from approximately residue 1 to residue 34 of SEQ ID NO: 34. The mature portion is useful on its own. The signal peptide region was predicted using the Neural Network SignalP V1.1 program (Nielsen et al., Int. J. Neural Syst. 8:581-599 (1997), herein incorporated by reference in its entirety). One of skill in the art will recognize that the actual cleavage site may be different than that predicted by the computer program.

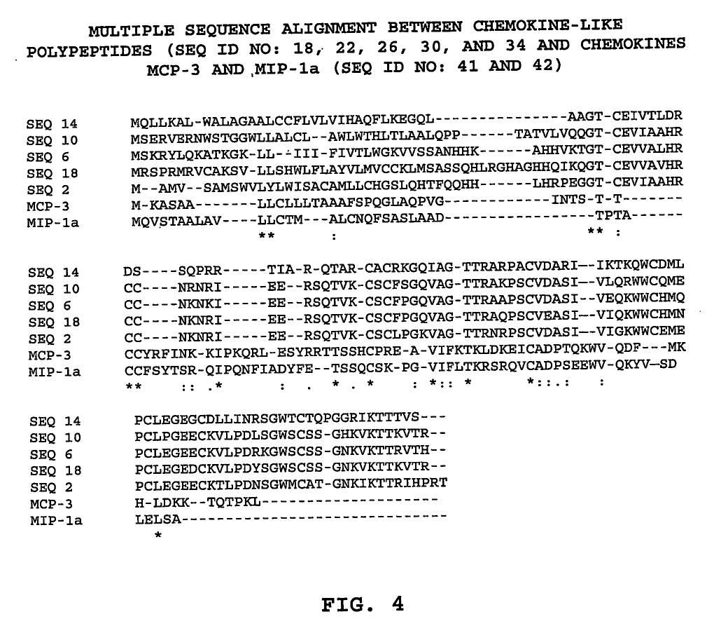

FIG. 4 shows a multiple sequence alignment between chemokine-like polypeptides (SEQ ID NO: 18, 22, 26, 30, and 34) and the chemokines MCP-3 (SEQ ID NO: 41) and MIP-1a (SEQ ID NO: 42). Regions of significant conservation are indicated in gray. For the alignment between SEQ ID NO: 18, 22, 26, 34, MCP-3 and MIP-1a, asterisks (*) represent identical residues, colons (:) represent conserved residues, and periods (.) represent semi-conserved residues. The alignment indicates that the chemokine-like polypeptides are highly homologous to each other and display significant homology to the CC-chemokines MCP-3 and MIP-1a.

The polypeptides of the invention, based on their homologies to chemokines, are expected to function in several biological processes, including leukocyte chemotaxis, integrin activation during leukocyte-endothelial interactions, leukocyte degranulation and mediator release, and angiogenesis or angiostasis.

The polypeptides, polynucleotides, antibodies and other compositions of the invention are expected to be useful in treating disorders including autoimmune diseases, graft rejection, infection, inflammation or allergy, neoplasia, and vascular diseases. Autoimmune diseases include rheumatoid artritis, systemic lupus erythematosis, and multiple sclerosis. Graft rejection includes heart allograft rejection and kidney allograft rejection. Infection includes acute and chronic bacterial and viral infections (especially HIV and myobacteria) and sepsis. Neoplasia includes leukocyte recruitment in cancer and angiogenesis. Vascular disease includes atherosclerosis, hypertension, and ischemia-reperfusion.

4.3 Adiponectin-Like Polypeptides and Polynucleotides

Adipose tissue primarily serves as an energy reservoir by storing fat and is involved in regulating available energy to the body. However, it has only recently become apparent that adipocytes synthesize and secrete many important proteins, including leptin, adipsin, complement components such as C3a and properdin, tumor necrosis factor (TNF)-α, plasminogen-activator inhibitor type 1 (PAI-1), and resistin. These adipocyte proteins are collectively called adipocytokines (Yamauchi et al., Nature Med. 7:941-946 (2001), herein incorporated by reference in its entirety).

Adiponectin (also known as adipocyte complement-related protein, Acrp30O), gelatin-binding protein (GBP28), or APM1) is such an adipocytokine that was identified by differential display cloning of preadipocytes and adipocytes in mouse cells. In humans, it was identified as an adipocyte-specific gene. There appears to be a large family of related proteins that share both sequence and structural homology including C1q, human type VIII and X collagens, precerebellin, and the hibernation-regulated proteins, hib 20, hib 25, and hib 27. Adiponectin (AdipoQ) has a modular design: a cleaved amino-terminal sequence, a region without homology to known proteins, a collagen-like region, and a C-terminal complement factor C1Q-like globular domain (Fruebis et al., Proc. Natl. Acad. Sci. USA 98:2005-2010 (2001), herein incorporated by reference in its entirety). The globular domain forms homotrimers like TNF-α, and the collagen-like domains can further form higher order structures.

Functionally, adiponectin was found to suppress TNF-α-induced monocyte adhesion to human aortic endothelial cells (Ouchi et al., Circulation 100:2473-2476 (1999), herein incorporated by reference in its entirety). They also reported that adiponectin suppressed the increased expression of VCAM-1, ICAM-1, and E-selectin, suggesting that adiponectin may attenuate the inflammatory responses associated with atherosclerosis. More recently, authors also reported that plasma levels of adiponectin were significantly lower in patients with coronary artery disease than in age and body mass index-matched normal subjects (Ouchi et al., Circulation 102:1296-1301 (2000), herein incorporated by reference in its entirety). It was further shown that adiponectin suppressed TNF-α-induced nuclear factor Kappa B (NF-κB) activation accompanied by cAMP accumulation. Adiponectin also inhibited myelomonocytic progenitor cell proliferation, at least in part due to apoptotic mechanisms in hematopoietic colony formation assays. In macrophages, adiponectin suppressed the expression of class A macrophage scavenger receptors (MSR) and altered cholesterol metabolism. In particular, adiponectin reduced intracellular cholesteryl ester content of the macrophages (Ouchi et al., Circulation 103:1057-63 (2001), herein incorporated by reference in its entirety). The findings suggested that adiponectin protein suppressed the transformation of macrophages to foam cells.

Insulin resistance induced by high-fat diet and associated with obesity is a major risk factor for diabetes and cardiovascular diseases. It has been shown that adipocytokines play a crucial role in these processes. TNF-α overproduced in adipose tissue contributes to insulin resistance. Leptin, another adipocytokine, which contributes to the regulation of food intake and energy expenditure, also affects insulin sensitivity and may lead to hypertension. Similarly, serum adiponectin concentrations are decreased in ob/ob mice, obese humans, diabetic patients, and patients with coronary artery diseases (Hotta et al. Arterioscler. Thromb. Vasc. Biol. 20:1595-1599 (2000), herein incorporated by reference in its entirety).

In mouse models, it was shown that acute treatment with a proteolytically generated globular domain of Acrp30 (gAcrp30) could lead to altered lipid metabolism. In particular, the gAcrp30 reduced plasma fatty acid (FFA) levels caused by administration of a high-fat test meal (Freubis et al., Proc. Natl. Acad. Sci. USA 98:2005-2010 (2001), herein incorporated by reference in its entirety). This effect was in part due to increased fatty acid oxidation by muscle. Low doses of gAcrp30 given to mice that were on high-fat/sucrose diet caused profound and sustainable weight reduction without affecting food intake. These data indicated that adiponectin as well as other adiponectin family members may be involved in energy homeostasis and their dysregulation may lead to pathological conditions.

Recently, Yamauchi et al. showed that decreased expression of adiponectin correlates with insulin resistance in mouse models of altered insulin sensitivity (Yamauchi et al., Nature Med. 7:941-946 (2001), herein incorporated by reference in its entirety). Adiponectin decreased the levels of triglycerides in muscle and liver in obese mice. These effects were due to increased fatty acid combustion and energy dissipation in muscle. The authors further showed that insulin resistance was completely reversed in lipoatrophic mice by administering combination of physiological doses of adiponectin and leptin, but only partially with either adiponectin or leptin alone.

The role of adiponectin was further studied in the adiponectin knock-out (KO) mice by Matsuda et al. (J. Biol. Chem 277:37487-37491 (2002), herein incorporated by reference in its entirety) and Kubota et al. (J. Biol. Chem. 277:25863-25866 (2002), herein incorporated by reference in its entirety). The adiponectin-deficient mice in each study showed severe neointimal thickening and increased proliferation of vascular smooth muscle cells in mechanically injured arteries. Adenovirus-mediated supplement of adiponectin attenuated the neotintimal proliferation, suggesting that adiponectin plays a direct role in neointimal thickening of arteries, a key feature of the restenosis phenomenon observed after balloon angioplasty. In cultured smooth muscle cells, adiponectin attenuated DNA synthesis induced a variety of growth factors such as PDGF, HB-EGF, bFGF and EGF and cell proliferation and migration induced by HB-EGF. In cultured endothelial cells, adiponectin attenuated HB-EGF expression stimulated by TNFα (Matsuda et al. J. Biol. Chem. 277:37487-37491 (2002), herein incorporated by reference in its entirety). Kubota et al. further showed that the levels of FFAs, triglycerides and total cholesterol of adipoenctin-deficient mice were significantly elevated indicating that the lipid metabolism of these mice was severely disrupted and the mice were hyperlipidemic (Kubota et al., J. Biol. Chem. 277:25863-25866 (2002), herein incorporated by reference in its entirety). Adiponectin therefore has antiatherogenic properties.

In a separate study of adiponectin-KO mice, Maeda et al found that there was delayed clearance of FFA in plasma, low levels of fatty acid transport protein 1 (FATP1) mRNA in muscle, high levels of TNFα mRNA in adipose tissue and high plasma TNFα concentrations. These KO mice exhibited severe diet-induced insulin resistence with reduced insulihn-receptor substrate 1 (IRS-1)-associated phosphatidyl inositol 3 (PI3)-kinase activity in the muscles. Adenovirus-mediated adiponectin expression in the KO mice reversed the increase of adipose TNFα mRNA and the diet-induced insulin resistance. In cultured myocytes, TNFα decreased FATP1 mRNA, IRS1-associated PI3-kinase activity and glucose uptake whereas adiponectin increased these parameters supporting the similar observations in mice (Maeda et al., Nature Med. 8:731-737 (2002), herein incorporated by reference in its entirety).

Hotta et al. have shown that plasma levels of adiponectin are decreased in Type 2 diabetes patients with coronary artery disease (CAD) complications and may cause the develoment of insulin resistance in these patients. In addition, the plasma adiponectin levels independently negatively correlated with serum triglyceridemia levels suggesting decreased adiponectin is associated with hypertriglyceridemia which is known to play a significant role in the deveopment of atherosclerosis. In addition, sex differences were observed in adiponectin concentrations in the diabetic subjects without CAD with higher higher levels in clinically normal women as well as in diabetic women suggesting that sex hormones including estrogen, progesterone and androgen may affect plasma adiponectin levels (Hotta et al., Arterioscler. Thromb. Vasc. Biol. 20:1595-1599 (2000), herein incorporated by reference in its entirety). The plasma levels of adiponectin is also reduced in cardiovascular patients with end stage renal disease and the incidence of cardiovascular death is higher in renal failure patients with low plasma adiponectins compared with those with higher plasma adiponectin levels (Zoccali et al., J Am Soc Nephrol 13:134-41 (2002), herein incorporated by reference in its entirety). These data clearly show that adiponectin is involved in metabolic disorders including diabetes cardiovascular disease with and without renal complications.

Based on these studies and others, therapeutics that increase plasma adiponectin should be useful in preventing metabolic disorders, diabetes, cardiovascular and other related disorders such as atherogenesis, hypertriglyceridemia, vascular stenosis after angioplasty. Thus, the adiponectin-like polypeptides and polynucleotides of the invention may be used to treat obesity, diabetes, lipoatrophy, coronary artery diseases, atherosclerosis, and other obesity and diabetes-related cardiovascular pathologies. Adiponectin-like polypeptides and polynucleotides of the invention may also be used in treatment of autoimmune diseases and inflammation, to modulate immune responses, and to treat transplant patients. Adiponectin-like polypetides may also be used in the treatment of tumors such as solid tumors and leukemia

This invention discloses two adiponectin-like polypeptides, SEQ ID NO: 44 and 50. The first adiponectin-like polypeptide of the invention (SEQ ID NO: 44) is an approximately 287-amino acid protein with a predicted molecular mass of approximately 31.5-kDa unglycosylated. The initial methionine starts at position 458 of SEQ ID NO: 43 and the putative stop codon begins at positions 1315 of SEQ ID NO: 43. A predicted approximately twenty one-residue signal peptide is encoded from approximately residue 1 through residue 21 of SEQ ID NO: 44. The mature protein without the signal peptide is useful on its own. It may be confirmed by expression in mammalian cells and sequencing of the cleared product. The signal peptide was predicted using Neural Network SignalP V1.1 program (Nielsen et al, Int. J. Neur. Syst. 8:581 (1997), herein incorporated by reference in its entirety). One of skill in the art will recognize that the cleavage site may be different than that predicted by the computer program. SEQ ID NO: 46 is the resulting peptide when the signal peptide is removed from SEQ ID NO: 44.

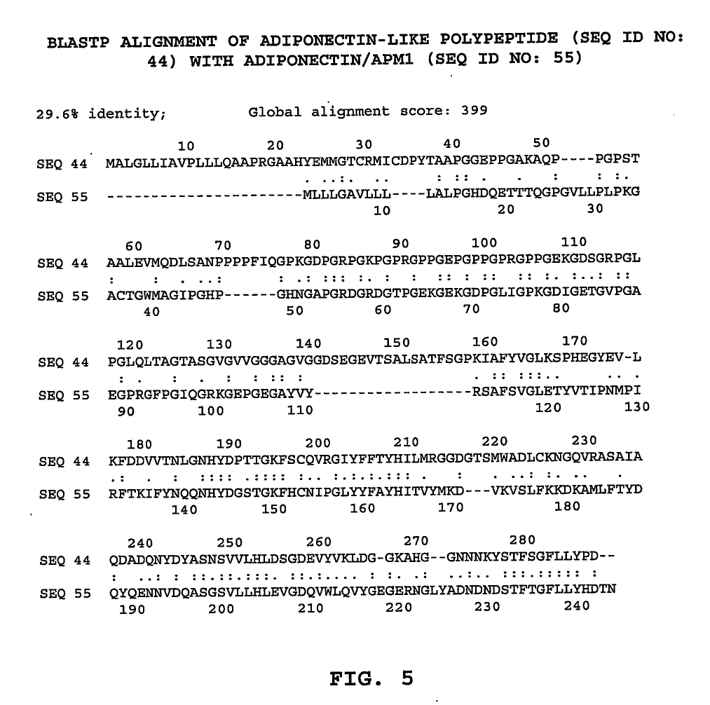

Protein database searches with the BLASTP algorithm (Altschul et al., J. Mol. Evol. 36:290-300 (1993) and Altschul et al., J. Mol. Biol. 21:403-10 (1990), herein incorporated by reference) indicate that SEQ ID NO: 43-44 and 46 are homologous to C1q domain containing proteins. FIG. 5 shows the BLASTP amino acid sequence alignment between adiponectin-like polypeptide SEQ ID NO: 44 and human adiponectin amino acid sequence ID NO: 55 (gi4757760, Apm1/adiponectin), indicating that the two sequences share 50% similarity over 233 amino acids and 36% identity over the same 233 amino acids, wherein A=Alanine, C=Cysteine, D=Aspartic Acid, E=Glutamic Acid, F=Phenylalanine, G=Glycine, H=Histidine, I=Isoleucine, K=Lysine, L=Leucine, M=Methionine, N=Asparagine, P=Proline, Q=Glutamine, R=Arginine, S=Serine, T=Threonine, V=Valine, W=Tryptophan, Y=Tyrosine. Gaps are presented as dashes. One of ordinary skill in the art accepts homology based on amino acid sequence identity as a credible method of determining the function of a polypeptide. See Henikoff, et al., Science, 278:609-614 (1997), herein incorporated by reference in its entirety.

Polypeptides of the invention encoded by SEQ ID NO: 44, 46-48, like adiponectin (gi4757760) may function to attenuate the inflammatory responses, for example by suppressing TNF-α-induced monocyte adhesion to human aortic endothelial cells in a manner similar to adiponectin (Ouchi et al., Circulation 100:2473-2476 (1999), herein incorporated by reference in its entirety), prevent or decrease neotintimal thickening of arteries observed in artherosclerosis and in restenosis after angioplasty, decrease scavenger receptor levels and reduce intracellular cholesteryl ester content resulting in the transformation of macrophages to foam cells (Ouchi et al., Circulation 103:1057-63 (2001), herein incorporated by reference in its entirety), modulate serum FFAs, total cholesterol and triglyceride levels (Kubota et al., J. Biol. Chem. 277:25863-25866 (2002); Hotta et al. Arterioscler. Thromb. Vasc. Biol. 20:1595-1599 (2000), both of which are herein incorporated by reference in their entirety), modulate the expression of cell adhesion molecules and integrins such as VCAM-1, ICAM-1, E-selectin associated with atherosclerosis, diabetes, cardiovascular, restenosis and other related metabolic disorders. Polypetides encoded by SEQ ID NO: 44, 46-48 may also function to modulate cancer development due to modulating myelomonocytic progenitor cell proliferation via apoptotic pathways, as is observed for adiponectin. Like adiponectin, polypeptides of SEQ ID NO:44, 46-48 may also function modulate glucose metabolism by affecting plasma glucose levels, glucose trasport and their catabolism in muscle and modulate insulin-resistence.

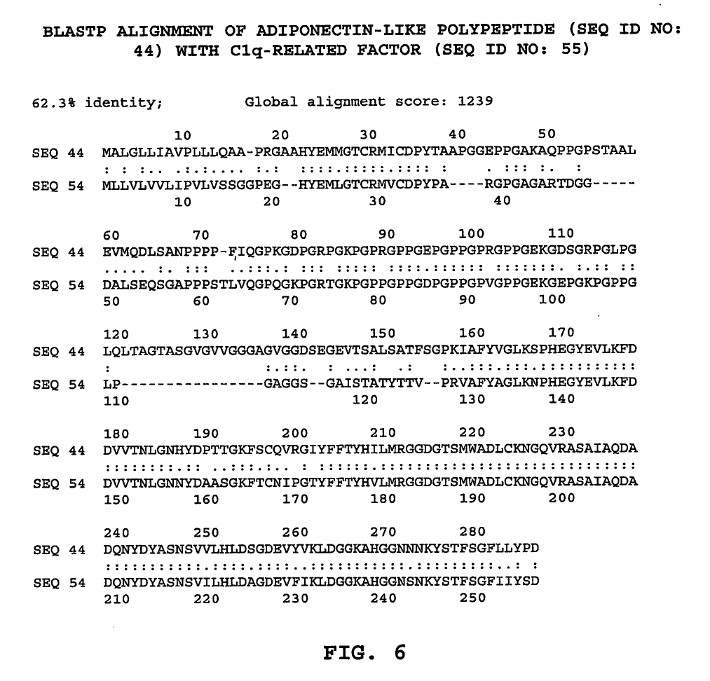

FIG. 6 shows the BLASTP amino acid sequence alignment between adiponectin-like polypeptide SEQ ID NO: 44 and SEQ ID NO: 54 (gi3747097, a C1q-related factor), indicating that the two sequences share 76% similarity over 206 amino acid residues and 67% identity over the same 206 amino acid residues, wherein A=Alanine, C=Cysteine, D=Aspartic Acid, E=Glutamic Acid, F=Phenylalanine, G=Glycine, H=Histidine, I=Isoleucine, K=Lysine, L=Leucine, M=Methionine, N=Asparagine, P=Proline, Q=Glutamine, R=Arginine, S=Serine, T=Threonine, V=Valine, W=Tryptophan, Y=Tyrosine. Gaps are presented as dashes.

Using the Pfam software program (Sonnhammer et al., Nucleic Acids Res., 26:320-322 (1998) herein incorporated by reference), adiponectin-like polypeptide of SEQ ID NO: 43-44 and 64 revealed highly significant structural homology to adiponectin in having conserved collagen and C1q domains (PFO1391 and PF00386 respectively) at E-values of 2.1e-06 and 7.7e-31. The exact sequences of the collagen and C1q domains are listed as SEQ ID NO: 47 and SEQ ID NO: 48 respectively. Further description of the Pfam models can be 15 found at the Pfam homepage website hosted by the Washington University at St. Louis. Using eMATRIX software package (Stanford University, Stanford, Calif.) (Wu et al., J. Comp. Biol., 6:219-235 (1999), herein incorporated by reference), adiponectin-like polypeptide of SEQ ID NO: 44 was determined to have following eMATRIX domain hits. The results in Table 6 describe the identity and location of significant eMATRIX domains present in the corresponding SEQ ID NO: 44.

TABLE 6

| No. | E-value | Score | Accession No. | Domain Description | Amino acids |
|---|---|---|---|---|---|
| 1 | 1.675e−24 | 18.26 | BL01113B | C1q domain proteins. | 174-210 |
| 2 | 1.871e−15 | 17.99 | BL01113A | C1q domain proteins. | 85-112 |
| 3 | 5.091e−14 | 17.99 | BL01113A | C1q domain proteins. | 82-109 |
| 4 | 3.250e−13 | 7.47 | BL01113D | C1q domain proteins. | 277-287 |
| 5 | 4.892e−13 | 17.99 | BL01113A | C1q domain proteins. | 76-103 |
| 6 | 6.108e−13 | 17.99 | BL01113A | C1q domain proteins. | 94-121 |
| 7 | 6.936e−13 | 19.33 | PR00007A | COMPLEMENT C1Q DOMAIN | 168-195 |
| | | | | SIGNATURE | |
| 8 | 9.250e−13 | 15.60 | PR00007C | COMPLEMENT C1Q DOMAIN | 243-265 |
| | | | | SIGNATURE | |
| 9 | 9.372e−13 | 14.16 | PR00007B | COMPLEMENT C1Q DOMAIN | 195-215 |
| | | | | SIGNATURE | |
| 10 | 9.757e−13 | 17.99 | BL01113A | C1q domain proteins. | 79-106 |
| 11 | 3.769e−12 | 17.99 | BL01113A | C1q domain proteins. | 88-115 |
| 12 | 6.308e−12 | 17.99 | BL01113A | C1q domain proteins. | 91-118 |
| 13 | 9.294e−12 | 13.18 | BL01113C | C1q domain proteins. | 243-263 |
| 14 | 5.500e−11 | 9.64 | PR00007D | COMPLEMENT C1Q DOMAIN | 275-286 |
| | | | | SIGNATURE | |
| 15 | 8.159e−11 | 17.99 | BL01113A | C1q domain proteins. | 70-97 |
| 16 | 9.795e−11 | 17.99 | BL01113A | C1q domain proteins. | 97-124 |
| 17 | 9.809e−10 | 17.99 | BL01113A | C1q domain proteins. | 73-100 |
| 18 | 6.019e−09 | 17.99 | BL01113A | C1q domain proteins. | 103-130 |

The polypeptide fragments corresponding to the C1q domains mentioned in Table 6 either together, individually or combinations thereof perform the functions observed with the full-length adiponectin mentioned earlier as seen with adiponectin in mice (Freubis et al. Proc. Natl. Acad. Sci. USA. 98:2005-2010 (2001), herein incorporated by reference in its entirety).

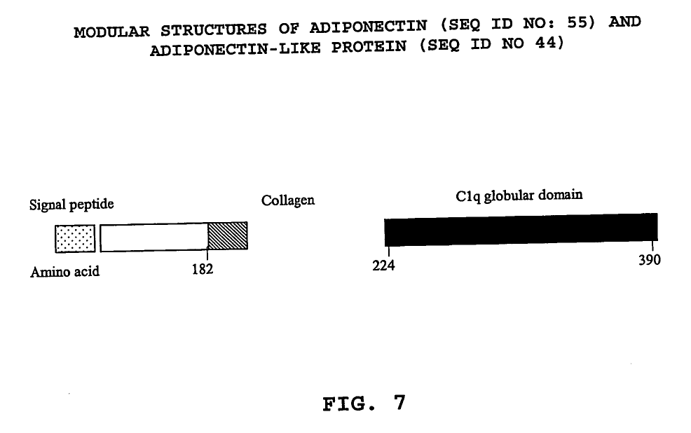

FIG. 7 shows the modular structures of both adiponectin (gi4757760) and SEQ ID NO: 44. Both sequences have a leading signal peptide, a unique domain followed by a collagen-like domain and the globular C1q domain.

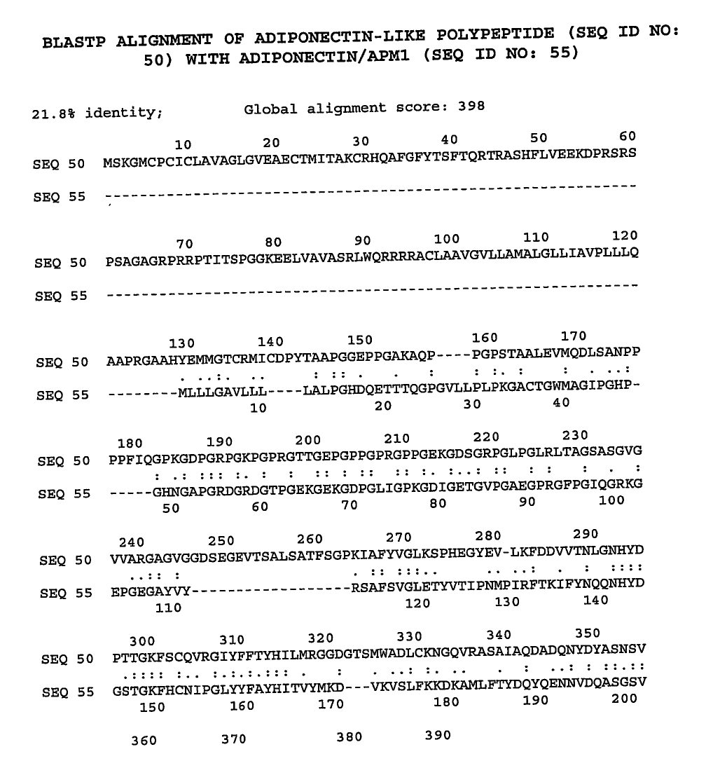

The second adiponectin-like polypeptide of the invention (SEQ ID NO: 50) is an approximately 392-amino acid protein with a predicted molecular mass of approximately 43.12 kDa unglycosylated. The initial methionine starts at position 88 of SEQ ID NO: 49 and the putative stop codon begins at positions 1263 of SEQ ID NO: 49. Protein database searches with the BLASTP algorithm (Altschul et al., J. Mol. Evol. 36:290-300 (1993) and Altschul et al., J. Mol. Biol. 21:403-10 (1990), herein incorporated by reference) indicate that SEQ ID NO: 50 is homologous to adiponectin. FIG. 8 shows the BLASTP amino acid sequence alignment between adiponectin-like polypeptide SEQ ID NO: 50 and human adiponectin amino acid sequence ID NO: 55 (gi4757760), indicating that the two sequences share 50% similarity over 233 amino acid residues and 36% identity over the same 233 amino acid residues, wherein A=Alanine, C=Cysteine, D=Aspartic Acid, E=Glutamic Acid, F=Phenylalanine, G=Glycine, H=Histidine, I=Isoleucine, K=Lysine, L=Leucine, M=Methionine, N=Asparagine, P=Proline, Q=Glutamine, R=Arginine, S=Serine, T=Threonine, V=Valine, W=Tryptophan, Y=Tyrosine. Gaps are presented as dashes.

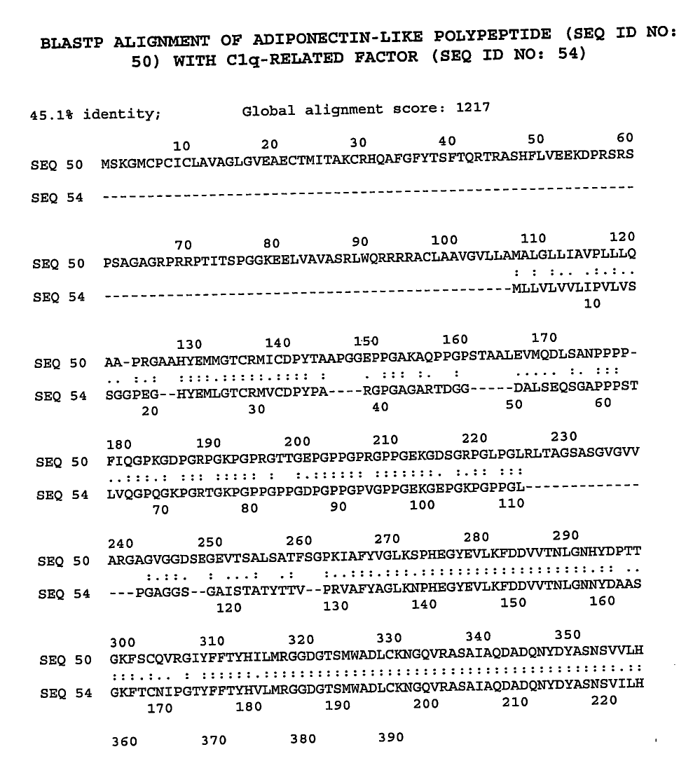

FIG. 9 shows the BLAST? amino acid sequence alignment between adiponectin-like polypeptide SEQ ID NO: 50 and SEQ ID NO: 54 (gi3747097, a C1q-related factor), indicating that the two sequences share 78% similarity over 200 amino acid residues and 67% identity over the same 200 amino acid residues, wherein A=Alanine, C=Cysteine, D=Aspartic Acid, E=Glutamic Acid, F=Phenylalanine, G=Glycine, H=Histidine, I=Isoleucine, K=Lysine, L=Leucine, M=Methionine, N=Asparagine, P=Proline, Q=Glutamine, R=Arginine, S=Serine, T=Threonine, V=Valine, W=Tryptophan, Y=Tyrosine. Gaps are presented as dashes

Using the Pfam software program (Sonnhammer et al., Nucleic Acids Res., 26:320-322 (1998) herein incorporated by reference), adiponectin-like polypeptide of SEQ ID NO: 50 revealed highly significant structural homology to adiponectin in having conserved collagen and C1q domains (PFO1391 and PF00386 respectively) at E-values of 2.1e-06 and 7.7e-31. The exact sequences of the collagen and C1q domains are listed as SEQ ID NO: 47 and SEQ ID NO: 48 respectively. Further description of the Pfam models can be found at the Pfam homepage website hosted by the Washington University at St. Louis.

Using eMATRIX software package (Stanford University, Stanford, Calif.) (Wu et al., J. Comp. Biol., 6:219-235 (1999), herein incorporated by reference), adiponectin-like polypeptide of SEQ ID NO: 47 was determined to have following eMATRIX domain hits. The results in Table 7 describe the identity and location of significant eMATRIX domains present in corresponding SEQ ID NO: 48.

TABLE 7

| No. | E-value | Score | Accession No. | Description | Amino acids |
|---|---|---|---|---|---|
| 1 | 1.675e−24 | 18.26 | BL01113B | C1q domain proteins. | 280-316 |
| 2 | 4.194e−15 | 17.99 | BL01113A | C1q domain proteins. | 200-227 |
| 3 | 3.250e−13 | 7.47 | BL01113D | C1q domain proteins. | 383-393 |
| 4 | 3.919e−13 | 17.99 | BL01113A | C1q domain proteins. | 191-218 |
| 5 | 6.936e−13 | 19.33 | PR00007A | COMPLEMENT C1Q DOMAIN | 274-301 |
| | | | | SIGNATURE | |
| 6 | 9.250e−13 | 15.60 | PR00007C | COMPLEMENT C1Q DOMAIN | 349-371 |
| | | | | SIGNATURE | |
| 7 | 9.372e−13 | 14.16 | PR00007B | COMPLEMENT C1Q DOMAIN | 301-321 |
| | | | | SIGNATURE | |
| 8 | 9.294e−12 | 13.18 | BL01113C | C1q domain proteins. | 349-369 |
| 9 | 5.500e−11 | 17.99 | BL01113A | C1q domain proteins. | 185-212 |
| 10 | 5.500e−11 | 9.64 | PR00007D | COMPLEMENT C1Q DOMAIN | 381-392 |
| | | | | SIGNATURE | |
| 11 | 6.727e−11 | 17.99 | BL01113A | C1q domain proteins. | 182-209 |
| 12 | 8.773e−11 | 17.99 | BL01113A | C1q domain proteins. | 203-230 |
| 13 | 3.681e−10 | 17.99 | BL01113A | C1q domain proteins. | 188-215 |
| 14 | 6.936e−10 | 17.99 | BL01113A | C1q domain proteins. | 176-203 |
| 15 | 7.319e−10 | 17.99 | BL01113A | C1q domain proteins. | 194-221 |
| 16 | 4.635e−09 | 17.99 | BL01113A | C1q domain proteins. | 209-236 |
| 17 | 5.500e−09 | 17.99 | BL01113A | C1q domain proteins. | 179-206 |

The polypeptide fragments corresponding to the C1q domains mentioned in Table 7 either together, individually or combinations thereof perform the functions observed with the full-length adiponectin mentioned earlier as seen with adiponectin in mice (Freubis et al. Proc. Natl. Acad. Sci. USA. 98:2005-2010 (2001), herein incorporated by reference in its entirety).

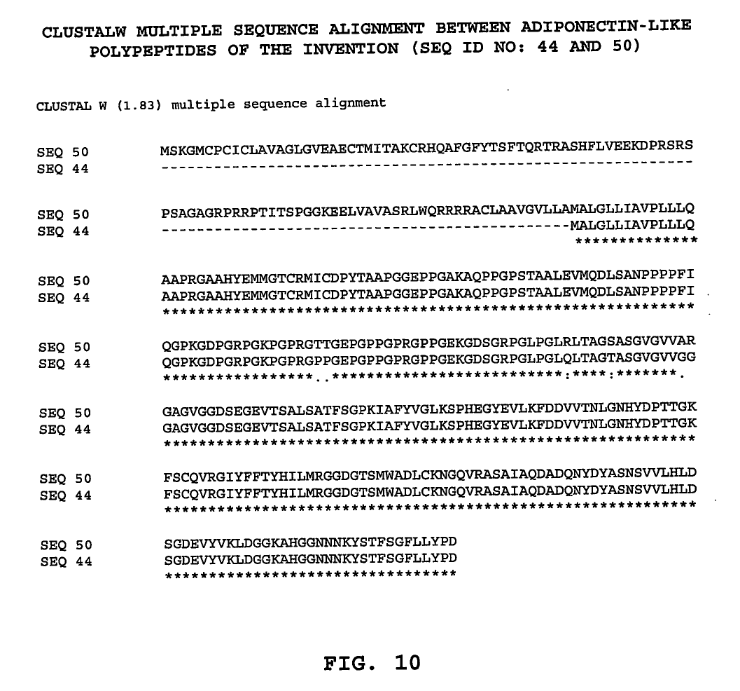

FIG. 10 shows a multiple sequence alignment between the two adiponectin-like polypeptides of the invention (SEQ ID NO: 44 and 50) and adiponectin (SEQ ID NO: 55), wherein asterisks (*) represent identical amino acids, colons (:) represent conservative substitutions, and periods (.) represent semi-conservative substitutions. Gaps are represented as dashes.

Polypeptides of the invention encoded by SEQ ID NO: 50, like adiponectin (gi4757760) may function to attenuate the inflammatory responses, for example by suppressing TNF-α-induced monocyte adhesion to human aortic endothelial cells in a manner similar to adiponectin (Ouchi et al, Circulation 100:2473-2476 (1999), herein incorporated by reference in its entirety), prevent or decrease neotintimal thickening of arteries observed in artherosclerosis and in restenosis after angioplasty, decrease scavenger receptor levels and reduce intracellular cholesteryl ester content resulting in the transformation of macrophages to foam cells (Ouchi et al, Circulation 103:1057-63 (2001), herein incorporated by reference in its entirety), modulate serum FFAs, total cholesterol and triglyceride levels (Kubota et al. J. Biol. Chem. 277(29):25863-25866 (2002); Hotta et al. Arterioscler. Thromb. Vasc. Biol. 20:1595-1599 (2000), both of which are herein incorporated by reference in their entirety), modulate the expression of cell adhesion molecules and integrins such as VCAM-1, ICAM-1, E-selectin associated with atherosclerosis, diabetes, cardiovascular, restenosis and other related metabolic disorders. Polypetides encoded by SEQ ID NO: 44, 46-48, 50, 52-53 may also function to modulate cancer development due to modulating myelomonocytic progenitor cell proliferation via apopotitic pathways, as is observed for adiponectin. Like adiponectin, polypeptides of SEQ ID NO: 44, 46-48, 50, 52-53 may also function modulate glucose metabolism by affecting plasma glucose levels, glucose transport and their catabolism in muscle and modulate insulin-resistence.

The adiponectin-like polypeptides and polynucleotides of the invention may be used to treat carbohydrate and lipid disorders including but not limited to obesity, diabetes, lipoatrophy, coronary artery diseases, atherosclerosis and other obesity and diabetes-related pathologies. Adiponectin-like polypetides and polynucleotides of the invention may also be used in the treatment of autoimmune diseases and iinflammation to modulate immune responses and to treat transplant patients.

4.4 Ly-6-Like Polypeptide

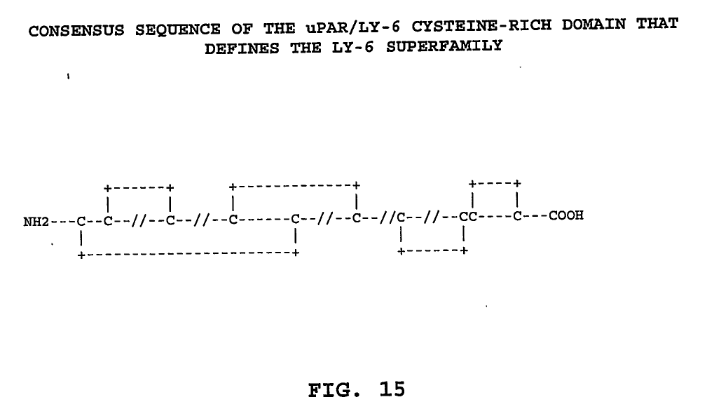

A variety of cell-surface proteins that are bound to the cell surface by either a glycosylphosphatidyl inositol (GPI) anchor or association with other cell surface proteins are part of the Ly-6 family of proteins. The characteristic feature of this family is a cysteine-rich domain that consists often cysteine residues that are involved in five disulfide bonds (Behrendt et al., J. Biol. Chem. 266:7842-7847 (1991); Ploug et al., J. Biol. Chem. 268:17539-17546 (1993), both of which are herein incorporated by reference). The residues between the cysteines are not conserved; however, the spacing between the cysteines is conserved (see FIG. 15). The cysteine-rich domain is named the uPAR/Ly-6 domain after two exemplary family members, the mouse Ly-6 antigen and human urolinase-type plasminogen activator receptor (uPAR).

Ly-6 antigen is a murine cell surface molecule that is orthologous to human CD59. CD59 is a widely distributed membrane-bound inhibitor of the cytolytic membrane attack complex (MAC) of complement. The MAC is formed by the sequential assembly of terminal complement proteins that is initiated by and directed against invading microorganisms and occasionally against host cells in certain autoimmune and inflammatory conditions (Fletcher et al., Structure 2:185-199 (1994); Yu et al., J. Exp. Med. 185:745-753 (1997), both of which are herein incorporated by reference). UPAR is the only member of the uPAR/Ly-6 family thus far that contains multiple repeats of the cysteine-rich domain and is a GPI-anchored protein that binds urokinase-type plasminogen activator (uPA) which converts plasminogen into plasmin and is involved in thrombolysis and extracellular matrix degradation (Behrendt et al., supra 1991; Ploug et al., supra 1993). Expression of uPA and uPAR has been associated with increased tumor cell invasion and metastasis in several malignancies including breast cancer (Guo et al, Cancer Res. 62:4678-4684 (2002), herein incorporated by reference). In general uPAR/Ly-6 proteins are likely to be involved in protein binding and are believed to function as receptor-like molecules. Thus, there exists a need for identifying further members of this family of proteins.

This invention relates to seven Ly-6-like polypeptides. The Ly-6-like polypeptide of SEQ ID NO: 58 is an approximately 98-amino acid protein with a predicted molecular mass of approximately 11-kDa unglycosylated. The initial methionine starts at position 6 of SEQ ID NO: 57 and the putative stop codon begins at position 300 of SEQ ID NO: 57. A signal peptide of 20 residues is predicted from approximately residue 1 to residue 20 of SEQ ID NO: 58. The extracellular portion is useful on its own. The signal peptide region was predicted using the Neural Network SignalP V1.1 program (Nielsen et al, Int. J. Neural Syst. 8:581-599 (1997)). One of skill in the art will recognize that the actual cleavage site may be different than that predicted by the computer program.

The Ly-6-like polypeptide of SEQ ID NO: 65 is an approximately 114-amino acid protein with a predicted molecular mass of approximately 13-kDa unglycosylated. The initial methionine starts at position 1 of SEQ ID NO: 64 and the putative stop codon begins at position 343 of SEQ ID NO: 64. A signal peptide of 21 residues is predicted from approximately residue 1 to residue 21 of SEQ ID NO: 65. The extracellular portion is useful on its own. The signal peptide region was predicted using the Neural Network SignalP V1.1 program (Nielsen et al, Int. J. Neural Syst. 8:581-599 (1997)). One of skill in the art will recognize that the actual cleavage site may be different than that predicted by the computer program.

A splice variant of SEQ ID NO: 65 is SEQ ID NO: 71. The splice site occurs after nucleotide 89 of SEQ ID NO: 64. The splice variant is an approximately 126 amino acid protein with a predicted molecular mass of approximately 14 kDa unglycosylated. The initial methionine starts at position 25 of SEQ ID NO: 70 and the putative stop codon begins at position 403 of SEQ ID NO: 70. A signal peptide of 21 residues is predicted from approximately residue 1 to residue 21 of SEQ ID NO: 71. The extracellular portion is useful on its own. The signal peptide region was predicted using the Neural Network SignalP V1.1 program (Nielsen et al, Int. J. Neural Syst. 8:581-599 (1997)). One of skill in the art will recognize that the actual cleavage site may be different than that predicted by the computer program.

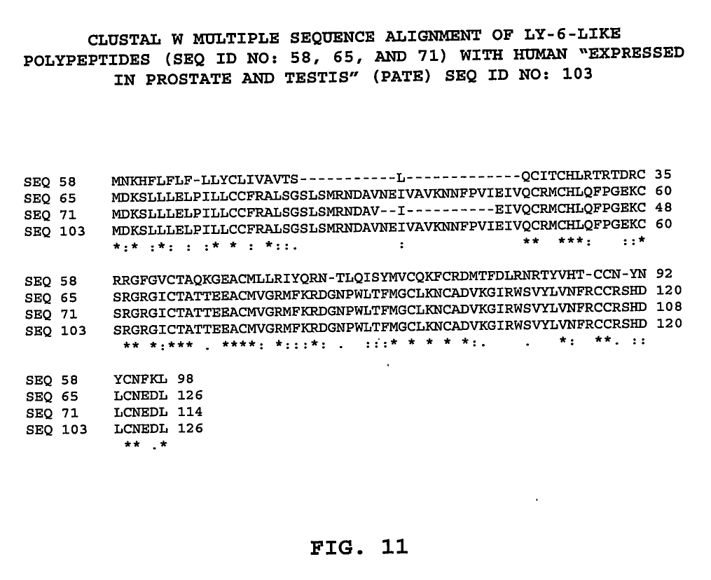

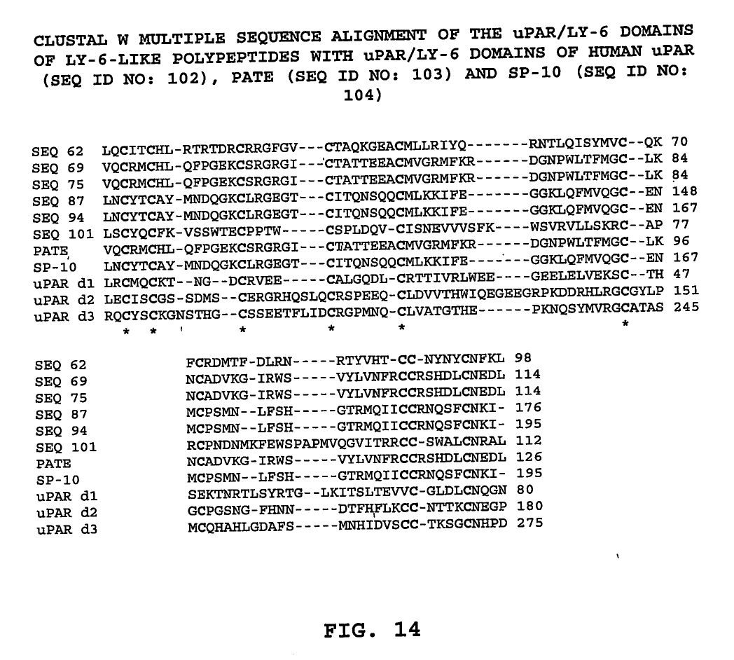

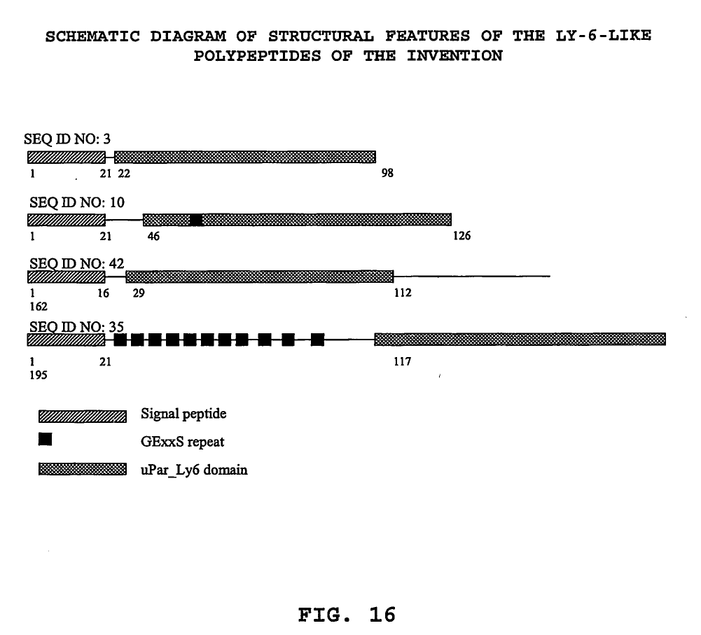

Protein database searches with the BLASTP algorithm (Altschul S. F. et al., J. Mol. Evol. 36:290-300 (1993) and Altschul S. F. et al., J. Mol. Biol. 21:403-10 (1990), herein incorporated by reference) indicate that SEQ ID NO: 58, 65 and 71 are homologous to human PATE (expressed in prostate and testis), a member of the Ly-6 superfamily that is expressed specifically in prostate cancer, normal prostate and testis, thereby being a potential candidate for immunotherapy of prostate cancer (Bera et al., Proc. Natl. Acad. Sci. USA 99:3058-3063 (2002), herein incorporated by reference). A multiple sequence alignment of SEQ ID NO: 58, 65 and 71 with human PATE is shown in FIG. 11. A distinctive feature of Ly-6 family members is the presence of a conserved cysteine-rich domain wherein cysteine residues are conserved and the spacing between the cysteines is mostly conserved. FIG. 15 depicts the consensus sequence for the uPAR/Ly-6 domain. SEQ ID NO: 58 contains a uPAR/Ly-6 cysteine-rich domain spanning residues 21 to 98 (SEQ ID NO: 62), SEQ ID NO: 65 contains a uPAR/Ly-6 cysteine-rich domain spanning residues 34 to 114 (SEQ ID NO: 69), and SEQ ID NO: 71 contains a uPAR/Ly-6 cysteine-rich domain spanning residues 46 to 114 (SEQ ID NO: 75) and are shown in FIG. 14 wherein the conserved cysteine residues are in bold and labeled with an asterisk (*). SEQ ID NO: 65 also contains a (GEXXS)n repeat (SEQ ID NO: 106) spanning residues 57 to 61 in the uPAR/Ly-6 cysteine-rich domain (FIG. 16).

The Ly-6-like polypeptide of SEQ ID NO: 78 is an approximately 155-amino acid protein with a predicted molecular mass of approximately 17-kDa unglycosylated. The initial methionine starts at position 95 of SEQ ID NO: 77 and the putative stop codon begins at position 560 of SEQ ID NO: 77. A signal peptide of 21 residues is predicted from approximately residue 1 to residue 21 of SEQ ID NO: 78. The extracellular portion is useful on its own. The signal peptide region was predicted using the Neural Network SignalP V1.1 program (Nielsen et al, Int. J. Neural Syst. 8:581-599 (1997)). One of skill in the art will recognize that the actual cleavage site may be different than that predicted by the computer program.

A splice variant of SEQ ID NO: 78 is SEQ ID NO: 83. The splice site occurs after nucleotide 381 of SEQ ID NO: 77. The splice variant is an approximately 176 amino acid protein with a predicted molecular mass of approximately 19 kDa unglycosylated. The initial methionine starts at position 95 of SEQ ID NO: 82 and the putative stop codon begins at position 623 of SEQ ID NO: 82. A signal peptide of 21 residues is predicted from approximately residue 1 to residue 21 of SEQ ID NO: 83. The extracellular portion is useful on its own. The signal peptide region was predicted using the Neural Network SignalP V1.1 program (Nielsen et al, Int. J. Neural Syst. 8:581-599 (1997)). One of ski in the art will recognize that the actual cleavage site may be different than that predicted by the computer program.

A splice variant of SEQ ID NO: 78 is SEQ ID NO: 90. The splice site occurs after nucleotide 438 of SEQ ID NO: 77. The splice variant is an approximately 195 amino acid protein with a predicted molecular mass of approximately 21 kDa unglycosylated. The initial methionine starts at position 177 of SEQ ID NO: 77 and the putative stop codon begins at position 762 of SEQ ID NO: 89. A signal peptide of 21 residues is predicted from approximately residue. 1 to residue 21 of SEQ ID NO: 90. The extracellular portion is useful on its own. The signal peptide region was predicted using the Neural Network SignalP V1.1 program (Nielsen et al, Int. J. Neural Syst. 8:581-599 (1997)). One of skill in the art will recognize that the actual cleavage site may be different than that predicted by the computer program.

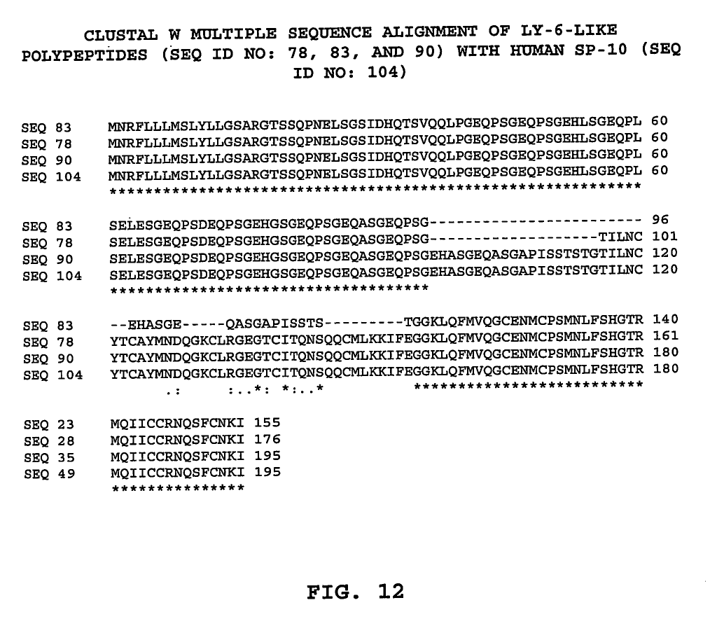

Protein database searches with the BLASTP algorithm (Altschul S. F. et al., J. Mol. Evol. 36:290-300 (1993) and Altschul S. F. et al., J. Mol. Biol. 21:403-10 (1990), herein incorporated by reference) indicate that SEQ ID NO: 78, 83 and 90 are homologous to human sperm antigen SP-10, a member of the Ly-6 superfamily that is detected specifically in the acrosome of developing round spermatids as well as associated with the acrosomal membrane and matrix of mature sperm. Functional assays have demonstrated that anti-SP-10 antisera inhibit sperm-egg interactions, thus SP-10 is a potential candidate for a contraceptive vaccine immunogen (Wright et al., Biol. Reprod. 49:316-325 (1993), herein incorporated by reference). A multiple sequence alignment of SEQ ID NO: 78, 83 and 90 with human SP-10 is shown in FIG. 12. SEQ ID NO: 83 contains a uPAR/Ly-6 cysteine-rich domain spanning residues 99 to 176 (SEQ ID NO: 87) and SEQ ID NO: 90 contains a uPAR/Ly-6 cysteine-rich domain spanning residues 118 to 195 (SEQ ID NO: 94), and are shown in FIG. 14 wherein the conserved cysteine residues are in bold and labeled with an asterisk (*). SEQ ID NO: 90 also contains eleven (11) (GEXXS)n repeats (SEQ ID NO: 106) spanning residues 41 to 105 and are located between the signal peptide and the uPAR/Ly-6 cysteine-rich domain (FIG. 16).

The Ly-6-like polypeptide of SEQ ID NO: 97 is an approximately 162-amino acid protein with a predicted molecular mass of approximately 18-kDa unglycosylated. The initial methionine starts at position 1 of SEQ ID NO: 96 and the putative stop codon begins at position 489 of SEQ ID NO: 96. A signal peptide of 16 residues is predicted from approximately residue 1 to residue 16 of SEQ ID NO: 97. The extracellular portion is useful on its own. The signal peptide region was predicted using the Neural Network SignalP V1.1 program (Nielsen et al, Int. J. Neural Syst. 8:581-599 (1997)). One of skill in the art will recognize that the actual cleavage site may be different than that predicted by the computer program.

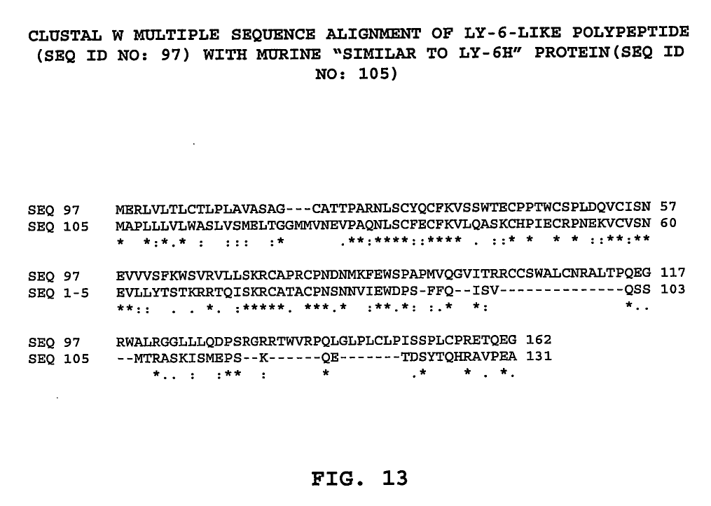

Protein database searches with the BLASTP algorithm (Altschul S. F. et al., J. Mol. Evol. 36:290-300 (1993) and Altschul S. F. et al., J. Mol. Biol. 21:403-10 (1990), herein incorporated by reference) indicate that SEQ ID NO: 97 is homologous to murine “similar to lymphocyte antigen Ly-6H precursor”, a member of the Ly-6 superfamily that plays a role in immune function (Mallya et al., Genomics 80:113-123 (2002), herein incorporated by reference). An alignment of SEQ ID NO: 97 with murine “similar to lymphocyte antigen Ly-6H precursor” is shown in FIG. 13. SEQ ID NO: 97 contains a uPAR/Ly-6 cysteine-rich domain spanning residues 28 to 112 (SEQ ID NO: 101) and is shown in FIG. 14 wherein the conserved cysteine residues are in bold and labeled with an asterisk (*).

The Ly-6-like polypeptides of the invention are expected to have similar functions as the Ly-6 family members described above. PATE is expressed specifically in prostate cancer, normal prostate and testis and therefore is a potential immunotherapeutic target for treatment of prostate cancer (Bera et al., supra 2002). Since SEQ ID NO: 58, 65, and 71 are homologous to PATE, it is believed that they will also be useful in treating prostate cancer as well as other diseases and disorders of the prostate and testis.

Human sperm antigen SP-10 is expressed in the developing acrosome of round spermatids and is later associated with the acrosomal membrane and matrix of mature sperm (Wright et al., supra 1993). SP-10 is believed to be useful as a vaccine for immunocontraception since anti-SP-10 antisera inhibits sperm-egg interactions (Wright et al., supra 1993) and may be involved in the mechanisms regulating spermatogenesis (Reddi et al., J. Reprod. Immunol. 53:25-36 (2002), herein incorporated by reference). SEQ ID NO: 78, 83, and 90 are believed to function similarly to SP-10 and therefore are potential immunocontraceptive vaccine candidates as well as potential regulators of spermatogenesis.

The human counterpart of murine Ly-6 is CD59 which plays a role in inhibiting the cytolytic membrane attack complex (MAC) of complement. MAC is activated and directed against invading microorganisms, but can also be directed agains host cells under certain conditions, most notably in some autoimmune and inflammatory conditions. In addition to causing cell lysis, at sublethal concentrations MAC on host cells can also mediate various inflammatory processes that elicit severe pathological effects (Yu et al., supra 1997). Host cells are normally protected from MAC by CD59, thus CD59 can be used as a therapeutic for autoimmune and inflammatory diseases. In addition, MAC-mediated tissue destruction is responsible for rejection of porcine organs and CD59 expression on transgenic animal organs has been shown to protect them from complement-mediated damage and prolongs their survival after trasplantation (McCurry et al., Nature Med. 1:423-427 (1995); Roush Science 270:234-235 (1995); Fodor et al., Proc. Natl. Acad. Sci. USA 91:11153-11157 (1994); Byrne et al., Transplantation 60:1149-1156 (1995), all of which are incorporated by reference). Thus, CD59 and SEQ ID NO: 97 are potential candidates to inhibit rejection of xenografts from humoral injury.

4.5 Definitions

It must be noted that as used herein and in the appended claims, the singular forms “a”, “an” and “the” include plural references unless the context clearly dictates otherwise.

The term “active” refers to those forms of the polypeptide that retain the biologic and/or immunologic activities of any naturally occurring polypeptide. According to the invention, the terms “biologically active” or “biological activity” refer to a protein or peptide having structural, regulatory or biochemical functions of a naturally occurring molecule. Likewise “biologically active” or “biological activity” refers to the capability of the natural, recombinant or synthetic polypeptide of the invention, or any peptide thereof, to induce a specific biological response in appropriate animals or cells and to bind with specific antibodies.

The term “activated cells” as used in this application are those cells which are engaged in extracellular or intracellular membrane trafficking, including the export of secretory or enzymatic molecules as part of a normal or disease process.

The terms “complementary” or “complementarity” refer to the natural binding of polynucleotides by base pairing. For example, the sequence 5′-AGT-3′ binds to the complementary sequence 3′-TCA-5′. Complementarity between two single-stranded molecules may be “partial” such that only some of the nucleic acids bind or it may be “complete” such that total complementarity exists between the single stranded molecules. The degree of complementarity between the nucleic acid strands has significant effects on the efficiency and strength of the hybridization between the nucleic acid strands.

The term “embryonic stem cells (ES)” refers to a cell that can give rise to many differentiated cell types in an embryo or an adult, including the germ cells. The term “germ line stem cells (GSCs)” refers to stem cells derived from primordial stem cells that provide a steady and continuous source of germ cells for the production of gametes. The term “primordial germ cells (PGCs)” refers to a small population of cells set aside from other cell lineages particularly from the yolk sac, mesenteries, or gonadal ridges during embryogenesis that have the potential to differentiate into germ cells and other cells. PGCs are the source from which GSCs and ES cells are derived. The PGCs, the GSCs and the ES cells are capable of self-renewal. Thus these cells not only populate the germ line and give rise to a plurality of terminally differentiated cells that comprise the adult specialized organs, but are able to regenerate themselves. The term “totipotent” refers to the capability of a cell to differentiate into all of the cell types of an adult organism. The term “pluripotent” refers to the capability of a cell to differentiate into a number of differentiated cell types that are present in an adult organism. A pluripotent cell is restricted in its differentiation capability in comparison to a totipotent cell.

The term “expression modulating fragment,” EMF, means a series of nucleotides that modulates the expression of an operably linked ORF or another EMF.

As used herein, a sequence is said to “modulate the expression of an operably linked sequence” when the expression of the sequence is altered by the presence of the EMF. EMFs include, but are not limited to, promoters, and promoter modulating sequences (inducible elements). One class of EMFs is nucleic acid fragments which induce the expression of an operably linked ORF in response to a specific regulatory factor or physiological event.

The terms “nucleotide sequence” or “nucleic acid” or “polynucleotide” or “oligonculeotide” are used interchangeably and refer to a heteropolymer of nucleotides or the sequence of these nucleotides. These phrases also refer to DNA or RNA of genomic or synthetic origin which may be single-stranded or double-stranded and may represent the sense or the antisense strand, to peptide nucleic acid (PNA) or to any DNA-like or RNA-like material. In the sequences, A is adenine, C is cytosine, G is guanine, and T is thymine, while N is A, T, G, or C. It is contemplated that where the polynucleotide is RNA, the T (thymine) in the sequence herein may be replaced with U (uracil). Generally, nucleic acid segments provided by this invention may be assembled from fragments of the genome and short oligonucleotide linkers, or from a series of oligonucleotides, or from individual nucleotides, to provide a synthetic nucleic acid which is capable of being expressed in a recombinant transcriptional unit comprising regulatory elements derived from a microbial or viral operon, or a eukaryotic gene.

The terms “oligonucleotide fragment” or a “polynucleotide fragment”, “portion,” or “segment” or “probe” or “primer” are used interchangeably and refer to a sequence of nucleotide residues which are at least about 5 nucleotides, more preferably at least about 7 nucleotides, more preferably at least about 9 nucleotides, more preferably at least about 11 nucleotides and most preferably at least about 17 nucleotides. The fragment is preferably less than about 500 nucleotides, preferably less than about 200 nucleotides, more preferably less than about 100 nucleotides, more preferably less than about 50 nucleotides and most preferably less than 30 nucleotides. Preferably the probe is from about 6 nucleotides to about 200 nucleotides, preferably from about 15 to about 50 nucleotides, more preferably from about 17 to 30 nucleotides and most preferably from about 20 to 25 nucleotides. Preferably the fragments can be used in polymerase chain reaction (PCR), various hybridization procedures or microarray procedures to identify or amplify identical or related parts of mRNA or DNA molecules. A fragment or segment may uniquely identify each polynucleotide sequence of the present invention. Preferably the fragment comprises a sequence substantially similar to a portion of SEQ ID NO: 1-3, 5, 8, 10, 21, 23, 25, 27, 29, 31, 33, 35, 37-40, 43, 45, 49, 51, 53, 56-57, 59, 76-77, 79, 82, 84, 88-89, 91, 95-96, or 98.

Probes may, for example, be used to determine whether specific mRNA molecules are present in a cell or tissue or to isolate similar nucleic acid sequences from chromosomal DNA as described by Walsh et al. (Walsh, P. S. et al., PCR Methods Appl. 1:241-250 (1992)). They may be labeled by nick translation, Klenow fill-in reaction, PCR, or other methods well known in the art. Probes of the present invention, their preparation and/or labeling are elaborated in Sambrook, J. et al., 1989, Molecular Cloning: A Laboratory Manual, Cold Spring Harbor Laboratory, NY; or Ausubel, F. M. et al., 1989, Current Protocols in Molecular Biology, John Wiley & Sons, New York N.Y., both of which are incorporated herein by reference in their entirety.

The nucleic acid sequences of the present invention also include the sequence information from any of the nucleic acid sequences of SEQ ID NO: 1-3, 5, 8, 10, 21, 23, 25, 27, 29, 31, 33, 35, 37-40, 43, 45, 49, 51, 53, 56-57, 59, 76-77, 79, 82, 84, 88-89, 91, 95-96, or 98. The sequence information can be a segment of SEQ ID NO: 1-3, 5, 8, 10, 21, 23, 25, 27, 29, 31, 33, 35, 37-40, 43, 45, 49, 51, 53, 56-57, 59, 76-77, 79, 82, 84, 88-89, 91, 95-96, or 98 that uniquely identifies or represents the sequence information of SEQ ID NO: 1-3, 5, 8, 10, 21, 23, 25, 27, 29, 31, 33, 35, 37-40, 43, 45, 49, 51, 53, 56-57, 59, 76-77, 79, 82, 84, 88-89, 91, 95-96, or 98. One such segment can be a twenty-mer nucleic acid sequence because the probability that a twenty-mer is fully matched in the human genome is 1 in 300. In the human genome, there are three billion base pairs in one set of chromosomes. Because 4²⁰ possible twenty-mers exist, there are 300 times more twenty-mers than there are base pairs in a set of human chromosomes. Using the same analysis, the probability for a seventeen-mer to be fully matched in the human genome is approximately 1 in 5. When these segments are used in arrays for expression studies, fifteen-mer segments can be used. The probability that the fifteen-mer is fully matched in the expressed sequences is also approximately one in five because expressed sequences comprise less than approximately 5% of the entire genome sequence.

Similarly, when using sequence information for detecting a single mismatch, a segment can be a twenty-five mer. The probability that the twenty-five mer would appear in a human genome with a single mismatch is calculated by multiplying the probability for a full match (1÷4²⁵) times the increased probability for mismatch at each nucleotide position (3×25). The probability that an eighteen mer with a single mismatch can be detected in an array for expression studies is approximately one in five. The probability that a twenty-mer with a single mismatch can be detected in a human genome is approximately one in five.

The term “open reading frame,” ORF, means a series of nucleotide triplets coding for amino acids without any termination codons and is a sequence translatable into protein.

The terms “operably linked” or “operably associated” refer to functionally related nucleic acid sequences. For example, a promoter is operably associated or operably linked with a coding sequence if the promoter controls the transcription of the coding sequence. While operably linked nucleic acid sequences can be contiguous and in the same reading frame, certain genetic elements e.g. repressor genes are not contiguously linked to the coding sequence but still control transcription/translation of the coding sequence.

The term “pluripotent” refers to the capability of a cell to differentiate into a number of differentiated cell types that are present in an adult organism. A pluripotent cell is restricted in its differentiation capability in comparison to a totipotent cell.

The terms “polypeptide” or “peptide” or “amino acid sequence” refer to an oligopeptide, peptide, polypeptide, or protein sequence or fragment thereof and to naturally occurring or synthetic molecules. A polypeptide “fragment,” “portion,” or “segment” is a stretch of amino acid residues of at least about 5 amino acids, preferably at least about 7 amino acids, more preferably at least about 9 amino acids and most preferably at least about 17 or more amino acids. The peptide preferably is not greater than about 200 amino acids, more preferably less than 150 amino acids and most preferably less than 100 amino acids. Preferably the peptide is from about 5 to about 200 amino acids. To be active, any polypeptide must have sufficient length to display biological and/or immunological activity.

The term “naturally occurring polypeptide” refers to polypeptides produced by cells that have not been genetically engineered and specifically contemplates various polypeptides arising from post-translational modifications of the polypeptide including, but not limited to, acetylation, carboxylation, glycosylation, phosphorylation, lipidation and acylation.

The term “translated protein coding portion” means a sequence which encodes for the full length protein which may include any leader sequence or a processing sequence.

The term “mature protein coding sequence” refers to a sequence which encodes a peptide or protein without any leader/signal sequence. The “mature protein portion” refers to that portion of the protein without the leader/signal sequence. The peptide may have the leader sequences removed during processing in the cell or the protein may have been produced synthetically or using a polynucleotide only encoding for the mature protein coding sequence. It is contemplated that the mature protein portion may or may not include an initial methionine residue. The initial methionine is often removed during processing of the peptide.

The term “derivative” refers to polypeptides chemically modified by such techniques as ubiquitination, labeling (e.g., with radionuclides or various enzymes), covalent polymer attachment such as pegylation (derivatization with polyethylene glycol) and insertion or substitution by chemical synthesis of amino acids such as ornithine, which do not normally occur in human proteins.

The term “variant” (or “analog”) refers to any polypeptide differing from naturally occurring polypeptides by amino acid insertions, deletions, and substitutions, created using, e.g., recombinant DNA techniques. Guidance in determining which amino acid residues may be replaced, added or deleted without abolishing activities of interest, may be found by comparing the sequence of the particular polypeptide with that of homologous peptides and minimizing the number of amino acid sequence changes made in regions of high homology (conserved regions) or by replacing amino acids with consensus sequence.

Alternatively, recombinant variants encoding these same or similar polypeptides may be synthesized or selected by making use of the “redundancy” in the genetic code. Various codon substitutions, such as the silent changes which produce various restriction sites, may be introduced to optimize cloning into a plasmid or viral vector or expression in a particular prokaryotic or eukaryotic system. Mutations in the polynucleotide sequence may be reflected in the polypeptide or domains of other peptides added to the polypeptide to modify the properties of any part of the polypeptide, to change characteristics such as ligand-binding affinities, interchain affinities, or degradation/turnover rate.

Preferably, amino acid “substitutions” are the result of replacing one amino acid with another amino acid having similar structural and/or chemical properties, i.e., conservative amino acid replacements. “Conservative” amino acid substitutions may be made on the basis of similarity in polarity, charge, solubility, hydrophobicity, hydrophilicity, and/or the amphipathic nature of the residues involved. For example, nonpolar (hydrophobic) amino acids include alanine, leucine, isoleucine, valine, proline, phenylalanine, tryptophan, and methionine; polar neutral amino acids include glycine, serine, threonine, cysteine, tyrosine, asparagine, and glutamine; positively charged (basic) amino acids include arginine, lysine, and histidine; and negatively charged (acidic) amino acids include aspartic acid and glutamic acid. “Insertions” or “deletions” are preferably in the range of about 1 to 20 amino acids, more preferably 1 to 10 amino acids. The variation allowed may be experimentally determined by systematically making insertions, deletions, or substitutions of amino acids in a polypeptide molecule using recombinant DNA techniques and assaying the resulting recombinant variants for activity.

Alternatively, where alteration of function is desired, insertions, deletions or non-conservative alterations can be engineered to produce altered polypeptides. Such alterations can, for example, alter one or more of the biological functions or biochemical characteristics of the polypeptides of the invention. For example, such alterations may change polypeptide characteristics such as ligand-binding affinities, interchain affinities, or degradation/turnover rate. Further, such alterations can be selected so as to generate polypeptides that are better suited for expression, scale up and the like in the host cells chosen for expression. For example, cysteine residues can be deleted or substituted with another amino acid residue in order to eliminate disulfide bridges.

The terms “purified” or “substantially purified” as used herein denotes that the indicated nucleic acid or polypeptide is present in the substantial absence of other biological macromolecules, e.g., polynucleotides, proteins, and the like. In one embodiment, the polynucleotide or polypeptide is purified such that it constitutes at least 95% by weight, more preferably at least 99% by weight, of the indicated biological macromolecules present (but water, buffers, and other small molecules, especially molecules having a molecular weight of less than 1000 daltons, can be present).

The term “isolated” as used herein refers to a nucleic acid or polypeptide separated from at least one other component (e.g., nucleic acid or polypeptide) present with the nucleic acid or polypeptide in its natural source. In one embodiment, the nucleic acid or polypeptide is found in the presence of (if anything) only a solvent, buffer, ion, or other components normally present in a solution of the same. The terms “isolated” and “purified” do not encompass nucleic acids or polypeptides present in their natural source.

The term “recombinant,” when used herein to refer to a polypeptide or protein, means that a polypeptide or protein is derived from recombinant (e.g., microbial, insect, or mammalian) expression systems. “Microbial” refers to recombinant polypeptides or proteins made in bacterial or fungal (e.g., yeast) expression systems. As a product, “recombinant microbial” defines a polypeptide or protein essentially free of native endogenous substances and unaccompanied by associated native glycosylation. Polypeptides or proteins expressed in most bacterial cultures, e.g., E. coli, will be free of glycosylation modifications; polypeptides or proteins expressed in yeast will have a glycosylation pattern in general different from those expressed in mammalian cells.

The term “recombinant expression vehicle or vector” refers to a plasmid or phage or virus or vector, for expressing a polypeptide from a DNA (RNA) sequence. An expression vehicle can comprise a transcriptional unit comprising an assembly of (1) a genetic element or elements having a regulatory role in gene expression, for example, promoters or enhancers, (2) a structural or coding sequence which is transcribed into mRNA and translated into protein, and (3) appropriate transcription initiation and termination sequences. Structural units intended for use in yeast or eukaryotic expression systems preferably include a leader sequence enabling extracellular secretion of translated protein by a host cell. Alternatively, where recombinant protein is expressed without a leader or transport sequence, it may include an amino terminal methionine residue. This residue may or may not be subsequently cleaved from the expressed recombinant protein to provide a final product.

The term “recombinant expression system” means host cells which have stably integrated a recombinant transcriptional unit into chromosomal DNA or carry the recombinant transcriptional unit extrachromosomally. Recombinant expression systems as defined herein will express heterologous polypeptides or proteins upon induction of the regulatory elements linked to the DNA segment or synthetic gene to be expressed. This term also means host cells which have stably integrated a recombinant genetic element or elements having a regulatory role in gene expression, for example, promoters or enhancers. Recombinant expression systems as defined herein will express polypeptides or proteins endogenous to the cell upon induction of the regulatory elements linked to the endogenous DNA segment or gene to be expressed. The cells can be prokaryotic or eukaryotic.

The term “secreted” includes a protein that is transported across or through a membrane, including transport as a result of signal sequences in its amino acid sequence when it is expressed in a suitable host cell. “Secreted” proteins include without limitation proteins secreted wholly (e.g., soluble proteins) or partially (e.g., receptors) from the cell in which they are expressed. “Secreted” proteins also include without limitation proteins that are transported across the membrane of the endoplasmic reticulum. “Secreted” proteins are also intended to include proteins containing non-typical signal sequences (e.g. Interleulin-1 Beta, see Krasney, P. A. and Young, P. R. Cytokine 4:134 -143 (1992)) and factors released from damaged cells (e.g. Interleukin-1 Receptor Antagonist, see Arend, W. P. et. al. Annu. Rev. Immunol. 16:27-55 (1998)).

Where desired, an expression vector may be designed to contain a “signal or leader sequence” which will direct the polypeptide through the membrane of a cell. Such a sequence may be naturally present on the polypeptides of the present invention or provided from heterologous protein sources by recombinant DNA techniques.

The term “stringent” is used to refer to conditions that are commonly understood in the art as stringent. Stringent conditions can include highly stringent conditions (i.e., hybridization to filter-bound DNA in 0.5 M NaHPO₄, 7% sodium dodecyl sulfate (SDS), 1 mM EDTA at 65° C., and washing in 0.1×SSC/0.1% SDS at 68° C.), and moderately stringent conditions (i.e., washing in 0.2×SSC/0.1% SDS at 42° C.).

In instances of hybridization of deoxyoligonucleotides, additional exemplary stringent hybridization conditions include washing in 6×SSC/0.05% sodium pyrophosphate at 37° C. (for 14-base oligonucleotides), 48° C. (for 17-base oligonucleotides), 55° C. (for 20-base oligonucleotides), and 60° C. (for 23-base oligonucleotides).

As used herein, “substantially equivalent” can refer both to nucleotide and amino acid sequences, for example a mutant sequence, that varies from a reference sequence by one or more substitutions, deletions, or additions, the net effect of which does not result in an adverse functional dissimilarity between the reference and subject sequences. Typically, S such a substantially equivalent sequence varies from one of those listed herein by no more than about 35% (i.e., the number of individual residue substitutions, additions, and/or deletions in a substantially equivalent sequence, as compared to the corresponding reference sequence, divided by the total number of residues in the substantially equivalent sequence is about 0.35 or less). Such a sequence is said to have 65% sequence identity to the listed sequence. In one embodiment, a substantially equivalent, e.g., mutant, sequence of the invention varies from a listed sequence by no more than 30% (70% sequence identity); in a variation of this embodiment, by no more than 25% (75% sequence identity); and in a further variation of this embodiment, by no more than 20% (80% sequence identity) and in a further variation of this embodiment, by no more than 10% (90% sequence identity) and in a further variation of this embodiment, by no more that 5% (95% sequence identity). Substantially equivalent, e.g., mutant, amino acid sequences according to the invention preferably have at least 80% sequence identity with a listed amino acid sequence, more preferably at least 90% sequence identity. Substantially equivalent nucleotide sequence of the invention can have lower percent sequence identities, taking into account, for example, the redundancy or degeneracy of the genetic code. Preferably, nucleotide sequence has at least about 65% identity, more preferably at least about 75% identity, and most preferably at least about 95% identity. For the purposes of the present invention, sequences having substantially equivalent biological activity and substantially equivalent expression characteristics are considered substantially equivalent. For the purposes of determining equivalence, truncation of the mature sequence (e.g., via a mutation which creates a spurious stop codon) should be disregarded. Sequence identity may be determined, e.g., using the Jotun Hein method (Hein, J. Methods Enzymol. 183:626-645 (1990)). Identity between sequences can also be determined by other methods known in the art, e.g. by varying hybridization conditions.

The term “totipotent” refers to the capability of a cell to differentiate into all of the cell types of an adult organism.

The term “transformation” means introducing DNA into a suitable host cell so that the DNA is replicable, either as an extrachromosomal element, or by chromosomal integration. The term “transfection” refers to the taking up of an expression vector by a suitable host cell, whether or not any coding sequences are in fact expressed. The tern “infection” refers to the introduction of nucleic acids into a suitable host cell by use of a virus or viral vector.

As used herein, an “uptake modulating fragment,” UMF, means a series of nucleotides which mediate the uptake of a linked DNA fragment into a cell. UMFs can be readily identified using known UMFs as a target sequence or target motif with the computer-based systems described below. The presence and activity of a UMF can be confirmed by attaching the suspected UMF to a marker sequence. The resulting nucleic acid molecule is then incubated with an appropriate host under appropriate conditions and the uptake of the marker sequence is determined. As described above, a UMF will increase the frequency of uptake of a linked marker sequence.

Each of the above terms is meant to encompass all that is described for each, unless the context dictates otherwise.

4.6 Nucleic Acids of the Invention

The isolated polynucleotides of the invention include, but are not limited to a polynucleotide comprising any of the nucleotide sequences of SEQ ID NO: 1-3, 5, 8, 10, 21, 23, 25, 27, 29, 31, 33, 35, 37-40, 43, 45, 49, 51, 53, 56-57, 59, 76-77, 79, 82, 84, 88-89, 91, 95-96, or 98; a fragment of SEQ ID NO: 1-3, 5, 8, 10, 21, 23, 25, 27, 29, 31, 33, 35, 37-40, 43, 45, 49, 51, 53, 56-57, 59, 76-77, 79, 82, 84, 88-89, 91, 95-96, or 98; a polynucleotide comprising the full length protein coding sequence of SEQ ID NO: 1-3, 5, 8, 10, 21, 23, 25, 27, 29, 31, 33, 35, 37-40, 43, 45, 49, 51, 53, 56-57, 59, 76-77, 79, 82, 84, 88-89, 91, 95-96, or 98 (for example coding for SEQ ID NO: 4, 7, 9, 12, 22, 24, 26, 28, 30, 32, 34, 44, 46, 50, 58, 61, 78, 81, 83, 86, 90, 93, 97, or 100); and a polynucleotide comprising the nucleotide sequence encoding the mature protein coding sequence of the polypeptides of any one of SEQ ID NO: 4, 6-7, 9, 11-12, 22, 24, 26, 28, 30, 32, 34, 36, 44, 46-48, 50, 52-53, 58, 60-62, 78, 80-81, 83, 85-87, 90, 92-94, 97, or 99-101. The polynucleotides of the present invention also include, but are not limited to, a polynucleotide that hybridizes under stringent conditions to (a) the complement of any of the nucleotides sequences of SEQ ID NO: 1-3, 5, 8, 10, 21, 23, 25, 27, 29, 31, 33, 35, 37-40, 43, 45, 49, 51, 53, 56-57, 59, 76-77, 79, 82, 84, 88-89, 91, 95-96, or 98; (b) a polynucleotide encoding any one of the polypeptides of SEQ ID NO: 4, 6-7, 9, 11-12, 22, 24, 26, 28, 30, 32, 34, 36, 44, 46-48, 50, 52-53, 58, 60-62, 78, 80-81, 83, 85-87, 90, 92-94, 97, or 99-101; (c) a polynucleotide which is an allelic variant of any polynucleotides recited above; (d) a polynucleotide which encodes a species homolog of any of the proteins recited above; or (e) a polynucleotide that encodes a polypeptide comprising a specific domain or truncation of the polypeptides of SEQ ID NO: 1-3, 5, 8, 10, 21, 23, 25, 27, 29, 31, 33, 35, 37-40, 43, 45, 49, 51, 53, 56-57, 59, 76-77, 79, 82, 84, 88-89, 91, 95-96, or 98. Domains of interest may depend on the nature of the encoded polypeptide; e.g., domains in receptor-like polypeptides include ligand-binding, extracellular, transmembrane, or cytoplasmic domains, or combinations thereof; domains in immunoglobulin-like proteins include the variable immunoglobulin-like domains; domains 10 in enzyme-like polypeptides include catalytic and substrate binding domains; and domains in ligand polypeptides include receptor-binding domains.

The polynucleotides of the invention include naturally occurring or wholly or partially synthetic DNA, e.g., cDNA and genomic DNA, and RNA, e.g., mRNA. The polynucleotides may include the entire coding region of the cDNA or may represent a portion of the coding region of the cDNA.

The present invention also provides genes corresponding to the cDNA sequences disclosed herein. The corresponding genes can be isolated in accordance with known methods using the sequence information disclosed herein. Such methods include the preparation of probes or primers from the disclosed sequence information for identification and/or amplification of genes in appropriate genomic libraries or other sources of genomic materials. Further 5′ and 3′ sequence can be obtained using methods known in the art. For example, full length cDNA or genomic DNA that corresponds to any of the polynucleotides of SEQ ID NO: 1-3, 5, 8, 10, 21, 23, 25, 27, 29, 31, 33, 35, 37-40, 43, 45, 49, 51, 53, 56-57, 59, 76-77, 79, 82, 84, 88-89, 91, 95-96, or 98 can be obtained by screening appropriate cDNA or genomic DNA libraries under suitable hybridization conditions using any of the polynucleotides of SEQ ID NO: 1-3, 5, 8, 10, 21, 23, 25, 27, 29, 31, 33, 35, 37-40, 43, 45, 49, 51, 53, 56-57, 59, 76-77, 79, 82, 84, 88-89, 91, 95-96, or 98 or a portion thereof as a probe. Alternatively, the polynucleotides of SEQ ID NO: 1-3, 5, 8, 10, 21, 23, 25, 27, 29, 31, 33, 35, 37-40, 43, 45, 49, 51, 53, 56-57, 59, 76-77, 79, 82, 84, 88-89, 91, 95-96, or 98 may be used as the basis for suitable primer(s) that allow identification and/or amplification of genes in appropriate genomic DNA or cDNA libraries.

The nucleic acid sequences of the invention can be assembled from ESTs and sequences (including cDNA and genomic sequences) obtained from one or more public databases, such as dbEST, gbpri and UniGene. The EST sequences can provide identifying sequence information, representative fragment or segment information, or novel segment information for the full-length gene.

The polynucleotides of the invention also provide polynucleotides including nucleotide sequences that are substantially equivalent to the polynucleotides recited above. Polynucleotides according to the invention can have, e.g., at least about 65%, at least about 70%, at least about 75%, at least about 80%, 81%, 82%, 83%, 84%, 85%, 86%, 87%, 88%, or 89%, more typically at least about 90%, 91%, 92%, 93%, or 94% and even more typically at least about 95%, 96%, 97%, 98% or 99% sequence identity to a polynucleotide recited above.

Included within the scope of the nucleic acid sequences of the invention are nucleic acid sequence fragments that hybridize under stringent conditions to any of the nucleotide sequences of SEQ ID NO: 1-3, 5, 8, 10, 21, 23, 25, 27, 29, 31, 33, 35, 37-40, 43, 45, 49, 51, 53, 56-57, 59, 76-77, 79, 82, 84, 88-89, 91, 95-96, or 98, or complements thereof, which fragment is greater than about 5 nucleotides, preferably 7 nucleotides, more preferably greater than 9 nucleotides and most preferably greater than 17 nucleotides. Fragments of, e.g. 15, 17, or 20 nucleotides or more that are selective for (i.e. specifically hybridize to any one of the polynucleotides of the invention) are contemplated. Probes capable of specifically hybridizing to a polynucleotide can differentiate polynucleotide sequences of the invention from other polynucleotide sequences in the same family of genes or can differentiate human genes from genes of other species, and are preferably based on unique nucleotide sequences.

The sequences falling within the scope of the present invention are not limited to these specific sequences, but also include allelic and species variations thereof Allelic and species variations can be routinely determined by comparing the sequence provided in SEQ ID NO: 1-3, 5, 8, 10, 21, 23, 25, 27, 29, 31, 33, 35, 37-40, 43, 45, 49, 51, 53, 56-57, 59, 76-77, 79, 82, 84, 88-89, 91, 95-96, or 98, a representative fragment thereof, or a nucleotide sequence at least 90% identical, preferably 95% identical, to SEQ ID NO: 1-3, 5, 8, 10, 21, 23, 25, 27, 29, 31, 33, 35, 37-40, 43, 45, 49, 51, 53, 56-57, 59, 76-77, 79, 82, 84, 88-89, 91, 95-96, or 98 with a sequence from another isolate of the same species. Furthermore, to accommodate codon variability, the invention includes nucleic acid molecules coding for the same amino acid sequences as do the specific ORFs disclosed herein. In other words, in the coding region of an ORF, substitution of one codon for another codon that encodes the same amino acid is expressly contemplated.

The nearest neighbor result for the nucleic acids of the present invention, including SEQ ID NO: 1-3, 5, 8, 10, 21, 23, 25, 27, 29, 31, 33, 35, 37-40, 43, 45, 49, 51, 53, 56-57, 59, 76-77, 79, 82, 84, 88-89, 91, 95-96, or 98, can be obtained by searching a database using an algorithm or a program. Preferably, a BLAST which stands for Basic Local Alignment Search Tool is used to search for local sequence alignments (Altshul, S. F., J Mol. Evol. 36 290-300 (1993) and Altschul S. F., et al. J. Mol. Biol. 21:403-410 (1990)).

Species homologs (or orthologs) of the disclosed polynucleotides and proteins are also provided by the present invention. Species homologs may be isolated and identified by making suitable probes or primers from the sequences provided herein and screening a suitable nucleic acid source from the desired species.

The invention also encompasses allelic variants of the disclosed polynucleotides or proteins; that is, naturally-occurring alternative forms of the isolated polynucleotide which also encodes proteins which are identical, homologous or related to that encoded by the polynucleotides.

The nucleic acid sequences of the invention are further directed to sequences which encode variants of the described nucleic acids. These amino acid sequence variants may be prepared by methods known in the art by introducing appropriate nucleotide changes into a native or variant polynucleotide. There are two variables in the construction of amino acid sequence variants: the location of the mutation and the nature of the mutation. Nucleic acids encoding the amino acid sequence variants are preferably constructed by mutating the polynucleotide to encode an amino acid sequence that does not occur in nature. These nucleic acid alterations can be made at sites that differ in the nucleic acids from different species (variable positions) or in highly conserved regions (constant regions). Sites at such locations will typically be modified in series, e.g., by substituting first with conservative choices (e.g., hydrophobic amino acid to a different hydrophobic amino acid) and then with more distant choices (e.g., hydrophobic amino acid to a charged amino acid), and then deletions or insertions may be made at the target site. Amino acid sequence deletions generally range from about 1 to 30 residues, preferably about 1 to 10 residues, and are typically contiguous. Amino acid insertions include amino- and/or carboxyl-terminal fusions ranging in length from one to one hundred or more residues, as well as intrasequence insertions of single or multiple amino acid residues. Intrasequence insertions may range generally from about 1 to 10 amino residues, preferably from 1 to 5 residues. Examples of terminal insertions include the heterologous signal sequences necessary for secretion or for intracellular targeting in different host cells and sequences such as FLAG or poly-histidine sequences useful for purifying the expressed protein.

In a preferred method, polynucleotides encoding the novel amino acid sequences are changed via site-directed mutagenesis. This method uses oligonucleotide sequences to alter a polynucleotide to encode the desired amino acid variant, as well as sufficient adjacent nucleotides on both sides of the changed amino acid to form a stable duplex on either side of the site being changed. In general, the techniques of site-directed mutagenesis are well known to those of skill in the art and this technique is exemplified by publications such as, Edelman et al., DNA 2:183 (1983). A versatile and efficient method for producing site-specific changes in a polynucleotide sequence was published by Zoller and Smith, Nucleic Acids Res. 10:6487-6500 (1982). PCR may also be used to create amino acid sequence variants of the novel nucleic acids. When small amounts of template DNA are used as starting material, primer(s) that differs slightly in sequence from the corresponding region in the template DNA can generate the desired amino acid variant. PCR amplification results in a population of product DNA fragments that differ from the polynucleotide template encoding the polypeptide at the position specified by the primer. The product DNA fragments replace the corresponding region in the plasmid and this gives a polynucleotide encoding the desired amino acid variant.

A further technique for generating amino acid variants is the cassette mutagenesis technique described in Wells, et al., Gene 34:315 (1985); and other mutagenesis techniques well known in the art, such as, for example, the techniques in Sambrook, et al., supra, and Current Protocols in Molecular Biology, Ausubel, et al. Due to the inherent degeneracy of the genetic code, other DNA sequences which encode substantially the same or a functionally equivalent amino acid sequence may be used in the practice of the invention for the cloning and expression of these novel nucleic acids. Such DNA sequences include those which are capable of hybridizing to the appropriate novel nucleic acid sequence under stringent conditions.

Polynucleotides encoding preferred polypeptide truncations of the invention can be used to generate polynucleotides encoding chimeric or fusion proteins comprising one or more domains of the invention and heterologous protein sequences.

The polynucleotides of the invention additionally include the complement of any of the polynucleotides recited above. The polynucleotide can be DNA (genomic, cDNA, amplified, or synthetic) or RNA. Methods and algorithms for obtaining such polynucleotides are well known to those of skill in the art and can include, for example, methods for determining hybridization conditions that can routinely isolate polynucleotides of the desired sequence identities.

In accordance with the invention, polynucleotide sequences comprising the mature protein coding sequences, coding for any one of SEQ ID NO: 4, 7, 9, 12, 22, 24, 26, 28, 30, 32, 34, 44, 46, 50, 58, 61, 78, 81, 83, 86, 90, 93, 97, or 100, or functional equivalents thereof, may be used to generate recombinant DNA molecules that direct the expression of that nucleic acid, or a functional equivalent thereof, in appropriate host cells. Also included are the cDNA inserts of any of the clones identified herein.

A polynucleotide according to the invention can be joined to any of a variety of other nucleotide sequences by well-established recombinant DNA techniques (see Sambrook, J. et al. (1989) Molecular Cloning: A Laboratory Manual, Cold Spring Harbor Laboratory, NY). Useful nucleotide sequences for joining to polynucleotides include an assortment of vectors, e.g., plasmids, cosmids, lambda phage derivatives, phagemids, and the like, that are well known in the art. Accordingly, the invention also provides a vector including a polynucleotide of the invention and a host cell containing the polynucleotide. In general, the vector contains an origin of replication functional in at least one organism, convenient restriction endonuclease sites, and a selectable marker for the host cell. Vectors according to the invention include expression vectors, replication vectors, probe generation vectors, and sequencing vectors. A host cell according to the invention can be a prokaryotic or eukaryotic cell and can be a unicellular organism or part of a multicellular organism.

The present invention further provides recombinant constructs comprising a nucleic acid having any of the nucleotide sequences of SEQ ID NO: 1-3, 5, 8, 10, 21, 23, 25, 27, 29, 31, 33, 35, 37-40, 43, 45, 49, 51, 53, 56-57, 59, 76-77, 79, 82, 84, 88-89, 91, 95-96, or 98 or a fragment thereof or any other polynucleotides of the invention. In one embodiment, the recombinant constructs of the present invention comprise a vector, such as a plasmid or viral vector, into which a nucleic acid having any of the nucleotide sequences of SEQ ID NO: 1-3, 5, 8, 10, 21, 23, 25, 27, 29, 31, 33, 35, 37-40, 43, 45, 49, 51, 53, 56-57, 59, 76-77, 79, 82, 84, 88-89, 91, 95-96, or 98 or a fragment thereof is inserted, in a forward or reverse orientation. In the case of a vector comprising one of the ORFs of the present invention, the vector may further comprise regulatory sequences, including for example, a promoter, operably linked to the ORF. Large numbers of suitable vectors and promoters are known to those of skill in the art and are commercially available for generating the recombinant constructs of the present invention. The following vectors are provided by way of example. Bacterial: pBs, phagescript, PsiX174, pBluescript SK, pBs KS, pNH8a, pNH16a, pNH18a, pNH46a (Stratagene); pTrc99A, pKK223-3, pKK233-3, pDR540, pRIT5 (Pharmacia). Eukaryotic: pWLneo, pSV2cat, pOG44, PXTI, pSG (Stratagene) pSVK3, pBPV, pMSG, and pSVL (Pharmacia).

The isolated polynucleotide of the invention may be operably linked to an expression control sequence such as the pMT2 or pED expression vectors disclosed in Kaufinan et al., Nucleic Acids Res. 19:4485-4490 (1991), in order to produce the protein recombinantly. Many suitable expression control sequences are known in the art. General methods of expressing recombinant proteins are also known and are exemplified in R. Kaufman, Methods in Enzymology 185:537-566 (1990). As defined herein “operably linked” means that the isolated polynucleotide of the invention and an expression control sequence are situated within a vector or cell in such a way that the protein is expressed by a host cell which has been transformed (transfected) with the ligated polynucleotide/expression control sequence.

Promoter regions can be selected from any desired gene using CAT (chloramphenicol transferase) vectors or other vectors with selectable markers. Two appropriate vectors are pKK232-8 and pCM7. Particular named bacterial promoters include lacI, lacZ, T3, T7, gpt, lambda PR, and trc. Eukaryotic promoters include CMV immediate early, HSV thymidine kinase, early and late SV40, LTRs from retrovirus, and mouse metallothionein-I. Selection of the appropriate vector and promoter is well within the level of ordinary skill in the art. Generally, recombinant expression vectors will include origins of replication and selectable markers permitting transformation of the host cell, e.g. the ampicillin resistance gene of E. coli and S. cerevisiae TRP1 gene, and a promoter derived from a highly expressed gene to direct transcription of a downstream structural sequence. Such promoters can be derived from operons encoding glycolytic enzymes such as 3-phosphoglycerate kinase (PGK), a-factor, acid phosphatase, or heat shock proteins, among others. The heterologous structural sequence is assembled in appropriate phase with translation initiation and termination sequences, and preferably, a leader sequence capable of directing secretion of translated protein into the periplasmic space or extracellular medium. Optionally, the heterologous sequence can encode a fusion protein including an amino terminal identification peptide imparting desired characteristics, e.g., stabilization or simplified purification of expressed recombinant product. Useful expression vectors for bacterial use are constructed by inserting a structural DNA sequence encoding a desired protein together with suitable translation initiation and termination signals in operable reading phase with a functional promoter. The vector will comprise one or more phenotypic selectable markers and an origin of replication to ensure maintenance of the vector and to, if desirable, provide amplification within the host. Suitable prokaryotic hosts for transformation include E. coli, Bacillus subtilis, Salmonella typhimurium and various species within the genera Pseudomonas, Streptomyces, and Staphylococcus, although others may also be employed as a matter of choice.

As a representative but non-limiting example, useful expression vectors for bacterial use can comprise a selectable marker and bacterial origin of replication derived from commercially available plasmids comprising genetic elements of the well known cloning vector pBR322 (ATCC 37017). Such commercial vectors include, for example, pKK223-3 (Pharmacia Fine Chemicals, Uppsala, Sweden) and GEM 1 (Promega Biotech, Madison, Wis., USA). These pBR322 “backbone” sections are combined with an appropriate promoter and the structural sequence to be expressed. Following transformation of a suitable host strain and growth of the host strain to an appropriate cell density, the selected promoter is induced or derepressed by appropriate means (e.g., temperature shift or chemical induction) and cells are cultured for an additional period. Cells are typically harvested by centrifugation, disrupted by physical or chemical means, and the resulting crude extract retained for further purification.

Polynucleotides of the invention can also be used to induce immune responses. For example, as described in Fan, et al., Nat. Biotech. 17:870-872 (1999), incorporated herein by reference, nucleic acid sequences encoding a polypeptide may be used to generate antibodies against the encoded polypeptide following topical administration of naked plasmid DNA or following injection, and preferably intramuscular injection of the DNA. The nucleic acid sequences are preferably inserted in a recombinant expression vector and may be in the form of naked DNA.

4.6.1 Antisense Nucleic Acids

Another aspect of the invention pertains to isolated antisense nucleic acid molecules that can hybridize to or are complementary to the nucleic acid molecule comprising the nucleotide sequence of SEQ ID NO: 1-3, 5, 8, 10, 21, 23, 25, 27, 29, 31, 33, 35, 37-40, 43, 45, 49, 51, 53, 56-57, 59, 76-77, 79, 82, 84, 88-89, 91, 95-96, or 98, or fragments, analogs or derivatives thereof. An “antisense” nucleic acid comprises a nucleotide sequence that is complementary to a “sense” nucleic acid encoding a protein (e.g., complementary to the coding strand of a double-stranded cDNA molecule or complementary to an mRNA sequence). In specific aspects, antisense nucleic acid molecules are provided that comprise a sequence complementary to at least about 10, 25, 50, 100, 250 or 500 nucleotides or an entire coding strand, or to only a portion thereof. Nucleic acid molecules encoding fragments, homologs, derivatives and analogs of a protein of any of SEQ ID NO: 4, 6-7, 9, 11-12, 22, 24, 26, 28, 30, 32, 34, 36, 44, 46-48, 50, 52-53, 58, 60-62, 78, 80-81, 83, 85-87, 90, 92-94, 97, or 99-101 or antisense nucleic acids complementary to a nucleic acid sequence of SEQ ID NO: 1-3, 5, 8, 10, 21, 23, 25, 27, 29, 31, 33, 35, 37-40, 43, 45, 49, 51, 53, 56-57, 59, 76-77, 79, 82, 84, 88-89, 91, 95-96, or 98 are additionally provided.

In one embodiment, an antisense nucleic acid molecule is antisense to a “coding region” of the coding strand of a nucleotide sequence of the invention. The term “coding region” refers to the region of the nucleotide sequence comprising codons which are translated into amino acid residues. In another embodiment, the antisense nucleic acid molecule is antisense to a “conceding region” of the coding strand of a nucleotide sequence of the invention. The term “conceding region” refers to 5′ and 3′ sequences which flank the coding region that are not translated into amino acids (i.e., also referred to as 5′ and 3′ untranslated regions).

Given the coding strand sequences (e.g. SEQ ID NO: 1-3, 5, 8, 10, 21, 23, 25, 27, 29, 31, 33, 35, 37-40, 43, 45, 49, 51, 53, 56-57, 59, 76-77, 79, 82, 84, 88-89, 91, 95-96, or 98) disclosed herein, antisense nucleic acids of the invention can be designed according to the rules of Watson and Crick or Hoogsteen base pairing. The antisense nucleic acid molecule can be complementary to the entire coding region of an mRNA of the invention, but more preferably is an oligonucleotide that is antisense to only a portion of the coding or noncoding region of an mRNA of the invention. For example, the antisense oligonucleotide can be complementary to the region surrounding the translation start site of an mRNA of the invention. An antisense oligonucleotide can be, for example, about 5, 10, 15, 20, 25, 30, 35, 40, 45, or 50 nucleotides in length. An antisense nucleic acid of the invention can be constructed using chemical synthesis or enzymatic ligation reactions using procedures known in the art. For example, an antisense nucleic acid (e.g., an antisense oligonucleotide) can be chemically synthesized using naturally occurring nucleotides or variously modified nucleotides designed to increase the biological stability of the molecules or to increase the physical stability of the duplex formed between the antisense and sense nucleic acids (e.g., phosphorothioate derivatives and acridine substituted nucleotides can be used).

Examples of modified nucleotides that can be used to generate the antisense nucleic acid include: 5-fluorouracil, 5-bromouracil, 5-chlorouracil, 5-iodouracil, hypoxanthine, xanthine, 4-acetylcytosine, 5-(carboxyhydroxymethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, and 2,6-diaminopurine. Alternatively, the antisense nucleic acid can be produced biologically using an expression vector into which a nucleic acid has been subcloned in an antisense orientation (i.e., RNA transcribed from the inserted nucleic acid will be of an antisense orientation to a target nucleic acid of interest, described further in the following section).

The antisense nucleic acid molecules of the invention are typically administered to a subject or generated in situ such that they hybridize with or bind to cellular mRNA and/or genomic DNA encoding a protein according to the invention to thereby inhibit expression of the protein (e.g., by inhibiting transcription and/or translation). The hybridization can be by conventional nucleotide complementarity to form a stable duplex, or, for example, in the case of an antisense nucleic acid molecule that binds to DNA duplexes, through specific interactions in the major groove of the double helix. An example of a route of administration of antisense nucleic acid molecules of the invention includes direct injection at a tissue site. Alternatively, antisense nucleic acid molecules can be modified to target selected cells and then administered systemically. For example, for systemic administration, antisense molecules can be modified such that they specifically bind to receptors or antigens expressed on a selected cell surface (e.g., by linking the antisense nucleic acid molecules to peptides or antibodies that bind to cell surface receptors or antigens). The antisense nucleic acid molecules can also be delivered to cells using the vectors described herein. To achieve sufficient nucleic acid molecules, vector constructs in which the antisense nucleic acid molecule is placed under the control of a strong pol II or pol III promoter are preferred.

In yet another embodiment, the antisense nucleic acid molecule of the invention is an alpha-anomeric nucleic acid molecule. An alpha-anomeric nucleic acid molecule forms specific double-stranded hybrids with complementary RNA in which, contrary to the usual alpha-units, the strands run parallel to each other. See, e.g., Gaultier, et al., Nucl. Acids Res. 15:6625-6641 (1987). The antisense nucleic acid molecule can also comprise a 2′-o-methylribonucleotide (see, e.g., Inoue, et al. Nucl. Acids Res. 15:6131-6148 (1987)) or a chimeric RNA-DNA analogue (see, e.g., Inoue, et al., FEBS Lett. 215:327-330 (1987).

4.6.2 Ribozymes and PNA Moieties

Nucleic acid modifications include, by way of non-limiting example, modified bases, and nucleic acids whose sugar phosphate backbones are modified or derivatized. These modifications are carried out at least in part to enhance the chemical stability of the modified nucleic acid, such that they can be used, for example, as antisense binding nucleic acids in therapeutic applications in a subject.

In one embodiment, an antisense nucleic acid of the invention is a ribozyme. Ribozymes are catalytic RNA molecules with ribonuclease activity that are capable of cleaving a single-stranded nucleic acid, such as an mRNA, to which they have a complementary region. Thus, ribozymes (e.g., hammerhead ribozymes as described in Haselhoff and Gerlach, Nature 334: 585-591 (1988)) can be used to catalytically cleave mRNA transcripts of the invention to thereby inhibit translation of mRNA of the invention. A ribozyme having specificity for a nucleic acid of the invention can be designed based upon the nucleotide sequence of a cDNA disclosed herein (e.g. SEQ ID NO: 1-3, 5, 8, 10, 21, 23, 25, 27, 29, 31, 33, 35, 37-40, 43, 45, 49, 51, 53, 56-57, 59, 76-77, 79, 82, 84, 88-89, 91, 95-96, or 98). For example, a derivative of a Tetrahymena L-19 IVS RNA can be constructed in which the nucleotide sequence of the active site is complementary to the nucleotide sequence to be cleaved in an mRNA of the invention. See, e.g., U.S. Pat. No. 4,987,071 to Cech, et al. and U.S. Pat. No. 5,116,742 to Cech, et al. Stem cell growth factor-like mRNA can also be used to select a catalytic RNA having a specific ribonuclease activity from a pool of RNA molecules. See, e.g., Bartel, et al., Science 261:1411-1418 (1993).

Alternatively, gene expression can be inhibited by targeting nucleotide sequences complementary to the regulatory region (e.g., the promoter and/or enhancers of the gene relating to the invention) to form triple helical structures that prevent transcription of the gene in target cells. See, e.g., Helene, Anticancer Drug Des. 6:569-84 (1991); Helene, et al., Ann. N.Y. Acad. Sci. 660:27-36 (1992); Maher, Bioassays 14:807-15 (1992).

In various embodiments, the nucleic acids of the invention can be modified at the base moiety, sugar moiety or phosphate backbone to improve, e.g., the stability, hybridization, or solubility of the molecule. For example, the deoxyribose phosphate backbone of the nucleic acids can be modified to generate peptide nucleic acids. See, e.g., Hyrup, et al., Bioorg. Med. Chem. 4:5-23 (1996). As used herein, the terms “peptide nucleic acids” or “PNAs” refer to nucleic acid mimics (e.g., DNA mimics) in which the deoxyribose phosphate backbone is replaced by a pseudopeptide backbone and only the four natural nucleobases are retained. The neutral backbone of PNAs has been shown to allow for specific hybridization to DNA and RNA under conditions of low ionic strength. The synthesis of PNA oligomers can be performed using standard solid phase peptide synthesis protocols as described in Hyrup, et al., 1996. supra; Perry-O'Keefe, et al., Proc. Natl. Acad. Sci. USA 93:14670-14675 (1996).

PNAs of the invention can be used in therapeutic and diagnostic applications. For example, PNAs can be used as antisense or antigene agents for sequence-specific modulation of gene expression by, e.g., inducing transcription or translation arrest or inhibiting replication. PNAs of the invention can also be used, for example, in the analysis of single base pair mutations in a gene (e.g., PNA directed PCR clamping; as artificial restriction enzymes when used in combination with other enzymes, e.g., S1 nucleases (see, Hyrup, et al., 1996. supra); or as probes or primers for DNA sequence and hybridization (see, Hyrup, et al., 1996, supra; Perry-O'Keefe, et al., 1996. supra).

In another embodiment, PNAs of the invention can be modified, e.g., to enhance their stability or cellular uptake, by attaching lipophilic or other helper groups to PNA, by the formation of PNA-DNA chimeras, or by the use of liposomes or other techniques of drug delivery known in the art. For example, PNA-DNA chimeras of the invention can be generated that may combine the advantageous properties of PNA and DNA. Such chimeras allow DNA recognition enzymes (e.g., RNase H and DNA polymerases) to interact with the DNA portion while the PNA portion would provide high binding affinity and specificity. PNA-DNA chimeras can be linked using linkers of appropriate lengths selected in terms of base stacking, number of bonds between the nucleobases, and orientation (see, Hyrup, et al., 1996. supra). The synthesis of PNA-DNA chimeras can be performed as described in Hyrup, et al., 1996. Supra, et al., Nucl Acids Res 24:3357-3363 (1996). For example, a DNA chain can be synthesized on a solid support using standard phosphoramidite coupling chemistry, and modified nucleoside analogs, e.g., 5′-(4-methoxytrityl)amino-5′-deoxy-thymidine phosphoramidite, can be used between the PNA and the 5′ end of DNA. See, e.g., Mag, et al., Nucl Acid Res 17:5973-5988 (1989). PNA monomers are then coupled in a stepwise manner to produce a chimeric molecule with a 5′ PNA segment and a 3′ DNA segment. See, e.g., Finn, et al., 1996. supra. Alternatively, chimeric molecules can be synthesized with a 5′ DNA segment and a 3′ PNA segment. See, e.g., Petersen, et al., Bioorg. Med. Chem. Lett. 5:1119-11124 (1975).

In other embodiments, the oligonucleotide may include other appended groups such as peptides (e.g., for targeting host cell receptors in vivo), or agents facilitating transport across the cell membrane (see, e.g., Letsinger, et al., Proc. Natl. Acad. Sci. U.S.A. 86:6553-6556 (1989); Lemaitre, et al., Proc. Natl. Acad. Sci. USA 84:648-652 (1987); PCT Publication No. WO88/09810) or the blood-brain barrier (see, e.g., PCT Publication No. WO 89/10134). In addition, oligonucleotides can be modified with hybridization-triggered cleavage agents (see, e.g., Krol, et al., BioTechniques 6:958-976 (1988)) or intercalating agents (see, e.g., Zon, Pharm. Res. 5:539-549 (1988)). To this end, the oligonucleotide can be conjugated to another molecule, e.g., a peptide, a hybridization triggered cross-linking agent, a transport agent, a hybridization-triggered cleavage agent, and the like.

4.7 Hosts

The present invention further provides host cells genetically engineered to contain the polynucleotides of the invention. For example, such host cells may contain nucleic acids of the invention introduced into the host cell using known transformation, transfection or infection methods. The present invention still further provides host cells genetically engineered to express the polynucleotides of the invention, wherein such polynucleotides are in operative association with a regulatory sequence heterologous to the host cell which drives expression of the polynucleotides in the cell.

The host cell can be a higher eukaryotic host cell, such as a mammalian cell, a lower eukaryotic host cell, such as a yeast cell, or the host cell can be a prokaryotic cell, such as a bacterial cell. Introduction of the recombinant construct into the host cell can be effected by calcium phosphate transfection, DEAE, dextran mediated transfection, or electroporation (Davis, L. et al., Basic Methods in Molecular Biology (1986)). The host cells containing one of polynucleotides of the invention, can be used in conventional manners to produce the gene product encoded by the isolated fragment (in the case of an ORF) or can be used to produce a heterologous protein under the control of the EMF.

Any host/vector system can be used to express one or more of the ORFs of the present invention. These include, but are not limited to, eukaryotic hosts such as HeLa cells, Cv-1 cell, COS cells, and Sf9 cells, as well as prokaryotic host such as E. coli and B. subtilis. The most preferred cells are those which do not normally express the particular polypeptide or protein or which expresses the polypeptide or protein at low natural level. Mature proteins can be expressed in mammalian cells, yeast, bacteria, or other cells under the control of appropriate promoters. Cell-free translation systems can also be employed to produce such proteins using RNAs derived from the DNA constructs of the present invention. Appropriate cloning and expression vectors for use with prokaryotic and eukaryotic hosts are described by Sambrook, et al., in Molecular Cloning: A Laboratory Manual, Second Edition, Cold Spring Harbor, N.Y. (1989), the disclosure of which is hereby incorporated by reference.

Various mammalian cell culture systems can also be employed to express recombinant protein. Examples of mammalian expression systems include the COS-7 lines of monkey kidney fibroblasts, described by Gluzman, Cell 23:175 (1981), and other cell lines capable of expressing a compatible vector, for example, the C127, 3T3, CHO, HeLa and BHK cell tines. Mammalian expression vectors will comprise an origin of replication, a suitable promoter, and also any necessary ribosome binding sites, polyadenylation site, splice donor and acceptor sites, transcriptional termination sequences, and 5′ flanking nontranscribed sequences. DNA sequences derived from the SV40 viral genome, for example, SV40 origin, early promoter, enhancer, splice, and polyadenylation sites may be used to provide the required nontranscribed genetic elements. Recombinant polypeptides and proteins produced in bacterial culture are usually isolated by initial extraction from cell pellets, followed by one or more salting-out, aqueous ion exchange or size exclusion chromatography steps. Protein refolding steps can be used, as necessary, in completing configuration of the mature protein. Finally, high performance liquid chromatography (HPLC) can be employed for final purification steps. Microbial cells employed in expression of proteins can be disrupted by any convenient method, including freeze-thaw cycling, sonication, mechanical disruption, or use of cell lysing agents.

A number of types of cells may act as suitable host cells for expression of the protein. Mammalian host cells include, for example, monkey COS cells, Chinese Hamster Ovary (CHO) cells, human kidney 293 cells, human epidermal A431 cells, human Colo205 cells, 3T3 cells, CV-1 cells, other transformed primate cell lines, normal diploid cells, cell strains derived from in vitro culture of primary tissue, primary explants, HeLa cells, mouse L cells, BHK, HL-60, U937, HaK or Jurkat cells.

Alternatively, it may be possible to produce the protein in lower eukaryotes such as yeast or in prokaryotes such as bacteria Potentially suitable yeast strains include Saccharomyces cerevisiae, Schizosaccharomycespombe, Kluyveromyces strains, Candida albicans, or any yeast strain capable of expressing heterologous proteins. Potentially suitable bacterial strains include Escherichia coli, Bacillus subtilis, Salmonella typhimurium, or any bacterial strain capable of expressing heterologous proteins. If the protein is made in yeast or bacteria, it may be necessary to modify the protein produced therein, for example by phosphorylation or glycosylation of the appropriate sites, in order to obtain the functional protein. Such covalent attachments may be accomplished using known chemical or enzymatic methods.

In another embodiment of the present invention, cells and tissues may be engineered to express an endogenous gene comprising the polynucleotides of the invention under the control of inducible regulatory elements, in which case the regulatory sequences of the endogenous gene may be replaced by homologous recombination. As described herein, gene targeting can be used to replace a gene's existing regulatory region with a regulatory sequence isolated from a different gene or a novel regulatory sequence synthesized by genetic engineering methods. Such regulatory sequences may be comprised of promoters, enhancers, scaffold-attachment regions, negative regulatory elements, transcriptional initiation sites, regulatory protein binding sites or combinations of said sequences. Alternatively, sequences which affect the structure or stability of the RNA or protein produced may be replaced, removed, added, or otherwise modified by targeting, including polyadenylation signals, mRNA stability elements, splice sites, leader sequences for enhancing or modifying transport or secretion properties of the protein, or other sequences which alter or improve the function or stability of protein or RNA molecules.

The targeting event may be a simple insertion of the regulatory sequence, placing the gene under the control of the new regulatory sequence, e.g., inserting a new promoter or enhancer or both upstream of a gene. Alternatively, the targeting event may be a simple deletion of a regulatory element, such as the deletion of a tissue-specific negative regulatory element. Alternatively, the targeting event may replace an existing element; for example, a tissue-specific enhancer can be replaced by an enhancer that has broader or different cell-type specificity than the naturally occurring elements. Here, the naturally occurring sequences are deleted and new sequences are added. In all cases, the identification of the targeting event may be facilitated by the use of one or more selectable marker genes that are contiguous with the targeting DNA, allowing for the selection of cells in which the exogenous DNA has integrated into the host cell genome. The identification of the targeting event may also be facilitated by the use of one or more marker genes exhibiting the property of negative selection, such that the negatively selectable marker is linked to the exogenous DNA, but configured such that the negatively selectable marker flanks the targeting sequence, and such that a correct homologous recombination event with sequences in the host cell genome does not result in the stable integration of the negatively selectable marker. Markers useful for this purpose include the Herpes Simplex Virus thymidine kinase (TK) gene or the bacterial xanthine-guanine phosphoribosyl-transferase (gpt) gene.

The gene targeting or gene activation techniques which can be used in accordance with this aspect of the invention are more particularly described in U.S. Pat. No. 5,272,071 to Chappel; U.S. Pat. No. 5,578,461 to Sherwin et al.; International Application No. PCT/US92/09627 (WO93/09222) by Selden et al.; and International Application No. PCT/US90/06436 (WO91/06667) by Skoultchi et al., each of which is incorporated by reference herein in its entirety.

4.7.1 Chimeric and Fusion Proteins

The invention also provides chimeric or fusion proteins. As used herein, a “chimeric protein” or “fusion protein” of the invention comprises a polypeptide of the invention operatively linked to another polypeptide. Within a fusion protein of the invention, the polypeptide according to the invention can correspond to all or a portion of a protein according to the invention. In one embodiment, a fusion protein comprises at least one biologically active portion of a protein according to the invention. In another embodiment, a fusion protein comprises at least two biologically active portions of a protein according to the invention. In yet another embodiment, a fusion protein comprises at least three biologically active portions of a protein according to the invention. Within the fusion protein, the term “operatively-linked” is intended to indicate that the polypeptide according to the invention and the other polypeptide are fused in-frame with one another. The other polypeptide can be fused to the N-terminus or C-terminus of the polypeptide according to the invention. For example, in one embodiment a fusion protein comprises a polypeptide according to the invention operably linked to the extracellular domain of a second protein.

In one embodiment, the fusion protein is a GST-fusion protein in which the polypeptide sequences according to the invention are fused to the C-terminus of the GST (glutathione S-transferase) sequences. Such fusion proteins can facilitate the purification of recombinant polypeptides according to the invention. In another embodiment, the fusion protein is a protein according to the invention containing a heterologous signal sequence at its N-terminus. In certain host cells (e.g., mammalian host cells), expression and/or secretion of the polypeptide according to the invention can be increased through use of a heterologous signal sequence.

In yet another embodiment, the fusion protein is an immunoglobulin fusion protein in which the polypeptide sequences of the invention are fused to sequences derived from a member of the immunoglobulin protein family. The immunoglobulin fusion proteins of the invention can be incorporated into pharmaceutical compositions and administered to a subject to inhibit an interaction between a ligand and a protein according to the invention on the surface of a cell, to thereby suppress signal transduction mediated by the protein according to the invention in vivo. The immunoglobulin fusion proteins can be used to affect the bioavailability of a cognate ligand. Inhibition of the ligand/protein interaction can be useful therapeutically for both the treatment of proliferative and differentiative disorders, as well as modulating (e.g. promoting or inhibiting) cell survival. Moreover, the immunoglobulin fusion proteins of the invention can be used as immunogens to produce antibodies in a subject, to purify ligands, and in screening assays to identify molecules that inhibit the interaction of a polypeptide according to the invention with a ligand.

A chimeric or fusion protein of the invention can be produced by standard recombinant DNA techniques. For example, DNA fragments coding for the different polypeptide sequences are ligated together in-frame in accordance with conventional techniques, e.g., by employing blunt-ended or stagger-ended termini for ligation, restriction enzyme digestion to provide for appropriate termini, filling-in of cohesive ends as appropriate, alkaline phosphatase treatment to avoid undesirable joining, and enzymatic ligation. In another embodiment, the fusion gene can be synthesized by conventional techniques including automated DNA synthesizers. Alternatively, PCR amplification of gene fragments can be carried out using anchor primers that give rise to complementary overhangs between two consecutive gene fragments that can subsequently be annealed and reamplified to generate a chimeric gene sequence (see, e.g., Ausubel, et al. (eds.) CURRENT PROTOCOLS IN MOLECULAR BIOLOGY, John Wiley & Sons, 1992). Moreover, many expression vectors are commercially available that already encode a fusion moiety (e.g., a GST polypeptide). A nucleic acid encoding a polypeptide of the invention can be cloned into such an expression vector such that the fusion moiety is linked in-frame to the protein of the invention.

4.8 Polypeptides of the Invention

The isolated polypeptides of the invention include, but are not limited to, a polypeptide comprising: the amino acid sequence set forth as any one of SEQ ID NO: 4, 6-7, 9, 11-12, 22, 24, 26, 28, 30, 32, 34, 36, 44, 46-48, 50, 52-53, 58, 60-62, 78, 80-81, 83, 85-87, 90, 92-94, 97, or 99-101, or an amino acid sequence encoded by any one of the nucleotide sequences SEQ ID NO: 1-3, 5, 8, 10, 21, 23, 25, 27, 29, 31, 33, 35, 37-40, 43, 45, 49, 51, 53, 56-57, 59, 76-77, 79, 82, 84, 88-89, 91, 95-96, or 98, or the corresponding full length or mature protein. Polypeptides of the invention also include polypeptides preferably with biological or immunological activity that are encoded by: (a) a polynucleotide having any one of the nucleotide sequences set forth in SEQ ID NO: 1-3, 5, 8, 10, 21, 23, 25, 27, 29, 31, 33, 35, 37-40, 43, 45, 49, 51, 53, 56-57, 59, 76-77, 79, 82, 84, 88-89, 91, 95-96, or 98, or (b) polynucleotides encoding any one of the amino acid sequences set forth as SEQ ID NO: 4, 6-7, 9, 11-12, 22, 24, 26, 28, 30, 32, 34, 36, 44, 46-48, 50, 52-53, 58, 60-62, 78, 80-81, 83, 85-87, 90, 92-94, 97, or 99-101; or (c) polynucleotides that hybridize to the complement of the polynucleotides of either (a) or (b) under stringent hybridization conditions. The invention also provides biologically active or immunologically active variants of any of the amino acid sequences set forth as SEQ ID NO: 4, 6-7, 9, 11-12, 22, 24, 26, 28, 30, 32, 34, 36, 44, 46-48, 50, 52-53, 58, 60-62, 78, 80-81, 83, 85-87, 90, 92-94, 97, or 99-101, or the corresponding full length or mature protein; and “substantial equivalents” thereof (e.g., with at least about 65%, at least about 70%, at least about 75%, at least about 80%, 81%, 82%, 83%, 84%, 85%, 86%, 87%, 88%, or 89%, more typically at least about 90%, 91%, 92%, 93%, or 94% and even more typically at least about 95%, 96%, 97%, 98% or 99%, most typically at least about 99% amino acid identity) that retain biological activity. Polypeptides encoded by allelic variants may have a similar, increased, or decreased activity compared to polypeptides comprising SEQ ID NO: 4, 6-7,9, 11-12, 22, 24, 26, 28, 30, 32, 34, 36, 44, 46-48, 50, 52-53, 58, 60-62, 78, 80-81, 83, 85-87, 90, 92-94, 97, or 99-101.

Fragments of the proteins of the present invention which are capable of exhibiting biological activity are also encompassed by the present invention. Fragments of the protein may be in linear form or they may be cyclized using known methods, for example, as described in H. U. Saragovi, et al., Bio/Technology 10:773-778 (1992) and in R. S. McDowell, et al., J. Amer. Chem. Soc. 114:9245-9253 (1992), both of which are incorporated herein by reference. Such fragments may be fused to carrier molecules such as immunoglobulins for many purposes, including increasing the valency of protein binding sites.

The present invention also provides both full-length and mature forms (for example, without a signal sequence or precursor sequence) of the disclosed proteins. The protein coding sequence is identified in the sequence listing by translation of the disclosed nucleotide sequences. The mature form of such protein may be obtained by expression of a full-length polynucleotide in a suitable mammalian cell or other host cell. The sequence of the mature form of the protein is also determinable from the amino acid sequence of the full-length form. Where proteins of the present invention are membrane bound, soluble forms of the proteins are also provided. In such forms, part or all of the regions causing the proteins to be membrane bound are deleted so that the proteins are fully secreted from the cell in which it is expressed.

Protein compositions of the present invention may further comprise an acceptable carrier, such as a hydrophilic, e.g., pharmaceutically acceptable, carrier.

The present invention further provides isolated polypeptides encoded by the nucleic acid fragments of the present invention or by degenerate variants of the nucleic acid fragments of the present invention. By “degenerate variant” is intended to denote nucleotide fragments which differ from a nucleic acid fragment of the present invention (e.g., an ORF) by nucleotide sequence but, due to the degeneracy of the genetic code, encode an identical polypeptide sequence. Preferred nucleic acid fragments of the present invention are the ORFs that encode proteins.

A variety of methodologies known in the art can be utilized to obtain any one of the isolated polypeptides or proteins of the present invention. At the simplest level, the amino acid sequence can be synthesized using commercially available peptide synthesizers. The synthetically-constructed protein sequences, by virtue of sharing primary, secondary or tertiary structural and/or conformational characteristics with proteins may possess biological properties in common therewith, including protein activity. This technique is particularly useful in producing small peptides and fragments of larger polypeptides. Fragments are useful, for example, in generating antibodies against the native polypeptide. Thus, they may be employed as biologically active or immunological substitutes for natural, purified proteins in screening of therapeutic compounds and in immunological processes for the development of antibodies.

The polypeptides and proteins of the present invention can alternatively be purified from cells which have been altered to express the desired polypeptide or protein. As used herein, a cell is said to be altered to express a desired polypeptide or protein when the cell, through genetic manipulation, is made to produce a polypeptide or protein which it normally does not produce dr which the cell normally produces at a lower level. One skilled in the art can readily adapt procedures for introducing and expressing either recombinant or synthetic sequences into eukaryotic or prokaryotic cells in order to generate a cell which produces one of the polypeptides or proteins of the present invention.

The invention also relates to methods for producing a polypeptide comprising growing a culture of host cells of the invention in a suitable culture medium, and purifying the protein from the cells or the culture in which the cells are grown. For example, the methods of the invention include a process for producing a polypeptide in which a host cell containing a suitable expression vector that includes a polynucleotide of the invention is cultured under conditions that allow expression of the encoded polypeptide. The polypeptide can be recovered from the culture, conveniently from the culture medium, or from a lysate prepared from the host cells and further purified. Preferred embodiments include those in which the protein produced by such process is a full length or mature form of the protein.

In an alternative method, the polypeptide or protein is purified from bacterial cells which naturally produce the polypeptide or protein. One skilled in the art can readily follow known methods for isolating polypeptides and proteins in order to obtain one of the isolated polypeptides or proteins of the present invention. These include, but are not limited to, immunochromatography, HPLC, size-exclusion chromatography, ion-exchange chromatography, and immuno-affinity chromatography. See, e.g., Scopes, Protein Purification: Principles and Practice, Springer-Verlag (1994); Sambrook, et al., in Molecular Cloning: A Laboratory Manual; Ausubel et al., Current Protocols in Molecular Biology. Polypeptide fragments that retain biological/immunological activity include fragments comprising greater than about 100 amino acids, or greater than about 200 amino acids, and fragments that encode specific protein domains.

The purified polypeptides can be used in in vitro binding assays which are well known in the art to identify molecules which bind to the polypeptides. These molecules include but are not limited to, for e.g., small molecules, molecules from combinatorial libraries, antibodies or other proteins. The molecules identified in the binding assay are then tested for antagonist or agonist activity in in vivo tissue culture or animal models that are well known in the art. In brief, the molecules are titrated into a plurality of cell cultures or animals and then tested for either cell/animal death or prolonged survival of the animal/cells.

In addition, the peptides of the invention or molecules capable of binding to the peptides may be complexed with toxins, e.g., ricin or cholera, or with other compounds that are toxic to cells. The toxin-binding molecule complex is then targeted to a tumor or other cell by the specificity of the binding molecule for SEQ ID NO: 4, 6-7, 9, 11-12, 22, 24, 26, 28, 30, 32, 34, 36, 44, 46-48, 50, 52-53, 58, 60-62, 78, 80-81, 83, 85-87, 90, 92-94, 97, or 99-101.

The protein of the invention may also be expressed as a product of transgenic animals, e.g., as a component of the milk of transgenic cows, goats, pigs, or sheep which are characterized by somatic or germ cells containing a nucleotide sequence encoding the protein.

The proteins provided herein also include proteins characterized by amino acid sequences similar to those of purified proteins but into which modification are naturally provided or “deliberately engineered. For example, modifications, in the peptide or DNA sequence, can be made by those skilled in the art using known techniques. Modifications of interest in the protein sequences may include the alteration, substitution, replacement, insertion or deletion of a selected amino acid residue in the coding sequence. For example, one or more of the cysteine residues may be deleted or replaced with another amino acid to alter the conformation of the molecule. Techniques for such alteration, substitution, replacement, insertion or deletion are well known to those skilled in the art (see, e.g., U.S. Pat. No. 4,518,584). Preferably, such alteration, substitution, replacement, insertion or deletion retains the desired activity of the protein. Regions of the protein that are important for the protein function can be determined by various methods known in the art including the alanine-scanning method which involved systematic substitution of single or strings of amino acids with alanine, followed by testing the resulting alanine-containing variant for biological activity. This type of analysis determines the importance of the substituted amino acid(s) in biological activity. Regions of the protein that are important for protein function may be determined by the eMATRIX program.

Other fragments and derivatives of the sequences of proteins which would be expected to retain protein activity in whole or in part and are useful for screening or other immunological methodologies may also be easily made by those skilled in the art given the disclosures herein. Such modifications are encompassed by the present invention.

The protein may also be produced by operably linking the isolated polynucleotide of the invention to suitable control sequences in one or more insect expression vectors, and employing an insect expression system. Materials and methods for baculovirus/insect cell expression systems are commercially available in kit form from, e.g., Invitrogen, San Diego, Calif., U.S.A. (the MaxBat™ kit), and such methods are well known in the art, as described in Summers and Smith, Texas Agricultural Experiment Station Bulletin No. 1555 (1987), incorporated herein by reference. As used herein, an insect cell capable of expressing a polynucleotide of the present invention is “transformed.”

The protein of the invention may be prepared by culturing transformed host cells under culture conditions suitable to express the recombinant protein. The resulting expressed protein may then be purified from such culture (i.e., from culture medium or cell extracts) using known purification processes, such as gel filtration and ion exchange chromatography. Purification of the protein of the invention may also include an affinity column containing agents which will bind to the protein of the invention; one or more column steps over such affinity resins as concanavalin A-agarose, heparin-toyopearl™ or Cibacrom blue 3GA Sepharose™; one or more steps involving hydrophobic interaction chromatography using such resins as phenyl ether, butyl ether, or propyl ether; or immunoaffinity chromatography.

Alternatively, the protein of the invention may also be expressed in a form which will facilitate purification. For example, it may be expressed as a fusion protein, such as those of maltose binding protein (MBP), glutathione-S-transferase (GST) or thioredoxin (TRX), or as a His tag. Kits for expression and purification of such fusion proteins are commercially available from New England BioLab (Beverly, Mass.), Pharmacia (Piscataway, N.J.) and Invitrogen, respectively. The protein of the invention can also be tagged with an epitope and subsequently purified by using a specific antibody directed to such epitope. One such epitope (“FLAG®”) is commercially available from Kodak (New Haven, Conn.).

Finally, one or more reverse-phase high performance liquid chromatography (RP-HPLC) steps employing hydrophobic RP-HPLC media, e.g., silica gel having pendant methyl or other aliphatic groups, can be employed to further purify the protein of the invention. Some or all of the foregoing purification steps, in various combinations, can also be employed to provide a substantially homogeneous isolated recombinant protein. The protein thus purified is substantially free of other mammalian proteins and is defined in accordance with the present invention as an “isolated protein.”

The polypeptides of the invention include analogs (variants). This embraces fragments of the polypeptides of the invention, as well polypeptides of the invention which comprise one or more amino acids deleted, inserted, or substituted. Also, analogs of the polypeptides of the invention embrace fusions of the polypeptides of the invention or modifications of the polypeptides of the invention, wherein the polypeptide or analog of the invention is fused to another moiety or moieties, e.g., targeting moiety or another therapeutic agent. Such analogs may exhibit improved properties such as activity and/or stability. Examples of moieties which may be fused to the polypeptide or an analog of the invention include, for example, targeting moieties which provide for the delivery of polypeptides of the invention to neurons, e.g., antibodies to central nervous system, or antibodies to receptor and ligands expressed on neuronal cells. Other moieties which may be fused to polypeptides of the invention include therapeutic agents which are used for treatment, for example anti-depressant drugs or other medications for neurological disorders. Also, polypeptides of the invention may be fused to neuron growth modulators, and other chemokines for targeted delivery.

4.8.1 Determining Polypeptide and Polynucleotide Identity and Similarity

Preferred identity and/or similarity are designed to give the largest match between the sequences tested. Methods to determine identity and similarity are codified in computer programs including, but are not limited to, the GCG program package, including GAP (Devereux, J., et al., Nucl. Acids Res. 12:387 (1984); Genetics Computer Group, University of Wisconsin, Madison, Wis., herein incorporated by reference), BLASTP, BLASTN, BLASTX, FASTA (Altschul, S. F. et al., J. Molec. Biol. 215:403-410 (1990), PSI-BLAST (Altschul S. F. et al., Nucl. Acids Res. 25:3389-3402, herein incorporated by reference), the eMatrix software (Wu et al., J. Comp. Biol., 6:219-235 (1999), herein incorporated by reference), eMotif software (Nevill-Manning et al, ISMB-97, 4,:202-209, herein incorporated by reference), the GeneAtlas software (Molecular Simulations Inc. (MSI), San Diego, Calif.) (Sanchez and Sali, Proc. Natl. Acad. Sci. USA, 95:13597-13602 (1998); Kitson DH, et al, (2000) “Remote homology detection using structural modeling—an evaluation” Submitted; Fischer and Eisenberg, Protein Sci. 5:947-955 (1996)), and the Kyte-Doolittle hydrophobocity prediction algorithm (J. Mol Biol, 157:105-31 (1982), incorporated herein by reference). The BLAST programs are publicly available from the National Center for Biotechnology Information (NCBI) and other sources (BLAST Manual, Altschul, S., et al. NCB NLM NIH Bethesda, Md. 20894; Altschul, S., et al., J. Mol. Biol. 215:403-410 (1990).

4.9 Gene Therapy

Mutations in the gene encoding the polypeptide of the invention may result in loss of normal function of the encoded protein. The invention thus provides gene therapy to restore normal activity of the polypeptides of the invention; or to treat disease states involving polypeptides of the invention. Delivery of a functional gene encoding polypeptides of the invention to appropriate cells is effected ex vivo, in situ, or in vivo by use of vectors, and more particularly viral vectors (e.g., adenovirus, adeno-associated virus, or a retrovirus), or ex vivo by use of physical DNA transfer methods (e.g., liposomes or chemical treatments). See, for example, Anderson, Nature, 392(Suppl.):25-20 (1998). For additional reviews of gene therapy technology see Friedmann, Science, 244:1275-1281 (1989); Verma, Scientific American: 68-84 (1990); and Miller, Nature, 357:455-460 (1992). Introduction of any one of the nucleotides of the present invention or a gene encoding the polypeptides of the present invention can also be accomplished with extrachromosomal substrates (transient expression) or artificial chromosomes (stable expression). Cells may also be cultured ex vivo in the presence of proteins of the present invention in order to proliferate or to produce a desired effect on or activity in such cells. Treated cells can then be introduced in vivo for therapeutic purposes. Alternatively, it is contemplated that in other human disease states, preventing the expression of or inhibiting the activity of polypeptides of the invention will be useful in treating the disease states. It is contemplated that antisense therapy or gene therapy could be applied to negatively regulate the expression of polypeptides of the invention.

Other methods inhibiting expression of a protein include the introduction of antisense molecules to the nucleic acids of the present invention, their complements, or their translated RNA sequences, by methods known in the art Further, the polypeptides of the present invention can be inhibited by using targeted deletion methods, or the insertion of a negative regulatory element such as a silencer, which is tissue specific.

The present invention still further provides cells genetically engineered in vivo to express the polynucleotides of the invention, wherein such polynucleotides are in operative association with a regulatory sequence heterologous to the host cell which drives expression of the polynucleotides in the cell. These methods can be used to increase or decrease the expression of the polynucleotides of the present invention.

Knowledge of DNA sequences provided by the invention allows for modification of cells to permit, increase, or decrease, expression of endogenous polypeptide. Cells can be modified (e.g., by homologous recombination) to provide increased polypeptide expression by replacing, in whole or in part, the naturally occurring promoter with all or part of a heterologous promoter so that the cells express the protein at higher levels. The heterologous promoter is inserted in such a manner that it is operatively linked to the desired protein encoding sequences. See, for example, PCT International Publication No. WO 94/12650, PCT International Publication No. WO 92/20808, and PCT International Publication No. WO 91/09955. It is also contemplated that, in addition to heterologous promoter DNA, amplifiable marker DNA (e.g., ada, dhfr, and the multifunctional CAD gene which encodes carbamyl phosphate synthase, aspartate transcarbamylase, and dihydroorotase) and/or intron DNA may be inserted along with the heterologous promoter DNA. If linked to the desired protein coding sequence, amplification of the marker DNA by standard selection methods results in co-amplification of the desired protein coding sequences in the cells.

In another embodiment of the present invention, cells and tissues may be engineered to express an endogenous gene comprising the polynucleotides of the invention under the control of inducible regulatory elements, in which case the regulatory sequences of the endogenous gene may be replaced by homologous recombination. As described herein, gene targeting can be used to replace a gene's existing regulatory region with a regulatory sequence isolated from a different gene or a novel regulatory sequence synthesized by genetic engineering methods. Such regulatory sequences may be comprised of promoters, enhancers, scaffold-attachment regions, negative regulatory elements, transcriptional initiation sites, regulatory protein binding sites or combinations of said sequences. Alternatively, sequences which affect the structure or stability of the RNA or protein produced may be replaced, removed, added, or otherwise modified by targeting. These sequences include polyadenylation signals, mRNA stability elements, splice sites, leader sequences for enhancing or modifying transort or secretion properties of the protein, or other sequences which alter or improve the function or stability of protein or RNA molecules.

The targeting event may be a simple insertion of the regulatory sequence, placing the gene under the control of the new regulatory sequence, e.g., inserting a new promoter or enhancer or both upstream of a gene. Alternatively, the targeting event may be a simple deletion of a regulatory element, such as the deletion of a tissue-specific negative regulatory element. Alternatively, the targeting event may replace an existing element; for example, a tissue-specific enhancer can be replaced by an enhancer that has broader or different cell-type specificity than the naturally occurring elements. Here, the naturally occurring sequences are deleted and new sequences are added. In all cases, the identification of the targeting event may be facilitated by the use of one or more selectable marker genes that are contiguous with the targeting DNA, allowing for the selection of cells in which the exogenous DNA has integrated into the cell genome. The identification of the targeting event may also be facilitated by the use of one or more marker genes exhibiting the property of negative selection, such that the negatively selectable marker is linked to the exogenous DNA, but configured such that the negatively selectable marker flanks the targeting sequence, and such that a correct homologous recombination event with sequences in the host cell genome does not result in the stable integration of the negatively selectable marker. Markers useful for this purpose include the Herpes Simplex Virus thymidine kinase (TK) gene or the bacterial xanthine-guanine phosphoribosyl-transferase (gpt) gene.

The gene targeting or gene activation techniques which can be used in accordance with this aspect of the invention are more particularly described in U.S. Pat. No. 5,272,071 to Chappel; U.S. Pat. No. 5,578,461 to Sherwin et al.; International Application No. PCT/US92/09627 (WO93/09222) by Selden et al.; and International Application No. PCT/US90/06436 (WO91/06667) by Skoultchi et al., each of which is incorporated by reference herein in its entirety.

4.10 Transgenic Animals

In preferred methods to determine biological functions of the polypeptides of the invention in vivo, one or more genes provided by the invention are either over expressed or inactivated in the germ line of animals using homologous recombination (Capecchi, Science 244:1288-1292 (1989)). Animals in which the gene is over expressed, under the regulatory control of exogenous or endogenous promoter elements, are known as transgenic animals. Animals in which an endogenous gene has been inactivated by homologous recombination are referred to as “knockout” animals. Knockout animals, preferably non-human mammals, can be prepared as described in U.S. Pat. No. 5,557,032, incorporated herein by reference. Transgenic animals are useful to determine the roles polypeptides of the invention play in biological processes, and preferably in disease states. Transgenic animals are useful as model systems to identify compounds that modulate lipid metabolism. Transgenic animals, preferably non-human mammals, are produced using methods as described in U.S. Pat. No 5,489,743 and PCT Publication No. WO94/28122, incorporated herein by reference.

Transgenic animals can be prepared wherein all or part of a promoter of the polynucleotides of the invention is either activated or inactivated to alter the level of expression of the polypeptides of the invention. Inactivation can be carried out using homologous recombination methods described above. Activation can be achieved by supplementing or even replacing the homologous promoter to provide for increased protein expression. The homologous promoter can be supplemented by insertion of one or more heterologous enhancer elements known to confer promoter activation in a particular tissue.

The polynucleotides of the present invention also make possible the development, through, e.g., homologous recombination or knock out strategies, of animals that fail to express functional polypeptides of the invention or that express a variant of the polypeptides of the invention. Such animals are useful as models for studying the in vivo activities of polypeptides of the invention as well as for studying modulators of the polypeptides of the invention.

4.11 Uses and Biological Activity

The polynucleotides and proteins of the present invention are expected to exhibit one or more of the uses or biological activities (including those associated with assays cited herein) identified herein. Uses or activities described for proteins of the present invention may be provided by administration or use of such proteins or of polynucleotides encoding such proteins (such as, for example, in gene therapies or vectors suitable for introduction of DNA). The mechanism underlying the particular condition or pathology will dictate whether the polypeptides of the invention, the polynucleotides of the invention or modulators (activators or inhibitors) thereof would be beneficial to the subject in need of treatment. Thus, “therapeutic compositions of the invention” include compositions comprising isolated polynucleotides (including recombinant DNA molecules, cloned genes and degenerate variants thereof) or polypeptides of the invention (including full length protein, mature protein and truncations or domains thereof), or compounds and other substances that modulate the overall activity of the target gene products, either at the level of target gene/protein expression or target protein activity. Such modulators include polypeptides, analogs, (variants), including fragments and fusion proteins, antibodies and other binding proteins; chemical compounds that directly or indirectly activate or inhibit the polypeptides of the invention (identified, e.g., via drug screening assays as described herein); antisense polynucleotides and polynucleotides suitable for triple helix formation; and in particular antibodies or other binding partners that specifically recognize one or more epitopes of the polypeptides of the invention.

The polypeptides of the present invention may likewise be involved in cellular activation or in one of the other physiological pathways described herein.

4.11.1 Research Uses and Utilities

The polynucleotides provided by the present invention can be used by the research community for various purposes. The polynucleotides can be used to express recombinant protein for analysis, characterization or therapeutic use; as markers for tissues in which the corresponding protein is preferentially expressed (either constitutively or at a particular stage of tissue differentiation or development or in disease states); as chromosome markers or tags (when labeled) to identify chromosomes or to map related gene positions; to compare with endogenous DNA sequences in patients to identify potential genetic disorders; as probes to hybridize and thus discover novel, related DNA sequences; as a source of information to derive PCR primers for genetic fingerprinting; as a probe to “subtract-out” known sequences in the process of discovering other novel polynucleotides; for selecting and making oligomers for attachment to a “gene chip” or other support, including for examination of expression patterns; to raise anti-protein antibodies using DNA immunization techniques; and as an antigen to raise anti-DNA antibodies or elicit another immune response. Where the polynucleotide encodes a protein which binds or potentially binds to another protein (such as, for example, in a receptor-ligand interaction), the polynucleotide can also be used in interaction trap assays (such as, for example, that described in Gyuris et al., Cell 75:791-803 (1993)) to identify polynucleotides encoding the other protein with which binding occurs or to identify inhibitors of the binding interaction.

The polypeptides provided by the present invention can similarly be used in assays to determine biological activity, including in a panel of multiple proteins for high-throughput screening; to raise antibodies or to elicit another immune response; as a reagent (including the labeled reagent) in assays designed to quantitatively determine levels of the protein (or its receptor) in biological fluids; as markers for tissues in which the corresponding polypeptide is preferentially expressed (either constitutively or at a particular stage of tissue differentiation or development or in a disease state); and, of course, to isolate correlative receptors or ligands. Proteins involved in these binding interactions can also be used to screen for peptide or small molecule inhibitors or agonists of the binding interaction.

The polypeptides of the invention are also useful for making antibody substances that are specifically immunoreactive with proteins according to the invention. Antibodies and portions thereof (e.g., Fab fragments) which bind to the polypeptides of the invention can be used to identify the presence of such polypeptides in a sample. Such determinations are carried out using any suitable immunoassay format, and any polypeptide of the invention that is specifically bound by the antibody can be employed as a positive control.

Any or all of these research utilities are capable of being developed into reagent grade or kit format for commercialization as research products.

Methods for performing the uses listed above are well known to those skilled in the art. References disclosing such methods include without limitation “Molecular Cloning: A Laboratory Manual”, 2d ed., Cold Spring Harbor Laboratory Press, Sambrook, J., E. F. Fritsch and T. Maniatis eds., 1989, and “Methods in Enzymology: Guide to Molecular Cloning Techniques”, Academic Press, Berger, S. L. and A. R. Kimmel eds., 1987.

4.11.2 Cytokine and Cell Proliferation/Fifferentiation Activity

A polypeptide of the present invention may exhibit activity relating to cytokine, cell proliferation (either inducing or inhibiting) or cell differentiation (either inducing or inhibiting) activity or may induce production of other cytokines in certain cell populations. A polynucleotide of the invention can encode a polypeptide exhibiting such attributes. Many protein factors discovered to date, including all known cytokines, have exhibited activity in one or more factor-dependent cell proliferation assays, and hence the assays serve as a convenient confirmation of cytokine activity. The activity of therapeutic compositions of the present invention is evidenced by any one of a number of routine factor dependent cell proliferation assays for cell lines including, without limitation, 32D, DA2, DA1G, T10, B9, B9/11, BaF3, MC9/G, M+(preB M+), 2E8, RB5, DA1, 123, T1165, HT2, CTLL2, TF1, Mo7e, CMK, HUVEC, and Caco. Therapeutic compositions of the invention can be used in the following:

Assays for T-cell or thymocyte proliferation include without limitation those described in: Current Protocols in Immunology, Ed by J. E. Coligan, A. M. Kruisbeek, D. H. Margulies, E. M. Shevach, W. Strober, Pub. Greene Publishing Associates and Wiley-Interscience (Chapter 3, In Vitro assays for Mouse Lymphocyte Function 3.1-3.19; Chapter 7, Immunologic studies in Humans); Takai, et al., J. Immunol. 137:3494-3500 (1986); Bertagnolli, et al., J. Immunol. 145:1706-1712 (1990); Bertagnolli, et al., Cellular Immunology 133:327-341 (1991); Bertagnolli, et al., J. Immunol. 149:3778-3783 (1992); Bowman, et al., J. Immunol. 152:1756-1761 (1994).

Assays for cytokine production and/or proliferation of spleen cells, lymph node cells or thymocytes include, without limitation, those described in: Polyclonal T cell stimulation, Kruisbeek, A. M. and Shevach, E. M. In Current Protocols in Immunology. J. E. e.a. Coligan eds. Vol 1 pp. 3.12.1-3.12.14, John Wiley and Sons, Toronto. 1994; and Measurement of mouse and human interferon-γ, Schreiber, R. D. In Current Protocols in Immunology. J. E. e.a. Coligan eds. Vol 1 pp. 6.8.1-6.8.8, John Wiley and Sons, Toronto. 1994.

Assays for proliferation and differentiation of hematopoietic and lymphopoietic cells include, without limitation, those described in: Measurement of Human and Murine Interleulin 2 and Interleukin 4, Bottornly, K., Davis, L. S. and Lipsky, P. E. In Current Protocols in Immunology. J. E. e.a. Coligan eds. Vol 1 pp. 6.3.1-6.3.12, John Wiley and Sons, Toronto. 1991; deVries, et al., J. Exp. Med. 173:1205-1211 (1991); Moreau, et al., Nature 336:690-692 (1988); Greenberger, et al., Proc. Natl. Acad. Sci. U.S.A. 80:2931-2938 (1983); Measurement of mouse and human interleukin 6—Nordan, R. In Current Protocols in Immunology. J. E. Coligan eds. Vol 1 pp. 6.6.1-6.6.5, John Wiley and Sons, Toronto. 1991; Smith, et al., Proc. Natl. Aced. Sci. U.S.A. 83:1857-1861 (1986); Measurement of human Interleukin 11—Bennett, F., Giannotti, J., Clark, S. C. and Turner, K. J. In Current Protocols in Immunology. J. E. Coligan eds. Vol 1 pp. 6.15.1 John Wiley and Sons, Toronto. 1991; Measurement of mouse and human Interleukin 9-Ciarletta, A., Giannotti, J., Clark, S. C. and Turner, K. J. In Current Protocols in Immunology. J. E. Coligan eds. Vol 1 pp. 6.13.1, John Wiley and Sons, Toronto. 1991.

Assays for T-cell clone responses to antigens (which will identify, among others, proteins that affect APC-T cell interactions as well as direct T-cell effects by measuring proliferation and cytokine production) include, without limitation, those described in: Current Protocols in Immunology, Ed by J. E. Coligan, A. M. Kruisbeek, D. H. Margulies, E. M. Shevach, W Strober, Pub. Greene Publishing Associates and Wiley-Interscience (Chapter 3, In Vitro assays for Mouse Lymphocyte Function; Chapter 6, Cytokines and their cellular receptors; Chapter 7, Immunologic studies in Humans); Weinberger, et al., Proc. Natl. Acad. Sci. USA 77:6091-6095 (1980); Weinberger, et al., Eur. J. Immun. 11:405-411 (1981); Takai, et al., J. Immunol. 137:3494-3500 (1986); Takai, et al., J. Immunol. 140:508-512 (1988).

4.11.3 Stem Cell Growth Factor Activity

A polypeptide of the present invention may exhibit stem cell growth factor activity and be involved in the proliferation, differentiation and survival of pluripotent and totipotent stem cells including primordial germ cells, embryonic stem cells, hematopoietic stem cells and/or germ line stem cells. Administration of the polypeptide of the invention to stem cells in vivo or ex vivo may maintain and expand cell populations in a totipotential or pluripotential state which would be useful for re-engineering damaged or diseased tissues, transplantation, and manufacture of bio-pharmaceuticals and the development of bio-sensors. The ability to produce large quantities of human cells has important working applications for the production of human proteins which currently must be obtained from non-human sources or donors, implantation of cells to treat diseases such as Parkinson's, Alzheimer's and other neurodegenerative diseases; tissues for grafting such as bone marrow, slin, cartilage, tendons, bone, muscle (including cardiac muscle), blood vessels, cornea, neural cells, gastrointestinal cells and others; and organs for transplantation such as kidney, liver, pancreas (including islet cells), heart and lung.

It is contemplated that multiple different exogenous growth factors and/or cytokines may be administered in combination with the polypeptide of the invention to achieve the desired effect, including any of the growth factors listed herein, other stem cell maintenance factors, and specifically including stem cell factor (SCF), leukemia inhibitory factor (LIF), Flt-3 ligand (Flt-3L), any of the interleukins, recombinant soluble IL-6 receptor fused to IL-6, macrophage inflammatory protein 1-alpha (MIP-1-alpha), G-CSF, GM-CSF, thrombopoietin (TPO), platelet factor 4 (PF-4), platelet-derived growth factor (PDGF), neural growth factors and basic fibroblast growth factor (bFGF).

Since totipotent stem cells can give rise to virtually any mature cell type, expansion of these cells in culture will facilitate the production of large quantities of mature cells. Techniques for culturing stem cells are known in the art and administration of polypeptides of the invention, optionally with other growth factors and/or cytolines, is expected to enhance the survival and proliferation of the stem cell populations. This can be accomplished by direct administration of the polypeptide of the invention to the culture medium. Alternatively, stroma cells transfected with a polynucleotide that encodes for the polypeptide of the invention can be used as a feeder layer for the stem cell populations in culture or in vivo. Stromal support cells for feeder layers may include embryonic bone marrow fibroblasts, bone marrow stromal cells, fetal liver cells, or cultured embryonic fibroblasts (see U.S. Pat. No. 5,690,926).

Stem cells themselves can be transfected with a polynucleotide of the invention to induce autocrine expression of the polypeptide of the invention. This will allow for generation of undifferentiated totipotential/pluripotential stem cell lines that are useful as is or that can then be differentiated into the desired mature cell types. These stable cell lines can also serve as a source of undifferentiated totipotential/pluripotential mRNA to create cDNA libraries and templates for polymerase chain reaction experiments. These studies would allow for the isolation and identification of differentially expressed genes in stem cell populations that regulate stem cell proliferation and/or maintenance.

Expansion and maintenance of totipotent stem cell populations will be useful in the treatment of many pathological conditions. For example, polypeptides of the present invention may be used to manipulate stem cells in culture to give rise to neuroepithelial cells that can be used to augment or replace cells damaged by illness, autoimmune disease, accidental damage or genetic disorders. The polypeptide of the invention may be useful for inducing the proliferation of neural cells and for the regeneration of nerve and brain tissue, i.e. for the treatment of central and peripheral nervous system diseases and neuropathies, as well as mechanical and traumatic disorders which involve degeneration, death or trauma to neural cells or nerve tissue. Furthermore, these cells can be cultured in vitro to form other differentiated cells, such as skin tissue that can be used for transplantation. In addition, the expanded stem cell populations can also be genetically altered for gene therapy purposes and to decrease host rejection of replacement tissues after grafting or implantation.

Expression of the polypeptide of the invention and its effect on stem cells can also be manipulated to achieve controlled differentiation of the stem cells into more differentiated cell types. A broadly applicable method of obtaining pure populations of a specific differentiated cell type from undifferentiated stem cell populations involves the use of a cell-type specific promoter driving a selectable marker. The selectable marker allows only cells of the desired type to survive. For example, stem cells can be induced to differentiate into cardiomyocytes (Wobus et al., Differentiation, 48:173-182 (1991); Klug, et al., J. Clin. Invest., 98:216-224 (1998)) or skeletal muscle cells (Browder, L. W. In: Principles of Tissue Engineering eds. Lanza, et al., Academic Press (1997)). Alternatively, directed differentiation of stem cells can be accomplished by culturing the stem cells in the presence of a differentiation factor such as retinoic acid and an antagonist of the polypeptide of the invention which would inhibit the effects of endogenous stem cell factor activity and allow differentiation to proceed.

In vitro cultures of stem cells can be used to determine if the polypeptide of the invention exhibits stem cell growth factor activity. Stem cells are isolated from any one of various cell sources (including hematopoietic stem cells and embryonic stem cells) and cultured on a feeder layer, as described by Thompson, et al. Proc. Natl. Acad. Sci, U.S.A., 92:7844-7848 (1995), in the presence of the polypeptide of the invention alone or in combination with other growth factors or cytokines. The ability of the polypeptide of the invention to induce stem cells proliferation is determined by colony formation on semi-solid support e.g. as described by Bernstein, et al., Blood, 77: 2316-2321 (1991).

4.11.4 Hematopoiesis Regulating Activity

A polypeptide of the present invention may be involved in regulation of hematopoiesis and, consequently, in the treatment of myeloid or lymphoid cell disorders. Even marginal biological activity in support of colony forming cells or of factor-dependent cell lines indicates involvement in regulating hematopoiesis, e.g. in supporting the growth and proliferation of erythroid progenitor cells alone or in combination with other cytokines, thereby indicating utility, for example, in treating various anemias or for use in conjunction with irradiation/chemotherapy to stimulate the production of erythroid precursors and/or erythroid cells; in supporting the growth and proliferation of myeloid cells such as granulocytes and monocytes/macrophages (i.e., traditional colony stimulating factor activity) useful, for example, in conjunction with chemotherapy to prevent or treat consequent myelo-suppression; in supporting the growth and proliferation of megakaryocytes and consequently of platelets thereby allowing prevention or treatment of various platelet disorders such as thrombocytopenia, and generally for use in place of or complimentary to platelet transfusions; and/or in supporting the growth and proliferation of hematopoietic stem cells which are capable of maturing to any and all of the above-mentioned hematopoietic cells and therefore find therapeutic utility in various stem cell disorders (such as those usually treated with transplantation, including, without limitation, aplastic anemia and paroxysmal nocturnal hemoglobinuria), as well as in repopulating the stem cell compartment post irradiation/chemotherapy, either in vivo or ex vivo (i.e., in conjunction with bone marrow transplantation or with peripheral progenitor cell transplantation (homologous or heterologous)) as normal cells or genetically manipulated for gene therapy.

Therapeutic compositions of the invention can be used in the following:

Suitable assays for proliferation and differentiation of various hematopoietic lines are cited above.

Assays for embryonic stem cell differentiation (which will identify, among others, proteins that influence embryonic differentiation hematopoiesis) include, without limitation, those described in: Johansson, et al. Cellular Biology 15:141-15 (1995); Keller, et al., Mol. Cell. Biol. 13:473-486 (1993); McClanahan, et al., Blood 81:2903-2915 (1993).

Assays for stem cell survival and differentiation (which will identify, among others, proteins that regulate lympho-hematopoiesis) include, without limitation, those described in: Methylcellulose colony forming assays, Freshney, M. G. In Culture of Hematopoietic Cells. R. I. Freshney, et al. eds. Vol pp. 265-268, Wiley-Liss, Inc., New York, N.Y. 1994; Hirayama, et al., Proc. Natl. Acad. Sci. USA 89:5907-5911 (1992); Primitive hematopoietic colony forming cells with high proliferative potential, McNiece, I. K. and Briddell, R. A. In Culture of Hematopoietic Cells. R. I. Freshney, et al. eds. Vol pp. 23-39, Wiley-Liss, Inc., New York, N.Y. 1994; Neben, et al., Experimental Hematology 22:353-359 (1994); Cobblestone area forming cell assay, Ploemacher, R. E. In Culture of Hematopoietic Cells. R. I. Freshney, et al. eds. Vol pp. 1-21, Wiley-Liss, Inc., New York, N.Y. 1994; Long term bone marrow cultures in the presence of stromal cells, Spooncer, E., Dexter, M. and Allen, T. In Culture of Hematopoietic Cells. R. I. Freshney, et al. eds. Vol pp. 163-179, Wiley-Liss, Inc., New York, N.Y. 1994; Long term culture initiating cell assay, Sutherland, H. J. In Culture of Hematopoietic Cells. R. I. Freshney, et al. eds. Vol pp. 139-162, Wiley-Liss, Inc., New York, N.Y. 1994.

4.11.5 Tissue Growth Activity

A polypeptide of the present invention also may be involved in bone, cartilage, tendon, ligament and/or nerve tissue growth or regeneration, as well as in wound healing and tissue repair and replacement, and in healing of burns, incisions and ulcers.

A polypeptide of the present invention which induces cartilage and/or bone growth in circumstances where bone is not normally formed has application in the healing of bone fractures and cartilage damage or defects in humans and other animals. Compositions of a polypeptide, antibody, binding partner, or other modulator of the invention may have prophylactic use in closed as well as open fracture reduction and also in the improved fixation of artificial joints. De novo bone formation induced by an osteogenic agent contributes to the repair of congenital, trauma induced, or oncologic resection induced craniofacial defects, and also is useful in cosmetic plastic surgery.

A polypeptide of this invention may also be involved in attracting bone-forming cells, stimulating growth of bone-forming cells, or inducing differentiation of progenitors of bone-forming cells. Treatment of osteoporosis, osteoarthritis, bone degenerative disorders, or periodontal disease, such as through stimulation of bone and/or cartilage repair or by blocking inflammation or processes of tissue destruction (collagenase activity, osteoclast activity, etc.) mediated by inflammatory processes may also be possible using the composition of the invention.

Another category of tissue regeneration activity that may involve the polypeptide of the present invention is tendon/ligament formation. Induction of tendon/ligament-like tissue or other tissue formation in circumstances where such tissue is not normally formed has application in the healing of tendon or ligament tears, deformities and other tendon or ligament defects in humans and other animals. Such a preparation employing a tendon/ligament-like tissue inducing protein may have prophylactic use in preventing damage to tendon or ligament tissue, as well as use in the improved fixation of tendon or ligament to bone or other tissues, and in repairing defects to tendon or ligament tissue. De novo tendon/ligament-like tissue formation induced by a composition of the present invention contributes to the repair of congenital, trauma induced, or other tendon or ligament defects of other origin, and is also useful in cosmetic plastic surgery for attachment or repair of tendons or ligaments. The compositions of the present invention may provide environment to attract tendon- or ligament-forming cells, stimulate growth of tendon- or ligament-forming cells, induce differentiation of progenitors of tendon- or ligament-forming cells, or induce growth of tendon/ligament cells or progenitors ex vivo for return in vivo to effect tissue repair. The compositions of the invention may also be useful in the treatment of tendinitis, carpal tunnel syndrome and other tendon or ligament defects. The compositions may also include an appropriate matrix and/or sequestering agent as a carrier as is well known in the art.

The compositions of the present invention may also be useful for proliferation of neural cells and for regeneration of nerve and brain tissue, i.e. for the treatment of central and peripheral nervous system diseases and neuropathies, as well as mechanical and traumatic disorders, which involve degeneration, death or trauma to neural cells or nerve tissue. More specifically, a composition of the invention may be used in the treatment of diseases of the peripheral nervous system, such as peripheral nerve injuries, peripheral neuropathy and localized neuropathies, and central nervous system diseases, such as Alzheimer's, Parkinson's disease, Huntington's disease, amyotrophic lateral sclerosis, and Shy-Drager syndrome. Further conditions which may be treated in accordance with the present invention include mechanical and traumatic disorders, such as spinal cord disorders, head trauma and cerebrovascular diseases such as stroke. Peripheral neuropathies resulting from chemotherapy or other medical therapies may also be treatable using a composition of the invention.

Compositions of the invention may also be useful to promote better or faster closure of non-healing wounds, including without limitation pressure ulcers, ulcers associated with vascular insufficiency, surgical and traumatic wounds, and the like.

Compositions of the present invention may also be involved in the generation or regeneration of other tissues, such as organs (including, for example, pancreas, liver, intestine, kidney, skin, and endothelium), muscle (smooth, skeletal or cardiac) and vascular (including vascular endothelium) tissue, or for promoting the growth of cells comprising such tissues. Part of the desired effects may be by inhibition or modulation of fibrotic scarring may allow normal tissue to regenerate. A polypeptide of the present invention may also exhibit angiogenic activity.

A composition of the present invention may also be useful for gut protection or regeneration and treatment of lung or liver fibrosis, reperfusion injury in various tissues, and conditions resulting from systemic cytokine damage.

A composition of the present invention may also be useful for promoting or inhibiting differentiation of tissues described above from precursor tissues or cells; or for inhibiting the growth of tissues described above.

Therapeutic compositions of the invention can be used in the following:

Assays for tissue generation activity include, without limitation, those described in: International Patent Publication No. WO95/16035 (bone, cartilage, tendon); International Patent Publication No. WO95/05846 (nerve, neuronal); International Patent Publication No. WO91/07491 (skin, endothelium).

Assays for wound healing activity include, without limitation, those described in: Winter, Epidermal Wound Healing, pp. 71-112 (Maibach, H. I. and Rovee, D. T., eds.), Year Book Medical Publishers, Inc., Chicago, as modified by Eaglstein and Mertz, J. Invest. Dermatol71:382-84 (1978).

4.11.6 Immune Function Stimulating or Suppressing Activity

A polypeptide of the present invention may also exhibit immune stimulating or immune suppressing activity, including without limitation the activities for which assays are described herein. A polynucleotide of the invention can encode a polypeptide exhibiting such activities. A protein may be useful in the treatment of various immune deficiencies and disorders (including severe combined immunodeficiency (SCID)), e.g., in regulating (up or down) growth and proliferation of T and/or B lymphocytes, as well as effecting the cytolytic activity of NK cells and other cell populations. These immune deficiencies may be genetic or be caused by viral (e.g., HIV) as well as bacterial or fungal infections, or may result from autoimmune disorders. More specifically, infectious diseases causes by viral, bacterial, fungal or other infection may be treatable using a protein of the present invention, including infections by HIV, hepatitis viruses, herpes viruses, mycobacteria, Leishmania spp., malaria spp. and various fungal infections such as candidiasis. Of course, in this regard, proteins of the present invention may also be useful where a boost to the immune system generally may be desirable, i.e., in the treatment of cancer.

Autoimmune disorders which may be treated using a protein of the present invention include, for example, connective tissue disease, multiple sclerosis, systemic lupus erythematosus, rheumatoid arthritis, autoimmune pulmonary inflammation, Guillain-Barre syndrome, autoimmune thyroiditis, insulin dependent diabetes mellitis, myasthenia gravis, graft-versus-host disease and autoimmune inflammatory eye disease. Such a protein (or antagonists thereof, including antibodies) of the present invention may also to be useful in the treatment of allergic reactions and conditions (e.g., anaphylaxis, serum sickness, drug reactions, food allergies, insect venom allergies, mastocytosis, allergic rhinitis, hypersensitivity pneumonitis, urticaria, angioedema, eczema, atopic dermatitis, allergic contact dermatitis, erythema multiforme, Stevens-Johnson syndrome, allergic conjunctivitis, atopic keratoconjunctivitis, venereal keratoconjunctivitis, giant papillary conjunctivitis and contact allergies), such as asthma (particularly allergic asthma) or other respiratory problems. Other conditions, in which immune suppression is desired (including, for example, organ transplantation), may also be treatable using a protein (or antagonists thereof) of the present invention. The therapeutic effects of the polypeptides or antagonists thereof on allergic reactions can be evaluated by in vivo animals models such as the cumulative contact enhancement test (Lastbom, et al., Toxicology 125: 59-66 (1998)), skin prick test (Hoffmann, et al., Allergy 54: 446-54 (1999)), guinea pig skin sensitization test (Vohr, et al., Arch. Toxocol. 73: 501-9), and murine local lymph node assay (Kimber, et al., J. Toxicol. Environ. Health 53: 563-79).

Using the proteins of the invention it may also be possible to modulate immune responses, in a number of ways. Down regulation may be in the form of inhibiting or blocking an immune response already in progress or may involve preventing the induction of an immune response. The functions of activated T cells may be inhibited by suppressing T cell responses or by inducing specific tolerance in T cells, or both. Immunosuppression of T cell responses is generally an active, non-antigen-specific, process which requires continuous exposure of the T cells to the suppressive agent. Tolerance, which involves inducing non-responsiveness or anergy in T cells, is distinguishable from immunosuppression in that it is generally antigen-specific and persists after exposure to the tolerizing agent has ceased. Operationally, tolerance can be demonstrated by the lack of a T cell response upon reexposure to specific antigen in the absence of the tolerizing agent.

Down regulating or preventing one or more antigen functions (including without limitation B lymphocyte antigen functions (such as, for example, B7)), e.g., preventing high level lymphokine synthesis by activated T cells, will be useful in situations of tissue, skin and organ transplantation and in graft-versus-host disease (GVHD). For example, blockage of T cell function should result in reduced tissue destruction in tissue transplantation. Typically, in tissue transplants, rejection of the transplant is initiated through its recognition as foreign by T cells, followed by an immune reaction that destroys the transplant. The administration of a therapeutic composition of the invention may prevent cytokine synthesis by immune cells, such as T cells, and thus acts as an immunosuppressant. Moreover, a lack of costimulation may also be sufficient to anergize the T cells, thereby inducing tolerance in a subject. Induction of long-term tolerance by B lymphocyte antigen-blocking reagents may avoid the necessity of repeated administration of these blocking reagents. To achieve sufficient immunosuppression or tolerance in a subject, it may also be necessary to block the function of a combination of B lymphocyte antigens.

The efficacy of particular therapeutic compositions in preventing organ transplant rejection or GVHD can be assessed using animal models that are predictive of efficacy in humans. Examples of appropriate systems which can be used include allogeneic cardiac grafts in rats and xenogeneic pancreatic islet cell grafts in mice, both of which have been used to examine the immunosuppressive effects of CTLA4Ig fusion proteins in vivo as described in Lenschow, et al., Science 257:789-792 (1992) and Turka, et al., Proc. Natl. Acad. Sci USA, 89:11102-11105 (1992). In addition, murine models of GVHD (see Paul ed., Fundamental Immunology, Raven Press, New York, 1989, pp. 846-847) can be used to determine the effect of therapeutic compositions of the invention on the development of that disease.

Blocking antigen function may also be therapeutically useful for treating autoimmune diseases. Many autoimmune disorders are the result of inappropriate activation of T cells that are reactive against self tissue and which promote the production of cytokines and autoantibodies involved in the pathology of the diseases. Preventing the activation of autoreactive T cells may reduce or eliminate disease symptoms. Administration of reagents which block stimulation of T cells can be used to inhibit T cell activation and prevent production of autoantibodies or T cell-derived cytokines which may be involved in the disease process. Additionally, blocking reagents may induce antigen-specific tolerance of autoreactive T cells which could lead to long-term relief from the disease. The efficacy of blocking reagents in preventing or alleviating autoimmune disorders can be determined using a number of well-characterized animal models of human autoimmune diseases. Examples include murine experimental autoimmune encephalitis, systemic lupus erythematosus in MRL/lpr/lpr mice or NZB hybrid mice, murine autoimmune collagen arthritis, diabetes mellitus in NOD mice and BB rats, and murine experimental myasthenia gravis (see Paul ed., Fundamental Immunology, Raven Press, New York, 1989, pp. 840-856).

Upregulation of an antigen function (e.g., a B lymphocyte antigen function), as a means of up regulating immune responses, may also be useful in therapy. Upregulation of immune responses may be in the form of enhancing an existing immune response or eliciting an initial immune response. For example, enhancing an immune response may be useful in cases of viral infection, including systemic viral diseases such as influenza, the common cold, and encephalitis.

Alternatively, anti-viral immune responses may be enhanced in an infected patient by removing T cells from the patient, costimulating the T cells in vitro with viral antigen-pulsed APCs either expressing a peptide of the present invention or together with a stimulatory form of a soluble peptide of the present invention and reintroducing the in vitro activated T cells into the patient. Another method of enhancing anti-viral immune responses would be to isolate infected cells from a patient, transfect them with a nucleic acid encoding a protein of the present invention as described herein such that the cells express all or a portion of the protein on their surface, and reintroduce the transfected cells into the patient. The infected cells would now be capable of delivering a costimulatory signal to, and thereby activate, T cells in vivo.

A polypeptide of the present invention may provide the necessary stimulation signal to T cells to induce a T cell mediated immune response against the transfected tumor cells. In addition, tumor cells which lack MHC class I or MHC class II molecules, or which fail to reexpress sufficient mounts of MHC class I or MHC class II molecules, can be transfected with nucleic acid encoding all or a portion of (e.g., a cytoplasmic-domain truncated portion) of an MHC class I alpha chain protein and β₂ microglobulin protein or an MC class II alpha chain protein and an MHC class II beta chain protein to thereby express MHC class I or MHC class II proteins on the cell surface. Expression of the appropriate class I or class II MHC in conjunction with a peptide having the activity of a B lymphocyte antigen (e.g., B7-1, B7-2, B7-3) induces a T cell mediated immune response against the transfected tumor cell. Optionally, a gene encoding an antisense construct which blocks expression of an MHC class II associated protein, such as the invariant chain, can also be cotransfected with a DNA encoding a peptide having the activity of a B lymphocyte antigen to promote presentation of tumor associated antigens and induce tumor specific immunity. Thus, the induction of a T cell mediated immune response in a human subject may be sufficient to overcome tumor-specific tolerance in the subject.

The activity of a protein of the invention may, among other means, be measured by the following methods:

Suitable assays for thymocyte or splenocyte cytotoxicity include, without limitation, those described in: Current Protocols in Immunology, Ed by J. E. Coligan, A. M. Kruisbeek, D. H. Margulies, E. M. Shevach, W. Strober, Pub. Greene Publishing Associates and Wiley-Interscience (Chapter 3, In Vitro assays for Mouse Lymphocyte Function 3.1-3.19; Chapter 7, Immunologic studies in Humans); Herrmann, et al., Proc. Natl. Acad. Sci. USA 78:2488-2492 (1981); Herrmann, et al., J. Immunol. 128:1968-1974 (1982); Handa, et al., J. Immunol. 135:1564-1572 (1985); Takai, et al., J. Immunol. 137:3494-3500 (1986); Takai, et al., J. Immunol. 140:508-512 (1988); Bowman, et al., J. Virology 61:1992-1998; Bertagnolli, et al., Cellular Immunology 133:327-341 (1991); Brown, et al., J. Immunol. 153:3079-3092 (1994).

Assays for T-cell-dependent immunoglobulin responses and isotype switching (which will identify, among others, proteins that modulate T-cell dependent antibody responses and that affect Th1/Th2 profiles) include, without limitation, those described in: Maliszewski, J. Immunol. 144:3028-3033 (1990); and Assays for B cell function: In vitro antibody production, Mond, J. J. and Brunswick, M. In Current Protocols in Immunology. J. E. e.a. Coligan eds. Vol 1 pp. 3.8.1-3.8.16, John Wiley and Sons, Toronto. 1994.

Mixed lymphocyte reaction (MLR) assays (which will identify, among others, proteins that generate predominantly Th1 and CTL responses) include, without limitation, those described in: Current Protocols in Immunology, Ed by J. E. Coligan, A. M. Kruisbeek, D. H. Margulies, E. M. Shevach, W. Strober, Pub. Greene Publishing Associates and Wiley-Interscience (Chapter 3, In Vitro assays for Mouse Lymphocyte Function 3.1-3.19; Chapter 7, Immunologic studies in Humans); Takai, et al., J. Immunol. 137:3494-3500 (1986); Takai, et al., J. Immunol. 140:508-512 (1988); Bertagnolli, et al., J. Immunol. 149:3778-3783 (1992).

Dendritic cell-dependent assays (which will identify, among others, proteins expressed by dendritic cells that activate naive T-cells) include, without limitation, those described in: Guery et al., J. Immunol. 134:536-544 (1995); Inaba et al., J. Exp. Med. 173:549-559 (1991); Macatonia, et al., J. Immunol. 154:5071-5079 (1995); Porgador, et al., J. Exp. Med. 182:255-260 (1995); Nair, et al., J. Virology 67:4062-4069 (1993); Huang, et al., Science 264:961-965 (1994); Macatonia, et al., J. Exp. Med. 169:1255-1264 (1989); Bhardwaj, et al., J. Clin. Invest. 94:797-807 (1994); and Inaba, et al., J. Exp. Med. 172:631-640 (1990).

Assays for lymphocyte survival/apoptosis (which will identify, among others, proteins that prevent apoptosis after superantigen induction and proteins that regulate lymphocyte homeostasis) include, without limitation, those described in: Darzynkiewicz et al., Cytometry 13:795-808 (1992); Gorczyca, et al., Leukemia 7:659-670 (1993); Gorczyca, et al., Cancer Res. 53:1945-1951 (1993); Itoh, et al., Cell 66:233-243 (1991); Zacharchuk, J. Immunol. 145:4037-4045 (1990); Zamai, et al., Cytometry 14:891-897 (1993); Gorczyca, et al., Int. J. Oncol. 1:639-648 (1992).

Assays for proteins that influence early steps of T-cell commitment and development include, without limitation, those described in: Antica, et al., Blood 84:111-117 (1994); Fine, et al., Cell. Immunol. 155:111-122, (1994); Galy, et al., Blood 85:2770-2778 (1995); Toki, et al., Proc. Nat. Acad Sci. USA 88:7548-7551 (1991).

4.11.7 Chemotactic/Chemokinetic Activity

A polypeptide of the present invention may be involved in chemotactic or chemokinetic activity for mammalian cells, including, for example, monocytes, fibroblasts, neutrophils, T-cells, mast cells, eosinophils, epithelial and/or endothelial cells. A polynucleotide of the invention can encode a polypeptide exhibiting such attributes Chemotactic and chemokinetic receptor activation can be used to mobilize or attract a desired cell population to a desired site of action. Chemotactic or chemokinetic compositions (e.g. proteins, antibodies, binding partners, or modulators of the invention) provide particular advantages in treatment of wounds and other trauma to tissues, as well as in treatment of localized infections. For example, attraction of lymphocytes, monocytes or neutrophils to tumors or sites of infection may result in improved immune responses against the tumor or infecting agent.

A protein or peptide has chemotactic activity for a particular cell population if it can stimulate, directly or indirectly, the directed orientation or movement of such cell population. Preferably, the protein or peptide has the ability to directly stimulate directed movement of cells. Whether a particular protein has chemotactic activity for a population of cells can be readily determined by employing such protein or peptide in any known assay for cell chemotaxis.

Therapeutic compositions of the invention can be used in the following:

Assays for chemotactic activity (which will identify proteins that induce′ or prevent chemotaxis) consist of assays that measure the ability of a protein to induce the migration of cells across a membrane as well as the ability of a protein to induce the adhesion of one cell population to another cell population. Suitable assays for movement and adhesion include, without limitation, those described in: Current Protocols in Immunology, Ed by J. E. Coligan, A. M. Kruisbeek, D. H. Marguiles, E. M. Shevach, W. Strober, Pub. Greene Publishing Associates and Wiley-Interscience (Chapter 6.12, Measurement of alpha and beta Chemokines 6.12.1-6.12.28; Taub, et al. J. Clin. Invest. 95:1370-1376 (1995); Lind, et al. APMIS 103:140-146 (1995); Muller, et al Eur. J. Immunol. 25:1744-1748; Gruber, et al. J. Immunol. 152:5860-5867 (1994); Johnston, et al. J. Immunol. 153:1762-1768 (1994).

4.11.8 Activin/Inhibin Activity

A polypeptide of the present invention may also exhibit activin- or inhibin-related activities. A polynucleotide of the invention may encode a polypeptide exhibiting such characteristics. Inhibins are characterized by their ability to inhibit the release of follicle stimulating hormone (FSH), while activins and are characterized by their ability to stimulate the release of follicle stimulating hormone (FSH). Thus, a polypeptide of the present invention, alone or in heterodimers with a member of the inhibin family, may be useful as a contraceptive based on the ability of inhibins to decrease fertility in female mammals and decrease spermatogenesis in male mammals. Administration of sufficient amounts of other inhibins can induce infertility in these mammals. Alternatively, the polypeptide of the invention, as a homodimer or as a heterodimer with other protein subunits of the inhibin group, may be useful as a fertility inducing therapeutic, based upon the ability of activin molecules in stimulating FSH release from cells of the anterior pituitary. See, for example, U.S. Pat. No. 4,798,885. A polypeptide of the invention may also be useful for advancement of the onset of fertility in sexually immature mammals, so as to increase the lifetime reproductive performance of domestic animals such as, but not limited to, cows, sheep and pigs.

The activity of a polypeptide of the invention may, among other means, be measured by the following methods.

Assays for activin/inhibin activity include, without limitation, those described in: Vale et al., Endocrinology 91:562-572 (1972); Ling et al., Nature 321:779-782 (1986); Vale et al., Nature 321:776-779 (1986); Mason et al., Nature 318:659-663 (1985); Forage et al., Proc. Natl. Acad. Sci. USA 83:3091-3095 (1986).

4.11.9 Hemostatic and Thrombolytic Activity

A polypeptide of the invention may also be involved in hemostatis or thrombolysis or thrombosis. A polynucleotide of the invention can encode a polypeptide exhibiting such attributes. Compositions may be useful in treatment of various coagulation disorders (including hereditary disorders, such as hemophilias) or to enhance coagulation and other hemostatic events in treating wounds resulting from trauma, surgery or other causes. A composition of the invention may also be useful for dissolving or inhibiting formation of thromboses and for treatment and prevention of conditions resulting therefrom (such as, for example, infarction of cardiac and central nervous system vessels (e.g., stroke).

Therapeutic compositions of the invention can be used in the following:

Assay for hemostatic and thrombolytic activity include, without limitation, those described in: Linet, et al., J. Clin. Pharmacol. 26:131-140 (1986); Burdick, et al., Thrombosis Res. 45:413-419 (1987); Humphrey, et al., Fibrinolysis 5:71-79 (1991); Schaub, Prostaglandins 35:467-474 (1988).

4.11.10 Cancer Diagnosis and Therapy

Polypeptides of the invention may be involved in cancer cell generation, proliferation or metastasis. Detection of the presence or amount of polynucleotides or polypeptides of the invention may be useful for the diagnosis and/or prognosis of one or more types of cancer. For example, the presence or increased expression of a polynucleotide/polypeptide of the invention may indicate a hereditary risk of cancer, a precancerous condition, or an ongoing malignancy. Conversely, a defect in the gene or absence of the polypeptide may be associated with a cancer condition. Identification of single nucleotide polymorphisms associated with cancer or a predisposition to cancer may also be useful for diagnosis or prognosis.

Cancer treatments promote tumor regression by inhibiting tumor cell proliferation, inhibiting angiogenesis (growth of new blood vessels that is necessary to support tumor growth) and/or prohibiting metastasis by reducing tumor cell motility or invasiveness. Therapeutic compositions of the invention may be effective in adult and pediatric oncology including in solid phase tumors/malignancies, locally advanced tumors, human soft tissue sarcomas, metastatic cancer, including lymphatic metastases, blood cell malignancies including multiple myeloma, acute and chronic leukemias, and lymphomas, head and neck cancers including mouth cancer, larynx cancer and thyroid cancer, lung cancers including small cell carcinoma and non-small cell cancers, breast cancers including small cell carcinoma and ductal carcinoma, gastrointestinal cancers including esophageal cancer, stomach cancer, colon cancer, colorectal cancer and polyps associated with colorectal neoplasia, pancreatic cancers, liver cancer, urologic cancers including bladder cancer and prostate cancer, malignancies of the female genital tract including ovarian carcinoma, uterine (including endometrial) cancers, and solid tumor in the ovarian follicle, kidney cancers including renal cell carcinoma, brain cancers including intrinsic brain tumors, neuroblastoma, astrocytic brain tumors, gliomas, metastatic tumor cell invasion in the central nervous system, bone cancers including osteomas, skin cancers including malignant melanoma, tumor progression of human skin keratinocytes, squamous cell carcinoma, basal cell carcinoma, hemangiopericytoma and Karposi's sarcoma.

Polypeptides, polynucleotides, or modulators of polypeptides of the invention (including inhibitors and stimulators of the biological activity of the polypeptide of the invention) may be administered to treat cancer. Therapeutic compositions can be administered in therapeutically effective dosages alone or in combination with adjuvant cancer therapy such as surgery, chemotherapy, radiotherapy, thermotherapy, and laser therapy, and may provide a beneficial effect, e.g. reducing tumor size, slowing rate of tumor growth, inhibiting metastasis, or otherwise improving overall clinical condition, without necessarily eradicating the cancer.

The composition can also be administered in therapeutically effective amounts as a portion of an anti-cancer cocktail. An anti-cancer cocktail is a mixture of the polypeptide or modulator of the invention with one or more anti-cancer drugs in addition to a pharmaceutically acceptable carrier for delivery. The use of anti-cancer cocktails as a cancer treatment is routine. Anti-cancer drugs that are well known in the art and can be used as a treatment in combination with the polypeptide or modulator of the invention include: Actinomycin D, Aminoglutethimide, Asparaginase, Bleomycin, Busulfan, Carboplatin, Carmustine, Chlorambucil, Cisplatin (cis-DDP), Cyclophosphamide, Cytarabine HCl (Cytosine arabinoside), Dacarbazine, Dactinomycin, Daunorubicin HCl, Doxorubicin HCl, Estramustine phosphate sodium, Etoposide (V16-213), Floxuridine, 5-Fluorouracil (5-Fu), Flutamide, Hydroxyurea (hydroxycarbamide), Ifosfamide, Interferon Alpha-2a, Interferon Alpha-2b, Leuprolide acetate (LHRH-releasing factor analog), Lomustine, Mechlorethamine HCl (nitrogen mustard), Melphalan, Mercaptopurine, Mesna, Methotrexate (MTX), Mitomycin, Mitoxantrone HCl, Octreotide, Plicamycin, Procarbazine HCl, Streptozocin, Tamoxifen citrate, Thioguanine, Thiotepa, Vinblastine sulfate, Vincristine sulfate, Amsacrine, Azacitidine, Hexamethylmelamine, Interleukin-2, Mitoguazone, Pentostatin, Semustine, Teniposide, and Vindesine sulfate.

In addition, therapeutic compositions of the invention may be used for prophylactic treatment of cancer. There are hereditary conditions and/or environmental situations (e.g. exposure to carcinogens) known in the art that predispose an individual to developing cancers. Under these circumstances, it may be beneficial to treat these individuals with therapeutically effective doses of the polypeptide of the invention to reduce the risk of developing cancers.

In vitro models can be used to determine the effective doses of the polypeptide of the invention as a potential cancer treatment. These in vitro models include proliferation assays of cultured tumor cells, growth of cultured tumor cells in soft agar (see Freshney, (1987) Culture of Animal Cells: A Manual of Basic Technique, Wily-Liss, New York, NY Ch 18 and Ch 21), tumor systems in nude mice as described in Giovanella, et al., J. Natl. Can. Inst., 52: 921-30 (1974), mobility and invasive potential of tumor cells in Boyden Chamber assays as described in Pilkington, et al., Anticancer Res., 17: 4107-9 (1997), and angiogenesis assays such as induction of vascularization of the chick chorioallantoic membrane or induction of vascular endothelial cell migration as described in Ribatta, et al., Intl. J. Dev. Biol., 40: 1189-97 (1999) and Li, et al., Clin. Exp. Metastasis, 17:423-9 (1999), respectively. Suitable tumor cells lines are available, e.g. from American Type Tissue Culture Collection catalogs.

4.11.11 Immunotargeting of CEA- and Ly-6-Like Polypeptides

4.11.11.1 Targeting Using CEA- and Ly-6-Like Vaccines

One embodiment the present invention provides a vaccine comprising a CEA- or Ly-6-like polypeptide to stimulate the immune system against CEA- or Ly-6-like polypeptides, thus targeting CEA- or Ly-6-like-expressing cells, such as a tumor, spermatocyte, or mature sperm. Use of a tumor antigen in a vaccine for generating cellular and humoral immunity for the purpose of anti-cancer therapy is well known in the art. For example, one type of tumor-specific vaccine uses purified idiotype protein isolated from tumor cells, coupled to keyhole limpet hemocyanin (KLH) and mixed with adjuvant for injection into patients with low-grade follicular lymphoma (Hsu, et al., Blood 89: 3129-3135 (1997)). U.S. Pat. No. 6,312,718 describes methods for inducing immune responses against malignant B cells, in particular lymphoma, chronic lymphocytic leukemia, and multiple myeloma. The methods described therein utilize vaccines that include liposomes having (1) at least one B-cell malignancy-associated antigen, (2) IL-2 alone, or in combination with at least one other cytokine or chemokine, and (3) at least one lipid molecule. Methods of vaccinating against CEA- or Ly-6-like polypeptides typically employ a CEA- or Ly-6-like polypeptide, including fragments, analogs and variants.

As another example, dendritic cells, one type of antigen-presenting cell, can be used in a cellular vaccine in which the dendritic cells are isolated from the patient, co-cultured with tumor antigen and then reinfused as a cellular vaccine (Hsu, et al., Nat. Med. 2:52-58 (1996)).

4.11.11.2 Targeting Using CEA- or LY-6-Like Nucleic Acids

However, in some embodiments, a nucleic acid encoding a CEA- or Ly-6-like polypeptide, or encoding a fragment, analog or variant thereof, within a recombinant vector is utilized. Such methods are known in the art. For example, immune responses can be induced by injection of naked DNA. Plasmid DNA that expresses bicistronic mRNA encoding both the light and heavy chains of tumor idiotype proteins, such as those from B cell lymphoma, when injected into mice, are able to generate a protective, anti-tumor response (Singh, et al., Vaccine 20:1400-1411 (2002)). CEA- or Ly-6-like viral vectors are particularly useful for delivering CEA- or Ly-6-like-encoding nucleic acids to cells. Examples of vectors include those derived from influenza, adenovirus, vaccinia, herpes symplex virus, fowlpox, vesicular stomatitis virus, canarypox, poliovirus, adeno-associated virus, and lentivirus and sindbus virus. Of course, non-viral vectors, such as liposomes or even naked DNA, are also useful for delivering CEA- or Ly-6-like-encoding nucleic acids to cells.

Combining this type of therapy with other types of therapeutic agents or treatments such as chemotherapy or radiation is also contemplated.

In some embodiments, a vector comprising a nucleic acid encoding the CEA- or Ly-6-like polypeptide (including a fragment, analog or variant) is introduced into a cell, such as a dendritic cell or a macrophage. When expressed in an antigen-presenting cell, CEA- or Ly-6-like antigens are presented to T cells eliciting an immune response against CEA- or Ly-6-like polypeptide. Such methods are also known in the art. Methods of introducing tumor antigens into antigen presenting cells and vectors useful therefor are described in U.S. Pat. No. 6,300,090. The vector encoding CEA- or Ly-6-like polypeptide may be introduced into the antigen presenting cells in vivo. Alternatively, antigen-presenting cells are loaded with CEA- or Ly-6-like polypeptide or a nucleic acid encoding CEA- or Ly-6-like polypeptide ex vivo and then introduced into a patient to elicit an immune response against CEA- or Ly-6-like polypeptide. In another alternative, the cells presenting CEA- or Ly-6-like antigen are used to stimulate the expansion of anti-CEA- or Ly-6-like cytotoxic T lymphocytes (CTL) ex vivo followed by introduction of the stimulated CTL into a patient. (U.S. Pat. No. 6,306,388)

Combining this type of therapy with other types of therapeutic agents or treatments such as chemotherapy or radiation is also contemplated.

4.11.11.3 Anti-CEA- or LY-Like Antibodies

Alternatively, immunotargeting involves the administration of components of the immune system, such as antibodies, antibody fragments, or primed cells of the immune system against the target. Methods of immunotargeting cancer cells using antibodies or antibody fragments are well known in the art. U.S. Pat. No. 6,306,393 describes the use of anti-CD22 antibodies in the immunotherapy of B-cell malignancies, and U.S. Pat. No. 6,329,503 describes immunotargeting of cells that express serpentine transmembrane antigens.

CEA- or Ly-6-like antibodies (including humanized or human monoclonal antibodies or fragments or other modifications thereof, optionally conjugated to cytotoxic agents) may be introduced into a patient such that the antibody binds to CEA- or Ly-6-like protein expressed by cancer cells and mediates the destruction of the cells and the tumor and/or inhibits the growth of the cells or the tumor. Without intending to limit the disclosure, mechanisms by which such antibodies can exert a therapeutic effect may include complement-mediated cytolysis, antibody-dependent cellular cytotoxicity (ADCC), modulating the physiologic function of CEA- or Ly-6-like protein, inhibiting binding or signal transduction pathways, modulating tumor cell differentiation, altering tumor angiogenesis factor profiles, modulating the secretion of immune stimulating or tumor suppressing cytokines and growth factors, modulating cellular adhesion, and/or by inducing apoptosis. CEA- or Ly-6-like antibodies conjugated to toxic or therapeutic agents, such as radioligands or cytosolic toxins, may also be used therapeutically to deliver the toxic or therapeutic agent directly to CEA- or Ly-6-like protein-bearing tumor cells.

CEA- or Ly-6-like antibodies may be used to suppress the immune system in patients receiving organ transplants or in patients with autoimmune diseases such as arthritis. Healthy immune cells would be targeted by these antibodies leading their death and clearance from the system, thus suppressing the immune system.

CEA- or Ly-6-like antibodies may be used as antibody therapy for solid tumors which express this action. Cancer immunotherapy using antibodies provides a novel approach to treating cancers associated with cells that specifically express the CEA- or Ly-6-like protein. CEA- or Ly-6-like antibody therapy may be particularly appropriate in advanced or metastatic cancers. Combining the antibody therapy method with a chemotherapeutic, radiation or surgical regimen may be preferred in patients that have not received chemotherapeutic treatment, whereas treatment with the antibody therapy may be indicated for patients who have received one or more chemotherapies. Additionally, antibody therapy can also enable the use of reduced dosages of concomitant chemotherapy, particularly in patients that do not tolerate the toxicity of the chemotherapeutic agent very well. Furthermore, treatment of cancer patients with CEA- or Ly-6-like antibody with tumors resistant to chemotherapeutic agents might induce sensitivity and responsiveness to these agents in combination.

Prior to anti-CEA- or Ly-6-like immunotargeting, a patient may be evaluated for the presence and level of CEA- or Ly-6-like expression by the cancer cells, preferably using immunohistochemical assessments of tumor tissue, quantitative CEA- or Ly-6-like imaging, quantitative RT-PCR, or other techniques capable of reliably indicating the presence and degree of CEA- or Ly-6-like expression. For example, a blood or biopsy sample may be evaluated by immunohistochemical methods to determine the presence of CEA- or Ly-6-like-expressing cells or to determine the extent of CEA- or Ly-6-like expression on the surface of the cells within the sample. Methods for immunohistochemical analysis of tumor tissues or released fragments of CEA- or Ly-6-like in the serum are well known in the art.

Anti-CEA- or Ly-6-like antibodies useful in treating cancers include those, which are capable of initiating a potent immune response against the tumor and those, which are capable of direct cytotoxicity. In this regard, anti-CEA- or Ly-6-like mAbs may elicit tumor cell lysis by either complement-mediated or ADCC mechanisms, both of which require an intact Fc portion of the immunoglobulin molecule for interaction with effector cell Fc receptor sites or complement proteins. In addition, anti-CEA- or Ly-6-like antibodies that exert a direct biological effect on tumor growth are useful in the practice of the invention. Potential mechanisms by which such directly cytotoxic antibodies may act include inhibition of cell growth, modulation of cellular differentiation, modulation of tumor angiogenesis factor profiles, and the induction of apoptosis. The mechanism by which a particular anti-CEA- or Ly-6-like antibody exerts an anti-tumor effect may be evaluated using any number of in vitro assays designed to determine ADCC, complement-mediated cell lysis, and so forth, as is generally known in the art.

The anti-tumor activity of a particular anti-CEA- or Ly-6-like antibody, or combination of anti-CEA- or Ly-6-like antibody, may be evaluated in vivo using a suitable animal model. For example, xenogenic lymphoma cancer models wherein human lymphoma cells are introduced into immune compromised animals, such as nude or SCI) mice. Efficacy may be predicted using assays, which measure inhibition of tumor formation, tumor regression or metastasis, and the like.

It should be noted that the use of murine or other non-human monoclonal antibodies, human/mouse chimeric mAbs may induce moderate to strong immune responses in some patients. In the most severe cases, such an immune response may lead to the extensive formation of immune complexes, which, potentially, can cause renal failure. Accordingly, preferred monoclonal antibodies used in the practice of the therapeutic methods of the invention are those which are either fully human or humanized and which bind specifically to the target CEA- or Ly-6-like antigen with high affinity but exhibit low or no antigenicity in the patient.

The method of the invention contemplates the administration of single anti-CEA- or Ly-6-like monoclonal antibodies (mAbs) as well as combinations, or “cocktails”, of different mAbs. Two or more monoclonal antibodies that bind to CEA- or Ly-6-like protein may provide an improved effect compared to a single antibody. Alternatively, a combination of an anti-CEA- or Ly-6-like antibody with an antibody that binds a different antigen may provide an improved effect compared to a single antibody. Such mAb cocktails may have certain advantages inasmuch as they contain mAbs, which exploit different effector mechanisms or combine directly cytotoxic mAbs with mabs that rely on immune effector functionality. Such mabs in combination may exhibit synergistic therapeutic effects. In addition, the administration of anti-CEA- or Ly-6-like mabs may be combined with other therapeutic agents, including but not limited to various chemotherapeutic agents, androgen-blockers, and immune modulators (e.g., IL-2, GM-CSF). The anti-CEA- or Ly-6-like mabs may be administered in their “naked” or unconjugated form, or may have therapeutic agents conjugated to them. Additionally, bispecific antibodies may be used. Such an antibody would have one antigenic binding domain specific for CEA- or Ly-6-like and the other antigenic binding domain specific for another antigen (such as CD20 for example). Finally, Fab CEA- or Ly-6-like antibodies or fragments of these antibodies (including fragments conjugated to other protein sequences or toxins) may also be used as therapeutic agents.

As another example, Ly-6-like antibodies are used to inhibit fertilization for immunocontraception. Antibodies directed against sperm-specific antigens are used as a pre-fertilizaton contraceptive by inhibiting sperm function or sperm-egg interaction and can be used to immunize both men and women. Anti-sperm antigen antibodies impair fertility by inhibiting sperm transport and/or gamete interaction by a variety of methods: inducing sperm aggregation, altering swimming patterns, imparing sperm penetration through the cervical mucus, immobilizing spermatozoa or invoking the complement cascade resulting in sperm lysis. Anti-sperm antigen antibodies also induce macrophages to phagocytose spermatozoa in the female reproductive tract, blocking the interaction between the receptor and ligand that control binding of the sperm to the zona pellucida, inhibiting penetration of the zona pellucida, as well as interfering with egg/sperm membrane adhesion and fusion (Diekman and Herr, Am. J. Reprod. Immunol. 37:111-117 (1997), herein incorporated by reference).

Studies of the human acrosomal sperm antigen SP-10 as a potential contraceptive immunogen have demonstrated that both polyclonal and monoclonal antibodies directed against SP-10 inhibit binding of capacitated sperm to the zona pellucida using the bovine in vitro fertilization model (Coonrod et al., J. Reprod. Fertil. 107:287-297 (1996), herein incorporated by reference). Oral contraceptives can be developed using attenuated Salmonella typhimunium expressing a recombinant form of the sperm antigen (Herr, Am. J. Reprod. Immunol. 35:184-189 (1996); Srinivasan et al., Biol. Reprod. 53:462-571 (1995), both of which are herein incorporated by reference). Alternatively, immunization can be accomplished using the sperm antigen conjugated to a promiscuous T-cell epitope, such as bovine RNase A, to direct the immune response in human and non-human primates (O'Rand and Lea, J. Reprod. Immunol. 36;51-59 (1997); O'Hem et al., Biol. Reprod. 52:331-339 (1995); Chen et al., J. Immunol. 147:3652-3678 (1991); Bagavant et al., Biol. Reprod. 56:764-770 (1997); Lou et al., J. Immunol. 155:2715-2720 (1995), all of which are herein incorporated by reference).

4.11.11.4 Peptides

CEA- or Ly-6-like peptides themselves, such as fragments of the extracellular region, may be used to target toxins or radioisotopes to tumor cells in vivo by binding to or interacting with CEA- or Ly-6-like polypeptides expressed on tumor or diseased cells. Much like an antibody, these fragments may specifically target cells expressing this antigen. Targeted delivery of these cytotoxic agents to the tumor cells would result in cell death and suppression of tumor growth. An example of the ability of an extracellular fragment binding to and activating its intact receptor (by homophilic binding) has been demonstrated with the CD84 receptor (Martin et al., J. Immunol. 167:3668-3676 (2001), herein incorporated by reference in its entirety).

Extracellular fragments of CEA- or Ly-6-like polypeptides may also be used to modulate immune cells expressing the protein. Extracellular domain fragments of CEA- or Ly-6-like proteins may bind to and activate its own receptor on the cell surface, which may result in stimulating the release of cytokines (such as interferon gamma from NK cells, T cells, B cells or myeloid cells, for example) that may enhance or suppress the immune system. Additionally, binding of these fragments to cells bearing CEA- or Ly-6-like polypeptides may result in the activation of these cells and also may stimulate proliferation. Some fragments may bind to intact CEA- or Ly-6-like polypeptides and block activation signals and cytokine release by immune cells. These fragments would then have an immunosuppressive effect. Fragments that activate and stimulate the immune system may have anti-tumor properties. These fragments may stimulate an immunological response that can result in immune-mediated tumor cell killing. The same fragments may result in stimulating the immune system to mount an enhanced response to foreign invaders such as viruses and bacteria Fragments that suppress the immune response may be useful in treating lymphoproliferative disorders, auto-immune diseases, graft-vs-host disease, and inflammatory diseases, such as emphysema

4.11.11.5 Other Binding Peptides or Small Molecules

Screening of organic compound or peptide libraries with recombinantly expressed CEA- or Ly-6-like protein may be useful for identification of therapeutic molecules that function to specifically bind to or even inhibit the activity of CEA- or Ly-6-like proteins. Synthetic and naturally occurring products can be screened in a number of ways deemed routine to those of skill in the art. Random peptide libraries are displayed on phage (phage display) or on bacteria, such as on E. coli. These random peptide display libraries can be used to screen for peptides which interact with a known target which can be a protein or a polypeptide, such as a ligand or receptor, a biological or synthetic macromolecule, or organic or inorganic substances. By way of example, diversity libraries, such as random or combinatorial peptide or nonpeptide libraries can be screened for molecules that specifically bind to CEA- or Ly-6-like polypeptides. Many libraries are known in the art that can be used, i.e. chemically synthesized libraries, recombinant (i.e. phage display libraries), and in vitro translation-based libraies. Techniques for creating and screening such random peptide display libraries are known in the art (Ladner et al., U.S. Pat. No. 5,223,409; Ladner et al., U.S. Pat. No. 4,946,778; Ladner et al., U.S. Pat. No. 5,403,484; Ladner et al., U.S. Pat. No. 5,571,698, all of which are herein incorporated by reference in their entirety) and random peptide display libraries and kits for screening such libraries are available commercially, for instance from Clontech (Palo Alto, Calif.), Invitrogen Inc. (San Diego, Calif.), New England Biolabs, Inc. (Beverly, Mass.), and Pharmacia KLB Biotechnology Inc. (Piscataway, N.J.). Random peptide display libraries can be screened using the CEA- or Ly-6-like sequences disclosed herein to identify proteins which bind to the CEA- or Ly-6-like polypeptides.

Examples of chemically synthesized libraries are described in Fodor et al., Science 251:767-773 (1991); Houghten et al., Nature 354:84-86 (1991); Lam et al., Nature 354:82-84 (1991); Medynsid, Bio/Technology 12:709-710 (1994); Gallop et al., J. Med. Chem. 37:1233-1251 (1994); Ohlmeyer et al., Proc. Natl. Acad. Sci. USA 90:10922-10926 (1993); Erb et al., Proc. Natl. Acad. Sci. USA 91:11422-11426 (1994); Houghten et al., Biotechniques 13:412 (1992); Jayawickreme et al., Proc. Natl. Acad. Sci. USA 91:1614-1618 (1994); Salmon et al., Proc. Natl. Acad. Sci. USA 90:11708-11712 (1993); PCT Publication No. WO 93/20242; Brenner and Lemer, Proc. Natl. Acad. Sci. USA 89:5381-5383 (1992), all of which are herein incorporated by reference in their entirety.

Examples of phage display libraries are described in Scott and Smith, Science 249:386-390 (1990); Devlin et al., Science 249:404-406 (1990); Christian et al., J. Mol. Biol. 227:711-718 (1992); Lenstra, J. Immunol Meth. 152:149-157 (1992); Kay et al., Gene 128:59-65 (1993); PCT Publication No. WO 94/18318, all of which are herein incorporated by reference in their entirety.

In vitro translation-based libraries include but are not limited to those described in PCT Publication No. WO 91/05058, and Mattheakis et al., Proc. Natl. Acad. Sci. USA 91:9022-9026 (1994), both of which are herein incorporated by reference in their entirety.

By way of examples of nonpeptide libraries, a benzodiazepine library (see for example, Bunin et al., Proc. Natl. Acad. Sci. USA 91:4708-4712 (1994), herein incorporated by reference in its entirety) can be adapted for use. Peptoid libraries (Simon et al., Proc. Natl. Acad. Sci. USA 89:9367-9371 (1992), herein incorporated by reference in its entirety) can also be used. Another example of a library that can be used, in which the amide functionalities in peptides have been permethylated to generate a chemically transformed combinatorial library, is described by Ostresh et al. (Proc. Natl. Acad. Sci. USA 91:11138-11142 (1994), herein incorporated by reference in its entirety).

Screening the libraries can be accomplished by any of a variety of commonly known methods. See, for example, the following references which disclose screening of peptide libraries: Parmley and Smith, Adv. Exp. Med. Biol. 251:215-218 (1989); Scott and Smith, Science 249:386-390 (1990); Fowlkes et al., Biotechniques 13:422-427 (1992); Oldenburg et al., Proc. Natl. Acad. Sci. USA 89:5393-5397 (1992); Yu et al., Cell 76:933-945 (1994); Staudt et al., Science 241:577-580 (1988); Bock et al., Nature 355:564-566 (1992); Tuerk et al., Proc. Natl. Acad. Sci. USA 89:6988-6992 (1992); Ellington et al., Nature 355:850-852 (1992); Rebar and Pabo, Science 263:671-673 (1993); and PCT Publication No. WO 94/18318, all of which are herein incorporated by reference in their entirety.

In a specific embodiment, screening can be carried out by contacting the library members with a CEA- or Ly-6-like protein (or nucleic acid or derivative) immobilized on a solid phase and harvesting those library members that bind to the protein (or nucleic acid or derivative). Examples of such screening methods, termed “panning” techniques are described by way of example in Parmley and Smith, Gene 73:305-318 (1988); Fowlkes et al., Biotechniques 13:422-427 (1992); PCT Publication No. WO 94/18318, all of which are herein incorporated by reference in their entirety, and in references cited hereinabove.

In another embodiment, the two-hybrid system for selecting interacting protein in yeast (Fields and Song, Nature 340:245-246 (1989); Chien et al., Proc. Natl. Acad. Sci. USA 88:9578-9582 (1991), both of which are herein incorporated by reference in their entirety) can be used to identify molecules that specifically bind to a CEA- or Ly-6-like protein or derivative.

These “binding polypeptides” or small molecules which interact with CEA- or Ly-6-like polypeptides can be used for tagging or targeting cells; for isolating homolog polypeptides by affinity purification; they can be directly or indirectly conjugated to drugs, toxins, radionuclides and the like. These binding polypeptides or small molecules can also be used in analytical methods such as for screening expression libraries and neutralizing activity, i.e., for blocling interaction between ligand and receptor, or viral binding to a receptor. The binding polypeptides or small molecules can also be used for diagnostic assays for determining circulating levels of CEA- or Ly-6-like polypeptides; for detecting or quantitating soluble CEA- or Ly-6-like polypeptides as marker of underlying pathology or disease. These binding polypeptides or small molecules can also act as CEA- or Ly-6-like “antagonists” to block CEA- or Ly-6-like binding and signal transduction in vitro and in vivo. These anti-CEA- or Ly-6-like binding polypeptides or small molecules would be useful for inhibiting CEA- or Ly-6-like activity or protein binding.

Binding polypeptides can also be directly or indirectly conjugated to drugs, toxins, radionuclides and the like, and these conjugates used for in vivo diagnostic or therapeutic applications. Binding peptides can also be fused to other polypeptides, for example an immunoglobulin constant chain or portions thereof, to enhance their half-life, and can be made multivalent (through, e.g. branched or repeating units) to increase binding affinity for the CEA- or Ly-6-like polypeptides. For instance, binding polypeptides of the present invention can be used to identify or treat tissues or organs that express a corresponding anti-complementary molecule (receptor or antigen, respectively, for instance). More specifically, binding polypeptides or bioactive fragments or portions thereof, can be coupled to detectable or cytotoxic molecules and delivered to a mammal having cells, tissues or organs that express the anti-complementary molecule.

Suitable detectable molecules may be directly or indirectly attached to the binding polypeptide, and include radionuclides, enzymes, substrates, cofactors, inhibitors, fluorescent markers, chemiluminescent markers, magnetic particles and the like. Suitable cytotoxic molecules may be directly or indirectly attached to the binding polypeptide, and include bacterial or plant toxins (for instance, diphtheria toxin, Pseudomonas exotoxin, ricin, abrin and the like), as well as therapeutic radionuclides, such as iodine-131, rhenium-188, or yttrium-90 (either directly attached to the binding polypeptide, or indirectly attached through a means of a chelating moiety, for instance). Binding polypeptides may also be conjugated to cytotoxic drugs, such as adriamycin. For indirect attachment of a detectable or cytotoxic molecule, the detectable or cytotoxic molecule can be conjugated with a member of a complementary/anticomplementary pair, where the other member is bound to the binding polypeptide. For these purposes, biotin/streptavidin is an exemplary complementary/anticomplementary pair.

In another embodiment, binding polypeptide-toxin fusion proteins can be used for targeted cell or tissue inhibition or ablation (for instance, to treat cancer cells or tissues). Alternatively, if the binding polypeptide has multiple functional domains (i.e., an activation domain or a ligand binding domain, plus a targeting domain), a fusion protein including only the targeting domain may be suitable for directing a detectable molecule, a cytotoxic molecule, or a complementary molecule to a cell or tissue type of interest. In instances where the domain only fusion protein includes a complementary molecule, the anti-complementary molecule can be conjugated to a detectable or cytotoxic molecule. Such domain-complementary molecule fusion proteins thus represent a generic targeting vehicle for cell/tissue-specific delivery of generic anti-complementary-detectable/cytotoxic molecule conjugates.

4.11.12 Receptor/Ligand Activity

A polypeptide of the present invention may also demonstrate activity as receptor, receptor ligand or inhibitor or agonist of receptor/ligand interactions. A polynucleotide of the invention can encode a polypeptide exhibiting such characteristics. Examples of such receptors and ligands include, without limitation, cytokine receptors and their ligands, receptor kinases and their ligands, receptor phosphatases and their ligands, receptors involved in cell-cell interactions and their ligands (including without limitation, cellular adhesion molecules (such as selectins, integrins and their ligands) and receptor/ligand pairs involved in antigen presentation, antigen recognition and development of cellular and humoral immune responses. Receptors and ligands are also useful for screening of potential peptide or small molecule inhibitors of the relevant receptor/ligand interaction. A protein of the present invention (including, without limitation, fragments of receptors and ligands) may themselves be useful as inhibitors of receptor/ligand interactions.

The activity of a polypeptide of the invention may, among other means, be measured by the following methods:

Suitable assays for receptor-ligand activity include without limitation those described in: Current Protocols in Immunology, Ed by J. E. Coligan, A. M. Kruisbeek, D. H. Margulies, E. M. Shevach, W. Strober, Pub. Greene Publishing Associates and Wiley-Interscience (Chapter 7.28, Measurement of Cellular Adhesion under static conditions 7.28.1-7.28.22), Takai, et al., Proc. Natl. Acad. Sci. USA 84:6864-6868 (1987); Bierer, et al., J. Exp. Med. 168:1145-1156 (1988); Rosenstein, et al., J. Exp. Med. 169:149-160 (1989); Stoltenborg, et al., J. Immunol. Methods 175:59-68 (1994); Stift, et al., Cell 80:661-670 (1995).

By way of example, the polypeptides of the invention may be used as a receptor for a ligand(s) thereby transmitting the biological activity of that ligand(s). Ligands may be identified through binding assays, affinity chromatography, dihybrid screening assays, BIAcore assays, gel overlay assays, or other methods known in the art.

Studies characterizing drugs or proteins as agonist or antagonist or partial agonists or a partial antagonist require the use of other proteins as competing ligands. The polypeptides of the present invention or ligand(s) thereof may be labeled by being coupled to radioisotopes, colorimetric molecules or a toxin molecules by conventional methods. (“Guide to Protein Purification” Murray P. Deutscher (ed) Methods in Enzymology Vol. 182 (1990) Academic Press, Inc. San Diego). Examples of radioisotopes include, but are not limited to, tritium and carbon-14. Examples of colorimetric molecules include, but are not limited to, fluorescent molecules such as fluorescamine, or rhodamine or other calorimetric molecules. Examples of toxins include, but are not limited, to ricin.

4.11.13 Drug Screening

This invention is particularly useful for screening chemical compounds by using the novel polypeptides or binding fragments thereof in any of a variety of drug screening techniques. The polypeptides or fragments employed in such a test may either be free in solution, affixed to a solid support, borne on a cell surface or located intracellularly. One method of drug screening utilizes eukaryotic or prokaryotic host cells which are stably transformed with recombinant nucleic acids expressing the polypeptide or a fragment thereof. Drugs are screened against such transformed cells in competitive binding assays. Such cells, either in viable or fixed form, can be used for standard binding assays. One may measure, for example, the formation of complexes between polypeptides of the invention or fragments and the agent being tested or examine the diminution in complex formation between the novel polypeptides and an appropriate cell line, which are well known in the art.

Sources for test compounds that may be screened for ability to bind to or modulate (i.e., increase or decrease) the activity of polypeptides of the invention include (1) inorganic and organic chemical libraries, (2) natural product libraries, and (3) combinatorial libraries comprised of either random or mimetic peptides, oligonucleotides or organic molecules.

Chemical libraries may be readily synthesized or purchased from a number of commercial sources, and may include structural analogs of known compounds or compounds that are identified as “hits” or “leads” via natural product screening.

The sources of natural product libraries are microorganisms (including bacteria and fungi), animals, plants or other vegetation, or marine organisms, and libraries of mixtures for screening may be created by: (1) fermentation and extraction of broths from soil, plant or marine microorganisms or (2) extraction of the organisms themselves. Natural product libraries include polyketides, non-ribosomal peptides, and (non-naturally occurring) variants thereof. For a review, see Science 282:63-68 (1998).

Combinatorial libraries are composed of large numbers of peptides, oligonucleotides or organic compounds and can be readily prepared by traditional automated synthesis methods, PCR, cloning or proprietary synthetic methods. Of particular interest are peptide and oligonucleotide combinatorial libraries. Still other libraries of interest include peptide, protein, peptidomimetic, multiparallel synthetic collection, recombinatorial, and polypeptide libraries. For a review of combinatorial chemistry and libraries created therefrom, see Myers, Curr. Opin. Biotechnol. 8:701-707 (1997). For reviews and examples of peptidomimetic libraries, see Al-Obeidi et al., Mol. Biotechnol, 9:205-23 (1998); Hruby, et al., Curr Opin Chem Biol, 1:114-19 (1997); Domer, et al., Bioorg Med Chem, 4:709-15 (1996) (alkylated dipeptides).

Identification of modulators through use of the various libraries described herein permits modification of the candidate “hit” (or “lead”) to optimize the capacity of the “hit” to bind a polypeptide of the invention. The molecules identified in the binding assay are then tested for antagonist or agonist activity in in vivo tissue culture or animal models that are well known in the art. In brief, the molecules are titrated into a plurality of cell cultures or animals and then tested for either cell/animal death or prolonged survival of the animal/cells.

The binding molecules thus identified may be complexed with toxins, e.g., ricin or cholera, or with other compounds that are toxic to cells such as radioisotopes. The toxin-binding molecule complex is then targeted to a tumor or other cell by the specificity of the binding molecule for a polypeptide of the invention. Alternatively, the binding molecules may be complexed with imaging agents for targeting and imaging purposes.

4.11.14 Assay for Receptor Activity

The invention also provides methods to detect specific binding of a polypeptide e.g. a ligand or a receptor. The invention also provides methods to detect specific binding of a polypeptide of the invention to a binding partner polypeptide, and in particular a ligand polypeptide using assays well known and routinely practiced in the art.

In one embodiment, receptor activity of the polypeptides of the invention is determined using a method that involves (1) forming a mixture comprising a polypeptide of the invention, and/or its agonists and antagonists (or agonist or antagonist drug candidates) and/or antibodies specific for the polypeptides of the invention; (2) incubating the mixture under conditions whereby, but for the presence of said polypeptide of the invention and/or agonists and antagonists (or agonist or antagonist drug candidates) and/or antibodies specific for the polypeptides of the invention, the ligand binds to the receptor; and (3) detecting the presence or absence of specific binding of the polypeptide of the invention to its ligand.

The art provides numerous assays particularly useful for identifying previously unknown binding partners for receptor polypeptides of the invention. For example, expression cloning using mammalian or bacterial cells, or dihybrid screening assays can be used to identify polynucleotides encoding binding partners. As another example, affinity chromatography with the appropriate immobilized polypeptide of the invention can be used to isolate polypeptides that recognize and bind polypeptides of the invention. There are a number of different libraries used for the identification of compounds, and in particular small molecules, that modulate (i.e., increase or decrease) biological activity of a polypeptide of the invention. Ligands for receptor polypeptides of the invention can also be identified by adding exogenous ligands, or cocktails of ligands to two cells populations that are genetically identical except for the expression of the receptor of the invention: one cell population expresses the receptor of the invention whereas the other does not. The response of the two cell populations to the addition of ligands(s) is then compared. Alternatively, an expression library can be co-expressed with the polypeptide of the invention in cells and assayed for an autocrine response to identify potential ligand(s). As still another example, BIAcore assays, gel overlay assays, or other methods known in the art can be used to identify binding partner polypeptides, including, (1) organic and inorganic chemical libraries, (2) natural product libraries, and (3) combinatorial libraries comprised of random peptides, oligonucleotides or organic molecules.

The role of downstream intracellular signaling molecules in the signaling cascade of the polypeptide of the invention can be determined. For example, a chimeric protein in which the cytoplasmic domain of the polypeptide of the invention is fused to the extracellular portion of a protein, whose ligand has been identified, is produced in a host cell. The cell is then incubated with the ligand specific for the extracellular portion of the chimeric protein, thereby activating the chimeric receptor. Known downstream proteins involved in intracellular signaling can then be assayed for expected modifications i.e. phosphorylation. Other methods known to those in the art can also be used to identify signaling molecules involved in receptor activity.

4.11.15 Leukemia

Leukemia and related disorders may be treated or prevented by administration of a therapeutic that promotes or inhibits function of the polynucleotides and/or polypeptides of the invention. Such leukemias and related disorders include but are not limited to acute leukemia, acute lymphocytic leukemia, acute myelocytic leukemia, myeloblastic, promyelocytic, myelomonocytic, monocytic, erythroleukemia, chronic leukemia, chronic myelocytic (granulocytic) leukemia and chronic lymphocytic leukemia (for a review of such disorders, see Fishman, et al., 1985, Medicine, 2d Ed., J.B. Lippincott Co., Philadelphia).

4.11.16 Nervous System Disorders

Nervous system disorders, involving cell types which can be tested for efficacy of intervention with compounds that modulate the activity of the polynucleotides and/or polypeptides of the invention, and which can be treated upon thus observing an indication of therapeutic utility, include but are not limited to nervous system injuries, and diseases or disorders which result in either a disconnection of axons, a diminution or degeneration of neurons, or demyelination. Nervous system lesions which may be treated in a patient (including human and non-human mammalian patients) according to the invention include but are not limited to the following lesions of either the central (including spinal cord, brain) or peripheral nervous systems:

(i) traumatic lesions, including lesions caused by physical injury or associated with surgery, for example, lesions which sever a portion of the nervous system, or compression injuries;

(ii) ischemic lesions, in which a lack of oxygen in a portion of the nervous system results in neuronal injury or death, including cerebral infarction or ischemia, or spinal cord infarction or ischemia;

(iii) infectious lesions, in which a portion of the nervous system is destroyed or injured as a result of infection, for example, by an abscess or associated with infection by human immunodeficiency virus, herpes zoster, or herpes simplex virus or with Lyme disease, tuberculosis, syphilis;

(iv) degenerative lesions, in which a portion of the nervous system is destroyed or injured as a result of a degenerative process including but not limited to degeneration associated with Parkinson's disease, Alzheimer's disease, Huntington's chorea, or amyotrophic lateral sclerosis;

(v) lesions associated with nutritional diseases or disorders, in which a portion of the nervous system is destroyed or injured by a nutritional disorder or disorder of metabolism including but not limited to, vitamin B12 deficiency, folic acid deficiency, Wernicke disease, tobacco-alcohol amblyopia, Marchiafava-Bignami disease (primary degeneration of the corpus callosum), and alcoholic cerebellar degeneration;

(vi) neurological lesions associated with systemic diseases including but not limited to diabetes (diabetic neuropathy, Bell's palsy), systemic lupus erythematosus, carcinoma, or sarcoidosis;

(vii) lesions caused by toxic substances including alcohol, lead, or particular neurotoxins; and

(viii) demyelinated lesions in which a portion of the nervous system is destroyed or injured by a demyelinating disease including but not limited to multiple sclerosis, monophasic demyelination, encephalomyelitis, panencephalaitis, Marchiafava-Bignami disease, Spongy degeneration, Alexander's disease, Canavan's disease, metachromatic leukodystrophy, Krabbe's disease, human immunodeficiency virus-associated myelopathy, transverse myelopathy or various etiologies, progressive multifocal leukoencephalopathy, Guillain-Barre Syndrome, and central pontine myelinolysis.

Therapeutics which are useful according to the invention for treatment of a nervous system disorder may be selected by testing for biological activity in promoting the survival or differentiation of neurons. For example, and not by way of limitation, therapeutics which elicit any of the following effects may be useful according to the invention:

(i) increased survival time of neurons in culture;

(ii) increased sprouting of neurons in culture or in vivo;

(iii) increased production of a neuron-associated molecule in culture or in vivo, e.g., choline acetyltransferase or acetylcholinesterase with respect to motor neurons; or

(iv) decreased symptoms of neuron dysfunction in vivo.

Such effects may be measured by any method known in the art. In preferred, non-limiting embodiments, increased survival of neurons may be measured by the method set forth in Arakawa et al. (J. Neurosci. 10:3507-3515 (1990)); increased sprouting of neurons may be detected by methods set forth in Pestronk, et al. (Exp. Neurol. 70:65-82 (1980)) or Brown, et al. (Ann. Rev. Neurosci. 4:1742 (1981)); increased production of neuron-associated molecules may be measured by bioassay, enzymatic assay, antibody binding, Northern blot assay, etc., depending on the molecule to be measured; and motor neuron dysfunction may be measured by assessing the physical manifestation of motor neuron disorder, e.g., weakness, motor neuron conduction velocity, or functional disability.

In specific embodiments, motor neuron disorders that may be treated according to the invention include but are not limited to disorders such as infarction, infection, exposure to toxin, trauma, surgical damage, degenerative disease or malignancy that may affect motor neurons as well as other components of the nervous system, as well as disorders that selectively affect neurons such as amyotrophic lateral sclerosis, and including but not limited to progressive spinal muscular atrophy, progressive bulbar palsy, primary lateral sclerosis, infantile and juvenile muscular atrophy, progressive bulbar paralysis of childhood (Fazio-Londe syndrome), poliomyelitis and the post polio syndrome, and Hereditary Motorsensory Neuropathy (Charcot-Marie-Tooth Disease).

4.11.17 Other Activities

A polypeptide of the invention may also exhibit one or more of the following additional activities or effects: inhibiting the growth, infection or function of, or killing, infectious agents, including, without limitation, bacteria, viruses, fungi and other parasites; effecting (suppressing or enhancing) bodily characteristics, including, without limitation, height, weight, hair color, eye color, skin, fat to lean ratio or other tissue pigmentation, or organ or body part size or shape (such as, for example, breast augmentation or diminution, change in bone form or shape); effecting biorhythms or circadian cycles or rhythms; effecting the fertility of male or female subjects; effecting the metabolism, catabolism, anabolism, processing, utilization, storage or elimination of dietary fat, lipid, protein, carbohydrate, vitamins, minerals, co-factors or other nutritional factors or component(s); effecting behavioral characteristics, including, without limitation, appetite, libido, stress, cognition (including cognitive disorders), depression (including depressive disorders) and violent behaviors; providing analgesic effects or other pain reducing effects; promoting differentiation and growth of embryonic stem cells in lineages other than hematopoietic lineages; hormonal or endocrine activity; in the case of enzymes, correcting deficiencies of the enzyme and treating deficiency-related diseases; treatment of hyperproliferative disorders (such as, for example, psoriasis); immunoglobulin-like activity (such as, for example, the ability to bind antigens or complement); and the ability to act as an antigen in a vaccine composition to raise an immune response against such protein or another material or entity which is cross-reactive with such protein.

4.11.18 Identification of Polymorphisms

The demonstration of polymorphisms makes possible the identification of such polymorphisms in human subjects and the pharmacogenetic use of this information for diagnosis and treatment. Such polymorphisms may be associated with, e.g., differential predisposition or susceptibility to various disease states (such as disorders involving inflammation or immune response) or a differential response to drug administration, and this genetic information can be used to tailor preventive or therapeutic treatment appropriately. For example, the existence of a polymorphism associated with a predisposition to inflammation or autoimmune disease makes possible the diagnosis of this condition in humans by identifying the presence of the polymorphism.

Polymorphisms can be identified in a variety of ways known in the art which all generally involve obtaining a sample from a patient, analyzing DNA from the sample, optionally involving isolation or amplification of the DNA, and identifying the presence of the polymorphism in the DNA. For example, PCR may be used to amplify an appropriate fragment of genomic DNA which may then be sequenced. Alternatively, the DNA may be subjected to allele-specific oligonucleotide hybridization (in which appropriate oligonucleotides are hybridized to the DNA under conditions permitting detection of a single base mismatch) or to a single nucleotide extension assay (in which an oligonucleotide that hybridizes immediately adjacent to the position of the polymorphism is extended with one or more labeled nucleotides). In addition, traditional restriction fragment length polymorphism analysis (using restriction enzymes that provide differential digestion of the genomic DNA depending on the presence or absence of the polymorphism) may be performed. Arrays with nucleotide sequences of the present invention can be used to detect polymorphisms. The array can comprise modified nucleotide sequences of the present invention in order to detect the nucleotide sequences of the present invention. In the alternative, any one of the nucleotide sequences of the present invention can be placed on the array to detect changes from those sequences.

Alternatively a polymorphism resulting in a change in the amino acid sequence could also be detected by detecting a corresponding change in amino acid sequence of the protein, e.g., by an antibody specific to the variant sequence.

4.11.19 Arthritis and Inflammation

The immunosuppressive effects of the compositions of the invention against rheumatoid arthritis are determined in an experimental animal model system. The experimental model system is adjuvant induced arthritis in rats, and the protocol is described by J. Holoshitz, et al., Science, 219:56 (1983), or by B. Waksman, et al., Int. Arch. Allergy Appl. Immunol., 23:129 (1963). Induction of the disease can be caused by a single injection, generally intradermally, of a suspension of killed Mycobacterium tuberculosis in complete Freund's adjuvant (CFA). The route of injection can vary, but rats may be injected at the base of the tail with an adjuvant mixture. The polypeptide is administered in phosphate buffered solution (PBS) at a dose of about 1-5 mg/kg. The control consists of administering PBS only.

The procedure for testing the effects of the test compound would consist of intradermally injecting killed Mycobacterium tuberculosis in CFA followed by immediately administering the test compound and subsequent treatment every other day until day 24. At 14, 15, 18, 20, 22, and 24 days after injection of Mycobacterium CFA, an overall arthritis score may be obtained as described by J. Holoskitz above. An analysis of the data would reveal that the test compound would have a dramatic affect on the swelling of the joints as measured by a decrease of the arthritis score.

Compositions of the present invention may also exhibit other anti-inflammatory activity. The anti-inflammatory activity may be achieved by providing a stimulus to cells involved in the inflammatory response, by inhibiting or promoting cell-cell interactions (such as, for example, cell adhesion), by inhibiting or promoting chemotaxis of cells involved in the inflammatory process, inhibiting or promoting cell extravasation, or by stimulating or suppressing production of other factors which more directly inhibit or promote an inflammatory response. Compositions with such activities can be used to treat inflammatory conditions including chronic or acute conditions), including without limitation intimation associated with infection (such as septic shock, sepsis or systemic inflammatory response syndrome (SIRS)), ischemia-reperfusion injury, endotoxin lethality, arthritis, complement-mediated hyperacute rejection, nephritis, cytokine or chemokine-induced lung injury, inflammatory bowel disease, Crohn's disease or resulting from over production of cytokines such as TNF or IL-1. Compositions of the invention may also be useful to treat anaphylaxis and hypersensitivity to an antigenic substance or material. Compositions of this invention may be utilized to prevent or treat conditions such as, but not limited to, sepsis, acute pancreatitis, endotoxin shock, cytokine induced shock, rheumatoid arthritis, chronic inflammatory arthritis, pancreatic cell damage from diabetes mellitus type 1, graft versus host disease, inflammatory bowel disease, inflamation associated with pulmonary disease, other autoimmune disease or inflammatory disease, or in the prevention of premature labor secondary to intrauterine infections.

4.11.20 Metabolic Disorders

A polynucleotide and polypeptide of the invention may also be involved in the prevention, diagnosis and management of metabolic disorders involving carbohydrates, lipids, amino acids, vitamins etc., including but not limited to diabetes mellitus, obesity, aspartylglusomarinuria, carbohydrate deficient glycoprotein syndrome (CDGS), cystinosis, diabetes insipidus, Fabry, fatty acid metabolism disorders, galactosemia, Gaucher, glucose-6-phosphate dehydrogenase (G6PD), glutaric aciduria, Hurler, Hurler-Scheie, Hunter, hypophosphatemia, I-cell, Krabbe, lactic acidosis, long chain 3 hydroxyacyl CoA dehydrogenase deficiency (LCHAD), lysosomal storage diseases, mannosidosis, maple syrup urine, Maroteaux-Lamy, metachromatic leukodystrophy, mitochondrial Morquio, mucopolysaccharidosis, neuro-metabolic, Niemann-Pick, organic acidemias, purine, phenylketonuria (PKU), Pompe, porphyria, pseudo-Hurler, pyruvate dehydrogenase deficiency, Sandhoff, Sanfilippo, Scheie, Sly, Tay-Sachs, trimethylaminuria (Fish-Malodor syndrome), urea cycle conditions, vitamin D deficiency rickets and related complications involving different organs including but not limited to liver, heart, kidney, eye, brain, muscle development etc. Hereditary and/or environmental factors known in the art can predispose an individual to developing metabolic disorders and conditions resulting therefrom. Under these circumstances, it maybe beneficial to treat these individual with therapeutically effective doses of the polypeptide of the invention to reduce the risk of developing the disorder. Examples of such disorders include diabetes mellitus, obesity and cardiovascular disease. Further, polynucleotide sequences encoding the invention may be used in Southern or Northern analysis, dot blot, or other membrane-based technologies; in PCR technologies; or in dip stick, pin, ELISA or chip assays utilizing fluids or tissues from patient biopsies to detect altered expression of the polynucleotides of the invention. Such qualitative or quantitative methods are well known in the art.

4.11.21 Cardiovascular Disease and Therapy

Polypeptides and polynucleotides of the invention may also be involved in the prevention, diagnosis and management of cardiovascular disorders such as coronary artery disease, atherosclerosis and hyper- and hypolipoproteinemia, hypertension, angina pectoris, myocardial infarction, congestive heart failure, cardiac arrythmias including paroxysmal arrythmias, restenosis after angioplasty, aortic aneurysm and related complications involving various organs including but not limited to kidney, eye, brain, heart etc. Polypeptides of the invention may also have direct and indirect effects on myocardial contractility, electrical activity of the heart, atrial fibrillation, atrial fluter, anomalous atrio-ventricular pathways, sino-atrial dysfunction, vascular insufficiency and arterial embolism. Hereditary and/or environmental factors known in the art can predispose an individual to developing metabolic disorders and conditions resulting therefrom. Under these circumstances, it maybe beneficial to treat these individual with therapeutically effective doses of the polypeptide of the invention to reduce the risk of developing the disorder. Examples of such disorders include but are not limited to coronary artery disease, atherosclerosis, hyper- and hypolipoproteinemia, hypertension, angina pectoris, myocardial infarction, cardiac arrythmias including paroxysmal arrythmias, diabetes mellitus, inflammatory glomerulonephritis, ischernic renal failure, extracellular matrix accumulation, fibrosis, hypertension, coronary vasoconstriction, ischemic heart disease, and lesions occurring in brain disorders such as stroke, trauma, infarcts, aneurysms.

The polynucleotide sequences encoding the invention may be used in Southern or Northern analysis, dot blot, or other membrane-based technologies; in PCR technologies; or in dip stick, pin, ELISA or chip assays utilizing fluids or tissues from patient biopsies to detect altered expression of the polynucleotides of the invention. Such qualitative or quantitative methods are well known in the art.

4.12 Therapeutic Methods

The compositions (including polypeptide fragments, analogs, variants and antibodies or other binding partners or modulators including antisense polynucleotides) of the invention have numerous applications in a variety of therapeutic methods. Examples of therapeutic applications include, but are not limited to, those exemplified herein.

4.12.1 Example

One embodiment of the invention is the administration of an effective amount of the polypeptides of the invention or other composition of the invention to individuals affected by a disease or disorder that can be modulated by regulating the peptides of the invention. While the mode of administration is not particularly important, parenteral administration is preferred. An exemplary mode of administration is to deliver an intravenous bolus. The dosage of polypeptides of the invention or other composition of the invention will normally be determined by the prescribing physician. It is to be expected that the dosage will vary according to the age, weight, condition and response of the individual patient. Typically, the amount of polypeptide administered per dose will be in the range of about 0.01 μg/kg to 100 mg/kg of body weight, with the preferred dose being about 0.1 μg/kg to 10 mg/kg of patient body weight. For parenteral administration, polypeptides of the invention will be formulated in an injectable form combined with a pharmaceutically acceptable parenteral vehicle. Such vehicles are well known in the art and examples include water, saline, Ringer's solution, dextrose solution, and solutions consisting of small amounts of the human serum albumin. The vehicle may contain minor amounts of additives that maintain the isotonicity and stability of the polypeptide or other active ingredient. The preparation of such solutions is within the skill of the art.

4.13 Pharmaceutical Formulations and Routes of Administration

A protein or other composition of the present invention (from whatever source derived, including without limitation from recombinant and non-recombinant sources and including antibodies and other binding partners of the polypeptides of the invention) maybe administered to a patient in need, by itself, or in pharmaceutical compositions where it is mixed with suitable carriers or excipient(s) at doses to treat or ameliorate a variety of disorders. Such a composition may optionally contain (in addition to protein or other active ingredient and a carrier) diluents, fillers, salts, buffers, stabilizers, solubilizers, and other materials well known in the art. The term “pharmaceutically acceptable” means a non-toxic material that does not interfere with the effectiveness of the biological activity of the active ingredient(s). The characteristics of the carrier will depend on the route of administration. The pharmaceutical composition of the invention may also contain cytokines, lymphokines, or other hematopoietic factors such as M-CSF, GM-CSF, TNF, IL-1, IL-2, IL-3, IL-4, IL-5, IL-6, IL-7, IL-8, IL-9, IL-10, IL-11, IL-12, IL-13, IL-14, IL-15, IFN, TNF0, TNF1, TNF2, G-CSF, Meg-CSF, thrombopoietin, stem cell factor, and erythropoietin. In further compositions, proteins of the invention may be combined with other agents beneficial to the treatment of the disease or disorder in question. These agents include various growth factors such as epidermal growth factor (EGF), platelet-derived growth factor (PDGF), transforming growth factors (TGF-α and TGF-β), insulin-like growth factor (IGF), as well as cytokines described herein.

The pharmaceutical composition may further contain other agents which either enhance the activity of the protein or other active ingredient or complement its activity or use in treatment. Such additional factors and/or agents may be included in the pharmaceutical composition to produce a synergistic effect with protein or other active ingredient of the invention, or to minimize side effects. Conversely, protein or other active ingredient of the present invention may be included in formulations of the particular clotting factor, cytokine, lymphokine, other hematopoietic factor, thrombolytic or anti-thrombotic factor, or anti- inflammatory agent to minimize side effects of the clotting factor, cytokine, lymphokine, other hematopoietic factor, thrombolytic or anti-thrombotic factor, or anti-inflammatory agent (such as IL-1Ra, IL-1 Hyl, IL-1 Hy2, anti-TNF, corticosteroids, immunosuppressive agents). A protein of the present invention may be active in multimers (e.g., heterodimers or homodimers) or complexes with itself or other proteins. As a result, pharmaceutical compositions of the invention may comprise a protein of the invention in such multimeric or complexed form.

As an alternative to being included in a pharmaceutical composition of the invention including a first protein, a second protein or a therapeutic agent may be concurrently administered with the first protein (e.g., at the same time, or at differing times provided that therapeutic concentrations of the combination of agents is achieved at the treatment site). Techniques for formulation and administration of the compounds of the instant application may be found in “Remington's Pharmaceutical Sciences,” Mack Publishing Co., Easton, Pa., latest edition. A therapeutically effective dose further refers to that amount of the compound sufficient to result in amelioration of symptoms, e.g., treatment, healing, prevention or amelioration of the relevant medical condition, or an increase in rate of treatment, healing, prevention or amelioration of such conditions. When applied to an individual active ingredient, administered alone, a therapeutically effective dose refers to that ingredient alone. When applied to a combination, a therapeutically effective dose refers to combined amounts of the active ingredients that result in the therapeutic effect, whether administered in combination, serially or simultaneously.

In practicing the method of treatment or use of the present invention, a therapeutically effective amount of protein or other active ingredient of the present invention is administered to a mammal having a condition to be treated. Protein or other active ingredient of the present invention may be administered in accordance with the method of the invention either alone or in combination with other therapies such as treatments employing cytokines, lymphokines or other hematopoietic factors. When co-administered with one or more cytokines, lympholines or other hematopoietic factors, protein or other active ingredient of the present invention may be administered either simultaneously with the cytokine(s), lymphokine(s), other hematopoietic factor(s), thrombolytic or anti-thrombotic factors, or sequentially. If administered sequentially, the attending physician will decide on the appropriate sequence of administering protein or other active ingredient of the present invention in combination with cytokine(s), lymphokine(s), other hematopoietic factor(s), thrombolytic or anti-thrombotic factors.

4.13.1 Routes of Administration

Suitable routes of administration may, for example, include oral, rectal, transmucosal, or intestinal administration; parenteral delivery, including intramuscular, subcutaneous, intramedullary injections, as well as intrathecal, direct intraventricular, intravenous, intraperitoneal, intranasal, or intraocular injections. Administration of protein or other active ingredient of the present invention used in the pharmaceutical composition or to practice the method of the present invention can be carried out in a variety of conventional ways, such as oral ingestion, inhalation, topical application or cutaneous, subcutaneous, intraperitoneal, parenteral or intravenous injection. Intravenous administration to the patient is preferred.

Alternately, one may administer the compound in a local rather than systemic manner, for example, via injection of the compound directly into arthritic joints or in fibrotic tissue, often in a depot or sustained release formulation. In order to prevent the scarring process frequently occurring as complication of glaucoma surgery, the compounds may be administered topically, for example, as eye drops. Furthermore, one may administer the drug in a targeted drug delivery system, for example, in a liposome coated with a specific antibody, targeting, for example, arthritic or fibrotic tissue. The liposomes will be targeted to and taken up selectively by the afflicted tissue.

The polypeptides of the invention are administered by any route that delivers an effective dosage to the desired site of action. The determination of a suitable route of administration and an effective dosage for a particular indication is within the level of skill in the art. Preferably for wound treatment, one administers the therapeutic compound directly to the site. Suitable dosage ranges for the polypeptides of the invention can be extrapolated from these dosages or from similar studies in appropriate animal models. Dosages can then be adjusted as necessary by the clinician to provide maximal therapeutic benefit.

4.13.2 Compositions/Formulations

Pharmaceutical compositions for use in accordance with the present invention thus may be formulated in a conventional manner using one or more physiologically acceptable carriers comprising excipients and auxiliaries which facilitate processing of the active compounds into preparations which can be used pharmaceutically. These pharmaceutical compositions may be manufactured in a manner that is itself known, e.g. by means of conventional mixing, dissolving, granulating, dragee-making, levigating, emulsifying, encapsulating, entrapping or lyophilizing processes. Proper formulation is dependent upon the route of administration chosen. When a therapeutically effective amount of protein or other active ingredient of the present invention is administered orally, protein or other active ingredient of the present invention will be in the form of a tablet, capsule, powder, solution or elixr. When administered in tablet form, the pharmaceutical composition of the invention may additionally contain a solid carrier such as a gelatin or an adjuvant. The tablet, capsule, and powder contain from about 5 to 95% protein or other active ingredient of the present invention, and preferably from about 25 to 90% protein or other active ingredient of the present invention. When administered in liquid form, a liquid carrier such as water, petroleum, oils of animal or plant origin such as peanut oil, mineral oil, soybean oil, or sesame oil, or synthetic oils may be added. The liquid form of the pharmaceutical composition may further contain physiological saline solution, dextrose or other saccharide solution, or glycols such as ethylene glycol, propylene glycol or polyethylene glycol. When administered in liquid form, the pharmaceutical composition contains from about 0.5 to 90% by weight of protein or other active ingredient of the present invention, and preferably from about 1 to 50% protein or other active ingredient of the present invention.

When a therapeutically effective amount of protein or other active ingredient of the present invention is administered by intravenous, cutaneous or subcutaneous injection, protein or other active ingredient of the present invention will be in the form of a pyrogen-free, parenterally acceptable aqueous solution. The preparation of such parenterally acceptable protein or other active ingredient solutions, having due regard to pH, isotonicity, stability, and the like, is within the skill in the art. A preferred pharmaceutical composition for intravenous, cutaneous, or subcutaneous injection should contain, in addition to protein or other active ingredient of the present invention, an isotonic vehicle such as Sodium Chloride Injection, Ringer's Injection, Dextrose Injection, Dextrose and Sodium Chloride Injection, Lactated Ringer's Injection, or other vehicle as known in the art. The pharmaceutical composition of the present invention may also contain stabilizers, preservatives, buffers, antioxidants, or other additives known to those of skill in the art. For injection, the agents of the invention may be formulated in aqueous solutions, preferably in physiologically compatible buffers such as Hanks's solution, Ringer's solution, or physiological saline buffer. For transmucosal administration, penetrants appropriate to the barrier to be permeated are used in the formulation. Such penetrants are generally known in the art.

For oral administration, the compounds can be formulated readily by combining the active compounds with pharmaceutically acceptable carriers well known in the art. Such carriers enable the compounds of the invention to be formulated as tablets, pills, dragees, capsules, liquids, gels, syrups, slurries, suspensions and the like, for oral ingestion by a patient to be treated. Pharmaceutical preparations for oral use can be obtained solid excipient, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP). If desired, disintegrating agents may be added, such as the cross-linked polyvinyl pyrrolidone, agar, or alginic acid or a salt thereof such as sodium alginate. Dragee cores are provided with suitable coatings. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compound doses.

Pharmaceutical preparations which can be used orally include push-fit capsules made of gelatin, as well as soft, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added. All formulations for oral administration should be in dosages suitable for such administration. For buccal administration, the compositions may take the form of tablets or lozenges formulated in conventional manner.

For administration by inhalation, the compounds for use according to the present invention are conveniently delivered in the form of an aerosol spray presentation from pressurized packs or a nebuliser, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of, e.g., gelatin for use in an inhaler or insufflator may be formulated containing a powder mix of the compound and a suitable powder base such as lactose or starch. The compounds may be formulated for parenteral administration by injection, e.g., by bolus injection or continuous infusion. Formulations for injection may be presented in unit dosage form, e.g., in ampules or in multi-dose containers, with an added preservative. The compositions may take such forms as suspensions, solutions or emulsions in oily or aqueous vehicles, and may contain formulatory agents such as suspending, stabilizing and/or dispersing agents.

Pharmaceutical formulations for parenteral administration include aqueous solutions of the active compounds in water-soluble form. Additionally, suspensions of the active compounds may be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. Optionally, the suspension may also contain suitable stabilizers or agents which increase the solubility of the compounds to allow for the preparation of highly concentrated solutions. Alternatively, the active ingredient maybe in powder form for constitution with a suitable vehicle, e.g., sterile pyrogen-free water, before use.

The compounds may also be formulated in rectal compositions such as suppositories or retention enemas, e.g., containing conventional suppository bases such as cocoa butter or other glycerides. In addition to the formulations described previously, the compounds may also be formulated as a depot preparation. Such long acting formulations may be administered by implantation (for example subcutaneously or intamuscularly) or by intramuscular injection. Thus, for example, the compounds may be formulated with suitable polymeric or hydrophobic materials (for example as an emulsion in an acceptable oil) or ion exchange resins, or as sparingly soluble derivatives, for example, as a sparingly soluble salt.

A pharmaceutical carrier for the hydrophobic compounds of the invention is a co-solvent system comprising benzyl alcohol, a nonpolar surfactant, a water-miscible organic polymer, and an aqueous phase. The co-solvent system may be the VPD co-solvent system. VPD is a solution of 3% w/v benzyl alcohol, 8% w/v of the nonpolar surfactant polysorbate 80, and 65% w/v polyethylene glycol 300, made up to volume in absolute ethanol. The VPD co-solvent system (VPD:5W) consists of VPD diluted 1:1 with a 5% dextrose in water solution. This co-solvent system dissolves hydrophobic compounds well, and itself produces low toxicity upon systemic administration. Naturally, the proportions of a co-solvent system may be varied considerably without destroying its solubility and toxicity characteristics. Furthermore, the identity of the co-solvent components may be varied: for example, other low-toxicity nonpolar surfactants may be used instead of polysorbate 80; the fraction size of polyethylene glycol may be varied; other biocompatible polymers may replace polyethylene glycol, e.g. polyvinyl pyrrolidone; and other sugars or polysaccharides may substitute for dextrose. Alternatively, other delivery systems for hydrophobic pharmaceutical compounds may be employed. Liposomes and emulsions are well known examples of delivery vehicles or carriers for hydrophobic drugs. Certain organic solvents such as dimethylsulfoxide also may be employed, although usually at the cost of greater toxicity. Additionally, the compounds may be delivered using a sustained-release system, such as semipermeable matrices of solid hydrophobic polymers containing the therapeutic agent. Various types of sustained-release materials have been established and are well known by those skilled in the art. Sustained-release capsules may, depending on their chemical nature, release the compounds for a few weeks up to over 100 days. Depending on the chemical nature and the biological stability of the therapeutic reagent, additional strategies for protein or other active ingredient stabilization may be employed.

The pharmaceutical compositions also may comprise suitable solid or gel phase carriers or excipients. Examples of such carriers or excipients include but are not limited to calcium carbonate, calcium phosphate, various sugars, starches, cellulose derivatives, gelatin, and polymers such as polyethylene glycols. Many of the active ingredients of the invention may be provided as salts with pharmaceutically compatible counter ions. Such pharmaceutically acceptable base addition salts are those salts which retain the biological effectiveness and properties of the free acids and which are obtained by reaction with inorganic or organic bases such as sodium hydroxide, magnesium hydroxide, ammonia, trialkylamine, dialkylamine, monoalkylamine, dibasic amino acids, sodium acetate, potassium benzoate, triethanol amine and the like.

The pharmaceutical composition of the invention may be in the form of a complex of the protein(s) or other active ingredient of present invention along with protein or peptide antigens. The protein and/or peptide antigen will deliver a stimulatory signal to both B and T lymphocytes. B lymphocytes will respond to antigen through their surface immunoglobulin receptor. T lymphocytes will respond to antigen through the T cell receptor (TCR) following presentation of the antigen by MHC proteins. MHC and structurally related proteins including those encoded by class I and class II MHC genes on host cells will serve to present the peptide antigen(s) to T lymphocytes. The antigen components could also be supplied as purified MHC-peptide complexes alone or with co-stimulatory molecules that can directly signal T cells. Alternatively antibodies able to bind surface immunoglobulin and other molecules on B cells as well as antibodies able to bind the TCR and other molecules on T cells can be combined with the pharmaceutical composition of the invention.

The pharmaceutical composition of the invention may be in the form of a liposome in which protein of the present invention is combined, in addition to other pharmaceutically acceptable carriers, with amphipathic agents such as lipids which exist in aggregated form as micelles, insoluble monolayers, liquid crystals, or lamellar layers in aqueous solution. Suitable lipids for liposomal formulation include, without limitation, monoglycerides, diglycerides, sulfatides, lysolecithins, phospholipids, saponin, bile acids, and the like. Preparation of such liposomal formulations is within the level of skill in the art, as disclosed, for example, in U.S. Pat. Nos. 4,235,871; 4,501,728; 4,837,028; and 4,737,323, all of which are incorporated herein by reference.

The amount of protein or other active ingredient of the present invention in the pharmaceutical composition of the present invention will depend upon the nature and severity of the condition being treated, and on the nature of prior treatments which the patient has undergone. Ultimately, the attending physician will decide the amount of protein or other active ingredient of the present invention with which to treat each individual patient. Initially, the attending physician will administer low doses of protein or other active ingredient of the present invention and observe the patient's response. Larger doses of protein or other active ingredient of the present invention may be administered until the optimal therapeutic effect is obtained for the patient, and at that point the dosage is not increased further. It is contemplated that the various pharmaceutical compositions used to practice the method of the present invention should contain about 0.01 μg to about 100 mg (preferably about 0.1 μg to about 10 mg, more preferably about 0.1 μg to about 1 mg) of protein or other active ingredient of the present invention per kg body weight. For compositions of the present invention which are useful for bone, cartilage, tendon or ligament regeneration, the therapeutic method includes administering the composition topically, systematically, or locally as an implant or device. When administered, the therapeutic composition for use in this invention is, of course, in a pyrogen-free, physiologically acceptable form. Further, the composition may desirably be encapsulated or injected in a viscous form for delivery to the site of bone, cartilage or tissue damage. Topical administration may be suitable for wound healing and tissue repair. Therapeutically useful agents other than a protein or other active ingredient of the invention which may also optionally be included in the composition as described above, may alternatively or additionally, be administered simultaneously or sequentially with the composition in the methods of the invention. Preferably for bone and/or cartilage formation, the composition would include a matrix capable of delivering the protein-containing or other active ingredient-containing composition to the site of bone and/or cartilage damage, providing a structure for the developing bone and cartilage and optimally capable of being reabsorbed into the body. Such matrices may be formed of materials presently in use for other implanted medical applications.

The choice of matrix material is based on biocompatibility, biodegradability, mechanical properties, cosmetic appearance and interface properties. The particular application of the compositions will define the appropriate formulation. Potential matrices for the compositions may be biodegradable and chemically defined calcium sulfate, tricalcium phosphate, hydroxyapatite, polylactic acid, polyglycolic acid and polyanhydrides. Other potential materials are biodegradable and biologically well-defined, such as bone or dermal collagen. Further matrices are comprised of pure proteins or extracellular matrix components. Other potential matrices are nonbiodegradable and chemically defined, such as sintered hydroxyapatite, bioglass, aluminates, or other ceramics. Matrices may be comprised of combinations of any of the above mentioned types of material, such as polylactic acid and hydroxyapatite or collagen and tricalcium phosphate. The bioceramics may be altered in composition, such as in calcium-aluminate-phosphate and processing to alter pore size, particle size, particle shape, and biodegradability. Presently preferred is a 50:50 (mole weight) copolymer of lactic acid and glycolic acid in the form of porous particles having diameters ranging from 150 to 800 microns. In some applications, it will be useful to utilize a sequestering agent, such as carboxymethyl cellulose or autologous blood clot, to prevent the protein compositions from disassociating from the matrix.

A preferred family of sequestering agents is cellulosic materials such as alkylcelluloses (including hydroxyalkylcelluloses), including methylcellulose, ethylcellulose, hydroxyethylcellulose, hydroxypropylcellulose, hydroxypropyl-methylcellulose, and carboxymethylcellulose, the most preferred being cationic salts of carboxymethylcellulose (CMC). Other preferred sequestering agents include hyaluronic acid, sodium alginate, poly(ethylene glycol), polyoxyethylene oxide, carboxyvinyl polymer and poly(vinyl alcohol). The amount of sequestering agent useful herein is 0.5-20 wt %, preferably 1-10 wt % based on total formulation weight, which represents the amount necessary to prevent desorption of the protein from the polymer matrix and to provide appropriate handling of the composition, yet not so much that the progenitor cells are prevented from infiltrating the matrix, thereby providing the protein the opportunity to assist the osteogenic activity of the progenitor cells. In further compositions, proteins or other active ingredient of the invention may be combined with other agents beneficial to the treatment of the bone and/or cartilage defect, wound, or tissue in question. These agents include various growth factors such as epidermal growth factor (EGF), platelet derived growth factor (PDGF), transforming growth factors (TGF-α and TGF-β), and insulin-like growth factor (IGF).

The therapeutic compositions are also presently valuable for veterinary applications. Particularly domestic animals and thoroughbred horses, in addition to humans, are desired patients for such treatment with proteins or other active ingredient of the present invention. The dosage regimen of a protein-containing pharmaceutical composition to be used in tissue regeneration will be determined by the attending physician considering various factors which modify the action of the proteins, e.g., amount of tissue weight desired to be formed, the site of damage, the condition of the damaged tissue, the size of a wound, type of damaged tissue (e.g., bone), the patient's age, sex, and diet, the severity of any infection, time of administration and other clinical factors. The dosage may vary with the type of matrix used in the reconstitution and with inclusion of other proteins in the pharmaceutical composition. For example, the addition of other known growth factors, such as IGF I (insulin like growth factor I), to the final composition, may also effect the dosage. Progress can be monitored by periodic assessment of tissue/bone growth and/or repair, for example, X-rays, histomorphometric determinations and tetracycline labeling.

Polynucleotides of the present invention can also be used for gene therapy. Such polynucleotides can be introduced either in vivo or ex vivo into cells for expression in a mammalian subject. Polynucleotides of the invention may also be administered by other known methods for introduction of nucleic acid into a cell or organism (including, without limitation, in the form of viral vectors or naked DNA). Cells may also be cultured ex vivo in the presence of proteins of the present invention in order to proliferate or to produce a desired effect on or activity in such cells. Treated cells can then be introduced in vivo for therapeutic purposes.

4.13.3 Effective Dosage

Pharmaceutical compositions suitable for use in the present invention include compositions wherein the active ingredients are contained in an effective amount to achieve its intended purpose. More specifically, a therapeutically effective amount means an amount effective to prevent development of or to alleviate the existing symptoms of the subject being treated. Determination of the effective amount is well within the capability of those skilled in the art, especially in light of the detailed disclosure provided herein. For any compound used in the method of the invention, the therapeutically effective dose can be estimated initially from appropriate in vitro assays. For example, a dose can be formulated in animal models to achieve a circulating concentration range that can be used to more accurately determine useful doses in humans. For example, a dose can be formulated in animal models to achieve a circulating concentration range that includes the IC₅₀ as determined in cell culture (i.e., the concentration of the test compound which achieves a half-maximal inhibition of the protein's biological activity). Such information can be used to more accurately determine useful doses in humans.

A therapeutically effective dose refers to that amount of the compound that results in amelioration of symptoms or a prolongation of survival in a patient. Toxicity and therapeutic efficacy of such compounds can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio between LD₅₀ and ED₅₀. Compounds which exhibit high therapeutic indices are preferred. The data obtained from these cell culture assays and animal studies can be used in formulating a range of dosage for use in human. The dosage of such compounds lies preferably within a range of circulating concentrations that include the ED₅₀ with little or no toxicity. The dosage may vary within this range depending upon the dosage form employed and the route of administration utilized. The exact formulation, route of administration and dosage can be chosen by the individual physician in view of the patient's condition. See, e.g., Fingl et al., 1975, in “The Pharmacological Basis of Therapeutics”, Ch. 1 p. 1. Dosage amount and interval may be adjusted individually to provide plasma levels of the active moiety which are sufficient to maintain the desired effects, or minimal effective concentration (MEC). The MEC will vary for each compound but can be estimated from in vitro data. Dosages necessary to achieve the MEC will depend on individual characteristics and route of administration. However, BPLC assays or bioassays can be used to determine plasma concentrations.

Dosage intervals can also be determined using MEC value. Compounds should be administered using a regimen which maintains plasma levels above the MEC for 10-90% of the time, preferably between 30-90% and most preferably between 50-90%. In cases of local administration or selective uptake, the effective local concentration of the drug may not be related to plasma concentration.

An exemplary dosage regimen for polypeptides or other compositions of the invention will be in the range of about 0.01 μg/kg to 100 mg/kg of body weight daily, with the preferred dose being about 0.1 μg/kg to 25 mg/kg of patient body weight daily, varying in adults and children. Dosing may be once daily, or equivalent doses may be delivered at longer or shorter intervals.

The amount of composition administered will, of course, be dependent on the subject being treated, on the subject's age and weight, the severity of the affliction, the manner of administration and the judgment of the prescribing physician.

4.13.4 Packaging

The compositions may, if desired, be presented in a pack or dispenser device which may contain one or more unit dosage forms containing the active ingredient. The pack may, for example, comprise metal or plastic foil, such as a blister pack. The pack or dispenser device may be accompanied by instructions for administration. Compositions comprising a compound of the invention formulated in a compatible pharmaceutical carrier may also be prepared, placed in an appropriate container, and labeled for treatment of an indicated condition.

4.14 Antibodies

Also included in the invention are antibodies to proteins, or fragments of proteins of the invention. The term “antibody” as used herein refers to immunoglobulin molecules and immunologically active portions of immunoglobulin (Ig) molecules, i.e., molecules that contain an antigen-binding site that specifically binds (immunoreacts with) an antigen. Such antibodies include, but are not limited to, polyclonal, monoclonal, chimeric, single chain, F_{ab}, F_{ab′} and F_{(ab′)2} fragments, and an F_{ab} expression library. In general, an antibody molecule obtained from humans relates to any of the classes IgG, IgM, IgA, IgE and IgD, which differ from one another by the nature of the heavy chain present in the molecule. Certain classes have subclasses as well, such as IgG₁, IgG₂, and others. Furthermore, in humans, the light chain may be a kappa chain or a lambda chain. Reference herein to antibodies includes a reference to all such classes, subclasses and types of human antibody species.

An isolated related protein of the invention may be intended to serve as an antigen, or a portion or fragment thereof, and additionally can be used as an immunogen to generate antibodies that immunospecifically bind the antigen, using standard techniques for polyclonal and monoclonal antibody preparation. The full-length protein can be used or, alternatively, the invention provides antigenic peptide fragments of the antigen for use as immunogens. An antigenic peptide fragment comprises at least 6 amino acid residues of the amino acid sequence of the full length protein, such as an amino acid sequence shown in SEQ ID NO: 4, 6-7, 9, 11-12, 22, 24, 26, 28, 30, 32, 34, 36, 44, 46-48, 50, 52-53, 58, 60-62, 78, 80-81, 83, 85-87, 90, 92-94, 97, or 99-101, or Tables 2-7 and encompasses an epitope thereof such that an antibody raised against the peptide forms a specific immune complex with the full length protein or with any fragment that contains the epitope. Preferably, the antigenic peptide comprises at least 10 amino acid residues, or at least 15 amino acid residues, or at least 20 amino acid residues, or at least 30 amino acid residues. Preferred epitopes encompassed by the antigenic peptide are regions of the protein that are located on its surface; commonly these are hydrophilic regions.

In certain embodiments of the invention, at least one epitope encompassed by the antigenic peptide is a surface region of the protein, e.g., a hydrophilic region. A hydrophobicity analysis of the human related protein sequence will indicate which regions of a related protein are particularly hydrophilic and, therefore, are likely to encode surface residues useful for targeting antibody production. As a means for targeting antibody production, hydropathy plots showing regions of hydrophilicity and hydrophobicity may be generated by any method well known in the art, including, for example, the Kyte Doolittle or the Hopp Woods methods, either with or without Fourier transformation. See, e.g., Hopp and Woods, Proc. Nat. Acad. Sci. USA 78: 3824-3828 (1981); Kyte and Doolittle, J. Mol. Biol. 157: 105-142 (1982), each of which is incorporated herein by reference in its entirety. Antibodies that are specific for one or more domains within an antigenic protein, or derivatives, fragments, analogs or homologs thereof, are also provided herein.

A protein of the invention, or a derivative, fragment, analog, homolog or ortholog thereof, may be utilized as an immunogen in the generation of antibodies that immunospecifically bind these protein components.

The term “specific for” indicates that the variable regions of the antibodies of the invention recognize and bind polypeptides of the invention exclusively (i.e., able to distinguish the polypeptide of the invention from other similar polypeptides despite sequence identity, homology, or similarity found in the family of polypeptides), but may also interact with other proteins (for example, S. aureus protein A or other antibodies in ELISA techniques) through interactions with sequences outside the variable region of the antibodies, and in particular, in the constant region of the molecule. Screening assays to determine binding specificity of an antibody of the invention are well known and routinely practiced in the art. For a comprehensive discussion of such assays, see Harlow et al. (Eds), Antibodies A Laboratory Manual; Cold Spring Harbor Laboratory; Cold Spring Harbor, N.Y. (1988), Chapter 6. Antibodies that recognize and bind fragments of the polypeptides of the invention are also contemplated, provided that the antibodies are first and foremost specific for, as defined above, full-length polypeptides of the invention. As with antibodies that are specific for full length polypeptides of the invention, antibodies of the invention that recognize fragments are those which can distinguish polypeptides from the same family of polypeptides despite inherent sequence identity, homology, or similarity found in the family of proteins.

Antibodies of the invention are useful for, for example, therapeutic purposes (by modulating activity of a polypeptide of the invention), diagnostic purposes to detect or quantitate a polypeptide of the invention, as well as purification of a polypeptide of the invention. Kits comprising an antibody of the invention for any of the purposes described herein are also comprehended. In general, a kit of the invention also includes a control antigen for which the antibody is immunospecific. The invention further provides a hybridoma that produces an antibody according to the invention. Antibodies of the invention are useful for detection and/or purification of the polypeptides of the invention.

Monoclonal antibodies binding to the protein of the invention may be useful diagnostic agents for the immunodetection of the protein. Neutralizing monoclonal antibodies binding to the protein may also be useful therapeutics for both conditions associated with the protein and also in the treatment of some forms of cancer where abnormal expression of the protein is involved. In the case of cancerous cells or leukemic cells, neutralizing monoclonal antibodies against the protein may be useful in detecting and preventing the metastatic spread of the cancerous cells, which may be mediated by the protein.

The labeled antibodies of the present invention can be used for in vitro, in vivo, and in situ assays to identify cells or tissues in which a fragment of the polypeptide of interest is expressed. The antibodies may also be used directly in therapies or other diagnostics. The present invention further provides the above-described antibodies immobilized on a solid support. Examples of such solid supports include plastics such as polycarbonate, complex carbohydrates such as agarose and Sepharose®, acrylic resins and such as polyacrylamide and latex beads. Techniques for coupling antibodies to such solid supports are well known in the art (Weir, D. M. et al., “Handbook of Experimental Immunology” 4th Ed., Blackwell Scientific Publications, Oxford, England, Chapter 10 (1986); Jacoby, W. D. et al., Meth. Enzym. 34 Academic Press, N.Y. (1974)). The immobilized antibodies of the present invention can be used for in vitro, in vivo, and in situ assays as well as for immuno-affinity purification of the proteins of the present invention.

Various procedures known within the art may be used for the production of polyclonal or monoclonal antibodies directed against a protein of the invention, or against derivatives, fragments, analogs homologs or orthologs thereof (see, for example, Antibodies: A Laboratory Manual, Harlow E, and Lane D, 1988, Cold Spring Harbor Laboratory Press, Cold Spring Harbor, NY, incorporated herein by reference). Some of these antibodies are discussed below.

4.14.1 Polyclonal Antibodies

For the production of polyclonal antibodies, various suitable host animals (e.g., rabbit, goat, mouse or other mammal) may be immunized by one or more injections with the native protein, a synthetic variant thereof, or a derivative of the foregoing. An appropriate immunogenic preparation can contain, for example, the naturally occurring immunogenic protein, a chemically synthesized polypeptide representing the immunogenic protein, or a recombinantly expressed immunogenic protein. Furthermore, the protein may be conjugated to a second protein known to be immunogenic in the mammal being immunized. Examples of such immunogenic proteins include but are not limited to keyhole limpet hemocyanin, serum albumin, bovine thyroglobulin, and soybean trypsin inhibitor. The preparation can further include an adjuvant. Various adjuvants used to increase the immunological response include, but are not limited to, Freund's (complete and incomplete), mineral gels (e.g., aluminum hydroxide), surface-active substances (e.g., lysolecithin, pluronic polyols, polyanions, peptides, oil emulsions, dinitrophenol, etc.), adjuvants usable in humans such as Bacille Calmette-Guerin and Corynebacterium parvum, or similar immunostimulatory agents. Additional examples of adjuvants that can be employed include MPL-TDM adjuvant (monophosphoryl Lipid A, synthetic trehalose dicorynomycolate).

The polyclonal antibody molecules directed against the immunogenic protein can be isolated from the mammal (e.g., from the blood) and further purified by well known techniques, such as affinity chromatography using protein A or protein G, which provide primarily the IgG fraction of immune serum. Subsequently, or alternatively, the specific antigen which is the target of the immunoglobulin sought, or an epitope thereof, may be immobilized on a column to purify the immune specific antibody by immunoaffinity chromatography. Purification of immunoglobulins is discussed, for example, by D. Wilkinson (The Scientist, published by The Scientist, Inc., Philadelphia Pa., Vol. 14, No. 8 (Apr. 17, 2000), pp. 25-28).

4.14.2 Monoclonal Antibodies

The term “monoclonal antibody” (MAb) or “monoclonal antibody composition”, as used herein, refers to a population of antibody molecules that contain only one molecular species of antibody molecule consisting of a unique light chain gene product and a unique heavy chain gene product. In particular, the complementarity determining regions (CDRs) of the monoclonal antibody are identical in all the molecules of the population. MAbs thus contain an antigen-binding site capable of immunoreacting with a particular epitope of the antigen characterized by a unique binding affinity for it.

Monoclonal antibodies can be prepared using hybridoma methods, such as those described by Kohler and Milstein, Nature, 256:495 (1975). In a hybridoma method, a mouse, hamster, or other appropriate host animal, is typically immunized with an immunizing agent to elicit lymphocytes that produce or are capable of producing antibodies that will specifically bind to the immunizing agent. Alternatively, the lymphocytes can be immunized in vitro.

The immunizing agent will typically include the protein antigen, a fragment thereof or a fusion protein thereof. Generally, either peripheral blood lymphocytes are used if cells of human origin are desired, or spleen cells or lymph node cells are used if non-human mammalian sources are desired. The lymphocytes are then fused with an immortalized cell line using a suitable fusing agent, such as polyethylene glycol, to form a hybridoma cell (Goding, Monoclonal Antibodies: Principles and Practice, Academic Press, (1986) pp. 59-103). Immortalized cell lines are usually transformed mammalian cells, particularly myeloma cells of rodent, bovine and human origin. Usually, rat or mouse myeloma cell lines are employed. The hybridoma cells can be cultured in a suitable culture medium that preferably contains one or more substances that inhibit the growth or survival of the unfused, immortalized cells. For example, if the parental cells lack the enzyme hypoxanthine guanine phosphoribosyl transferase (HGPRT or HPRT), the culture medium for the hybridomas typically will include hypoxanthine, aminopterin, and thymidine (“HAT medium”), which substances prevent the growth of HGPRT-deficient cells.

Preferred immortalized cell lines are those that fuse efficiently, support stable high level expression of antibody by the selected antibody-producing cells, and are sensitive to a medium such as HAT medium. More preferred immortalized cell lines are murine myeloma lines, which can be obtained, for instance, from the Salk Institute Cell Distribution Center, San Diego, Calif. and the American Type Culture Collection, Manassas, Va. Human myeloma and mouse-human heteromyeloma cell lines also have been described for the production of human monoclonal antibodies (Kozbor, J. Immunol., 133:3001 (1984); Brodeur et al., Monoclonal Antibody Production Techniques and Applications, Marcel Dekker, Inc., New York, (1987) pp. 51-63).

The culture medium in which the hybridoma cells are cultured can then be assayed for the presence of monoclonal antibodies directed against the antigen. Preferably, the binding specificity of monoclonal antibodies produced by the hybridoma cells is determined by immunoprecipitation or by an in vitro binding assay, such as radioimmunoassay (RIA) or enzyme-linked immunoabsorbent assay (ELISA). Such techniques and assays are known in the art. The binding affinity of the monoclonal antibody can, for example, be determined by the Scatchard analysis of Munson and Pollard, Anal. Biochem., 107:220 (1980). Preferably, antibodies having a high degree of specificity and a high binding affinity for the target antigen are isolated.

After the desired hybridoma cells are identified, the clones can be subcloned by limiting dilution procedures and grown by standard methods. Suitable culture media for this purpose include, for example, Dulbecco's Modified Eagle's Medium (DMEM) and RPMI-1640 medium. Alternatively, the hybridoma cells can be grown in vivo as ascites in a mammal.

The monoclonal antibodies secreted by the subclones can be isolated or purified from the culture medium or ascites fluid by conventional immunoglobulin purification procedures such as, for example, protein A-Sepharose, hydroxylapatite chromatography, gel electrophoresis, dialysis, or affinity chromatography.

The monoclonal antibodies can also be made by recombinant DNA methods, such as those described in U.S. Pat. No. 4,816,567. DNA encoding the monoclonal antibodies of the invention can be readily isolated and sequenced using conventional procedures (e.g. by using oligonucleotide probes that are capable of binding specifically to genes encoding the heavy and light chains of murine antibodies). The hybridoma cells of the invention serve as a preferred source of such DNA. Once isolated, the DNA can be placed into expression vectors, which are then transfected into host cells such as simian COS cells, Chinese hamster ovary (CHO) cells, or myeloma cells that do not otherwise produce immunoglobulin protein, to obtain the synthesis of monoclonal antibodies in the recombinant host cells. The DNA also can be modified, for example, by substituting the coding sequence for human heavy and light chain constant domains in place of the homologous murine sequences (U.S. Pat. No. 4,816,567; Morrison, Nature 368:812-13 (1994)) or by covalentlyjoining to the immunoglobulin coding sequence all or part of the coding sequence for a non-immunoglobulin polypeptide. Such a non-immunoglobulin polyp eptide can be substituted for the constant domains of an antibody of the invention, or can be substituted for the variable domains of one antigen-combining site of an antibody of the invention to create a chimeric bivalent antibody.

4.14.3 Humanized Antibodies

The antibodies directed against the protein antigens of the invention can further comprise humanized antibodies or human antibodies. These antibodies are suitable for administration to humans without engendering an immune response by the human against the administered immunoglobulin. Humanized forms of antibodies are chimeric immunoglobulins, immunoglobulin chains or fragments thereof (such as Fv, Fab, Fab′, F(ab′)₂ or other antigen-binding subsequences of antibodies) that are principally comprised of the sequence of a human immunoglobulin, and contain minimal sequence derived from a non-human immunoglobulin. Humanization can be performed following the method of Winter and co-workers (Jones et al., Nature, 321:522-525 (1986); Riechmann, et al., Nature, 332:323-327 (1988); Verhoeyen, et al., Science, 239:1534-1536 (1988)), by substituting rodent CDRs or CDR sequences for the corresponding sequences of a human antibody. (See also U.S. Pat. No. 5,225,539). In some instances, Fv framework residues of the human immunoglobulin are replaced by corresponding non-human residues. Humanized antibodies can also comprise residues that are found neither in the recipient antibody nor in the imported CDR or framework sequences. In general, the humanized antibody will comprise substantially all of at least one, and typically two, variable domains, in which all or substantially all of the CDR regions correspond to those of a non-human immunoglobulin and all or substantially all of the framework regions are those of a human immunoglobulin consensus sequence. The humanized antibody optimally also will comprise at least a portion of an immunoglobulin constant region (Fc), typically that of a human immunoglobulin (Jones et al., 1986; Riechmann et al., 1988; and Presta, Curr. Op. Struct. Biol., 2:593-596 (1992)).

4.14.4 Human Antibodies

Fully human antibodies relate to antibody molecules in which essentially the entire sequences of both the light chain and the heavy chain, including the CDRs, arise from human genes. Such antibodies are termed “human antibodies” or “fully human antibodies” herein. Human monoclonal antibodies can be prepared by the trioma technique; the human B-cell hybridoma technique (see Kozbor, et al., Immunol Today 4: 72 (1983)) and the EBV hybridoma technique to produce human monoclonal antibodies (see Cole, et al., 1985 In: MONOCLONAL ANTIBODIES AND CANCER THERAPY, Alan R. Liss, Inc., pp. 77-96). Human monoclonal antibodies may be utilized in the practice of the present invention and may be produced by using human hybridomas (see Cote, et al., Proc Natl Acad Sci USA 80: 2026-2030 (1983)) or by transforming human B-cells with Epstein Barr Virus in vitro (see Cole, et al., 1985 In: MONOCLONAL ANTIODIES AND CANCER THERAPY, Alan R. Liss, Inc., pp. 77-96).

In addition, human antibodies can also be produced using additional techniques, including phage display libraries (Hoogenboom and Winter, J. Mol. Biol., 227:381 (1991); Marks et al., J. Mol. Biol., 222:581 (1991)). Similarly, human antibodies can be made by introducing human immunoglobulin loci into transgenic animals, e.g., mice in which the endogenous immunoglobulin genes have been partially or completely inactivated. Upon challenge, human antibody production is observed, which closely resembles that seen in humans in all respects, including gene rearrangement, assembly, and antibody repertoire. This approach is described, for example, in U.S. Pat. Nos. 5,545,807; 5,545,806; 5,569,825; 5,625,126; 5,633,425; 5,661,016, and in Marks et al. (Bio/Technology 10,:779-783 (1992)); Lonberg et al. (Nature 368:856-859 (1994)); Morrison (Nature 368:812-13 (1994)); Fishwild et al, (Nature Biotechnology, 14:845-51 (1996)); Neuberger (Nature Biotechnology, 14:826 (1996)); and Lonberg and Huszar (Intern. Rev. Immunol. 13:65-93 (1995)).

Human antibodies may additionally be produced using transgenic nonhuman animals which are modified so as to produce fully human antibodies rather than the animal's endogenous antibodies in response to challenge by an antigen. (See PCT publication WO94/02602). The endogenous genes encoding the heavy and light immunoglobulin chains in the nonhuman host have been incapacitated, and active loci encoding human heavy and light chain immunoglobulins are inserted into the host's genome. The human genes are incorporated, for example, using yeast artificial chromosomes containing the requisite human DNA segments. An animal which provides all the desired modifications is then obtained as progeny by crossbreeding intermediate transgenic animals containing fewer than the full complement of the modifications. The preferred embodiment of such a nonhuman animal is a mouse, and is termed the Xenomouse™ as disclosed in PCT publications WO 96/33735 and WO 96/34096. This animal produces B cells which secrete fully human immunoglobulins. The antibodies can be obtained directly from the animal after immunization with an immunogen of interest, as, for example, a preparation of a polyclonal antibody, or alternatively from immortalized B cells derived from the animal, such as hybridomas producing monoclonal antibodies. Additionally, the genes encoding the immunoglobulins with human variable regions can be recovered and expressed to obtain the antibodies directly, or can be further modified to obtain analogs of antibodies such as, for example, single chain Fv molecules.

An example of a method of producing a nonhuman host, exemplified as a mouse, lacking expression of an endogenous immunoglobulin heavy chain is disclosed in U.S. Pat. No. 5,939,598. It can be obtained by a method including deleting the J segment genes from at least one endogenous heavy chain locus in an embryonic stem cell to prevent rearrangement of the locus and to prevent formation of a transcript of a rearranged immunoglobulin heavy chain locus, the deletion being effected by a targeting vector containing a gene encoding a selectable marker; and producing from the embryonic stem cell a transgenic mouse whose somatic and germ cells contain the gene encoding the selectable marker.

A method for producing an antibody of interest, such as a human antibody, is disclosed in U.S. Pat. No. 5,916,771. It includes introducing an expression vector that contains a nucleotide sequence encoding a heavy chain into one mammalian host cell in culture, introducing an expression vector containing a nucleotide sequence encoding a light chain into another mammalian host cell, and fusing the two cells to form a hybrid cell. The hybrid cell expresses an antibody containing the heavy chain and the light chain.

In a further improvement on this procedure, a method for identifying a clinically relevant epitope on an immunogen, and a correlative method for selecting an antibody that binds immunospecifically to the relevant epitope with high affinity, are disclosed in PCT publication WO 99/53049.

4.14.5 FAB Fragments and Single Chain Antibodies

According to the invention, techniques can be adapted for the production of single-chain antibodies specific to an antigenic protein of the invention (see e.g., U.S. Pat. No. 4,946,778). In addition, methods can be adapted for the construction of F_{ab} expression libraries (see e.g., Huse, et al., Science 246:1275-1281 (1989)) to allow rapid and effective identification of monoclonal F_{ab} fragments with the desired specificity for a protein or derivatives, fragments, analogs or homologs thereof. Antibody fragments that contain the idiotypes to a protein antigen may be produced by techniques known in the art including, but not limited to: (i) an F_{(ab′)2} fragment produced by pepsin digestion of an antibody molecule; (ii) an F_{ab} fragment generated by reducing the disulfide bridges of an F_{(ab′)2} fragment; (iii) an F_{ab} fragment generated by the treatment of the antibody molecule with papain and a reducing agent and (iv) Fᵥ fragments.

4.14.6 Bispecific Antibodies

Bispecific antibodies are monoclonal, preferably human or humanized, antibodies that have binding specificities for at least two different antigens. In the present case, one of the binding specificities is for an antigenic protein of the invention. The second binding target is any other antigen, and advantageously is a cell-surface protein or receptor or receptor subunit.

Methods for making bispecific antibodies are known in the art. Traditionally, the recombinant production of bispecific antibodies is based on the co-expression of two immunoglobulin heavy-chain/light-chain pairs, where the two heavy chains have different specificities (Milstein and Cuello, Nature, 305:537-539 (1983)). Because of the random assortment of immunoglobulin heavy and light chains, these hybridomas (quadromas) produce a potential mixture of ten different antibody molecules, of which only one has the correct bispecific structure. The purification of the correct molecule is usually accomplished by affinity chromatography steps. Similar procedures are disclosed in WO 93/08829, published 13 May 1993, and in Traunecker et al., EMBO J., 10:3655-3659 (1991).

Antibody variable domains with the desired binding specificities (antibody-antigen combining sites) can be fused to immunoglobulin constant domain sequences. The fusion preferably is with an immunoglobulin heavy-chain constant domain, comprising at least part of the hinge, CH2, and CH3 regions. It is preferred to have the first heavy-chain constant region (CH1) containing the site necessary for light-chain binding present in at least one of the fusions. DNAs encoding the immunoglobulin heavy-chain fusions and, if desired, the immunoglobulin light chain, are inserted into separate expression vectors, and are co-transfected into a suitable host organism. For further details of generating bispecific antibodies see, for example, Suresh et al., Methods in Enzymology, 121:210 (1986).

According to another approach described in WO 96/27011, the interface between a pair of antibody molecules can be engineered to maximize the percentage of heterodimers which are recovered from recombinant cell culture. The preferred interface comprises at least a part of the CH3 region of an antibody constant domain. In this method, one or more small amino acid side chains from the interface of the first antibody molecule are replaced with larger side chains (e.g. tyrosine or tryptophan). Compensatory “cavities” of identical or similar size to the large side chain(s) are created on the interface of the second antibody molecule by replacing large amino acid side chains with smaller ones (e.g. alanine or threonine). This provides a mechanism for increasing the yield of the heterodimer over other unwanted end-products such as homodimers.

Bispecific antibodies can be prepared as full-length antibodies or antibody fragments (e.g. F(ab′)₂ bispecific antibodies). Techniques for generating bispecific antibodies from antibody fragments have been described in the literature. For example, bispecific antibodies can be prepared using chemical linkage. Brennan et al., Science 229:81 (1985) describe a procedure wherein intact antibodies are proteolytically cleaved to generate F(ab′)₂ fragments. These fragments are reduced in the presence of the dithiol complexing agent sodium arsenite to stabilize vicinal dithiols and prevent intermolecular disulfide formation. The Fab′ fragments generated are then converted to thionitrobenzoate (TNB) derivatives. One of the Fab′-TNB derivatives is then reconverted to the Fab′-thiol by reduction with mercaptoethylamine and is mixed with an equimolar amount of the other Fab′-TNB derivative to form the bispecific antibody. The bispecific antibodies produced can be used as agents for the selective immobilization of enzymes.

Additionally, Fab′ fragments can be directly recovered from E. coli and chemically coupled to form bispecific antibodies. Shalaby et al., J. Exp. Med. 175:217-225 (1992) describe the production of a fully humanized bispecific antibody F(ab′)₂ molecule. Each Fab′ fragment was separately secreted from E. coli and subjected to directed chemical coupling in vitro to form the bispecific antibody. The bispecific antibody thus formed was able to bind to cells overexpressing the ErbB2 receptor and normal human T cells, as well as trigger the lytic activity of human cytotoxic lymphocytes against human breast tumor targets.

Various techniques for making and isolating bispecific antibody fragments directly from recombinant cell culture have also been described. For example, bispecific antibodies have been produced using leucine zippers. Kostelny et al., J. Immunol. 148:1547-1553 (1992). The leucine zipper peptides from the Fos and Jun proteins were linked to the Fab′ portions of two different antibodies by gene fusion. The antibody homodimers were reduced at the hinge region to form monomers and then re-oxidized to form the antibody heterodimers. This method can also be utilized for the production of antibody homodimers. The “diabody” technology described by Hollinger et al., Proc. Natl. Acad. Sci. USA 90:6444-6448 (1993) has provided an alternative mechanism for making bispecific antibody fragments. The fragments comprise a heavy-chain variable domain (V_{H}) connected to a light-chain variable domain (V_{L}) by a linker which is too short to allow pairing between the two domains on the same chain. Accordingly, the V_{H} and V_{L} domains of one fragment are forced to pair with the complementary V_{L} and V_{H} domains of another fragment, thereby forming two antigen-binding sites. Another strategy for making bispecific antibody fragments by the use of single-chain Fv (sFv) dimers has also been reported. See, Gruber et al., J. Immunol. 152:5368 (1994).

Antibodies with more than two valencies are contemplated. For example, trispecific antibodies can be prepared. Tutt et al., J. Immunol. 147:60 (1991).

Exemplary bispecific antibodies can bind to two different epitopes, at least one of which originates in the protein antigen of the invention. Alternatively, an anti-antigenic arm of an immunoglobulin molecule can be combined with an arm which binds to a triggering molecule on a leukocyte such as a T-cell receptor molecule (e.g. CD2, CD3, CD28, or B7), or Fc receptors for IgG (FcγR), such as FcγRI (CD64), FcγRII (CD32) and FcγRIII (CD16) so as to focus cellular defense mechanisms to the cell expressing the particular antigen. Bispecific antibodies can also be used to direct cytotoxic agents to cells which express a particular antigen. These antibodies possess an antigen-binding arm and an arm which binds a cytotoxic agent or a radionuclide chelator, such as EOTUBE, DPTA, DOTA, or TETA. Another bispecific antibody of interest binds the protein antigen described herein and further binds tissue factor (TF).

4.14.7 Heteroconjugate Antibodies

Heteroconjugate antibodies are also within the scope of the present invention. Heteroconjugate antibodies are composed of two covalently joined antibodies. Such antibodies have, for example, been proposed to target immune system cells to unwanted cells (U.S. Pat. No. 4,676,980), and for treatment of HIV infection (WO 91/00360; WO 92/200373; EP 03089). It is contemplated that the antibodies can be prepared in vitro using known methods in synthetic protein chemistry, including those involving crosslinking agents. For example, immunotoxins can be constructed using a disulfide exchange reaction or by forming a thioether bond. Examples of suitable reagents for this purpose include iminothiolate and methyl-4-mercaptobutyrimidate and those disclosed, for example, in U.S. Pat. No. 4,676,980.

4.14.8 Effector Function Engineering

It can be desirable to modify the antibody of the invention with respect to effector function, so as to enhance, e.g., the effectiveness of the antibody in treating cancer. For example, cysteine residue(s) can be introduced into the Fc region, thereby allowing interchain disulfide bond formation in this region. The homodimeric antibody thus generated can have improved internalization capability and/or increased complement-mediated cell killing and antibody-dependent cellular cytotoxicity (ADCC). See Caron et al., J. Exp Med., 176: 1191-1195 (1992) and Shopes, J. Immunol., 148: 2918-2922 (1992). Homodimeric antibodies with enhanced anti-tumor activity can also be prepared using heterobifunctional cross-linkers as described in Wolff et al. Cancer Research, 53: 2560-2565 (1993). Alternatively, an antibody can be engineered that has dual Fc regions and can thereby have enhanced complement lysis and ADCC capabilities. See Stevenson et al., Anti-Cancer Drug Design, 3: 219-230 (1989).

4.14.9 Immunoconjugates

The invention also pertains to immunoconjugates comprising an antibody conjugated to a cytotoxic agent such as a chemotherapeutic agent, toxin (e.g., an enzymatically active toxin of bacterial, fungal, plant, or animal origin, or fragments thereof), or a radioactive isotope (i.e., a radioconjugate).

Chemotherapeutic agents useful in the generation of such immunoconjugates have been described above. Enzymatically active toxins and fragments thereof that can be used include diphtheria A chain, nonbinding active fragments of diphtheria toxin, exotoxin A chain (from Pseudomonas aeruginosa), ricin A chain, abrin A chain, modeccin A chain, alpha-sarcin, Aleurites fordii proteins, dianthin proteins, Phytolaca americana proteins (PAPL PAPII, and PAP-S), momordica charantia inhibitor, curcin, crotin, sapaonaria officinalis inhibitor, gelonin, mitogellin, restrictocin, phenomycin, enomycin, and the tricothecenes. A variety of radionuclides are available for the production of radioconjugated antibodies. Examples include ²¹²Bi, ¹³¹I, ¹³¹In, ⁹⁰Y, and ¹⁸⁶Re.

Conjugates of the antibody and cytotoxic agent are made using a variety of bifunctional protein-coupling agents such as N-succinimidyl-3-(2-pyridyldithiol) propionate (SPDP), iminothiolane (IT), bifunctional derivatives of imidoesters (such as dimethyl adipimidate HCL), active esters (such as disuccinimidyl suberate), aldehydes (such as glutareldehyde), bis-azido compounds (such as bis (p-azidobenzoyl) hexanediamine), bis-diazonium derivatives (such as bis-(p-diazoniumbenzoyl)-ethylenediamine), diisocyanates (such as tolyene 2,6-diisocyanate), and bis-active fluorine compounds (such as 1,5-difluoro-2,4-dinitrobenzene). For example, a ricin immunotoxin can be prepared as described in Vitetta et al., Science, 238: 1098 (1987). Carbon-14-labeled 1-isothiocyanatobenzyl-3-methyldiethylene triaminepentaacetic acid (MX-DTPA) is an exemplary chelating agent for conjugation of radionucleotide to the antibody. See WO94/11026.

In another embodiment, the antibody can be conjugated to a “receptor” (such streptavidin) for utilization in tumor pretargeting wherein the antibody-receptor conjugate is administered to the patient, followed by removal of unbound conjugate from the circulation using a clearing agent and then administration of a “ligand” (e.g. avidin) that is in turn conjugated to a cytotoxic agent.

4.15 Computer Readable Sequences

In one application of this embodiment, a nucleotide sequence of the present invention can be recorded on computer readable media As used herein, “computer readable media” refers to any medium which can be read and accessed directly by a computer. Such media include, but are not limited to: magnetic storage media, such as floppy discs, hard disc storage medium, and magnetic tape; optical storage media such as CD-ROM; electrical storage media such as RAM and ROM; and hybrids of these categories such as magnetic/optical storage media. A skilled artisan can readily appreciate how any of the presently known computer readable mediums can be used to create a manufacture comprising computer readable medium having recorded thereon a nucleotide sequence of the present invention. As used herein, “recorded” refers to a process for storing information on computer readable medium. A skilled artisan can readily adopt any of the presently known methods for recording information on computer readable medium to generate manufactures comprising the nucleotide sequence information of the present invention.

A variety of data storage structures are available to a skilled artisan for creating a computer readable medium having recorded thereon a nucleotide sequence of the present invention. The choice of the data storage structure will generally be based on the means chosen to access the stored information. In addition, a variety of data processor programs and formats can be used to store the nucleotide sequence information of the present invention on computer readable medium. The sequence information can be represented in a word processing text file, formatted in commercially-available software such as WordPerfect and Microsoft Word, or represented in the form of an ASCII file, stored in a database application, such as DB2, Sybase, Oracle, or the like. A skilled artisan can readily adapt any number of data processor structuring formats (e.g. text file or database) in order to obtain computer readable medium having recorded thereon the nucleotide sequence information of the present invention.

By providing any of the nucleotide sequences SEQ ID NO: 1-3, 5, 8, 10, 21, 23, 25, 27, 29, 31, 33, 35, 37-40, 43, 45, 49, 51, 53, 56-57, 59, 76-77, 79, 82, 84, 88-89, 91, 95-96, or 98, or a representative fragment thereof; or a nucleotide sequence at least 95% identical to any of the nucleotide sequences of SEQ ID NO: 1-3, 5, 8, 10, 21, 23, 25, 27, 29, 31, 33, 35, 37-40, 43, 45, 49, 51, 53, 56-57, 59, 76-77, 79, 82, 84, 88-89, 91, 95-96, or 98 in computer readable form, a skilled artisan can routinely access the sequence information for a variety of purposes. Computer software is publicly available which allows a skilled artisan to access sequence information provided in a computer readable medium. The examples which follow demonstrate how software which implements the BLAST (Altschul et al., J. Mol. Biol. 215:403-410 (1990)) and BLAZE (Brutlag et al., Comp. Chem. 17:203-207 (1993)) search algorithms on a Sybase system is used to identify open reading frames (ORFs) within a nucleic acid sequence. Such ORFs may be protein-encoding fragments and may be useful in producing commercially important proteins such as enzymes used in fermentation reactions and in the production of commercially useful metabolites.

As used herein, “a computer-based system” refers to the hardware means, software means, and data storage means used to analyze the nucleotide sequence information of the present invention. The minimum hardware means of the computer-based systems of the present invention comprises a central processing unit (CPU), input means, output means, and data storage means. A skilled artisan can readily appreciate that any one of the currently available computer-based systems are suitable for use in the present invention. As stated above, the computer-based systems of the present invention comprise a data storage means having stored therein a nucleotide sequence of the present invention and the necessary hardware means and software means for supporting and implementing a search means. As used herein, “data storage means” refers to memory which can store nucleotide sequence information of the present invention, or a memory access means which can access manufactures having recorded thereon the nucleotide sequence information of the present invention.

As used herein, “search means” refers to one or more programs which are implemented on the computer-based system to compare a target sequence or target structural motif with the sequence information stored within the data storage means. Search means are used to identify fragments or regions of a known sequence which match a particular target sequence or target motif. A variety of known algorithms are disclosed publicly and a variety of commercially available software for conducting search means are and can be used in the computer-based systems of the present invention. Examples of such software include, but are not limited to, Smith-Waterman, MacPattern (EMBL), BLASTN and BLASTA (NPOLYPEPTIDEIA). A skilled artisan can readily recognize that any one of the available algorithms or implementing software packages for conducting homology searches can be adapted for use in the present computer-based systems. As used herein, a “target sequence” can be any nucleic acid or amino acid sequence of six or more nucleotides or two or more amino acids. A skilled artisan can readily recognize that the longer a target sequence is, the less likely a target sequence will be present as a random occurrence in the database. The most preferred sequence length of a target sequence is from about 10 to 100 amino acids, or from about 30 to 300 nucleotide residues. However, it is well recognized that searches for commercially important fragments, such as sequence fragments involved in gene expression and protein processing, may be of shorter length.

As used herein, “a target structural motif,” or “target motif,” refers to any rationally selected sequence or combination of sequences in which the sequence(s) are chosen based on a three-dimensional configuration which is formed upon the folding of the target motif. There are a variety of target motifs known in the art. Protein target motifs include, but are not limited to, enzyme active sites and signal sequences. Nucleic acid target motifs include, but are not limited to, promoter sequences, hairpin structures and inducible expression elements (protein binding sequences).

4.16 Triple Relix Formation

In addition, the fragments of the present invention, as broadly described, can be used to control gene expression through triple helix formation or antisense DNA or RNA, both of which methods are based on the binding of a polynucleotide sequence to DNA or RNA. Polynucleotides suitable for use in these methods are usually 20 to 40 bases in length and are designed to be complementary to a region of the gene involved in transcription (triple helix—see Lee et al., Natl. Acids Res. 6:3073 (1979); Cooney et al., Science 15241:456 (1988); and Dervan et al., Science 251:1360 (1991)) or to the mRNA itself (antisense—Olmno, J. Neurochem. 56:560 (1991); Oligodeoxynucleotides as Antisense Inhibitors of Gene Expression, CRC Press, Boca Raton, Fla. (1988)). Triple helix-formation optimally results in a shut-off of RNA transcription from DNA, while antisense RNA hybridization blocks translation of an mRNA molecule into polypeptide. Both techniques have been demonstrated to be effective in model systems. Information contained in the sequences of the present invention is necessary for the design of an antisense or triple helix oligonucleotide.

4.17 Diagnostic Assays and Kits

The present invention further provides methods to identify the presence or expression of one of the ORFs of the present invention, or homolog thereof, in a test sample, using a nucleic acid probe or antibodies of the present invention, optionally conjugated or otherwise associated with a suitable label.

In general, methods for detecting a polynucleotide of the invention can comprise contacting a sample with a compound that binds to and forms a complex with the polynucleotide for a period sufficient to form the complex, and detecting the complex, so that if a complex is detected, a polynucleotide of the invention is detected in the sample. Such methods can also comprise contacting a sample under stringent hybridization conditions with nucleic acid primers that anneal to a polynucleotide of the invention under such conditions, and amplifying annealed polynucleotides, so that if a polynucleotide is amplified, a polynucleotide of the invention is detected in the sample.

In general, methods for detecting a polypeptide of the invention can comprise contacting a sample with a compound that binds to and forms a complex with the polypeptide for a period sufficient to form the complex, and detecting the complex, so that if a complex is detected, a polypeptide of the invention is detected in the sample.

In detail, such methods comprise incubating a test sample with one or more of the antibodies or one or more of the nucleic acid probes of the present invention and assaying for binding of the nucleic acid probes or antibodies to components within the test sample.

Conditions for incubating a nucleic acid probe or antibody with a test sample vary. Incubation conditions depend on the format employed in the assay, the detection methods employed, and the type and nature of the nucleic acid probe or antibody used in the assay. One skilled in the art will recognize that any one of the commonly available hybridization, amplification or immunological assay formats can readily be adapted to employ the nucleic acid probes or antibodies of the present invention. Examples of such assays can be found in Chard, T., An Introduction to Radioimmunoassay and Related Techniques, Elsevier Science Publishers, Amsterdam, The Netherlands (1986); Bullock, G. R. et al., Techniques in Immunocytochemistry, Academic Press, Orlando, Fla. Vol. 1 (1982), Vol. 2 (1983), Vol. 3 (1985); Tijssen, P., Practice and Theory of immunoassays: Laboratory Techniques in Biochemistry and Molecular Biology, Elsevier Science Publishers, Amsterdam, The Netherlands (1985). The test samples of the present invention include cells, protein or membrane extracts of cells, or biological fluids such as sputum, blood, serum, plasma, or urine. The test sample used in the above-described method will vary based on the assay format, nature of the detection method and the tissues, cells or extracts used as the sample to be assayed. Methods for preparing protein extracts or membrane extracts of cells are well known in the art and can be readily be adapted in order to obtain a sample which is compatible with the system utilized.

In another embodiment of the present invention, kits are provided which contain the necessary reagents to carry out the assays of the present invention. Specifically, the invention provides a compartment kit to receive, in close confinement, one or more containers which comprises: (a) a first container comprising one of the probes or antibodies of the present invention; and (b) one or more other containers comprising one or more of the following: wash reagents, reagents capable of detecting presence of a bound probe or antibody.

In detail, a compartment kit includes any kit in which reagents are contained in separate containers. Such containers include small glass containers, plastic containers or strips of plastic or paper. Such containers allows one to efficiently transfer reagents from one compartment to another compartment such that the samples and reagents are not cross-contaminated, and the agents or solutions of each container can be added in a quantitative fashion from one compartment to another. Such containers will include a container which will accept the test sample, a container which contains the antibodies used in the assay, containers which contain wash reagents (such as phosphate buffered saline, Tris-buffers, etc.), and containers which contain the reagents used to detect the bound antibody or probe. Types of detection reagents include labeled nucleic acid probes, labeled secondary antibodies, or in the alternative, if the primary antibody is labeled, the enzymatic, or antibody binding reagents which are capable of reacting with the labeled antibody. One skilled in the art will readily recognize that the disclosed probes and antibodies of the present invention can be readily incorporated into one of the established kit formats which are well known in the art.

4.18 Medical Imaging

The novel polypeptides and binding partners of the invention are useful in medical imaging of sites expressing the molecules of the invention (e.g., where the polypeptide of the invention is involved in the immune response, for imaging sites of inflammation or infection). See, e.g., Kunkel et al., U.S. Pat. NO. 5,413,778. Such methods involve chemical attachment of a labeling or imaging agent, administration of the labeled polypeptide to a subject in a pharmaceutically acceptable carrier, and imaging the labeled polypeptide in vivo at the target site.

4.19 Screening Assays

Using the isolated proteins and polynucleotides of the invention, the present invention further provides methods of obtaining and identifying agents which bind to a polypeptide encoded by an ORF corresponding to any of the nucleotide sequences set forth in SEQ ID NO: 1-3, 5, 8, 10, 21, 23, 25, 27, 29, 31, 33, 35, 37-40, 43, 45, 49, 51, 53, 56-57, 59, 76-77, 79, 82, 84, 88-89, 91, 95-96, or 98, or bind to a specific domain of the polypeptide encoded by the nucleic acid. In detail, said method comprises the steps of:

(a) contacting an agent with an isolated protein encoded by an ORF of the present invention, or nucleic acid of the invention; and

(b) determining whether the agent binds to said protein or said nucleic acid.

In general, therefore, such methods for identifying compounds that bind to a polynucleotide of the invention can comprise contacting a compound with a polynucleotide of the invention for a time sufficient to form a polynucleotide/compound complex, and detecting the complex, so that if a polynucleotide/compound complex is detected, a compound that binds to a polynucleotide of the invention is identified.

Likewise, in general, therefore, such methods for identifying compounds that bind to a polypeptide of the invention can comprise contacting a compound with a polypeptide of the invention for a time sufficient to form a polypeptide/compound complex, and detecting the complex, so that if a polypeptide/compound complex is detected, a compound that binds to a polynucleotide of the invention is identified.

Methods for identifying compounds that bind to a polypeptide of the invention can also comprise contacting a compound with a polypeptide of the invention in a cell for a time sufficient to form a polypeptide/compound complex, wherein the complex drives expression of a receptor gene sequence in the cell, and detecting the complex by detecting reporter gene sequence expression, so that if a polypeptide/compound complex is detected, a compound that binds a polypeptide of the invention is identified.

Compounds identified via such methods can include compounds which modulate the activity of a polypeptide of the invention (that is, increase or decrease its activity, relative to activity observed in the absence of the compound). Alternatively, compounds identified via such methods can include compounds which modulate the expression of a polynucleotide of the invention (that is, increase or decrease expression relative to expression levels observed in the absence of the compound). Compounds, such as compounds identified via the methods of the invention, can be tested using standard assays well known to those of skill in the art for their ability to modulate activity/expression.

The agents screened in the above assay can be, but are not limited to, peptides, carbohydrates, vitamin derivatives, or other pharmaceutical agents. The agents can be selected and screened at random or rationally selected or designed using protein modeling techniques.

For random screening, agents such as peptides, carbohydrates, pharmaceutical agents and the like are selected at random and are assayed for their ability to bind to the protein encoded by the ORF of the present invention. Alternatively, agents may be rationally selected or designed. As used herein, an agent is said to be “rationally selected or designed” when the agent is chosen based on the configuration of the particular protein. For example, one skilled in the art can readily adapt currently available procedures to generate peptides, pharmaceutical agents and the like, capable of binding to a specific peptide sequence, in order to generate rationally designed antipeptide peptides, for example see Hurby et al., Application of Synthetic Peptides: Antisense Peptides,” In Synthetic Peptides, A User's Guide, W. H. Freeman, NY (1992), pp. 289-307, and Kaspczak et al., Biochemistry 28:9230-8 (1989), or pharmaceutical agents, or the like.

In addition to the foregoing, one class of agents of the present invention, as broadly described, can be used to control gene expression through binding to one of the ORFs or EMFs of the present invention. As described above, such agents can be randomly screened or rationally designed/selected. Targeting the ORF or EMF allows a skilled artisan to design sequence specific or element specific agents, modulating the expression of either a single ORF or multiple ORFs which rely on the same EMF for expression control. One class of DNA binding agents are agents which contain base residues which hybridize or form a triple helix formation by binding to DNA or RNA. Such agents can be based on the classic phosphodiester, ribonucleic acid backbone, or can be a variety of sulfhydryl or polymeric derivatives which have base attachment capacity.

Agents suitable for use in these methods usually contain 20 to 40 bases and are designed to be complementary to a region of the gene involved in transcription (triple helix—see Lee et al., Nucl. Acids Res. 6:3073 (1979); Cooney et al., Science 241:456 (1988); and Dervan et al., Science 251:1360 (1991)) or to the mRNA itself (antisense—Okano, J. Neurochem. 56:560 (1991); Oligodeoxynucleotides as Antisense Inhibitors of Gene Expression, CRC Press, Boca Raton, Fla. (1988)). Triple helix-formation optimally results in a shut-off of RNA transcription from DNA, while antisense RNA hybridization blocks translation of an mRNA molecule into polypeptide. Both techniques have been demonstrated to be effective in model systems. Information contained in the sequences of the present invention is necessary for the design of an antisense or triple helix oligonucleotide and other DNA binding agents.

Agents which bind to a protein encoded by one of the ORFs of the present invention can be used as a diagnostic agent. Agents which bind to a protein encoded by one of the ORFs of the present invention can be formulated using known techniques to generate a pharmaceutical composition.

4.20 Use of Nucleic Acids as Probes

Another aspect of the subject invention is to provide for polypeptide-specific nucleic acid hybridization probes capable of hybridizing with naturally occurring nucleotide sequences. The hybridization probes of the subject invention may be derived from any of the nucleotide sequences SEQ ID NO: 1-3, 5, 8, 10, 21, 23, 25, 27, 29, 31, 33, 35, 37-40, 43, 45, 49, 51, 53, 56-57, 59, 76-77, 79, 82, 84, 88-89, 91, 95-96, or 98. Because the corresponding gene is only expressed in a limited number of tissues, a hybridization probe derived from of any of the nucleotide sequences SEQ ID NO: 1-3, 5, 8, 10, 21, 23, 25, 27, 29, 31, 33, 35, 37-40, 43, 45, 49, 51, 53, 56-57, 59, 76-77, 79, 82, 84, 88-89, 91, 95-96, or 98 can be used as an indicator of the presence of RNA of cell type of such a tissue in a sample.

Any suitable hybridization technique can be employed, such as, for example, in situ hybridization. PCR as described in U.S. Pat. Nos. 4,683,195 and 4,965,188 provides additional uses for oligonucleotides based upon the nucleotide sequences. Such probes used in PCR may be of recombinant origin, may be chemically synthesized, or a mixture of both.

The probe will comprise a discrete nucleotide sequence for the detection of identical sequences or a degenerate pool of possible sequences for identification of closely related genomic sequences.

Other means for producing specific hybridization probes for nucleic acids include the cloning of nucleic acid sequences into vectors for the production of mRNA probes. Such vectors are known in the art and are commercially available and may be used to synthesize RNA probes in vitro by means of the addition of the appropriate RNA polymerase as T7 or SP6 RNA polymerase and the appropriate radioactively labeled nucleotides. The nucleotide sequences may be used to construct hybridization probes for mapping their respective genomic sequences. The nucleotide sequence provided herein may be mapped to a chromosome or specific regions of a chromosome using well known genetic and/or chromosomal mapping techniques. These techniques include in situ hybridization, linkage analysis against known chromosomal markers, hybridization screening with libraries or flow-sorted chromosomal preparations specific to known chromosomes, and the like. The technique of fluorescent in situ hybridization of chromosome spreads has been described, among other places, in Verma et al (1988) Human Chromosomes: A Manual of Basic Techniques, Pergamon Press, New York N.Y.

Fluorescent in situ hybridization of chromosomal preparations and other physical chromosome mapping techniques may be correlated with additional genetic map data Examples of genetic map data can be found in the 1994 Genome Issue of Science (265:1981f). Correlation between the location of a nucleic acid on a physical chromosomal map and a specific disease (or predisposition to a specific disease) may help delimit the region of DNA associated with that genetic disease. The nucleotide sequences of the subject invention may be used to detect differences in gene sequences between normal, carrier or affected individuals.

4.21 Preparation of support Bound Oligonucleotides

Oligonucleotides, i.e., small nucleic acid segments, may be readily prepared by, for example, directly synthesizing the oligonucleotide by chemical means, as is commonly practiced using an automated oligonucleotide synthesizer.

Support bound oligonucleotides may be prepared by any of the methods known to those of skill in the art using any suitable support such as glass, polystyrene or Teflon. One strategy is to precisely spot oligonucleotides synthesized by standard synthesizers. Immobilization can be achieved using passive adsorption (Inouye & Hondo, J. Clin Microbiol 28:1462-72 (1990)); using UV light (Nagata et al., 1985; Dahlen et al., 1987; Morrissey & Collins, Mol. Cell Probes 3:189-207 (1989)) orby covalent binding of base modified DNA (Keller et al., 1988; 1989); all references being specifically incorporated herein.

Another strategy that may be employed is the use of the strong biotin-streptavidin interaction as a linker. For example, Broude et al. Proc. Natl. Acad. Sci USA 91:3072-6 (1994) describe the use of biotinylated probes, although these are duplex probes, that are immobilized on streptavidin-coated magnetic beads. Streptavidin-coated beads may be purchased from Dynal, Oslo. Of course, this same linking chemistry is applicable to coating any surface with streptavidin. Biotinylated probes maybe purchased from various sources, such as, e.g., Operon Technologies (Alameda, Calif.).

Nunc Laboratories (Naperville, Ill.) is also selling suitable material that could be used. Nunc Laboratories have developed a method by which DNA can be covalently bound to the microwell surface termed Covalink NH. CovaLink NH is a polystyrene surface grafted with secondary amino groups (>NH) that serve as bridge-heads for further covalent coupling. CovaLink Modules may be purchased from Nunc Laboratories. DNA molecules may be bound to CovalLink exclusively at the 5′-end by a phosphoramidate bond, allowing immobilization of more than 1 pmol of DNA (Rasmussen et al., Anal Biochem 198:138-42 (1991)).

The use of CovaLink NH strips for covalent binding of DNA molecules at the 5′-end has been described Rasmussen et al., 1991). In this technology, a phosphoramidate bond is employed (Chu et al., Nucleic Acids 11:6513-29 (1983)). This is beneficial as immobilization using only a single covalent bond is preferred The phosphoramidate bondjoins the DNA to the CovaLink NH secondary amino groups that are positioned at the end of spacer arms covalently grafted onto the polystyrene surface through a 2 nm long spacer arm. To link an oligonucleotide to CovaLink NH via an phosphoramidate bond, the oligonucleotide terminus must have a 5′-end phosphate group. It is, perhaps, even possible for biotin to be covalently bound to CovaLink and then streptavidin used to bind the probes.

More specifically, the linkage method includes dissolving DNA in water (7.5 ng/ul) and denaturing for 10 min at 95° C. and cooling on ice for 10 min. Ice-cold 0.1 M 1-methylimidazole, pH 7.0 (1-MeIm₇), is then added to a final concentration of 10 mM 1-MeIm₇. A ss DNA solution is then dispensed into CovaLink NH strips (75 ul/well) standing on ice.

Carbodiimide 0.2 M 1-ethyl-3-(3-dimethylaminopropyl)-carbodiimide (EDC), dissolved in 10 mM 1-MeIm₇, is made fresh and 25 ul added per well. The strips are incubated for 5 hours at 50° C. After incubation the strips are washed using, e.g., Nunc-Immuno Wash; first the wells are washed 3 times, then they are soaked with washing solution for 5 min., and finally they are washed 3 times (where in the washing solution is 0.4 N NaOH, 0.25% SDS heated to 50° C.).

It is contemplated that a further suitable method for use with the present invention is that described in PCT Patent Application WO 90/03382 (Southern & Maskos), incorporated herein by reference. This method of preparing an oligonucleotide bound to a support involves attaching a nucleoside 3′-reagent through the phosphate group by a covalent phosphodiester link to aliphatic hydroxyl groups carried by the support. The oligonucleotide is then synthesized on the supported nucleoside and protecting groups removed from the synthetic oligonucleotide chain under standard conditions that do not cleave the oligonucleotide from the support. Suitable reagents include nucleoside phosphoramidite and nucleoside hydrogen phosphorate.

An on-chip strategy for the preparation of DNA probe for the preparation of DNA probe arrays may be employed. For example, addressable laser-activated photodeprotection may be employed in the chemical synthesis of oligonucleotides directly on a glass surface, as described byFodor et al. Science 251:767-73 (1991)), incorporated herein by reference. Probes may also be immobilized on nylon supports as described by Van Ness et al. Nucleic Acids Res. 19:3345-50 (1991); or linked to Teflon using the method of Duncan & Cavalier, Anal Biochem 169:104-8 (1988); all references being specifically incorporated herein.

To link an oligonucleotide to a nylon support, as described by Van Ness et al. (1991), requires activation of the nylon surface via alkylation and selective activation of the 5′-amine of oligonucleotides with cyanuric chloride.

One particular way to prepare support bound oligonucleotides is to utilize the light-generated synthesis described by Pease et al., Proc. Natl. Acad. Sci USA 91:5022-6 (1994). These authors used current photolithographic techniques to generate arrays of immobilized oligonucleotide probes (DNA chips). These methods, in which light is used to direct the synthesis of oligonucleotide probes in high-density, miniaturized arrays, utilize photolabile 5′-protected N-acyl-deoxynucleoside phosphoramidites, surface linker chemistry and versatile combinatorial synthesis strategies. A matrix of 256 spatially defined oligonucleotide probes may be generated in this manner.

4.22 Preparation of Nucleic Acid Fragments

The nucleic acids may be obtained from any appropriate source, such as cDNAs, genomic DNA, chromosomal DNA, microdissected chromosome bands, cosmid or YAC inserts, and RNA, including mRNA without any amplification steps. For example, Sambrook et al. (1989) describes three protocols for the isolation of high molecular weight DNA from mammalian cells (p. 9.14-9.23).

DNA fragments may be prepared as clones in M13, plasmid or lambda vectors and/or prepared directly from genomic DNA or cDNA by PCR or other amplification methods. Samples may be prepared or dispensed in multiwell plates. About 100-1000 ng of DNA samples may be prepared in 2-500 ml of final volume.

The nucleic acids would then be fragmented by any of the methods known to those of skill in the art including, for example, using restriction enzymes as described at 9.24-9.28 of Sambrook et al. (1989), shearing by ultrasound and NaOH treatment.

Low pressure shearing is also appropriate, as described by Schriefer et al. Nucleic Acids Res. 18:7455-6 (1990). In this method, DNA samples are passed through a small French pressure cell at a variety of low to intermediate pressures. A lever device allows controlled application of low to intermediate pressures to the cell. The results of these studies indicate that low-pressure shearing is a useful alternative to sonic and enzymatic DNA fragmentation methods.

One particularly suitable way for fragmenting DNA is contemplated to be that using the two base recognition endonuclease, CviJI, described by Fitzgerald et al. Nucleic Acids Res. 20:3753-62 (1992). These authors described an approach for the rapid fragmentation and fractionation of DNA into particular sizes that they contemplated to be suitable for shotgun cloning and sequencing.

The restriction endonuclease CviJI normally cleaves the recognition sequence PuGCPy between the G and C to leave blunt ends. Atypical reaction conditions, which alter the specificity of this enzyme (CviJI**), yield a quasi-random distribution of DNA fragments form the small molecule pUC19 (2688 base pairs). Fitzgerald et al. (1992) quantitatively evaluated the randomness of this fragmentation strategy, using a CviJI** digest of pUC19 that was size fractionated by a rapid gel filtration method and directly ligated, without end repair, to a lac Z minus M13 cloning vector. Sequence analysis of 76 clones showed that CviJI** restricts pyGCPy and PuGCPu, in addition to PuGCPy sites, and that new sequence data is accumulated at a rate consistent with random fragmentation.

As reported in the literature, advantages of this approach compared to sonication and agarose gel fractionation include: smaller amounts of DNA are required (0.2-0.5 μg instead of 2-5 μg); and fewer steps are involved (no preligation, end repair, chemical extraction, or agarose gel electrophoresis and elution are needed).

Irrespective of the manner in which the nucleic acid fragments are obtained or prepared, it is important to denature the DNA to give single stranded pieces available for hybridization. This is achieved by incubating the DNA solution for 2-5 minutes at 80-90° C. The solution is then cooled quickly to 2° C. to prevent renaturation of the DNA fragments before they are contacted with the chip. Phosphate groups must also be removed from genomic DNA by methods known in the art

4.23 Preparation of DNA Arrays

Arrays may be prepared by spotting DNA samples on a support such as a nylon membrane. Spotting may be performed by using arrays of metal pins (the positions of which correspond to an array of wells in a microtiter plate) to repeated by transfer of about 20 nl of a DNA solution to a nylon membrane. By offset printing, a density of dots higher than the density of the wells is achieved. One to 25 dots may be accommodated in 1 mm², depending on the type of label used. By avoiding spotting in some preselected number of rows and columns, separate subsets (subarrays) may be formed. Samples in one subarray may be the same genomic segment of DNA (or the same gene) from different individuals, or may be different, overlapped genomic clones. Each of the subarrays may represent replica spotting of the same samples. In one example, a selected gene segment may be amplified from 64 patients. For each patient, the amplified gene segment may be in one 96-well plate (all 96 wells containing the same sample). A plate for each of the 64 patients is prepared. By using a 96-pin device, all samples may be spotted on one 8×12 cm membrane. Subarrays may contain 64 samples, one from each patient. Where the 96 subarrays are identical, the dot span may be 1 mm2 and there may be a 1 mm space between subarrays.

Another approach is to use membranes or plates (available from NUNC, Naperville, Ill.) which may be partitioned by physical spacers e.g. a plastic grid molded over the membrane, the grid being similar to the sort of membrane applied to the bottom of multiwell plates, or hydrophobic strips. A fixed physical spacer is not preferred for imaging by exposure to flat phosphor-storage screens or x-ray films.

The present invention is illustrated in the following examples. Upon consideration of the present disclosure, one of skill in the art will appreciate that many other embodiments and variations may be made in the scope of the present invention. Accordingly, it is intended that the broader aspects of the present invention not be limited to the disclosure of the following examples. The present invention is not to be limited in scope by the exemplified embodiments which are intended as illustrations of single aspects of the invention, and compositions and methods which are functionally equivalent are within the scope of the invention. Indeed, numerous modifications and variations in the practice of the invention are expected to occur to those skilled in the art upon consideration of the present preferred embodiments. Consequently, the only limitations which should be placed upon the scope of the invention are those which appear in the appended claims.

All references cited within the body of the instant specification are hereby incorporated by reference in their entirety.

### 5. EXAMPLES

### Example 1

### Isolation of Novel Nucleic Acids from cDNA Libraries of Human Cells

Novel nucleic acids were obtained from various human cDNA libraries using standard PCR, sequencing by hybridization sequence signature analysis, and Sanger sequencing techniques. The inserts of the library were amplified with PCR using primers specific for vector sequences flanking the inserts. These samples were spotted onto nylon membranes and interrogated with oligonucleotide probes to give sequence signatures. The clones were clustered into groups of similar or identical sequences, and single representative clones were selected from each group for gel sequencing. The 5′ sequence of the amplified inserts were then deduced using the reverse M13 sequencing primer in a typical Sanger sequencing protocol. PCR products were purified and subjected to fluorescent dye terminator cycle sequencing. Single-pass gel sequencing was done using a 377 Applied Biosystems (ABI) sequencer. These inserts was identified as a novel sequence not previously obtained from this library and not previously reported in public databases.

### Example 2

### Assemblage of SEQ ID NO: 2,3, or 8

The novel nucleic acids (SEQ ID NO: 1, 2, 3, or 8) of the invention were assembled from sequences that were obtained from cDNA libraries by methods described in Example 1 above, and in some cases obtained from one or more public databases. SEQ ID NO: 1 was the contig for SEQ ID NO: 2, 3, and 8, and is disclosed in PCT Publication No. WO 01/54477. The final sequences were assembled using the EST sequences as seed. Then a recursive algorithm was used to extend the seed into an extended assemblage, by pulling additional sequences from different databases (i.e. Nuvelo's database containing EST sequences, dbEST, gb pri, and UniGene) that belong to this assemblage. The algorithm terminated when there was no additional sequences from the above databases that would extend the assemblage. Inclusion of component sequences into the assemblage was based on a BLASTN hit to the extending assemblage with BLAST score greater than 300 and percent identity greater than 95%.

Using PHRAP (Univ. of Washington) or CAP4 (Paracel), a fill-length gene cDNA sequence and its corresponding protein sequence were generated from the assemblage. Any frame shifts and incorrect sop codons were corrected by hand editing. During editing, the sequence was checked using FASTY and BLAST against Genbank (i.e. dbEST, gb pri, UniGene, Genpept). Other computer programs which may have been used in the editing process were phredPhrap and Consed (Ulniversity of Washington) and ed-ready, ed-ext and cg-zip-2 (Nuvelo, Inc., Sunnyvale, Calif.). The full-length nucleotide sequences are shown in the Sequence Listing as SEQ ID NO: 2, 3 or 8; and the full-length amino acid sequences are shown in the Sequence Listing as SEQ ID NO: 4 or 9.

### Example 3

### Assemblage of SEQ ID NO: 37-40

The contigs of the present invention, designated as SEQ ID NO: 37-40 were assembled using an EST sequence from Nuvelo's database as a seed. A recursive algorithm was used to extend the seed EST into an extended assemblage, by pulling additional sequences from different databases (e.g., Nuvelo's database containing EST sequences, dbEST, gb pri, and UniGene version, and exons from public domain genomic sequences predicted by GenScan) that belong to this assemblage. The algorithm terminated when there were no additional sequences from the databases that will extend the assemblage. Further, the inclusion of component sequences into the assemblage was based on a BLASTN hit to the extending assemblage with BLAST score greater than 300 and percent identity greater than 95%. These sequences are designated as SEQ ID NO: 37-40 in the attached sequence listing. Additional functional information can be found in U.S. application Ser. No. 10/084,643 filed Feb. 26, 2002 entitled “Novel Nucleic Acids and Polypeptides”, Attorney Docket No. 21272-502; PCT Application Serial No. PCT/US00/35017 filed Dec. 22, 2000 entitled “Novel Contigs Obtained from Various Libraries”, Attorney Docket No. 784CIP3A/PCT; PCT Application Serial No. PCT/US01/02623 filed Jan. 25, 2001 entitled “Novel Contigs Obtained from Various Libraries”, Attorney Docket No. 785CIP3/PCT; PCT Application Serial No. PCT/US01/03800 filed Feb. 5, 2001 entitled “Novel Contigs Obtained from Various Libraries”, Attorney Docket No. 787CIP3/PCT; PCT Application Serial No. PCT/US01/08656 filed Apr. 18, 2001 entitled “Novel Contigs Obtained from Various Libraries”, Attorney Docket No. 791CIP3/PCT; all of which are incorporated herein by reference in their entirety.

### Example 3

### Assemblage of SEQ ID NO: 17, 21, 25, 29, or 33

The novel nucleic acids (SEQ ID NO: 17, 21, 25, 29, or 33) of the invention were assembled from sequences that were obtained from cDNA libraries by methods described in Example 1 above, and in some cases obtained from one or more public databases. The final sequences were assembled using the EST sequences as seed. Then a recursive algorithm was used to extend the seed into an extended assemblage, by pulling additional sequences from different databases (i.e. Nuvelo's database containing EST sequences, dbEST, gb pri, and UniGene) that belong to this assemblage. The algorithm terminated when there was no additional sequences from the above databases that would extend the assemblage. Inclusion of component sequences into the assemblage was based on a BLASTN hit to the extending assemblage with BLAST score greater than 300 and percent identity greater than 95%.

Using PHRAP (Univ. of Washington) or CAP4 (Paracel), a full-length gene cDNA sequence and its corresponding protein sequence were generated from the assemblage. Any frame shifts and incorrect sop codons were corrected by hand editing. During editing, the sequence was checked using FASTY and BLAST against Genbank (i.e. dbEST, gb pri, UniGene, Genpept). Other computer programs which may have been used in the editing process were phredPhrap and Consed (University of Washington) and ed-ready, ed-ext and cg-zip-2 (Nuvelo, Inc.). The full-length nucleotide sequences are shown in the Sequence Listing as SEQ ID NO: 17, 21, 25, 29, or 33; and the full-length amino acid sequences are shown in the Sequence Listing as SEQ ID NO: 18, 22, 26, 30, or 34.

### Example 4

### Assemblage of SEQ ID NO: 43 and 49

The novel nucleic acids of SEQ ID NO: 43 and 49 were obtained from various human cDNA libraries using standard PCR, sequencing by hybridization sequence signature analysis, and Sanger sequencing techniques. The inserts of the library were amplified with PCR using primers specific for vector sequences flanking the inserts. These samples were spotted onto nylon membranes and interrogated with oligonucleotide probes to give sequence signatures. The clones were clustered into groups of similar or identical sequences, and single representative clones were selected from each group for gel sequencing. The 5′ sequences of the amplified inserts were then deduced using the reverse M13 sequencing primer in a typical Sanger sequencing protocol. PCR products were purified and subjected to fluorescent dye terminator cycle sequencing. Single-pass gel sequencing was done using a 377 Applied Biosystems (ABI) sequencer. These inserts was identified as a novel sequence not previously obtained from this library and not previously reported in public databases. The novel sequences obtained from the sequencing efforts together with sequences from from one or more public databases were assembled into contigs using the EST sequences as seed. Then a recursive algorithm was used to extend the seed into an extended assemblage, by pulling additional sequences from different databases (i.e. Nuvelo's database containing EST sequences, Genpept121, dbEST121, gb pri121, and UniGene121 that belong to this assemblage. The algorithm terminated when there was no additional sequences from the above databases that would extend the assemblage. Inclusion of component sequences into the assemblage was based on a BLASTN hit to the extending assemblage with BLAST score greater than 300 and percent identity greater than 95%.

Using PHRAP (Univ. of Washington) or CAP4 (Paracel), a full-length gene cDNA sequence and its corresponding protein sequence were generated from the assemblage. Any frame shifts and incorrect sop codons were corrected by hand editing. During editing, the sequence was checked using FASTY and BLAST against Genbank (i.e. dbEST121, gbpri121, UniGene121, Genpept121). Other computer programs which may have been used in the editing process, were phredPhrap and Consed (University of Washington) and ed-ready, ed-ext and cg-zip-2 (Nuvelo, Inc.). The full-length nucleotide sequences are shown in the Sequence Listing as SEQ ID NO: 43 and 49; and the full-length amino acid sequences are shown in the sequence listing as SEQ ID NO: 44 and 50.

The nearest neighbor results for the assembled contigs were obtained by a FASTA search against Genpept, using FASTXY algorithm. FASTXY is an improved version of FASTA alignment, which allows in-codon frame shifts. The nearest neighbor results showed the closest homologue for each assemblage from Genpept121 (and contain the translated amino acid sequences for which the assemblages encodes). The nearest neighbor results are set forth in Table 8 below:

TABLE 8

| SEQ | | | Smith- | |
|---|---|---|---|---|
| ID | Accession | | Waterman | |
| NO: | No. | Description | Score | % Identity |
| 43 | XP_092478.2 | Homo sapiens similar to | 604 | 100% |
| | | gliacolin | | |
| 49 | XP_092478.2 | Homo sapiens similar to | 604 | 97% |
| | | gliacolin | | |

The predicted amino acid sequences for SEQ ID NO: 43 and 49 were obtained by using a software program called FASTY (available from the biochemistry department at the University of Virginia) which selects a polypeptide based on a comparison of translated novel polynucleotide to known polynucleotides (W. R. Pearson, Methods in Enzymology, 183:63-98 (1990), incorporated herein by reference) and are disclosed as SEQ ID NO: 44 and 50.

Further annotation of SEQ ID NO: 49 and 50 can be found in U.S. patent application Ser. No. 60/365,091 filed Mar. 14, 2002 entitled “Novel Nucleic Acids and Polypeptides”, Attorney docket no. 815, SEQ ID NO: 44, herein incorporated by reference in its entirety.

### Example 5

### Identification of SEQ ED NO: 57

Assembly of the novel nucleotide sequence of SEQ ID NO: 57 was accomplished using a contig sequence SEQ ID NO: 56 as a seed. The seed was extended by gel sequencing (377 Applied Biosystems (ABI) sequencer) using primers to extend the 3′ end (primer extension). The DNA from the full-length clone was then isolated, sonicated and recloned for gel sequencing. Each fragment was sequenced by gel sequencing (377 ABI sequencer) and the sequences were assembled to arrive at the complete sequence. A polypeptide (SEQ ID NO: 58) was predicted to be encoded by SEQ ID NO: 57 as set forth below. The polypeptide was predicted using a software program called BLASTX which selects a polypeptide based on a comparison of the translated novel polynucleotide to known polynucleotides. The initial methionine starts at position 6 of SEQ ID NO: 57 and the putative stop codon, TAA, begins at position 300 of the nucleotide sequence SEQ ID NO: 57.

### Example 6

### Identification of SEQ ID NO: 64 and 70

Assembly of the novel nucleotide sequence of SEQ ID NO: 64 and 170 was accomplished using a contig sequence SEQ ID NO: 63 as a seed. The seed was extended by gel sequencing (377 Applied Biosystems (ABI) sequencer) using primers to extend the 3′ end (primer extension). The DNA from the full-length clone was then isolated, sonicated and recloned for gel sequencing. Each fragment was sequenced by gel sequencing (377 ABI sequencer) and the sequences were assembled to arrive at the complete sequence. A polypeptide (SEQ ID NO: 65 or 71) was predicted to be encoded by SEQ ID NO: 64 or 70, respectively as set forth below. The polypeptide was predicted using a software program called BLASTX which selects a polypeptide based on a comparison of the translated novel polynucleotide to known polynucleotides. The initial methionine starts at position 1 of SEQ ID NO: 64 and the putative stop codon, TAG, begins at position 343 of the nucleotide sequence SEQ ID NO: 64. The initial methionine starts at position 25 of SEQ ID NO: 70 and the putative stop codon, TAG, begins at position 403 of the nucleotide sequence SEQ ID NO: 70.

### Example 7

### Identificaiton of SEQ ID NO: 77, 82, and 89

Assembly of the novel nucleotide sequence of SEQ ID NO: 77 and 82 was accomplished using a contig sequence SEQ ID NO: 76 as a seed. The seed was extended by gel sequencing (377 Applied Biosystems (ABI) sequencer) using primers to extend the 3′ end (primer extension). The DNA from the full-length clone was then isolated, sonicated and recloned for gel sequencing. Each fragment was sequenced by gel sequencing (377 ABI sequencer) and the sequences were assembled to arrive at the complete sequence. A polypeptide (SEQ ID NO: 78 or 83) was predicted to be encoded by SEQ ID NO: 22 or 27, respectively as set forth below. The polypeptide was predicted using a software program called BLASTX which selects a polypeptide based on a comparison of the translated novel polynucleotide to known polynucleotides. The initial methionine starts at position 95 of SEQ ID NO: 77 and the putative stop codon, TAG, begins at position 560 of the nucleotide sequence SEQ ID NO: 77. The initial methionine starts at position 95 of SEQ ID NO: 82 and the putative stop codon, TAG, begins at positon 623 of SEQ ID NO: 82.

Assembly of the novel nucleotide sequence of SEQ ID NO: 89 was accomplished using a contig sequence SEQ ID NO: 88 as a seed. The seed was extended by gel sequencing (377 Applied Biosystems (ABI) sequencer) using primers to extend the 3′ end (primer extension). The DNA from the full-length clone was then isolated, sonicated and recloned for gel sequencing. Each fragment was sequenced by gel sequencing (377 ABI sequencer) and the sequences were assembled to arrive at the complete sequence. A polypeptide (SEQ ID NO: 90) was predicted to be encoded by SEQ ID NO: 89 as set forth below. The polypeptide was predicted using a software program called BLASTX which selects a polypeptide based on a comparison of the translated novel polynucleotide to known polynucleotides. The initial methionine starts at position 177 of SEQ ID NO: 89 and the putative stop codon, TAG, begins at position 762 of the nucleotide sequence SEQ ID NO: 89.

### Example 8

### Tissue Expression of Full-length Polynucleotides of the Invention

By checking the Nuvelo proprietary database established from screening by hybridization, SEQ ID NO: 2 was found to be expressed in the following human tissue/cell cDNA (see Table 9):

TABLE 9

| | | Total No. of | |
|---|---|---|---|
| | | Clones in the | No. of Positive |
| Library Name | Tissue Origin | Library | Clones |
| ABR006 | adult brain | 108,204 | 9 |
| FBR006 | fetal brain | 151,893 | 5 |
| ABR008 | adult brain | 145,661 | 1 |
| FSK002 | fetal skin | 72,628 | 1 |
| SPC001 | whole organ | 61,905 | 1 |

SEQ ID NO: 2 was further analyzed for their presence in the public dbEST database and their tissue source. SEQ ID NO: 2 was found to be expressed in following tissues: adult brain and nervous_normal.

By checking the Nuvelo proprietary database established from screening by hybridization, SEQ ID NO: 17 was found to be expressed in the following human tissue/cell cDNA (see Table 10):

TABLE 10

| | | | |
|---|---|---|---|
| | | Total No. of | |
| | | Clones in the | No. of Positive |
| Library Name | Tissue Origin | Library | Clones |

| | | | |
|---|---|---|---|
| IB2002 | infant brain | 265743 | 37 |
| IB2003 | infant brain | 201294 | 26 |
| HFB001 | fetal brain | 74494 | 22 |
| IBS001 | infant brain | 33191 | 3 |
| LUC001 | leukocytes | 210372 | 3 |
| ABR001 | adult brain | 30163 | 2 |
| ABD003 | adult brain | 83268 | 2 |
| IBM002 | infant brain | 13952 | 1 |

SEQ ID NO: 17 was further analyzed for their presence in the public dbEST database and their tissue source. SEQ ID NO: 17 was found to be expressed in the following tissues: Pineal gland, Soares infant brain 1NIB, and Infant Brain, Bento Soares.

SEQ ID NO: 17 was mapped to human chromosome 3 by BLAST analysis with human genome sequences.

By checking the Nuvelo proprietary database established from screening by hybridization, SEQ ID NO: 21 was found to be expressed in the following human tissue/cell cDNA (see Table 11):

TABLE 11

| | | Total No. of | |
|---|---|---|---|
| | | Clones in the | No. of Positive |
| Library Name | Tissue Origin | Library | Clones |
| HFB001 | fetal brain | 74494 | 3 |
| IB2002 | infant brain | 265743 | 2 |
| ABR008 | adult brain | 145661 | 1 |
| IB2003 | infant brain | 201294 | 1 |

SEQ ID NO: 21 was further analyzed for their presence in the public dbEST database and their tissue source. SEQ ID NO: 21 was found to be expressed in the following tissues: Schizophrenic brain frontal lobe, hippocampus, testis (cell line), Soares infant brain 1NIB, and frontal lobe.

SEQ ID NO: 21 was mapped to human chromosome 12 by BLAST analysis with human genome sequences.

By checking the Nuvelo proprietary database established from screening by hybridization, SEQ ID NO: 25 was found to be expressed in the following human tissue/cell cDNA (see Table 12):

TABLE 12

| | | Total No. of | |
|---|---|---|---|
| | | Clones in the | No. of Positive |
| Library Name | Tissue Origin | Library | Clones |
| ABT004 | adult brain | 31910 | 1 |
| THA002 | thalamus | 32817 | 1 |
| FUC001 | umbilical cord | 125570 | 1 |
| MMG001 | mammary gland | 131991 | 1 |

SEQ ID NO: 25 was further analyzed for their presence in the public dbEST database and their tissue source. SEQ ID NO: 25 was found to be expressed in the following tissues: Chriocarcinoma and fetal_heart_NbHH19W.

SEQ ID NO: 25 was mapped to human chromosome 1 by BLAST analysis with human genome sequences.

By checking the Nuvelo proprietary database established from screening by hybridization, SEQ ID NO: 29 was found to be expressed in the following human tissue/cell cDNA (see Table 13):

TABLE 13

| | | Total No. of | |
|---|---|---|---|
| | | Clones in the | No. of Positive |
| Library Name | Tissue Origin | Library | Clones |
| HFB001 | fetal brain | 74494 | 8 |
| CVX001 | cervix | 125473 | 7 |
| SPC001 | whole organ | 61905 | 6 |
| THR001 | thyroid gland | 124110 | 6 |
| FUC001 | umbilical cord | 125570 | 6 |
| AKT002 | adult kidney | 149669 | 5 |
| MEL004 | melanoma | 30503 | 4 |
| ABT004 | adult brain | 31910 | 4 |
| FBT002 | fetal brain | 35745 | 4 |
| IB2003 | infant brain | 201294 | 4 |
| FLS002 | fetal liver-spleen | 709733 | 4 |
| FBR004 | fetal brain | 27560 | 3 |
| ABR006 | adult brain | 108204 | 3 |
| THM001 | thymus | 113947 | 3 |
| OBE01 | adipocytes/Obesity | 132217 | 3 |
| BMD001 | bone marrow | 342599 | 3 |
| FLG001 | whole organ | 28154 | 2 |
| ABR001 | adult brain | 30163 | 2 |
| IBS001 | infant brain | 33191 | 2 |
| FMS002 | fetal muscle | 40223 | 2 |
| FSK002 | fetal skin | 72628 | 2 |
| ADR002 | adrenal gland | 90185 | 2 |
| PIT004 | pituitary gland | 120274 | 2 |
| FSK001 | fetal skin | 127263 | 2 |
| SIN001 | whole organ | 142562 | 2 |
| AOV001 | ovary | 259409 | 2 |
| ALG001 | adult lung | 28271 | 1 |
| SKM001 | whole organ | 28327 | 1 |
| PRT001 | whole organ | 28649 | 1 |
| FBR001 | fetal brain | 28664 | 1 |
| UTR001 | uterus | 29595 | 1 |
| THA002 | thalamus | 32817 | 1 |
| FLV001 | fetal liver | 33189 | 1 |
| NTD001 | neuron | 35080 | 1 |
| ESO002 | esophagus | 36840 | 1 |
| FLG004 | fetal lung | 41090 | 1 |
| LFB001 | lung, fibroblast | 41616 | 1 |
| PLA003 | placenta | 80877 | 1 |
| LPC001 | lymphocyte | 97546 | 1 |
| STM001 | bone marrow | 181899 | 1 |
| IB2002 | infant brain | 265743 | 1 |
| FLS001 | fetal liver-spleen | 555770 | 1 |

SEQ ID NO: 29 was further analyzed for their presence in the public dbEST database and their tissue source. SEQ ID NO: 29 was found to be expressed in the following tissues: Prostate, NCI_CGAP_Sub8, Soares_fetal_liver_spleen_{—}1NFLS_S1, fetal brain, rhabdomyosarcoma, hypothalamus, squamous cell carcinoma (4 pooled), Stratagene neuroepithelium, Multiple sclerosis lesions, pooled germ cell tumors, 5 tissues (senescent fibroblasts, placenta, total fetus, parathyroid timor, ovary tumor), Soares adult brain N2b5HB55Y, Soares_testis_NHT, Soares infant brain 1NIB, anaplastic oligodendroglioma, and testis, B-cell and fetal lung.

SEQ ID NO: 29 was mapped to human chromosome 22 by BLAST analysis with human genome sequences.

By checking the Nuvelo proprietary database established from screening by hybridization, SEQ ID NO: 33 was found to be expressed in the following human tissue/cell cDNA (see Table 14):

TABLE 14

| | | Total No. of | |
|---|---|---|---|
| | | Clones in the | No. of Positive |
| Library Name | Tissue Origin | Library | Clones |
| FMS002 | fetal muscle | 40223 | 1 |

SEQ ID NO: 33 was further analyzed for their presence in the public dbEST database and their tissue source. SEQ ID NO: 33 was found to be expressed in the following tissues: adult brain, adrenal cortex carcinoma cell line, hippocampus, hypothalamus, Soares_pineal_gland_N3BPG, Soares_total_fetus_Nb2HF8_{—}9w, and testis, B-cell and fetal lung.

SEQ ID NO: 33 was mapped to human chromosome 3 by BLAST analysis with human genome sequences.

By checking the Nuvelo proprietary database established from screening by hybridization, SEQ ID NO: 43 was expressed in following human tissue/cell cDNA (see Table 15):

TABLE 15

| | No. of Positive | Total No. of Clones | |
|---|---|---|---|
| Library Name | Clones | in the Library | Tissue Origin |
| ABT004 | 2 | 31910 | Adult brain |
| FLG | 1 | 27360 | Fetal lung |
| ABR006 | 1 | 108204 | Adult brain |

SEQ ID NO: 43 was further analyzed for its presence in the public dbEST database and their tissue source. SEQ ID NO: 43 was found to be expressed in following tissues: LT1_FL013_Fbran (fetal brain) and NIH_MGC_{—}96 (hypothalamus).

The gene for SEQ ID NO: 43 was mapped to human chromosome 2 by BLAST analysis with human genome sequences.

By checking the Nuvelo proprietary database established from screening by hybridization, SEQ ID NO: 49 was found to be expressed in following human tissue/cell cDNA (see Table 16):

TABLE 16

| | | Total No. of | |
|---|---|---|---|
| | No. of Positive | Clones in the | |
| Library Name | Clones | Library | Tissue Origin |
| ABT004 | 2 | 31910 | Adult brain |
| FLG | 1 | 27360 | Fetal lung |
| ABR006 | 1 | 108204 | Adult brain |

SEQ ID NO: 49 was further analyzed for their presence in the public dbEST database and their tissue source. SEQ ID NO: 49 was found expressed in LT1_FL013_Fbran (fetal brain) and NIH_MGC_{—}96 (hypothalamus).

The gene for SEQ ID NO: 49 was mapped to chromosome 2 by BLAST analysis with human genome sequences.

By checking the Nuvelo proprietary database established from screening by hybridization, SEQ ID NO: 57 was found to be expressed in the following human tissue/cell cDNA (see Table 17):

TABLE 17

| | No. of Positive | Total No. of Clones | |
|---|---|---|---|
| Library Name | Clones | in the Library | Tissue Origin |
| ADR002 | 1 | 90185 | Adrenal gland |
| SUP008 | 1 | 37997 | Mixed tissues |

The gene corresponding to SEQ ID NO: 57 (Genomic_ID gi17939957) was mapped to human chromosome 11q by BLAST analysis with human genome sequences.

By checking the Nuvelo proprietary database established from screening by hybridization, SEQ ID NO: 70 was found to be expressed in the following human tissue/cell cDNA (see Table 18):

TABLE 18

| | No. of Positive | Total No. of Clones | |
|---|---|---|---|
| Library Name | Clones | in the Library | Tissue Origin |
| ATS001 | 1 | 26744 | Testis |

SEQ ID NO: 70 was further analyzed for its presence in the public dbEST database and its tissue source. SEQ ID NO: 70 was found to be expressed in the following tissues: normal prostate (NCI_CGAP_Pr22), and Soares_testis_NHT.

The gene corresponding to SEQ ID NO: 70 (Genomic_ID gi8117631) was mapped to human chromosome 11q24 by BLAST analysis with human genome sequences.

SEQ ID NO: 89 was analyzed for its presence in the public dbEST database and its tissue source. SEQ ID NO: 89 was found to be expressed in the following tissues: placenta (Soares_placenta_{—}8 to 9 weeks_{—}2NbHP8 to 9W), fetal liver/spleen (Soares_fetal_liver_spleen_{—}1NFLS_S1), adult brain medulla (NIH_MGC_{—}119), hippocampus (NIH_MGC_{—}95), testis cell line (NIH_MGC_{—}97), normal testis (TN), germ cell tumors. (NCI_CGAP_{—GC}6), testis (Soares_testis_NHT), and mixed testis, B-cell and fetal lung (Soares_NFL_T_G).

The gene corresponding to SEQ ID NO: 89 (Genomic_ID gi8118990) was mapped to human chromosome 11q25 by BLAST analysis with human genome sequences.

By checking the Nuvelo proprietary database established from screening by hybridization, SEQ ID NO: 96 was found to be expressed in the following human tissue/cell cDNA (see Table 19):

TABLE 19

| | No. of Positive | Total No. of Clones | |
|---|---|---|---|
| Library Name | Clones | in the Library | Tissue Origin |
| THA002 | 1 | 32817 | Thalamus |

The gene corresponding to SEQ ID NO: 96 (Genomic_ID gi22417443) was mapped to human chromosome 8 by BLAST analysis with human genome sequences.

### EXAMPLE 9

### Expression Analysis of SEQ ID NO: 43 and 49

First strand human cDNA libraries from multiple tissues are screened with gene specific primers for SEQ ID NO: 43 (5′-TACCGCGAGCCCGAC-3′ and 5′-CTAATCCGGGTACAGAAG-3′ (SEQ ID NO: 107 and 108, respectively)). First strand human cDNA libraries from multiple tissues are screened with gene specific primers for SEQ ID NO: 49 (5′-TACAGGTCCCTTAC-3′ and 5′-CTAATCCGGGTACAGAAG-3′ (SEQ ID NO: 109 and 110, respectively)). The commercial panels (Clontech) screened are: Panel I (heart, brain, placenta, lung, liver, skeletal muscle, kidney and pancreas), Panel II (Spleen, thymus, prostate, testis, ovary, small intestine, colon and adipocyte from a marathon ready cDNA library), immune panel (spleen, lymph node, thymus, tonsil, bone marrow, fetal liver, peripheral blood leukocyte) and a blood fraction panel (mononuclear, resting CD8+, resting CD4+, resting CD14+, resting CD19+, activated mononuclear cells, activated CD4+ and activated CD8+). PCR is performed for a total of 30 cycles using the following conditions: an initial denaturation at 94° C. for 3 min, followed by 5 cycles of 30 s at 94° C., 30 sec at 68° C. and 1 min at 72° C., followed by 5 cycles of 30 s at 94° C., 30 sec at 64° C. and 1 min at 72° C., followed by 20 cycles of 30 s at 94° C., 30 sec at 60° C. and 1 min at 72° C. followed by an extension of 10 min at 72° C. The amplification product is detected by analysis on agarose gels stained with ethidium bromide.

### Example 10

### Cellular Localization of SEQ ID NO: 43 and 49

SEQ ID NO: 43 specific primers corresponding to the translational start region and the carboxy-terminal region, excluding the stop codon of the SEQ ID NO: 1 sequence, are used (5′-TACCGCGAGCCCGAC-3′ and 5′-CTAATCCGGGTACAGAAG-3′ (SEQ ID NO: 107 and 108, respectively)). PCR amplification of the 864 nt product is performed using the following conditions; an initial denaturation at 94° C. for 3 min, followed by 5 cycles of 30 s at 94° C., 30 sec at 66° C. and 1 min at 72° C., followed by 5 cycles of 30 s at 94° C., 30 sec at 62° C. and 1 min at 72° C., followed by 20 cycles of 30 s at 94° C., 30 sec at 58° C. and 1 min at 72° C. followed by an extension of 10 min at 72° C. These primers generate a fragment of DNA corresponding to the entire coding region of the SEQ ID NO: 43, flanked by Hind III and Xho I sites. The PCR product is digested accordingly to generate overhang ends that are ligated to the Hind III and Xho I sites of pcDNA3.1/myc-His(+)A (Invitrogen). The resultant mammalian expression plasmid (adiponectin-like/myc-His) allows for expression of the adiponectin-like protein coding sequence fused in-frame with the myc-6His epitope at the carboxy terminus.

Similarly, SEQ ID NO: 49 specific primers corresponding to the translational start region and the carboxy-terminal region, including the stop codon of the SEQ ID NO: 49 sequence, are used (5′-TACAGGTCCCTTAC-3′ and 5′-CTAATCCGGGTACAGAAG-3′ (SEQ ID NO: 109 and 110, respectively) to generate 1182 nt product. The 1182 nt PCR product is then used for the preparation of mammalian expression plasmid as described above.

The mammalian expression vectors are transfected into COS-7 cells. Briefly, cells in a 10 cm dish with 8 ml of medium are incubated with 16 μl of Fugene-6 and 4 μg of DNA for 12 h. The medium is then replaced with serum-free DMEM and incubated for an additional 48 h prior to harvesting. After the conditioned medium is collected from transfected COS-7 cells, cells were washed twice with PBS and then scraped from plates. Upon centrifugation, the cells are resuspended in PBS containing 0.5 μg/ml leupeptin, 0.7 μg/ml pepstatin, and 0.2 μg/ml aprotinin. After a brief sonication, the cytosolic fraction is separated from the insoluble membrane fraction by centrifugation. Purification of proteins from the cytosolic and from the media took place at 4 C in the presence of 100 μl of Ni-NTA resin (Qiagen). The resin is washed twice with 50 mM Tris-HCl (pH 7.5), 300 mM NaCl, and 5 mM imidazole.

To determine the cellular localization of the adiponectin-like/myc-His-tagged proteins, Western blot analysis is performed on cytosolic, membrane, and medium fractions using an anti-myc antibody. The predicted molecular mass of the tagged adiponectin/myc-His-tagged proteins are 31.5 kDa and 43.2 kDa corresponding to SEQ ID NO: 43 and 49 respectively. However, the electrophoretic mobility of the proteins can show altered mass suggesting that adiponectin-like/myc-His-tagged protein is post-translationaly modified.

### Example 11

### Chromosomal Localization of SEQ ID NO: 43 and 49

To determine the chromosomal localization of SEQ ID NO: 43 and 49, gene specific PCR primers (5′-TACCGCGAGCCCGAC-3′ and 5′-CTAATCCGGGTACAGAAG-3′; 5′-TACAGGTCCCTTAC-3′ and 5′-CTAATCCGGGTACAGAAG-3′ (SEQ ID NO: 107-110, respectively)) are screened against the NIGMS human/rodent somatic cell hybrid mapping panel #2. PCR amplification of the 864 nt product is performed using the following conditions; an initial denaturation at 94° C. for 3 min, followed by 5 cycles of 30 s at 94° C., 30 sec at 68° C. and 1 min at 72° C., followed by 5 cycles of 30 s at 94° C., 30 sec at 64° C. and 1 min at 72° C., followed by 20 cycles of 30 s at 94° C., 30 sec at 60° C. and 1 min at 72° C. followed by an extension of 10 min at 72° C. All products are separated by 3% agarose gel electrophoresis and visualized via ethidium bromide staining. SEQ ID NO: 43 and 49 is mapped to chromosome 2.

### Example 12

### Expresson Study of the Polynucleotides of the Invention

The expression of SEQ ID NO: 1-3, 5, 8, 10, 21, 23, 25, 27, 29, 31, 33, 35, 37-40, 43, 45, 49, 51, 53, 56-57, 59, 76-77, 79, 82, 84, 88-89, 91, 95-96, or 98 in various tissues is analyzed using a semi-quantitative polymerase chain reaction-based technique. Human cDNA libraries are used as sources of expressed genes from tissues of interest (adult bladder, adult brain, adult heart, adult kidney, adult lymph node, adult liver, adult lung, adult ovary, adult placenta, adult rectum, adult spleen, adult testis, bone marrow, thymus, thyroid gland, fetal kidney, fetal liver, fetal liver-spleen, fetal skin, fetal brain, fetal leukocyte and macrophage). Gene-specific primers are used to amplify portions of SEQ ID NO: 1-3, 5, 8, 10, 21, 23, 25, 27, 29, 31, 33, 35, 37-40, 43, 45, 49, 51, 53, 56-57, 59, 76-77, 79, 82, 84, 88-89, 91, 95-96, or 98 sequences from the samples. Amplified products are separated on an agarose gel, transferred and chemically linked to a nylon filter. The filter is then hybridized with a radioactively labeled (³³P-dCTP) double-stranded probe generated from SEQ ID NO: 1-3, 5, 8, 10, 21, 23, 25, 27, 29, 31, 33, 35, 37-40, 43, 45, 49, 51, 53, 56-57, 59, 76-77, 79, 82, 84, 88-89, 91, 95-96, or 98 using a Klenow polymerase, random-prime method. The filters are washed (high stringency) and used to expose a phosphorimaging screen for several hours. Bands indicate the presence of cDNA including SEQ ID NO: 1-3, 5, 8, 10, 21, 23, 25, 27, 29, 31, 33, 35, 37-40, 43, 45, 49, 51, 53, 56-57, 59, 76-77, 79, 82, 84, 88-89, 91, 95-96, or 98 sequences in a specific library, and thus mRNA expression in the corresponding cell type or tissue.

### Example 13

### Expression of the Polynucleotides of the Invention in Cells

Chinese Hamster Ovary (CHO) cells or other suitable cell types are grown in DMEM (ATCC) and 10% fetal bovine serum (FBS) (Gibco) to 70% confluence. Prior to transfection, the media is changed to DMEM and 0.5% FBS. Cells are transfected with cDNAs for SEQ ID NO: 1-3, 5, 8, 10, 21, 23, 25, 27, 29, 31, 33, 35, 37-40, 43, 45, 49, 51, 53, 56-57, 59, 76-77, 79, 82, 84, 88-89, 91, 95-96, or 98, or with pBGal vector by the FuGENE-6 transfection reagent (Boehringer). In summary, 4 μl of FuGENE-6 is diluted in 100 μl of DMEM and incubated for 5 min. Then, this is added to 1 μl of DNA and incubated for 15 min before adding it to a 35 mm dish of CHO cells. The CHO cells are incubated at 37° C. with 5% CO₂. After 24 h, media and cell lysates are collected, centrifuged and dialyzed against assay buffer (15 mM Tris pH 7.6, 134 mM NaCl, 5 mM glucose, 3 mM CaCl₂ and MgCl₂).

### Example 14

### A. Expression of Full-length Polypeptides of the Invention in Cells

Chinese Hamster Ovary (CHO) cells or other suitable cell types are grown in DMEM (ATCC) and 10% fetal bovine serum (FBS) (Gibco) to 70% confluence. Prior to transfection, the media is changed to DMEM and 0.5% FBS. Cells are transfected with cDNAs for SEQ ID NO: 4, 7, 9, 12, 22, 24, 26, 28, 30, 32, 34, 44, 46, 50, 58, 61, 78, 81, 83, 86, 90, 93, 97, or 100, or with pBGal vector by the FuGENE-6 transfection reagent (Boehringer). In summary, 4 μl of FuGENE-6 is diluted in 100 μl of DMEM and incubated for 5 min. Then, this is added to 1 μl of DNA and incubated for 15 min before adding it to a 35 mm dish of CHO cells. The CHO cells are incubated at 37° C. with 5% CO₂. After 24 h, media and cell lysates are collected, centrifuged and dialyzed against assay buffer (15 mM Tris pH 7.6, 134 mM NaCl, 5 mM glucose, 3 mM CaCl₂ and MgCl₂).

### Example 15

### Expression of Full-length Polypeptides of the Invention in E. coli

SEQ ID NO: 4, 7, 9, 12, 22, 24, 26, 28, 30, 32, 34, 44, 46, 50, 58, 61, 78, 81, 83, 86, 90, 93, 97, or 100 is expressed in E. coli by subcloning the entire coding region into a prokaryotic expression vector. The expression vector (pQE16) used is from the QIAexpression® prokaryotic protein expression system (QIAGEN). The features of this vector that make it useful for protein expression include: an efficient promoter (phage T5) to drive transcription, expression control provided by the lac operator system, which can be induced by addition of IPTG (isopropyl-β-D-thiogalactopyranoside), and an encoded histidine, His6, tag comprising a stretch of 6 histidine amino acid residues which can bind very tightly to a nickel atom. The vector can be used to express a recombinant protein with a His6 tag fused to its carboxyl terminus, allowing rapid and efficient purification using Ni-coupled affinity columns.

PCR is used to amplify the coding region which is then ligated into digested pQE16 vector. The ligation product is transformed by electroporation into electrocompetent E.coli cells (strain M5 [pREP4] from QIAGEN), and the transformed cells are plated on ampicillin-containing plates. Colonies are screened for the correct insert in the proper orientation using a PCR reaction employing a gene-specific primer and a vector-specific primer. Positives are then sequenced to ensure correct orientation and sequence. To express the polypeptide of the invention, a colony containing a correct recombinant clone is inoculated into L-Broth containing 100 μg/ml of ampicillin, 25 μg/ml of kanamycin, and the culture is allowed to grow overnight at 37° C. The saturated culture is then diluted 20-fold in the same medium and allowed to grow to an optical density at 600 nm of 0.5. At this point, IPTG is added to a final concentration of 1 mM to induce protein expression. The culture is allowed to grow for 5 more hours, and then the cells are harvested by centrifugation at 3000×g for 15 minutes.

The resultant pellet is lysed using a mild, nonionic detergent in 20 mM Tris HCl (pH 7.5) (B-PER™ Reagent from Pierce), or by sonication until the turbid cell suspension turned translucent. The lysate obtained is further purified using a nickel-containing column (Ni-NTA spin column from QIAGEN) under non-denaturing conditions. Briefly, the lysate is brought up to 300 mM NaCl and 10 mM imidazole and centrifuged at 700×g through the spin column to allow the His-tagged recombinant protein to bind to the nickel column. The column is then washed twice with Wash Buffer (50 mM NaH₂PO₄, pH 8.0; 300 mM NaCl; 20 mM imidazole) and is eluted with Elution Buffer (50 mM NaH₂PO₄, pH 8.0; 300 mM NaCl; 250 mM imidazole). All the above procedures are performed at 4° C. The presence of a purified protein of the predicted size is confirmed with SDS-PAGE.

### Example 16

### Expression and Purification of Polypeptides of the Invention from Insect Cells

Polypeptides of the invention are expressed in insect cells as follows:

An open reading frame expressing a polypeptide of the invention is cloned by PCR into a pIB/V5-His TOPO TA cloning vector (Invitrogen Corporation) either with a Myc/His tag or without any tags. Insect cells (High Five™, Invitrogen) are transfected with the plasmid DNA containing the tagged or untagged version of the polypeptide of the invention by using the InsectSelect™System (Invitrogen). The expression of the polypeptide of the invention is determined by transient expression. The medium containing an expressed polypeptide of the invention is separated on SDS-PAGE and the expressed polypeptide of the invention is identified by Western blot analysis. For large-scale production of a polypeptide of the invention, resistant cells are expanded into flasks containing Ultimate InsectSerum-Free medium (Invitrogen). The cells are shaken at ˜100 mph at 27° C. for 4 days. The conditioned media containing the protein for purification are collected by centrifugation.

### Example 17

### Production of Antibodies Specific to the Polypeptides of the Invention

Cells expressing a polypeptide of the invention are identified using antibodies specific to the polypeptide of the invention. Polyclonal antibodies are produced by DNA vaccination or by injection of peptide antigens into rabbits or other hosts. An animal, such as a rabbit, is immunized with a peptide from the extracellular region of the polypeptide of the invention conjugated to a carrier protein, such as BSA (bovine serum albumin) or KLH keyhole limpet hemocyanin). The rabbit is initially immunized with conjugated peptide in complete Freund's adjuvant, followed by a booster shot every two weeks with injections of conjugated peptide in incomplete Freund's adjuvant. Antibodies of the invention are affnity purified from rabbit serum using a peptide of the invention coupled to Affi-Gel 10 (Bio-Rad), and stored in phosphate-buffered saline (PBS) with 0.1% sodium azide. To determine that the polyclonal antibodies are specific for the polypeptide of the invention, an expression vector encoding the polypeptide of the invention is introduced into mammalian cells. Western blot analysis of protein extracts of non-transfected cells and the cells expressing the polypeptide of the invention is performed using the polyclonal antibody sample as the primary antibody and a horseradish peroxidase-labeled anti-rabbit antibody as the secondary antibody. Detection of a band corresponding to the molecular weight of the polypeptide of the invention in the cells expressing the polypeptide of the invention and lack thereof in the control cells indicates that the polyclonal antibodies are specific for said polypeptide of the invention.

Monoclonal antibodies are produced by injecting mice with a peptide of the invention, with or without adjuvant. Subsequently, the mouse is boosted every 2 weeks until an appropriate immune response has been identified (typically 1-6 months), at which point the spleen is removed. The spleen is minced to release splenocytes, which are fused (in the presence of polyethylene glycol) with murine myeloma cells. The resulting cells (hybridomas) are grown in culture and selected for antibody production by clonal selection. The antibodies are secreted into the culture supernatant, facilitating the screening process, such as screening by an enzyme-linked immunosorbent assay (ELISA). Alternatively, humanized monoclonal antibodies are produced either by engineering a chimeric murine/human monoclonal antibody in which the murine-specific antibody regions are replaced by the human counterparts and produced in mammalian cells, or by using transgenic “knock out” mice in which the native antibody genes have been replaced by human antibody genes and immunizing the transgenic mice as described above.

### Example 18

### Multiplex Analysis of Protein Prosphorylation and Cytokine/Chemokine Activation After Treatment with Polypeptides of the Invention

### A. Secretion Levels of the Polypeptide of the Invention

The full-length open reading frame of the polypeptide of the invention (ie. SEQ ID NO: 1-3, 5, 8, 10, 21, 23, 25, 27, 29, 31, 33, 35, 37-40, 43, 45, 49, 51, 53, 56-57, 59, 76-77, 79, 82, 84, 88-89, 91, 95-96, or 98) is cloned into the mammalian expression vector pCDNA3.1V5-His-Topo (Invitrogen, Carlsbad, Calif.) to generate a C-terminal V5-His tagged expression construct. The resulting plasmid is transiently transfected into COS7L cells using the Fugene-6 transfection reagent (Roche Biosciences). The presence of the V5-His tagged protein is determined in both culture supernatant and cell lysate by Western blotting using anti-V5 antibodies and chemiluminescence visualization. The percent secretion is determined by comparing the amount of protein in the supernatant to the amount of protein in the cell lysate.

### B. Detection of Intracellular Protein Phosphorylation

The assay described below, a Bio-Plex (Bio-Rad, Hercules, Calif.) phosphorylation assay, is one of several methods employed for measuring protein phosphorylation in order to assess potential functions of secreted proteins in the particular cell type tested. Briefly, purified antibodies against various protein kinases, JNK, p38MAPK, erk, Stat3, and IκBα, are conjugated to microsphere sets according to the manufacturer's protocol. Culture supernatant from COS7L cells, transiently transfected with an expression plasmid containing a V5-His tagged fusion protein of the polypeptide of the invention (see Example 33A), is harvested and 10 μl of the culture supernatant is added to a panel of target cell lines for 15 min at 37° C. Cells are lysed and the lysate is clarified. The conjugated microspheres are incubated with 25 μl of cell lysate in a final volume of 50 μl in a 96-well plate overnight at room temperature with constant shaling. After incubation, the microspheres are washed with Tris buffered saline (TBS) containing 0.02% Tween-20 (TBST). Protein phosphorylation is detected by incubating the microspheres with 25 μl of a mixture of biotinylated antibodies against the phosphorylated forms of the protein kinases, for example, anti-phospho-Stat3, in TBST containing 5% mouse serum at room temperature for 30 min with constant shaking. The microspheres are washed with TBST and further incubated with 2 μg/ml of streptavidin-phycoerythrin (PE). The resulting microspheres with the reaction complex are analyzed using the Luminex Reader (Luminex Co., Austin, Tex.).

### C. Detection of Cytokine/Chemokine Levels

Cytokine and chemoline levels are determined using the assay described below, the Luminex Multi-plex bead assay, which is very similar to a typical sandwich ELISA assay, but utilizes Luminex microspheres conjugated to anti-cytoline and anti-chemokine antibodies (Vignali, J. Immunol. Methods 243:243-255 (2000), herein incorporated by reference). Briefly, purified antibodies against a variety of cytokines and chemokines are conjugated to microsphere sets (Luminex Co., Austin, Tex.) according to the manufacturer's protocol. Culture supernatant from COS7L cells, transiently transfected with an expression plasmid containing a V5-His tagged fusion protein of the invention (see Example 33A), is harvested and 25 μl of the culture supernatant is added to a panel of target cell lines and incubated overnight at 37° C. Condition media is then harvested. The conjugated microspheres are incubated with 50 μl in a 96-well filter plate at room temperature for 30 min with constant shaking. After incubation, the microspheres are washed and incubated with 50 μl (1 μg/ml) of biotinylated anti-cytokine or anti-chemokine antibodies in phosphate buffered saline (PBS) containing 0.5% Tween-20, 0.2% BSA, 5% mouse serum at room temperature for 30 min. The microspheres are washed and further incubated with 2 μg/ml of Streptavidin-PE. The resulting microspheres with the reaction complex are analyzed using the Luminex Reader (Luminex Co., Austin, Tex.).

### Example 19

### Calcium Mobilization Assay

Many extracellular signals to intracellular targets are mediated by increases in free calcium levels in the cytoplasm. Calcium mobilization from intracellular stores can be detected in many cell types by loading the cells with a Ca²⁺ sensitive indicator such as fura-2-AM. The increase in fluorescence is detected by a fluorescence plate reader. Cells will be incubated in media containing 5 μM Fura-2 AM, 5 μM Pluronic F-127 for 30 min. After the addition of adiponectin-like protein the Fura-2 intensity will be monitored approximately every 20 sec by a fluorescent plate reader (Molecular Dynamics) and compared to the intensity of cells with basal calcium levels.

### Example 20

### Fatty Acit Oxidation Assay

The oxidation of palmitate or oleate in culture C2C12 skeletal muscle cells (ATCC; CRL-1772) upon exposure to adiponectin-like protein is measured according to published procedures (Barger et al., J. Clin. Invest. 105:1723-1730 (2000)). In summary, nearly confluent C2C12 myocytes are kept in differentiation medium (DMEM, 2.5% horse serum) for 7 days, at which time formation of myotubes is maximal. [1-¹⁴C]oleic acid (1 μCi/ml) is added to the cells and incubated for 90 minutes at 37° C. in the absence/presence of adiponectin-like protein. In some of the assays a proteolytically cleaved adiponectin-like protein (cleaved between lysine 190-glycine 191) may be employed. During the experiment the C2C12 cells are incubated in a closed system containing Whatman paper to collect the ¹⁴CO₂ gas released during fatty acid oxidation. After the incubation the Whatman paper is removed and the amount of ¹⁴C radioactivity is determined by liquid scintillation counting.

### Example 21

### Macrophage Phagocytosis Assay

Human macrophages are incubated in the presence/absence of adiponectin-like protein for 24 hours at 37° C. in 96-well plates. Fluobrite fluorescent-microspheres (0.75G; Polyscience, Warrington, Pa.) are added to each well, followed by one hour incubation at 37° C. Nonadherent latex beads are removed by gentle washing and the cells are incubated for an additional 30 minutes to complete phagocytosis. The cells are harvested by short-time treatment with EDTA and trypsin and washed vigorously three times with PBS to remove noningested beads. The amount of ingested beads will be measured with a FACScan.

### Example 22

### Glucose Uptake Assay

The adiponectin-like proteins influence carbohydrate and lipid metabolism. One of the ways by which the adiponectin-like proteins affect the development of insulin resistence is by altering glucose metabolism. To evaluate the effect of the polypeptides of the invention on glucose uptake, differentiated rat L6 myotube cells are cultured in 96-well plate for a minimum of 5 days in DMEM with 3% horse serum. The cells are incubated in 100 μl serum free media containing 25 mM glucose at 37 C in 5% CO₂ with or without adiponectin-homolog proteins of SEQ ID NO: 44 or 50 at a concentration of 30 μg/ml for 4-5 hours, followed by a subsequent incubation with insulin (100 nM) for 1 hour. The cells are then washed with serum containing media twice to remove glucose. The cells are further incubated with 10 μM [1,2-³H]2-deoxyglucose in 50 μl HBS for 20 min at 30 C. The overlayed media is removed and the cells are washed twice with 200 μl of HBS buffer to remove the excess 2-Deoxy-D-[1-³H]2-glucose from the cells. The cells are lysed with 100 μl of 1 M NaOH by incubation for 30 min. The supernatants from the cells are collected and stored. 5 μl of supernatant is transferred to a 96-well plate for radioactive counting in the 96-well scintillation counter for measuring the ³H uptake by the cells. The ³H uptake by cells reflects the glucose uptake induced by adiponectin by the cells. (Sarabia et. al., Biochem Cell Biol 68:536-542 (1990); Yu et al., J. Biol. Chem. 276: 19994-19998 (2001)).

### Example 23

### Expression Levels of CEA- or LY-6-like mRNA in Tumor Cell Lines and Tumor Tissue

Expression of CEA- or Ly-6-like mRNA is determined in various tumor cell lines and tumor tissues, including lymphoma, leukemia, melanoma, breast cancer, ovarian cancer, lung cancer, brain cancer, colon cancer, prostate cancer, pancreatic cancer, gastric cancer, etc. Poly-A messenger RNA is isolated from the cell lines and subjected to quantitative, real-time PCR analysis (Simpson, et al., Molec. Vision. 6: 178-183 (2000)) to determine the relative copy number of CEA- or Ly-6-like mRNA expressed per cell in each line. Elongation factor 1 mRNA expression is used as a positive control and normalization factors in all samples.

Expression of CEA- or Ly-6-like mRNA is determined in various healthy and tumor tissues . Poly-A mRNA is isolated from various tissues and subjected to quantitative, real-time PCR analysis, as described above, to determine the relative expression of CEA- or Ly-6-like mRNA in the sample.

### Example 24

### In Vitro Antibody-dependent Cytotoxicity Assay

The ability of a CEA- or Ly-6-like protein-specific antibody to induce antibody-dependent cell-mediated cytoxicity (ADCC) is determined in vitro. ADCC is performed using the CytoTox 96 Non-Radioactive Cytoxicity Assay (Promega; Madison, Wis.) (Hornick et al., Blood 89:4437-4447, (1997)) as well as effector and target cells. Peripheral blood mononuclear cells (PBMC) or neutrophilic polymorphonuclear leukocytes (PMN) are two examples of effector cells that can be used in this assay. PBMC are isolated from healthy human donors by Ficoll-Paque gradient centrifugation, and PMN are purified by centrifugation through a discontinuous percoll gradient (70% and 62%) followed by hypotonic lysis to remove residual erythrocytes. RA1 B cell lymphoma cells (for example) are used as target cells.

RA1 cells are suspended in RPMI 1640 medium supplemented with 2% fetal bovine serum and plated in 96-well V-bottom microtitier plates at 2×10⁴ cells/well. CEA- or Ly-6-like protein-specific antibody is added in triplicate to individual wells at 1 μg/ml, and effector cells are added at various effector:target cell ratios (12.5:1 to 50:1). The plates are incubated for 4 hours at 37° C. The supernatants are then harvested, lactate dehydrogenase release determined, and percent specific lysis calculated using the manufacture's protocols.

### Example 25

### Toxin-conjugated CEA- or LY-6-like Protein-specific Antibodies

Antibodies to CEA- or Ly-6-like protein are conjugated to toxins and the effect of such conjugates in animal models of cancer is evaluated. Chemotherapeutic agents, such as calicheamycin and carboplatin, or toxic peptides, such as ricin toxin, are used in this approach. Antibody-toxin conjugates are used to target cytotoxic agents specifically to cells bearing the antigen. The antibody-toxin binds to these antigen-bearing cells, becomes internalized by receptor-mediated endocytosis, and subsequently destroys the targeted cell. In this case, the antibody-toxin conjugate targets CEA- or Ly-6-like protein-expressing cells, such as B cell lymphomas, and deliver the cytotoxic agent to the tumor resulting in the death of the tumor cells.

One such example of a toxin that may be conjugated to an antibody is carboplatin. The mechanism by which this toxin is conjugated to antibodies is described in Ota et al., Asia-Oceania J. Obstet. Gynaecol. 19: 449-457 (1993). The cytotoxicity of carboplatin-conjugated CEA- or Ly-6-like protein-specific antibodies is evaluated in vitro, for example, by incubating CEA- or Ly-6-like protein-expressing target cells (such as the RA1 B cell lymphoma cell line) with various concentrations of conjugated antibody, medium alone, carboplatin alone, or antibody alone. The antibody-toxin conjugate specifically targets and kills cells bearing the CEA- or Ly-6-like protein antigen, whereas, cells not bearing the antigen, or cells treated with medium alone, carboplatin alone, or antibody alone, show no cytotoxicity.

The antitumor efficacy of carboplatin-conjugated CEA- or Ly-6-like protein-specific antibodies is demonstrated in in vivo murne tumor models. Five to six week old, athymic nude mice are engrafted with tumors subcutaneously or through intravenous injection. Mice are treated with the CEA- or Ly-6-like protein-carboplatin conjugate or with a non-specific antibody-carboplatin conjugate. Tumor xenografts in the mouse bearing the CEA- or Ly-6-like protein antigen are targeted and bound to by the CEA- or Ly-6-like protein-carboplatin conjugate. This results in tumor cell killing as evidenced by tumor necrosis, tumor shrinkage, and increased survival of the treated mice.

Other toxins are conjugated to CEA- or Ly-6-like protein-specific antibodies using methods known in the art. An example of a toxin conjugated antibody in human clinical trials is CMA-676, an antibody to the CD33 antigen in AML which is conjugated with calichearnicin toxin (Larson, Semin. Hematol. 38(Suppl 6):24-31 (2001)).

### Example 26

### Radioimmunotherapy Using CEA- or LY-6-like Protein-specific Antibodies

Animal models are used to assess the effect of antibodies specific to CEA- or Ly-6-like protein as vectors in the delivery of radionuclides in radioimmunotherapy to treat lymphoma, hematological malignancies, and solid tumors. Human tumors are propagated in 5-6 week old athymic nude mice by injecting a carcinoma cell line or tumor cells subcutaneously. Tumor-bearing animals are injected intravenously with radio-labeled anti-CEA- or Ly-6-like protein antibody (labeled with 30-40 μCi of ¹³¹I, for example) (Behr, et al., Int. J. Cancer 77: 787-795 (1988)). Tumor size is measured before injection and on a regular basis (i.e. weekly) after injection and compared to tumors in mice that have not received treatment. Anti-tumor efficacy is calculated by correlating the calculated mean tumor doses and the extent of induced growth retardation. To check tumor and organ histology, animals are sacrificed by cervical dislocation and autopsied. Organs are fixed in 10% formalin, embedded in paraffin, and thin sectioned. The sections are stained with hematoxylin-eosin.

### Example 27

### Immunotherapy Using CEA- or LY-6-like Protein-specific Antibodies

Animal models are used to evaluate the effect of CEA- or Ly-6-like protein-specific antibodies as targets for antibody-based immunotherapy using monoclonal antibodies. Human myeloma cells are injected into the tail vein of 5-6 week old nude mice whose natural killer cells have been eradicated. To evaluate the ability of CEA- or Ly-6-like protein-specific antibodies in preventing tumor growth, mice receive an intraperitoneal injection with CEA- or Ly-6-like protein-specific antibodies either 1 or 15 days after tumor inoculation followed by either a daily dose of 20 μg or 100 μg once or twice a week, respectively (Ozaki, et al., Blood 90:3179-3186 (1997)). Levels of human IgG (from the immune reaction caused by the human tumor cells) are measured in the murine sera by ELISA.

The effect of CEA- or Ly-6-like protein-specific antibodies on the proliferation of myeloma cells is examined in vitro using a ³H-thymidine incorporation assay (Ozaki et al., supra). Cells are cultured in 96-well plates at 1×10⁵ cells/ml in 100 μl/well and incubated with various amounts of CEA- or Ly-6-like protein antibody or control IgG (up to 100 μg/ml) for 24 h. Cells are incubated with 0.5 μCi ³H-thymidine (New England Nuclear, Boston, Mass.) for 18 h and harvested onto glass filters using an automatic cell harvester (Packard, Meriden, Conn.). The incorporated radioactivity is measured using a liquid scintillation counter.

The cytotoxicity of the CEA- or Ly-6-like protein monoclonal antibody is examined by the effect of complements on myeloma cells using a ⁵¹Cr-release assay (Ozali et al., supra). Myeloma cells are labeled with 0.1 mCi ⁵¹Cr-sodium chromate at 37° C. for 1 h. ⁵¹Cr-labeled cells are incubated with various concentrations of CEA- or Ly-6-like protein monoclonal antibody or control IgG on ice for 30 min. Unbound antibody is removed by washing with medium. Cells are distributed into 96-well plates and incubated with serial dilutions of baby rabbit complement at 37° C. for 2 h. The supernatants are harvested from each well and the amount of ⁵¹Cr released is measured using a gamma counter. Spontaneous release of ⁵¹Cr is measured by incubating cells with medium alone, whereas maximum ⁵¹Cr release is measured by treating cells with 1% NP-40 to disrupt the plasma membrane. Percent cytotoxicity is measured by dividing the difference of experimental and spontaneous ⁵¹Cr release by the difference of maximum and spontaneous ⁵¹Cr release.

Antibody-dependent cell-mediated cytotoxicity (ADCC) for the CEA- or Ly-6-like protein monoclonal antibody is measured using a standard 4 h ⁵¹Cr-release assay (Ozaki et al., supra). Splenic mononuclear cells from SCID mice are used as effector cells and cultured with or without recombinant interleulin-2 (for example) for 6 days. ⁵¹Cr-labeled target myeloma cells (1×10⁴ cells) are placed in 96-well plates with various concentrations of anti-CEA- or Ly-6-like protein monoclonal antibody or control IgG. Effector cells are added to the wells at various effector to target ratios (12.5:1 to 50:1). After 4 h, culture supernatants are removed and counted in a gamma counter. The percentage of cell lysis is determined as above.

### Example 28

### CEA- or LY-6-like Protein-specific Antibodies as Immunosuppressants

Animal models are used to assess the effect of CEA- or Ly-6-like protein-specific antibodies to suppress autoimmune diseases, such as arthritis or other inflammatory conditions, or rejection of organ transplants. Immunosuppression is tested by injecting mice with horse red blood cells (HRBCs) and assaying for the levels of HRBC-specific antibodies (Yang, et al., Int. Immunopharm. 2:389-397 (2002)). Animals are divided into five groups, three of which are injected with anti-TLR9 antibodies for 10 days, and 2 of which receive no treatment. Two of the experimental groups and one control group are injected with either Earle's balanced salt solution (EBSS) containing 5-10×10⁷ HRBCs or EBSS alone. Anti-CEA- or Ly-6-like protein antibody treatment is continued for one group while the other groups receive no antibody treatment. After 6 days, all animals are bled by retro-orbital puncture, followed by cervical dislocation and spleen removal. Splenocyte suspensions are prepared and the serum is removed by centrifugation for analysis.

Immunosupression is measured by the number of B cells producing HRBC-specific antibodies. The Ig isotype (for example, IgM, IgG1, IgG2, etc.) is determined using the IsoDetect™ Isotyping kit (Stratagene, La Jolla, Calif.). Once the Ig isotype is known, murine antibodies against HRBCs are measured using an ELISA procedure. 96-well plates are coated with HRBCs and incubated with the anti-HRBC antibody-containing sera isolated from the animals. The plates are incubated with alkaline phosphatase-labeled secondary antibodies and color development is measured on a microplate reader (SPECTRAmax 250, Molecular Devices) at 405 nm usingp-nitrophenyl phosphate as a substrate.

Lymphocyte proliferation is measured in response to the T and B cell activators concanavalin A and lipopolysaccharide, respectively (Jiang, et al., J. Immunol. 154:3138-3146 (1995). Mice are randomly divided into 2 groups, 1 receiving anti-CEA- or Ly-6-like protein antibody therapy for 7 days and 1 as a control. At the end of the treatnent, the animals are sacrificed by cervical dislocation, the spleens are removed, and splenocyte suspensions are prepared as above. For the ex vivo test, the same number of splenocytes are used, whereas for the in vivo test, the anti-CEA- or Ly-6-like protein antibody is added to the medium at the beginning of the experiment. Cell proliferation is also assayed using the ³H-thymidine incorporation assay described above (Ozaki, et al., Blood 90: 3179 (1997)).

### Example 29

### Cytokine Secretion in Response to CEA- or LY-6-like Protein Peptide Fragments

Assays are carried out to assess activity of fragments of the CEA- or Ly-6-like protein, such as the Ig domain, to stimulate cytokine secretion and to stimulate immune responses in NK cells, B cells, T cells, and myeloid cells. Such immune responses can be used to stimulate the immune system to recognize and/or mediate tumor cell killing or suppression of growth. Similarly, this immune stimulation can be used to target bacterial or viral infections. Alternatively, fragments of the CEA- or Ly-6-like protein that block activation through the CEA- or Ly-6-like protein receptor may be used to block immune stimulation in natural killer (NK), B, T, and myeloid cells.

Fusion proteins containing fragments of the CEA- or Ly-6-like protein, such as the Ig domain (CEA- or Ly-6-like-Ig), are made by inserting a CD33 leader peptide, followed by a CEA- or Ly-6-like protein domain fused to the Fc region of human IgG1 into a mammalian expression vector, which is stably transfected into NS-1 cells, for example. The fusion proteins are secreted into the culture supernatant, which is harvested for use in cytokine assays, such as interferon-γ (IFN-γ) secretion assays (Martin, et al., J. Immunol. 167:3668-3676 (2001)).

PBMCs are activated with a suboptimal concentration of soluble CD3 and various concentrations of purified, soluble anti-CEA- or Ly-6-like protein monoclonal antibody or control IgG. For CEA- or Ly-6-like protein-Ig cytokine assays, anti-human Fc Ig at 5 or 20 μg/ml is bound to 96-well plates and incubated overnight at 4° C. Excess antibody is removed and either CEA- or Ly-6-like protein-Ig or control Ig is added at 20-50 μg/ml and incubated for 4 h at room temperature. The plate is washed to remove excess fusion protein before adding cells and anti-CD3 to various concentrations. Supernatants are collected after 48 h of culture and IFN-γ levels are measured by sandwich ELISA, using primary and biotinylated secondary anti-human IFN-γ antibodies as recommended by the manufacturer.

### Example 30

### Diagnostic Methods Using CEA- or LY-6-like Protein-specific Antibodies to Detect CEA- or LY-6-like Protein Expression

Expression of CEA- or Ly-6-like protein in tissue samples (normal or diseased) is detected using anti-CEA- or Ly-6-like protein antibodies. Samples are prepared for immunohistochemical (IHC) analysis by fixing the tissue in 10% formalin embedding in paraffin, and sectioning using standard techniques. Sections are stained using the CEA- or Ly-6-like protein-specific antibody followed by incubation with a secondary horse radish peroxidase (HRP)-conjugated antibody and visualized by the product of the HRP enzymatic reaction.

Expression of CEA- or Ly-6-like protein on the surface of cells within a blood sample is detected by flow cytometry. Peripheral blood mononuclear cells (PBMC) are isolated from a blood sample using standard techniques. The cells are washed with ice-cold PBS and incubated on ice with the CEA- or Ly-6-like protein-specific polyclonal antibody for 30 min. The cells are gently pelleted, washed with PBS, and incubated with a fluorescent anti-rabbit antibody for 30 min. on ice. After the incubation, the cells are gently pelleted, washed with ice cold PBS, and resuspended in PBS containing 0.1% sodium azide and stored on ice until analysis. Samples are analyzed using a FACScalibur flow cytometer (Becton Dickinson) and CELLQuest software (Becton Dickinson). Instrument setting are determined using FACS-Brite calibration beads (Becton-Dickinson).

Tumors expressing CEA- or Ly-6-like protein are imaged using CEA- or Ly-6-like protein-specific antibodies conjugated to a radionuclide, such as ¹²³I, and injected into the patient for targeting to the tumor followed by X-ray or magnetic resonance imaging.

### Example 31

### Tumor Imaging Using CEA- or LY-6-like Protein-specific Antibodies

CEA- or Ly-6-like protein-specific antibodies are used for imaging CEA- or Ly-6-like like protein-expressing cells in vivo. Six-week-old athymic nude mice are irradiated with 400 rads from a cesium source. Three days later the irradiated mice are inoculated with 4×10⁷ RA1 cells and 4×10⁶ human fetal lung fibroblast feeder cells subcutaneously in the thigh When the tumors reach approximately 1 cm in diameter, the mice are injected intravenously with an inoculum containing 100 μCi/10 μg of ¹³¹I-labeled CEA- or Ly-6-like protein-specific antibody. At 1, 3, and 5 days postinjection, the mice are anesthetized with a subcutaneous injection of 0.8 mg sodium pentobarbital. The immobilized mice are then imaged in a prone position with a Spectrum 91 camera equipped with a pinhole collimator (Raytheon Medical Systems; Melrose Park, Ill.) set to record 5,000 to 10,000 counts using the Nuclear MAX Plus image analysis software package (MEDX Inc.; Wood Dale, Ill.) (Homick, et al., Blood 89:44374-447 (1997)).

### Example 32

### Effect of LY-6-like Antibodies on Murine Sperm Function and Egg Binding

### A. Acrosome Reaction Procedure

The effect of anti-Ly-6 antibodies on the acrosome reaction of murine sperm is examined according to the procedure outlined in Chauhan and Naz (Mol. Reprod. Dev. 60:425-432 (2001), herein incorporated by reference). Briefly, motile sperm are collected by the swimming up procedure from cauda epididymides and are capacitated in the presence of anti-Ly-6 antibodies/control Ig by incubating for 2 h at 37° C. in 5% CO₂ and 95% air in Biggers-Whittens-Whittingham (BWW) medium containing 1% BSA (BWW-BSA medium). The acrosome reaction is induced by incubating with the calcium ionophore A23187 in a final concentration of 10 μM for 30 min (Byrd et al., Gamete Res. 22:109-122 (1989), herein incorporated by reference). The sperm are washed to remove the calcium ionophore, fixed in 7.5% formalin, spread over poly-L-lysine coated slides, air dried, and stained with 0.04% (w/v) Coomassie blue G-250 in 3.5% perchloric acid to analyze the status of the acrosome reaction (Thaler and Cardullo, Biochemistry 34:7788-7795 (1995), herein incorporated by reference).

### B. Sperm-egg Binding Assay

Sexually mature male and female CD-1 mice are used for these experiments according to the procedure outlined in Chauhan and Naz (Mol. Reprod. Dev. 60:425-432 (2001)). The female mice are superovulated by interperitoneal injection of 7 IU equine gonadotropin (eCG), oocytes are collected after 12 h of eCG administration and the cumulus cells are removed by using 0.1% hyaluronidase (Naz and Zhu, Biol. Reprod. 59:1095-1100 (1998), herein incorporated by reference). Cauda epididymal sperm are collected, capacitated for 1.5 h and incubated in microdrops of BWW-BSA medium under mineral oil with different concentrations of anti-Ly-6-like antibodies/control Ig for 1 h at 37° C. in 5% CO₂ atmosphere. The eggs (n-4-10) are added to microdrops, and the mixture is incubated for 20 min at 37° C. in 5% CO₂, 95% air mixture. The eggs are washed removing the loosely bound sperm and the number of tightly bound sperm in a single plane of view are counted.

We claim:

1. An isolated polynucleotide comprising a nucleotide sequence selected from the group consisting of SEQ ID NO: 1-3, 5, 8, 10, 21, 23, 25, 27, 29, 31, 33, 35, 37-40, 43, 45, 49, 51, 53, 56-57, 59, 76-77, 79, 82, 84, 88-89, 91, 95-96, or 98 or the mature protein coding portion thereof.

2. An isolated polynucleotide encoding a polypeptide with biological activity, wherein said polynucleotide hybridizes to the polynucleotide of claim 1 under stringent hybridization conditions (0.5 M NaBPO₄, 7% sodium dodecyl sulfate (SDS), 1 mM EDTA at 65° C.).

3. The polynucleotide of claim 1 wherein said polynucleotide is DNA.

4. An isolated polynucleotide which comprises the complement of any one of the polynucleotides of claim 1.

5. A vector comprising the polynucleotide of claim 1.

6. An expression vector comprising the polynucleotide of claim 1.

7. A host cell genetically engineered to comprise the polynucleotide of claim 1.

8. A host cell genetically engineered to comprise the polynucleotide of claim 1 operatively associated with a regulatory sequence that modulates expression of the polynucleotide in the host cells.

9. An isolated polypeptide, wherein the polypeptide is selected from the group consisting of:
(a) a polypeptide encoded by any one of the polynucleotides of claim 1; and
(b) a polypeptide encoded by a polynucleotide hybridizing under stringent conditions with any one of SEQ ID NO: 4, 6-7, 9, 11-12, 22, 24, 26, 28, 30, 32, 34, 36, 44, 46-48, 50, 52-53, 58, 60-62, 78, 80-81, 83, 85-87, 90, 92-94, 97, or 99-101.

10. An isolated polypeptide comprising an amino acid sequence selected from the group consisting of any one of the polypeptides of SEQ ID NO: 4, 6-7, 9, 11-12, 22, 24, 26, 28, 30, 32, 34, 36, 44, 46-48, 50, 52-53, 58, 60-62, 78, 80-81, 83, 85-87, 90, 92-94, 97, or 99-101.

11. A composition comprising the polypeptide of claim 9 or 10 and a carrier.

12. An antibody directed against the polypeptide of claim 9 or 10.

13. A method for detecting the polynucleotide of claim 1 in a sample, comprising the steps of:
(a) contacting the sample with polynucleotide probe that specifically hybridizes to the polynucleotide under conditions which permit formation of a probe/polynucleotide complex; and
(b) detecting the presence of a probe/polynucleotide complex, wherein the presence of the complex indicates the presence of a polynucleotide.

14. A method for detecting the polynucleotide of claim 1 in a sample, comprising the steps of:
(a) contacting the sample under stringent hybridization conditions with nucleic acid primers that anneal to the polynucleotide of claim 1 under such conditions; and
(b) amplifying the polynucleotide or fragment thereof, so that if the polynucleotide or fragment is amplified, the polynucleotide is detected.

15. The method of claim 14, wherein the polynucleotide is an RNA molecule that encodes the polypeptide of claim 9 or 10, and the method further comprises reverse transcribing an annealed RNA molecule into a cDNA polynucleotide.

16. A method of detecting the presence of the polypeptide of claim 9 or 10 having the amino acid sequence of any one of SEQ ID NO: 4, 6-7, 9, 11-12, 22, 24, 26, 28, 30, 32, 34, 36, 44, 46-48, 50, 52-53, 58, 60-62, 78, 80-81, 83, 85-87, 90, 92-94, 97, or 99-101, or a fragment thereof in a cell, tissue or fluid sample comprising:
(a) contacting said cell, tissue or fluid sample with an antibody or fragment of claim 10 under conditions which permit the formation of an antibody/polypeptide complex; and
(b) detecting the presence of an antibody/polypeptide complex, wherein the presence of the antibody/polypeptide complex indicates the presence of any of the polypeptides of claim 10.

17. A method for identifying a compound that binds to a polypeptide of any one of SEQ ID NO: 4, 6-7, 9, 11-12, 22, 24, 26, 28, 30, 32, 34, 36, 44, 46-48, 50, 52-53, 58, 60-62, 78, 80-81, 83, 85-87, 90, 92-94, 97, or 99-101, comprising:
(a) contacting a compound with the polypeptide of any of SEQ ID NO: 4, 6-7, 9, 11-12, 22, 24, 26, 28, 30, 32, 34, 36, 44, 46-48, 50, 52-53, 58, 60-62, 78, 80-81, 83, 85-87, 90, 92-94, 97, or 99-101 for a time sufficient to form a polynucleotide/compound complex; and
(b) detecting the complex, so that if a polypeptide/compound complex is detected, a compound that binds to any one of SEQ ID NO: 4, 6-7, 9, 11-12, 22, 24, 26, 28, 30, 32, 34, 36, 44, 46-48, 50, 52-53, 58, 60-62, 78, 80-81, 83, 85-87, 90, 92-94, 97, or 99-101 is identified.

18. A method for identifying a compound that binds to any one of the polypeptides of SEQ ID NO: 4, 6-7, 9, 11-12, 22, 24, 26, 28, 30, 32, 34, 36, 44, 46-48, 50, 52-53, 58, 60-62, 78, 80-81, 83, 85-87, 90, 92-94, 97, or 99-101, comprising:
(a) contacting a compound with the polypeptide of any one of SEQ ID NO: 4, 6-7, 9, 11-12, 22, 24, 26, 28, 30, 32, 34, 36, 44, 46-48, 50, 52-53, 58, 60-62, 78, 80-81, 83, 85-87, 90, 92-94, 97, or 99-101, in a cell, for a time sufficient to form a polypeptide/compound complex, wherein the complex drives the expression of a reporter gene sequence in the cell; and
(b) detecting the complex by detecting reporter gene sequence expression, so that if a polypeptide/compound complex is detected, a compound that binds to any one of the polypeptides of SEQ ID NO: 4, 6-7, 9, 11-12, 22, 24, 26, 28, 30, 32, 34, 36, 44, 46-48, 50, 52-53, 58, 60-62, 78, 80-81, 83, 85-87, 90, 92-94, 97, or 99-101 is identified.

19. A method of producing the polypeptides of claim 9 or 10, comprising:
(a) culturing the host cell of claim 7 or 8 for a period of time sufficient to express the polypeptide; and
(b) isolating the polypeptide from the cell or culture media in which the cell is grown.

20. A kit comprising any one of the polypeptides of claim 9 or 10.

21. A nucleic acid array comprising the polynucleotide of claim 1 attached to a surface.

22. The polypeptide of claim 9 or 10 wherein the polypeptide is provided on a polypeptide array.
